(19) United States
(12) Patent Application Publication
(10) Pub. No.: US 2024/0124887 A1
(43) Pub. Date: Apr. 18, 2024

(54) IMMUNE REGULATORS INVOLVED IN DEFENSE AGAINST PLANT DISEASES CAUSED BY LIBERIBACTER SPECIES

(71) Applicant: The Regents of the University of California, Oakland, CA (US)

(72) Inventors: Hailing Jin, Riverside, CA (US); Chien Yu Huang, Riverside, CA (US)

(73) Assignee: The Regents of the University of California, Oakland, CA (US)

(21) Appl. No.: 18/264,890

(22) PCT Filed: Feb. 9, 2022
(86) PCT No.: PCT/US2022/070589

§ 371 Date: Aug. 9, 2023

Related U.S. Application Data
(60) Provisional application No. 63/147,452, filed on Feb. 9, 2021.

(51) Int. Cl. C12N 15/82

(52) U.S. Cl. CPC C12N 15/8281

(57) ABSTRACT

The present disclosure provides methods and compositions for increasing resistance of plants to a disease caused by infection with bacteria of a Liberibacter species.

### CROSS-REFERENCE TO RELATED APPLICATIONS

This application claims priority benefit of U.S. Provisional Application No. 63/147,452, filed Feb. 9, 2021, which is incorporated by reference for all purposes.

### BACKGROUND

Citrus Greening Disease (Huanglongbing (HLB)), which is associated with the bacteria Candidatus Liberibacter asiaticus' (CLas) and is vectored by the Asian citrus psyllid (ACP), is the most devastating disease of citrus and has resulted in a significant reduction in citrus quality and quantity. HLB causes billions of dollars in losses of citrus products every year, and seriously impacts the viability of the citrus industry. Partial control is mainly achieved by removal of infected trees and chemical treatment against the insect vector. No efficient and sustainable disease control methods for HLB have been found. In Florida, more than 80% of the citrus groves have been infected by CLas since the first detection of HLB positive trees in 2005. Since then HLB has spread to Texas and California. Removing all of the infected trees is no longer a practical management strategy. Further, applying pesticides can only suppress the disease temporarily and is not an environmentally-friendly method as a long term solution.

Another important disease caused by a Liberibacter species is Potato Zebra Chip (ZC) disease (also called Potato Zebra complex disease). ZC disease is associated with Candidatus Liberibacter solanacearum (CLso), which is transmitted by potato psyllids (e.g., Bactericera cockerelli). ZC disease reached epidemic level in northern Texas in 2006 and has spread to Arizona, California, Colorado, Idaho, Oregon, Kansas, Nebraska, and New Mexico. ZC disease has caused millions of dollars loss to the potato industry in the southwestern United States, particularly Texas. In addition to potatoes, other solanaceous crops, including tomato, eggplant and pepper, can also be infected.

### BRIEF SUMMARY

Through comparative analysis of small RNA pools between HLB-resistant/tolerant variety US942 and and HLB-susceptible variety Cleopatra, we identified regulators that responded to HLB in US942 but not in Cleopatra. We predicted and annotated the possible immune negative and positive regulators, and repressed or evaluated the expression level in US942 and in another HLB-tolerant citrus relative, Sydney hybrid (Microcitrus virgata), which has a distinct genetic and geographic background compared to Cleopatra. Because the functional validation of candidate regulators in tree crops is always challenging and time-consuming, we developed a rapid functional screening method, using a similar parallel C. Liberibacter solanacearum (CLso)/potato psyllid/Nicotiana benthamiana interaction system to mimic the natural transmission and infection circuit of the HLB cost-effective screening method allows for rapid identification and functional characterization of regulators involved in plant immune responses against HLB. We performed functional testing in this pathosystem to identify positive defense regulators or negative immune suppressors. Accordingly, provided herein are methods and compositions for increasing the expression of positive defense regulators and/or inhibiting the expression of negative immune regulators to enhance resistance of a plant to Liberibacter infections, e.g., resistance to HLB, or to potato zebra chip disease.

In one aspect, provided herein is a method of enhancing resistance to HLB, the method comprising genetically modifying a plant, e.g., a plant of the citrus family or a solanaceous crop, to decrease expression of an endogenous gene encoding a negative regulator of immune response polypeptide, wherein the negative regulator of immune response polypeptide is a polypeptide listed in Table 1. In some embodiments, the negative regulator of immune response polypeptide is VAD1, PRT6, PUB26, PAO1, LIN2, CRWN, or GPX8. In some embodiments, decreasing expression of the negative regulator polypeptide comprises contacting the plant with siRNA that targets an endogenous nucleic acid encoding the negative regulator polypeptide. In some embodiments, decreasing expression of the negative regulator polypeptide comprises viral vector-mediated gene silencing. In some embodiments, decreasing expression of the negative regulator polypeptide comprises knocking out expression of the endogenous gene encoding the negative regulator. In some embodiments, the method comprises gene editing the endogenous gene to decrease or knockout expression, e.g., using CRISPR/CAS gene editing. In some embodiments, the negative regulator of immune response polypeptide comprises an amino acid sequence that is identical to, or is at least 70%, 75%, 80%, or 85% identical to, or at least 90% or at least 95% identical to, a polypeptide sequence listed in Table 3. In some embodiments, the negative regulator of immune response polypeptide comprises an amino acid sequence that is identical to, or is at least 70%, 75%, 80%, or 85% identical, or at least 90% or at least 95% identical, to a VAD1, PRT76 PUB26, PAO1, LIN2, CRWN, or GPX8 polypeptide sequence as set forth in Table 3, In some embodiments, the plant is a Citrus maxima, Citrus medica, Citrus micrantha, Citrus reticulata, Citrus aurantiifolia, Citrus aurantium, Citrus latifolia, Citrus limon, Citrus limonia, Citrus pardisi, Cirrus clementina, Citrus unshiu, Citrus sinensis, Citrus tangerina, Citrus ichangensis, Atalantia buxifolia, or Poncirus trifoliata plant. In some embodiments, the plant a variety of potato or tomato. In some embodiments, the plant is a pepper variety.

In a further aspect, provided herein is a method of enhancing resistance to HLB, the method comprising genetically modifying a plant e.g., a plant of the citrus family or a solanaceous crop, to overexpress a gene encoding a positive defense regulator polypeptide set forth in Table 2. In some embodiments, the positive defense regulator peptide is BRAP2, NDR1-like, or PSL4. In some embodiments, the method comprises genetically modifying a plant to overexpress a polypeptide comprising an amino acid sequence that is identical to, or has at least 70%, 75%, 80%, or 85% identity; or at least 90% or 95% identity, to a polypeptide set forth in Table 4. In some embodiments, the polypeptide is identical to, or has at least 70%, 75%, 80%, or 85% identity, or at least 90% identity, or at least 95% identity to a BRAP2, NDR1-like, or PSL4 polypeptide sequence set forth in Table 4. In some embodiments, the polypeptide is endogenous to the plant. Alternatively, the polypeptide can be heterologous to the plant. In some embodiments, the plant is a Citrus maxima, Citrus medica, Citrus micrantha, Citrus reticulata, Citrus aurantiifolia, Citrus aurantium, Citrus latifolia, Citrus limon, Citrus limonia, Citrus paradisi, Citrus clementina, Citrus unshiu, Citrus sinensis, Citrus tangerina, Citrus ichangensis. Atalantia buxifolia, or Poncirus trifoliata plant. In some embodiments, the plant a variety of potato or tomato. In some embodiments, the plant is a pepper variety.

In a further aspect, the disclosure provides a plant having enhanced resistance to HLB generated by a method targeting a gene as described herein, e.g., in the preceding two paragraphs.

### BRIEF DESCRIPTION OF THE FIGURES

FIG. 1a-c: Nb/pysliid/CLso pathosystem combined with viral-induced gene silencing (VIGS) showed that VAD is a negative regulator in response to CLso infection. a) Two-week-old AT plants were exposed to CLso positive potato psyllids for 5 days and VAD expression was knocked down by VIGS. Silencing RB gene (iRB control) was used as a control in non-silenced plants. b) Details of leaves from panel a. c) CLso bacteria titer measured by probe-based qPCR in 50 ng host genomic DNA. The significant difference is analyzed by student's t-test (*P<0.01).

FIG. 2a-d: VAD knock-down Carrizo plants showed higher expression of defense marker genes including pathogenesis-related PR-2 and Chitinase (CHI). a. One cutting plant from VAD knock-down Carrizo plant. The VAD is knock down by RNA silencing. The Carrizo plant was introduced VAD harpin RNA expression vector pHellsgate8. b. The expression level of VAD in VAD silencing Carrizo plant was analyzed by qRT-PCR and normalized to Ubiqutin gene (CsUbi). The significant difference is analyzed by T test (*P<0.01). c and d. The expression level of defense marker genes, PR2 (c) and CHI (d) in VAD silencing Carrizo plant was analyzed by qRT-PCR and normalized to Ubiqutin gene (CsUbi). The significant difference is analyzed by T test (*P<0.01).

FIG. 3a-c: Nb/pysllid/CLso pathosystem combined with VIGS showed that PAO1 is a negative regulator in response to CLso infection. a) Two-week-old Nb plants were exposed to CLso positive potato psyllids for 5 days and PAO1 expression was knocked down by VIGS. Silencing RB gene (iRB control) was used as a control in non-silenced plants, b) Details of leaves from panel a. c) CLso bacteria titer measured by probe-based qPCR in 50 ng host genomic DNA. The significant difference is analyzed by student's t-test(*P<0.05).

FIG. 4a-c: Nb/pysllid/CLso pathosystem combined with VIGS showed that CRWN is a negative regulator in response to CLso infection. a) Two-week-old Ab plants were exposed to CLso positive potato psyllids for 5 days and CRWN expression was knocked down by VIGS. Silencing RB gene (iRB control) was used as a control in non-silenced plants. b) Details of leaves from panel a. c) CLso bacteria titer measured by probe-based qPCR in 50 ng host genomic DNA. The significant difference is analyzed by student's t-test(*P<0.05).

FIG. 5a-c: Nb/pysllid/CLso pathosystem combined with VIGS showed that GPX8 is a negative regulator in response to CLso infection. a) Two-week-old Ab plants were exposed to CLso positive potato psyllids for 5 days and GPX8 expression was knocked down by VIGS. Silencing RB gene (iRB control) was used as a control in non-silenced plants. b) Details of leaves from panel a. c) CLso bacteria titer measured by probe-based qPCR in 50 ng host genomic DNA. The sinniticant difference is analyzed by student's t-test(*P<0.05).

FIG. 6a-b: Nb/pysllid/CLso pathosystem combined with VIGS showed that PRT6 is a negative regulator in response to CLso infection. a) Two-week-old Nb plants were exposed to CLso positive potato psyllids for 5 days and PRT6 expression was knocked down by VIGS. Silencing RB gene (iRB control) was used as a control in non-silenced plants. b) CLso bacteria titer measured by probe-based qPCR in 50 ng host genomic DNA.

FIG. 7a-c: Nb/pysllid/CLso pathosystem combined with VIGS showed that PUB26 is a negative regulator in response to CLso infection. a) Two-week-old Nb plants were exposed to CLso positive potato psyllids for 5 days and PUB26 expression was knocked down by VIGS. Silencing RB gene (iRB control) was used as a control in non-silenced plants. b) Details of leaves from panel a. c) CLso bacteria titer measured by probe-based qPCR in 50 ng host genomic DNA.

FIG. 8a-c: Nb/pysllid/CLso pathosystem combined with VIGS showed that LIN2 is a negative regulator in response to CLso infection. a) Two-week-old Nb plants were exposed to CLso positive potato psyllids for 5 days and LIN2 expression was knocked down by VIGS. Silencing RB gene (iRB control) was used as a control in non-silenced plants. b) Details of leaves from panel a. c) CLso bacteria titer measured by probe-based qPCR in 50 ng host genomic DNA. The significant difference is analyzed by student's t-test(*P<0.05).

FIG. 9a-c: Nb/pysllid/CLso pathosystem combined with VIGS showed that BRAP is a positive regulator in response to CLso infection. a) Two-week-old Nb plants were exposed to CLso positive potato psyllids for 5 days and BRAP expression was knocked down by VIGS. Silencing RB gene (iRB control) was used as a control in non-silenced plants. b) Details of leaves from panel a. c) CLso bacteria titer measured by probe-based qPCR in 50 ng host genomic DNA. The significant difference is analyzed by student's t-test(*P<0.05).

FIG. 10a-b: Nb/pysllid/CLso pathosystem combined with VIGS showed that PSL4 is a positive regulator in response to CLso infection. a) Two-week-old Nb plants were exposed to CLso positive potato psyllids for 5 days and PSL4 expression was knocked down by VIGS. Silencing RB gene (iRB control) was used as a control in non-silenced plants. b) CLso bacteria titer measured by probe-based qPCR in 50 ng host genomic DNA.

FIG. 11a-b: Nb/pysllid/CLso pathosystem combined with VIGS showed that NDR1-like is a positive regulator in response to CLso infection. a) Two-week-old Nb plants were exposed to CLso positive potato psyllids for 5 days and PSL4 expression was knocked down by VIGS. Silencing RB gene (iRB control) was used as a control in non-silenced plants. b) CLso bacteria titer measured by probe-based qPCR in 50 ng host genomic DNA. The significant difference is analyzed by student's t-test(*P<0.05).

### DETAILED DESCRIPTION

The present disclosure provides targets for modulating the immune response pathways to enhance resistance to HLB.

The invention employs various routine recombinant nucleic acid techniques. Generally, the nomenclature and the laboratory procedures in recombinant DNA technology described below are commonly employed in the art. Many manuals that provide direction for performing recombinant DNA manipulations are available. e.g., Sambrook & Russell, Molecular Cloning, A Laboratory Manual (3rd Ed, 2001); and Current Protocols in Molecular Biology (Ausubel, et al., John Wiley and Sons, New York, 2009-2014).

As used herein, the terms “citrus greening disease” and “Huanglongbing (HLB)” refer to a bacterial infection of plants (e.g., citrus plants) caused by bacteria in the genus Candidatus Liberibacter (CandidatusLiberibacter asiaticus, Candidatus Liberibacter africanus, and Candidatus Liberibacter americanus). The infection is vectored and transmitted by the Asian citrus psyllid, Diaphorina citri, and the African citrus psyllid, Trioza erytreae. Three different types of HLB are currently known: the heat-tolerant Asian form, and the heat-sensitive African and American forms.

The term “HLB-resistant/tolerant” or “HLB resistance/tolerance” refers to an increase in the ability of a citrus plant comprising one or more genetic modifications described herein to prevent or resist HLB infection or HLB-induced symptoms of infection in response to a corresponding control citrus plant that does not comprise the genetic modification(s). An “HLB-resistant” plant thus can have increased tolerance to HLB compared to the control citrus plant. Accordingly, unless otherwise specified, the term “HLB-resistant” includes plants that are tolerant to HLB, e.g., can the citrus plant can grow and produce fruit despite being infected with HLB. The term “HLB-resistant/tolerant” and “HLB-resistant” are thus used interchangeably herein to refer to a plant that has an increase in the ability to prevent HLB infection or has a reduction in one or more HLB-induced symptoms of infection.

The term “negative immune suppressor” or “negative immune response regulator” or “negative regulator of immune response” refers to a gene, or a polypeptide encoded by the gene, that decreases host defense responses, i.e., reduces one or more aspect of a plant immune response to CLas infection, such that the plant has increased susceptibility to HLB. A listing of negative immune suppressors is provided in Table 1. Illustrative polypeptide sequences are provided in Table 3.

The term “positive defense regulator” refers to a gene, or a polypeptide encoded by the gene, that enhances host defense responses, i.e., enhances one or more aspect of a plant immune response to CLas infection, such that the plant has increased resistance/tolerance to HLB. A listing of positive defense regulators is in Table 2. Illustrative polypeptide sequences are provided in Table 4.

The term “nucleic acid” or “polynucleotide” refers to a single or double-stranded polymer of deoxyribonucleotide or ribonucleotide bases read from the 5′ to the 3′ end. Nucleic acids may also include modified nucleotides that permit correct read through by a polymerase and do not significantly alter expression of a polypeptide encoded by that nucleic acid.

The phrase “nucleic acid encoding” or “polynucleotide encoding” refers to a nucleic acid which directs the expression of a specific protein or peptide. The nucleic acid sequences include both the DNA strand sequence that is transcribed into RNA and the RNA sequence that is translated into protein. The nucleic acid sequences include both the full length nucleic acid sequences as well as non-full length sequences derived from the full length sequences. It should be further understood that the sequence includes the degenerate codons of the native sequence or sequences which may be introduced to provide codon preference in a specific host cell.

Two nucleic acid sequences or polypeptides are said to be “identical” if the sequence of nucleotides or amino acid residues, respectively, in the two sequences is the same when aligned for maximum correspondence as described below. “Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. When percentage of sequence identity is used in reference to proteins or peptides, it is recognized that residue positions that are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill ii the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservauve substitutions is calculated according to, e.g., the algorithm of Meyers & Miller, Computer Applic. Biol Sci. 4:11-17 (1988) e.g., as implemented in the program PC/GENE (Intelligenetics, Mountain View, California, USA).

The term “substantial identity” or “substantially identical,” as used in the context of polynucleotide or polypeptide sequences, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 90%, 91%, 92%93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math 2:482 (1981), by the homology alignment algorithm of Needleman and Wunsch J. Mol. Biol. 48:443 (1970), by the search for similarity method of Pearson and Lipman Proc. Natl. Acad. Sci. (U. S. A.) 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, WI), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the sane length in a database sequence. T is referred to as the neighborhood word score threshold (Altschul et al, supra). These initial neighborhood word hits acts as seeds for initiating searches to find longer HSPs containing then. The word hits are then extended in both directions along each sequence for as far as the cumulative alignment score can be increased. Cumulative scores are calculated using, for nucleotide sequences, the parameters M (reward score for a pair of matching; residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)). For purposes of this application, amino acid sequence identity is determined using BLASTP with default parameters.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

The term “complementary to” is used herein to mean that a polynucleotide sequence is complementary to all or a portion of a reference polynucleotide sequence. In some embodiments, a polynucleotide sequence is complementary to at least 15, at least 20, at least 25, at least 30, at least 40, at least 50, at least 75, at least 100, at least 125, at least 150, at least 175, at least 200, or more contiguous nucleotides of a reference polynucleotide sequence. In some embodiments, a polynucleotide sequence is “substantially complementary” to a reference polynucleotide sequence if at least 70%, at least 75%, at least 80%, at least 85%, at least 90%, or at least 95% of the polynucleotide sequence is complementary to the reference polynucleotide sequence.

A polynucleotide sequence is “heterologous” to an organism or a second polynucleotide sequence if it originates from a foreign species, or, if from the same species, is modified from its original form. For example, when a promoter is said to be operably linked to a heterologous coding sequence, it means that the coding sequence is derived from one species whereas the promoter sequence is derived another, different species; or, if both are derived from the same species, the coding sequence is not naturally associated with the promoter (e.g., is a genetically engineered coding sequence, e.g., from a different gene in the same species, or an allele from a different ecotype or variety).

An “expression cassette” refers to a nucleic acid construct that, when introduced into a host cell, results in transcription and/or translation of an RNA or polypeptide, respectively. Antisense or sense constructs that are not or cannot be translated are expressly included by this definition. In the case of both expression of transgenes and suppression of endogenous genes (e.g., by antisense, or sense suppression) one of skill will recognize that the inserted polynucleotide sequence need not be identical, but may be only substantially identical to a sequence of the gene from which it was derived.

The term “promoter,” as used herein, refers to a polynucleotide sequence capable of driving transcription of a coding sequence in a cell. Thus, promoters used in the polynucleotide constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. A “plant promoter” is a promoter capable of initiating transcription in plant cells. A “constitutive promoter” is one that is capable of initiating transcription in nearly all tissue types, whereas a “tissue-specific promoter” initiates transcription only in one or a few particular tissue types. An “inducible promoter” is one that initiates transcription only under particular environmental conditions or developmental conditions.

The term “plant” includes whole plants, shoot vegetative organs and/or structures (e.g., leaves, stems and tubers), roots, flowers and floral organs (e.g., bracts, sepals, petals, stamens, carpels, anthers), ovules (including egg and central cells), seed (including zygote, embryo, endosperm and seed coat), fruit (e.g., the mature ovary), seedlings, plant tissue (e.g., vascular tissue, ground tissue, and the like), cells (e.g., guard cells, egg cells, trichomes and the like), and progeny of same.

### DETAILED DESCRIPTION OF THE INVENTION

### Introduction

As described in the Examples section below, negative regulators of the immune response to Liberibacter infection, e.g., HLB or potato zebra chip disease, and positive defense regulators of the immune response against Liberibacter infection were identified using a screening technique, Described herein are methods and compositions for enhancing citrus plant resistance/tolerance to HLB by genetically modifying the citrus plant to silence, inhibit, or decrease expression or activity of a negative regulator of the immune response; and/or genetically modifying the citrus plant to increase expression or activity of a positive defense regulator. Similarly, a solanaceous crop plant, such as potato or tomato, can be modified to decrease and/or increase expression of an immune regulator polypeptide described herein.

In any of the compositions or methods described in the present disclosure, any plant species can be used, but in preferred embodiments, the plant is a member of the citrus family, e.g., a Citrus maxima, Citrus medica, Citrus micrantha, Citrus retliculata, Citrus aurantifolia, Citrus aurantium, Citrus latifolia, Citrus limon, Citrus limonia, Citrus paradisi, Citrus clementina, Citrus unshiu, Citrus sinensis, Citrus tangerina, Citrus ichangensis,

Atalantia buxifolia, or Poncirus trifoliata plant. In some embodiments, the plant a variety of potato or tomato. In some embodiments, the plant is a pepper variety.

### Negative Regulators of the Immune Response

In some embodiments, provided herein are methods and compositions to inhibit expression of one or more negative regulators of plant immunity genes as set forth in Table 1. Illustrative polypeptide sequences for various citrus species are provided in Table 3.

TABLE 1
Negative regulators of plant immune responses against
HLB. These genes were targeted by small RNAs induced
by CLas infection in US942 but not in Cleopatra

| Gene | Annotation | Target by sRNAs |
|---|---|---|
| Ciclev10019258m | VAD1 | 942si2047 |
| Ciclev10013610m | Proteolysis 6, PRT6 | 942si2003 and 942si2020 |
| Ciclev10010604m | OLIGOPEPTIDE | 942si2003 and 942si2005 |
| Ciclev10027961m; | TRANSPORTER | |
| Ciclev10014497m | 1; OPT1; YSL6 | |
| Ciclev10028522m | PUB26 | 942si2009 and 942si2049 |
| Ciclev10027096m | DMR6 | 942si2012 |
| Ciclev10031331m | PAO1 | 942si2013 and 942si2025 |
| Ciclev10000377m; | TPS5 | 942si2008 and 942si2032 |
| Ciclev10000246m; | | |
| Ciclev10000247m; | | |
| Ciclev10000248m | | |
| Ciclev10030586m | ACA11 | 942si2009 |
| Ciclev10030706m | MKP1 | 942si2014 |
| Ciclev10001632m; | CRT1, CRT1a | 942si2016 |
| Ciclev10001298m | | |
| Ciclev10011903m | CPO-I, | 942si2019 |
| | HEMF1, LIN2 | |
| Ciclev10024751m; | LINC4, CRWN | 942si2020 |
| Ciclev10024754m; | | |
| Ciclev10024753m | | |
| Ciclev10022871m; | GPX8 | 942si2023 |
| Ciclev10022795m | | |
| Ciclev10014207m; | LOX2 | 942si2024 |
| Ciclev10014574m | | |
| Ciclev10027664m | PI4K ALPHA | 942si2035 |

Expression or activity of the negative regulator of immune response proteins described herein can be inhibited or knocked out using known methods Thus, one, or more than one, of the genes provided in Table 1 can be knocked out or mutated to enhance HLB resistance. For example, in some embodiments, the native gene that encodes a polypeptide identical to or substantially identical (e.g., at least 70, 75, 80, 85, 90% identical, or at least 95% identical) to a VAD1, PRT6, OPT1, YSL6, PUB26, DMR6, PAO1, TPS5, ACA11, MPK1, CRT1, LTN2, CRWN (LINC4), GPX8, LOX2, or PI4K polypeptide sequence as set forth in Table 3 is mutated or knocked out in a citurs family plant. In some embodiments, the native gene that encodes a polypeptide identical or substantially identical (e.g., at least 70, 75, 80, 85, 90% identical, or at least 95% identical) to a VAD1, PRT6, PUB26, PAO1, LIN2, CRWN (LINC4), or GPX8 polypeptide sequence as set forth in Table 3 is mutated or knocked out in a citrus family plant. Gene sequences can be readily identified in other citrus species in view of known genome sequences and the conserved nature of the proteins.

In some embodiments, the gene sequence is knocked out in the plant. “Knocked out” in the context of this application means that the plant does not make the particular protein encoded by the gene. “Knocked down” means that the level of expression or the level of the protein or activity of the protein is reduced in a plant relative to a corresponding control wildtype plant. Knock outs and knock downs can be generated in a variety of ways. For example, a knock out plant can be generated by a deletion of all or a substantial part (e.g., majority) or the coding sequence for a polypeptide identical or substantially identical to a protein encoded by a gene set forth in Table 1, or to any one of the VAD1, PRT6, OPT1, YSL6, PUB26, DMR6, PAO1, TPS5, ACA11, MPK1, CRT1, LIN2, CRWN (LINC4), GPX8, LOX2, or PI4K polypeptide sequences set forth in Table 3. In some embodiments, a promoter sequence may be modified or deleted such that expression is eliminated or reduced. In some embodiments, knock out or knock down of the target is achieved by introduction of a mutation that prevents translation or transcription (e.g., a mutation that introduces a stop codon early in the coding sequence or that disrupts transcription). A knock out or knock down can also be achieved by silencing or other suppression methods, e.g., such that the plant expresses substantially less of the native protein (e.g., less than 50, 25, 10, 5, or 1% of native expression). A knockout or knockdown can also be achieved by CRISPR-CAS-mediated mutations and deletion, or by the use of alternative gene editing techniques further described below.

In some embodiments, a mutation introduced into the protein is a single amino acid change that reduces or eliminates the protein's activity. Alternatively, the mutation can include any number (e.g., 1, 2, 3, 4, 5, 6, 7, 8, 9, or 10 or more) of amino acid changes, deletions or insertions that reduce or eliminate the protein activity.

Methods for introducing genetic mutations into plant genes and selecting plants with desired traits are well known and can be used to introduce mutations or to knock out or knock down expression or activity of a protein. For instance, seeds or other plant material can be treated with a mutagenic insertional polynucleotide (e.g., transposon, T-DNA, etc.) or chemical substance, according to standard techniques, Such chemical substances include, but are not limited to, the following: diethyl sulfate, ethylene imine, ethyl methanesulfonate and N-nitroso-N-ethylurea. Alternatively, ionizing radiation from sources such as, X-rays or gamma rays can be used. Plants having mutated protein can then be identified, for example, by phenotype or by molecular techniques.

Modified protein chains can also be readily designed utilizing various recombinant DNA techniques well known to those skilled in the art and described for instance, in Sambrook et al, supra. Hydroxylamine can also be used to introduce single base mutations into the coding region of the gene (Sikorski et al, Meth. Enzymol., 194:302-318 (1991)). For example, the chains can vary from the naturally occurring sequence at the primary structure level by amino acid substitutions, additions, deletions, and the like. These modifications can be used in a number of combinations to produce the final modified protein chain.

Alternatively, homologous recombination can be used to induce targeted gene modifications or knockouts by specifically targeting the gene in vivo (see, generally, Grewal and Klar, Genetics, 146:1221-1238 (1997) and Xu et al., Genes Dev., 10:2411-2422 (1996)). Homologous recombination has been demonstrated in plants (Puchta et al., Experientia, 50:277-284 (1994); Swoboda et al., EMBO J., 13:484-489 (1994); Offringa et al., Proc. Natl. Acad. Sci. USA, 90:7346-7350 (1993); and Kempin et al., Nature, 389:802-803 (1997)).

In applying homologous recombination technology to a gene, mutations in selected portions of gene sequences (including; 5′ upstream, 3′ downstream, and intragenic regions) can be made in vitro and then introduced into the desired plant using standard techniques. Since the efficiency of homologous recombination is known to be dependent on the vectors used, use of dicistronic gene targeting vectors as described by Mountford et al., Proc. Natl. Acad. Sci. USA, 91:4303-4307 (1994); and Vaulont et al., Transgenic Res., 4:247-255 (1995) are conveniently used to increase the efficiency of selecting for altered PP2A subunit A protein gene expression in transgenic plants. The mutated gene will interact with the target wild-type gene in such a way that homologous recombination and targeted replacement of the wild-type gene will occur in transgenic plant cells, resulting in suppression of target protein activity.

Any of a number of genome-editing techniques known to those of skill in the art can be used to mutate or knock out the target protein. The particular genome-editing technique used is not critical, so long as it provides site-specific mutation of a desired nucleic acid sequence. Exemplary genome-editing proteins include targeted nucleases such as engineered zinc finger nucleases (ZFNs), transcription-activator-like effector nucleases (TALENs), and engineered meganucleases. In addition, systems which rely on an engineered guide RNA (a gRNA) to guide an endonuclease to a target cleavage site can be used. The most commonly used of these systems is the CRISPR/Cas system with an engineered guide RNA to guide the Cas-9 or Cas12 endonuclease to the target cleavage site.

CRISPR (Clustered Regularly Interspaced Short Palindromic Repeats)/Cas (CRISPR-associated) system, are adaptive defense systems in prokaryotic organisms that cleave foreign DNA. CRISPR loci in microbial hosts contain a combination of CRISPR-associated (Cas) genes as well as non-coding RNA elements which determine the specificity of the CRISPR-mediated nucleic acid cleavage. Three types (I-III) of CRISPR systems have been identified across a wide range of bacterial hosts. In a typical system, a Cas endonuclease (e.g., Cas9) is guided to a desired site in the genome using small guide RNAs that target sequence-specific single- or double-stranded DNA sequences. The CRISPR/Cas system has been used to induce site-specific mutations, including deletions, in plants (see Miao et al. 2013 Cell Research 23:1233-1236).

The basic CRISPR system uses two non-coding guide RNAs (crRNA and tracrRNA) which form a crRNA:tracrRNA complex that directs the nuclease to the target DNA via Wastson-Crick base-pairing between the crRNA and the target DNA, Thus, the guide RNAs can be modified to recognize any desired target DNA sequence. More recently, it has been shown that a Cas nuclease can be targeted to the target gene location with a chimeric single-guide RNA (sgRNA) that contains both the crRNA and tracRNA elements. It has been shown that Cas9 or Cas12 and the like, can be targeted to desired gene locations in a variety of organisms with a chimeric sgRNA (Cong et al. 2013 Science 339:819-23).

Zinc finger nucleases (ZFNs) are engineered proteins comprising a zinc finger DNA-binding domain fused to a nucleic acid cleavage domain, e.g., a nuclease. The zinc finger binding domains provide specificity and can be engineered to specifically recognize any desired target DNA sequence. For a review of the construction and use of ZFNs in plants and other organisms, see Urnov et al. 2010 Nat Rev Genet. 11(9):636-46.

Transcription activator like effectors (TALEs) are proteins secreted by certain species of Xanthomonas to modulate gene expression in host plants and to facilitate bacterial colonization and survival. TALEs act as transcription factors and modulate expression of resistance genes in the plants. Recent studies of TALEs have revealed the code linking the repetitive region of TALEs with their target DNA-binding sites. TALEs comprise a highly conserved and repetitive region consisting of tandem repeats of mostly 33 or 34 amino acid segments. The repeat monomers differ from each other mainly at amino acid positions 12 and 13. A strong correlation between unique pairs of amino acids at positions 12 and 13 and the corresponding nucleotide in the TALE-binding site have been found. The simple relationship between amino acid sequence and DNA recognition of the TALE binding domain allows for the design DNA binding domains of any desired specificity.

TALEs can be linked to a non-specific DNA cleavage domain to prepare genome-editing proteins, referred to as TALENs. As in the case of ZFNs, a restriction endonuclease, such as FokI, can be conveniently used. For a description of the use of TALENs in plants, see Mahfouz et al. 2011 Proc Natl Acad Sci USA. 108:2623-8 and Mahfouz 2011 GM Crops. 2:99-103.

Meganucleases are endonucleases that have a recognition site of 12 to 40 base pairs. As a result, the recognition site occurs rarely in any given genome. By modifying the recognition sequence through protein engineering, the targeted sequence can be changed and the nuclease can be used to cleave a desired target sequence. (See Seligman, Et al. 2002 Nucleic Acids Research 30: 3870-9 WO06097853, WO6097784, WO04067736, or US20070117128).

In addition to the methods described above, other methods for introducing genetic mutations into plant genes and selecting plants with desired traits are known. For instance, seeds or other plant material can be treated with a mutagenic chemical substance, according to standard techniques. Such chemical substances include, diethyl sulfate, ethylene imine, ethyl methanesulfonate (EMS) and N-nitroso-N-ethylurea. Alternatively, ionizing radiation from sources such as, X-rays or gamma rays can be used.

Also provided are methods of suppressing expression or activity of a polypeptide identical to, or substantially identical, e.g., at least 70, 75, 80, 85, or 90% identical; or at least to 95% identical, to a protein encoded by a gene set forth in Table 1 or a to a VAD1, PRT6, OPT1, YSL6, PUB26, DMR6, PAO1, TPS5, ACA11, MPK1, CRT1, LIN2, CRWN (LINC4), GPX8, LOX2, or PI4K polypeptide sequence as set forth in Table 3, in a citrus plant using expression cassettes that express RNA molecules (or fragments thereof) that inhibit endogenous target gene expression or activity in a plant cell Suppressing or silencing gene function refers generally to the suppression of levels of mRNA or protein expressed by the endogenous gene and/or the level of the protein functionality in a cell. The terms do not require specific mechanism and could include RNAi (e.g., short interfering RNA (siRNA) and microRNA (miRNA)), anti-sense, cosuppression, viral-suppression, hairpin suppression, stem-loop suppression, and the like.

A number of methods can be used to suppress or silence gene expression in a plant. The ability to suppress gene function in a variety of organisms, including plants, using double stranded RNA is well known. Expression cassettes encoding RNAI typically comprise a polynucleotide sequence at least substantially identical to the target gene linked to a complementary polynucleotide sequence. The sequence and its complement are often connected through a linker sequence that allows the transcribed RNA molecule to fold over such that the two sequences hybridize to each other.

RNAi (e.g., siRNA, miRNA) appears to function by base-pairing to complementary RNA or DNA target sequences. When bound to RNA, the inhibitory RNA molecules trigger either RNA cleavage or translational inhibition of the target sequence. When bound to DNA target sequences, it is thought that inhibitory RNAs can mediate DNA methylation of the target sequence. The consequence of these events, regardless of the specific mechanism, is that gene expression is inhibited. RNA silencing can also be achieved by expressing the target gene or part of the target gene in a virus vector, such as tobacco rattle virus (TRV), Potato virus X (PVX), or Citrus Tristeza Virus (CTV), which can trigger virus-induced gene silencing (VIGS) of the target gene.

MicroRNAs (miRNAs) are non-coding RNAs of about 19 to about 24 nucleotides in length that are processed from longer precursor transcripts that form stable hairpin structures.

In addition, antisense technology can be employed. To accomplish this, a nucleic acid segment at least substantially identical to the desired gene is cloned and operably linked to a promoter such that the antisense strand of RNA will be transcribed. The expression cassette is then transformed into a plant and the antisense strand of RNA is produced. In plant cells, it has been suggested that antisense RNA inhibits gene expression by preventing the accumulation of mRNA which encodes the protein of interest.

Another method of suppression is sense suppression. Introduction of expression cassettes in which a nucleic acid is configured in the sense orientation with respect to the promoter has been shown to be an effective means by which to block the transcription of target genes.

For these techniques, the introduced sequence in the expression cassette need not have absolute identity to the target gene. In addition, the sequence need not be full length, relative to either the primary transcription product or fully processed mRNA. One of skill in the art will also recognize that using these technologies families of genes can be suppressed with a transcript. For instance, if a transcript is designed to have a sequence that is conserved among a family of genes, then multiple members of a gene family can be suppressed. Conversely, if the goal is to only suppress one member of a homologous gene family, then the transcript should be targeted to sequences with the most variance between family members.

Gene expression can also be inactivated using recombinant DNA techniques by transforming plant cells with constructs comprising transposons or T-DNA sequences. Mutants prepared by these methods are identified according to standard techniques. For instance, mutants can be detected by PCR or by detecting the presence or absence of PP2A subunit A mRNA, e.g., by northern blots or reverse transcription PCR (RT-PCR).

Catalytic RNA molecules or ribozymes can also be used to inhibit expression of embryo-specific genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA cleaving activity upon them, thereby increasing the activity of the constructs. The design and use of target RNA-specific ribozymes is well known.

The recombinant construct encoding a genome-editing protein or a nucleic acid that suppresses expression may be introduced into the plant cell using standard genetic engineering techniques, well known to those of skill in the art. In the typical embodiment, recombinant expression cassettes can be prepared according to well-known techniques. In the case of CRISPR/Cas nuclease, the expression cassette may transcribe the guide RNA, as well.

In some embodiments, the genome-editing protein itself, is introduced into the plant cell. In these embodiments, the introduced genome-editing protein is provided in sufficient quantity to modify the cell but does not persist after a contemplated period of time has passed or after one or more cell divisions. In such embodiments, no further steps are needed to remove or segregate away the genome editing protein and the modified cell.

In these embodiments, the genome editing protein is prepared in vitro prior to introduction to a plant cell using well known recombinant expression systems (bacterial expression, in vitro translation, yeast cells, insect cells and the like). After expression, the protein is isolated, refolded if needed, purified and optionally treated to remove any purification tags, such as a His-tag. Once crude, partially purified, or more completely purified genome editing proteins are obtained, they may be introduced to a plant cell via electroporation, by bombardment with protein coated particles, by chemical transfection or by some other means of transport across a cell membrane.

### Positive Regulators of the Immune Response

In some embodiments, provided herein are methods and compositions to enhance expression of one or more positive defense plant genes as set forth in Table 2. Illustrative polypeptide sequences various citrus species are provided in Table 4.

TABLE 2
Positive regulators of plant immune responses against
HLB. These genes were targeted by small RNAs down-regulated
by CLas infection in US942 but not in Cleopatra

| Gene | Annotation | Target by sRNAs |
|---|---|---|
| Ciclev10008403m | BRAP2 | 942si1001 |
| Ciclev10019811m | CYP450, CYP93 | 942si1020 |
| Ciclev10012768m | NDR1-like; NHL1 | 942si1026 |
| Ciclev10014526m | PSL4 | 942si1003 |
| Ciclev10028533m | LYM2 | 942si1003 |
| Ciclev10017680m | SOT12 | 942si1005 |
| Ciclev10002823m; | AHUS5, EMB1637, | 942si1006 |
| Ciclev10002741m; | SCE1, SCE1A | |
| Ciclev10002866m | | |
| Ciclev10031485m | GLY1, SFD1 | 942si1009 |
| Ciclev10011175m | PAL1 | 942si1009 |
| Ciclev10012055m | WRKY70 | 942si1017 |
| Ciclev10033608m | EFR-like | 942si1002 |

Expression of the proteins described herein can be increased using known techniques. Any one, or more than one, of the genes provided in Table 4 can be overexpressed in a plant to enhance HLB resistance. Thus, in some embodiments, a plant can be genetically modified to overexpress the gene native to the plant or to express a corresponding heterologous gene from another species. In some embodiments, a citrus plant is engineered to overexpress a polypeptide identical to or substantially identical (e.g., at least 70, 75, 80, 85, 90% identical, or at least 95% identical) to a BRAP2, CYP93, NDR1-like, PSL4, LYM2, SOTI2, SCE1, GLY1, PAL1, WRKY70, or EFR-like polypeptide sequence as set forth in Table 4. In some embodiments, a citrus plant is engineered to overexpress a polypeptide identical to or substantially identical (e.g., at least 70, 75, 80, 85, 90% identical, or at least 95% identical) to a BRAP2, NDR1-like, or PSL4 polypeptide sequence as set forth in Table 4. Gene sequences can be readily identified in other citrus species in view of known genome sequences and the conserved nature of the proteins.

In some embodiments, a citrus plant is genetically modified to introduce a recombinant expression cassette for expressing a native or heterologous BRAP2, CYP93, NDR1-like, PSL4, LYM2, SOT12, SCE1, GLY1, PAL1, WRKY70 or EFR-like polypeptide. It should be recognized that transgenic plants encompass the plant or plant cell in which the expression cassette is introduced as well as progeny of such plants or plant cells that contain the expression cassette, including the progeny that have the expression cassette stably integrated in a chromosome.

In some embodiments, the transgenic plant can have increased expression (e.g., at least 5%, 10%, 50% or more) of the BRAP2, CYP93, NDR1-like, PSL4, LYM2, SOT12, SCE1, GLY1, PAL1, WRKY70, or EFR-like polypeptide compared to a corresponding control plant that has not been genetically modified to over express the protein.

In some embodiments, a gene editing technique, such as CRISPR/Cas, can be employed to increase epression of the BRAP2, CYP93, NDR1-like, PSL4, LYM2, SOT12, SCE1, GLY1, PAL1, WRKY70, or EFR-like polypeptide, e.g., by introducing additional copies of the protein-coding sequence into the plant genome.

In some embodiments, a recombinant expression vector comprising the protein-coding sequence driven by a promoter may be introduced into the genome of the desired plant; or be introduced by CRISPR-CAS knock-in, as noted above; or be expressed by a viral vector, such as a CTV viral vector. In some embodiments, a polynucleotide encoding the polypeptide may be introduced into the plant, e.g., by recombination, such that expression is controlled by a promoter endogenous to the plant. Thus, for example, in some embodiments, the DNA construct may be introduced directly into the genomic DNA of the plant cell using techniques such as electroporation and microinjection of plant cell protoplasts, or the DNA construct can be introduced directly to plant tissue using ballistic methods, such as DNA particle bombardment. Alternatively, the DNA construct may be combined with suitable T-DNA flanking regions and introduced into a conventional Agrobacterium tumefaciens host vector. While transient expression of the polypeptide is encompassed by the invention, generally expression will be from insertion of expression cassettes into the plant genome, e.g., such that at least some plant offspring also contain the integrated expression cassette.

### Expression Cassettes

Plant expression cassettes (e.g., for expression of a positive defense protein as described herein, or alternatively, for expression of inhibitory nucleic acids or gene editing proteins to inhibit or ablate expression of a negative immune response regulator as described herein) can contain the polynucleotide operably linked to a promoter (e.g., one conferring inducible or constitutive, environmentally- or developmentally-regulated, or cell- or tissue-specific/selective expression), a transcription initiation start site, a ribosome binding site, an PNA processing signal, a transcription termination site, and/or a polyadenylation signal.

A number of promoters can be used. A plant promoter fragment can be employed which will direct expression of the desired polynucleotide in all tissues of a plant. In some embodiments, promoters described herein comprise from 500 to 2 kb, or from 500 to 1 kb, or 500 to 2.5 kb, upstream (5′) from where gene transcription is initiated. Such promoters are referred to herein as “constitutive” promoters and are active under most environmental conditions and state of development or cell differentiation. Examples of constitutive promoters include the cauliflower mosaic virus (CaMV) 35S transcription initiation region.

Alternatively, the plant promoter can direct expression of the polynucleotide under environmental control. Such promoters are referred to here as “inducible” promoters, Environmental conditions that may affect transcription by inducible promoters include biotic stress, abiotic stress, saline stress, drought stress, pathogen attack, anaerobic conditions, cold stress, heat stress, hypoxia stress, or the presence of light.

In addition, chemically inducible promoters can be used. Examples include those that are induced by benzyl sulfonamide, tetracycline, abscisic acid, dexamethasone, ethanol or cyclohexenol.

Examples of promoters under developmental control include promoters that initiate transcription only, or preferentially, in certain tissues such as leaves, roots, fruit, seeds, or flowers. These promoters are sometimes called tissue-preferred promoters. The operation of a promoter may also vary depending on its location in the genome. Thus, a developmentally regulated promoter may become fully or partially constitutive in certain locations. A developmentally regulated promoter can also be modified, if necessary, for weak expression.

Selecting for Plants with Enhanced HLB Resistance/Tolerance

Plants with enhanced HLB resistance/tolerance can be selected in many ways. One of ordinary skill in the art will recognize that the following methods are but a few of the possibilities. One of skill in the art will recognize that resistance responses of plants vary depending on many factors, including the plant. Generally, enhanced resistance is measured by the reduction or elimination of disease symptoms (e.g., reduction in the number or size of lesions or reduction in the amount of fungal biomass on the plant or a part of the plant) in response to CLas infection when compared to a control plant. In some cases, however, enhanced resistance can also be measured by the production of the hypersensitive response (HR) of the plant (see, e.g., Staskawicz et al. (1995) Science 268(5211): 661-7). Plants with enhanced pathogen resistance can produce an enhanced hypersensitive response relative to control plants.

Enhanced HLB resistance can also be determined by measuring the increased expression of a gene operably linked to a positive defense regulator or decreased expression or activity of a negative immune regulator protein. Measurement of such expression can be measured by quantifying the accumulation of RNA or subsequent protein product (e.g., using northern or western blot techniques, respectively (see, e.g., Sambrook et al. and Ausubel et al.).

### Examples

The following examples are provided to illustrate, but not limit the claimed invention.

This example describes the identification of positive defense regulators and negative immune response regulators. The experimental methodology used to identify and test the function of the positive and negative regulators is described by Huang et al., (2020) Plant Biotechnol. J. doi.oprg/10.1111/pbi.13502, which is incorporated by reference. Huang et al, describes an effective host/vector/pathogen interaction system using a close relative of CLas. C. Liberibacter solanacearum (CLso), which infects solanaceous plants, the potato psyllid, a major pest of potatoes and tomatoes, and Nicotiana benthamiana, the ideal hosts for virus-induced gene silencing (VIGS) experiments. VIGS is an effective silencing method to knock down expression of plant endogenous genes using a viral (TRV) vector. This system is very similar to the natural citrus/psyllid/CLas interaction system and can be used to rapidly characterize the function of candidate regulators in plant defense responses against C. Liberibacter species.

Through comparing the sRNA profiles of uninfected HLB-tolerant hybrid US-942 and uninfected HLB-sensitive mandarin Cleopatra, conserved miRNAs that were constitutively more abundant in US-942 than in the HLB-susceptible Cleopatra were discovered. Additional miRNAs that were constitutively less abundant in US-942 than in Cleopatra were also discovered. We predicted and annotated the possible immune negative and positive regulators, evaluated the expression level in US942 and Cleopatra and in another HLB-tolerant citrus relative, Sydney hybrid (Microcitrus virgata) with distinct genetic and geographic background. We also performed functional testing in Nicotiana benthamiana (Nb)/potato psyllid/Candidatus Liberibacter solanacearum (CLso) pathosystem described by Huang et al., 2020, supra. BRAP2, CYP93, NDR1-like, PSL4, LYM2, SOT12, SCE1, GLY1, PAL1, WRKY70, and EFR-like were identified as positive immune response regulators; and VAD1, PRT6, OPT1, YSL6, PUB26, DMR6, PAO1, TPS5, ACA11, MPK1CRT1, LIN2, CRWN (LINC4), GPX8. LOX2, and PI4K were identified as negative immune response regulators.

The function of candidate regulators in defense responses against CLso was performed by TRV-based VIGS to knock down the Nb orthologous/homologous genes listed in Table 1 and 2 in Nb plants infected with CLso. The two-week-old Nb plants were exposed to CLso positive potato psyllid nymphs for 5 days. Three to four days after psyllid nymph removal, Agrobacterium tumefaciens carrying the TRV vector contained in a 200 to 300 bp gene fragment to silence the targeted gene was used to inoculate Nb leaves by infiltration. After 17 days of infiltration, the yellowing symptoms and vascular tissue greening of the plants were observed and compared to siRB control. The plant leaf tissue was collected for (Iso DNA detection and target gene expression was analyzed by quantitative real-time polymerase chain reaction. A TRV construct containing apiece of Solanum bulbocastanum-specific late-blight resistance gene RB was used as a negative control (siRB). Nb does not have an orthologous gene and thus does not contain a target RB gene.

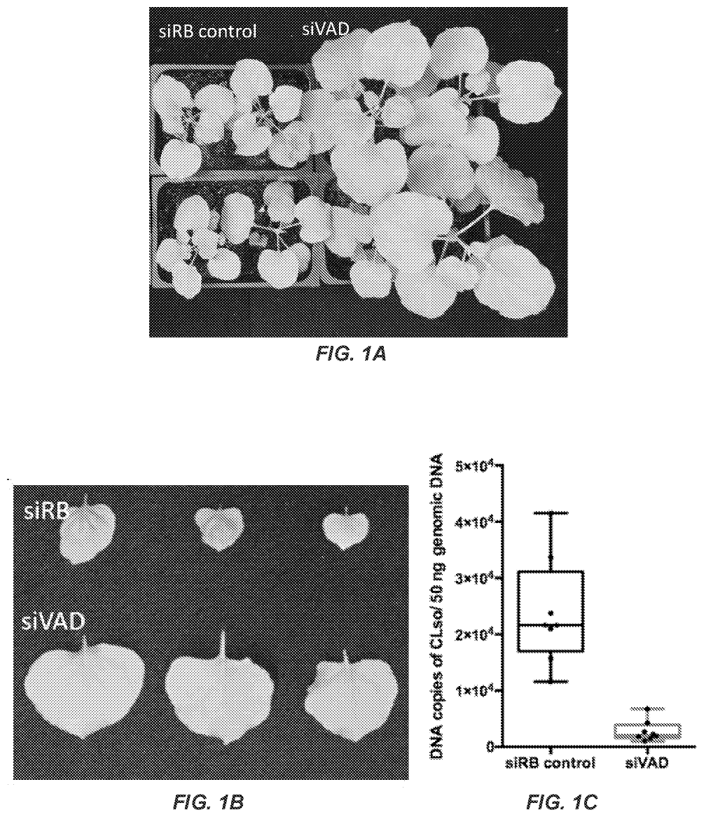

FIG. 1a-c provide data illustrating that mutant plants with VIGS-knocked down VAD expression displayed decreased CLso bacteria titers, measured by probe-based qPCR in 50 ng of host genomic DNA, compared to control plants in which the RB gene was silenced.

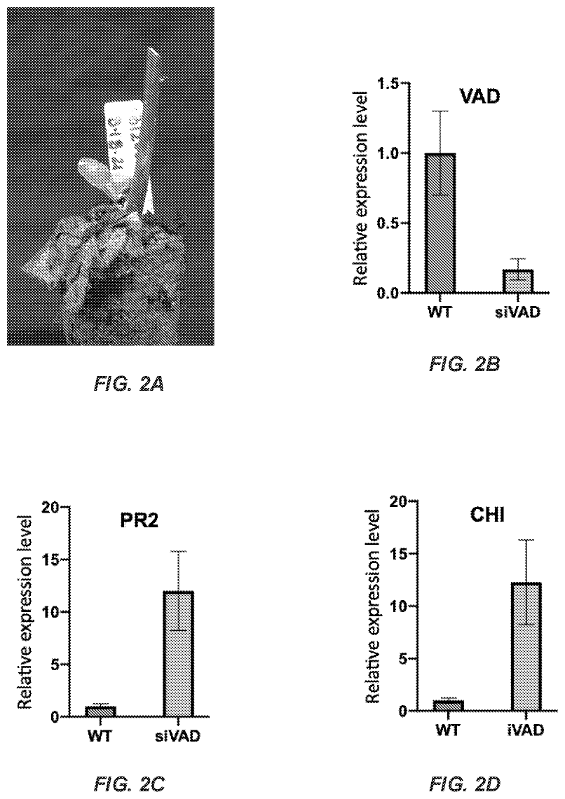

FIG. 2a-d provide data illustrating that VAD knocked-down Carrizo plants (knock down by RNA silencing) exhibited higher expression of defense marker genes including pathogeneis-related (PR-2) and Chitinase (CHI).

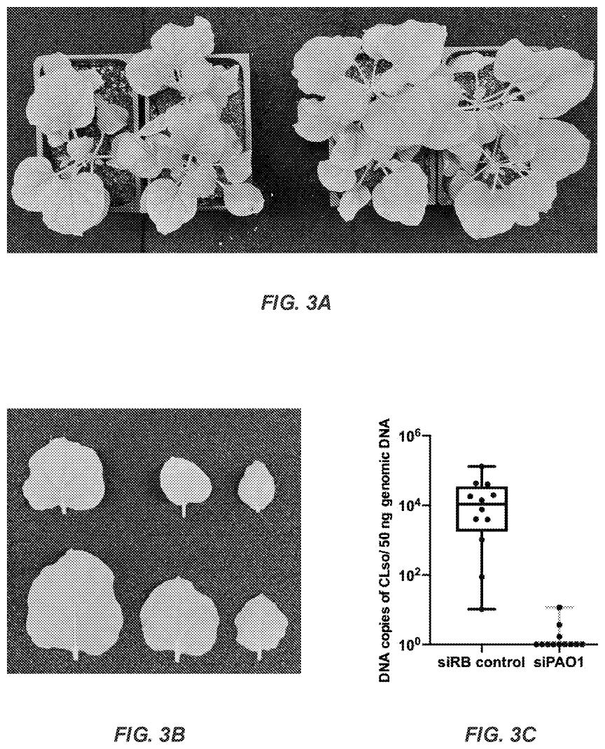

FIG. 3a-c provide data illustrating that PAO1 is a negative regulator in response to CLso infection. PAO1 is a polyamine oxidase that regulates reactive oxygen species homeostasis. Mutant plants with VIGS-knocked down PAO1 expression displayed decreased CLso bacteria titers, measured by probe-based qPCR in 50 ng of host genomic DNA, compared to control plants in which the RB gene was silenced.

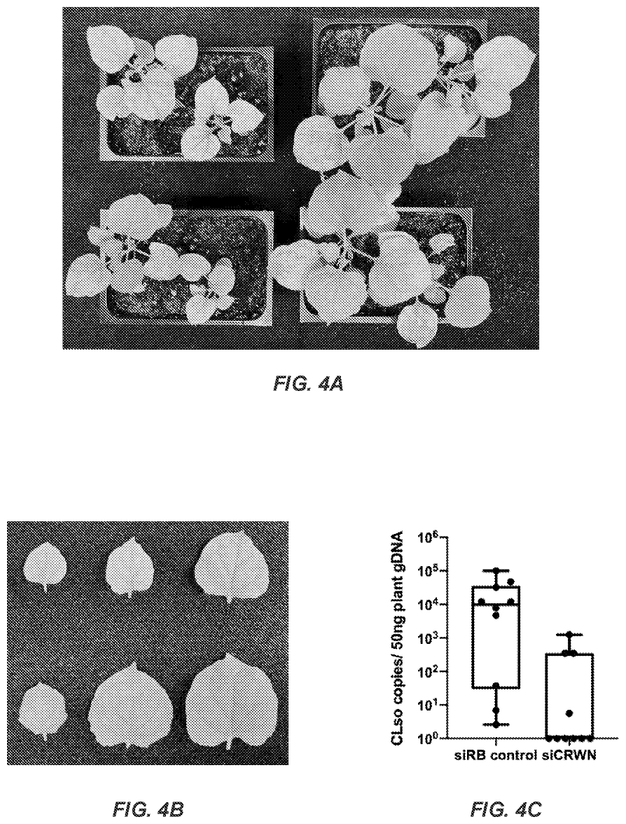

FIG. 4a-c provide data illustrating that CRWN is a negative regulator in response to CLso infection. CRWN is a nuclear lamina protein. Loss of CRWN protein induces the expression of the salicylic acid biosynthetic gene. Mutant plants with VIGS-knocked down CRWN expression displayed decreased CLso bacteria titers, measured by probe-based qPCR in 50 ng of host genomic DNA, compared to control plants in which the RB gene was silenced.

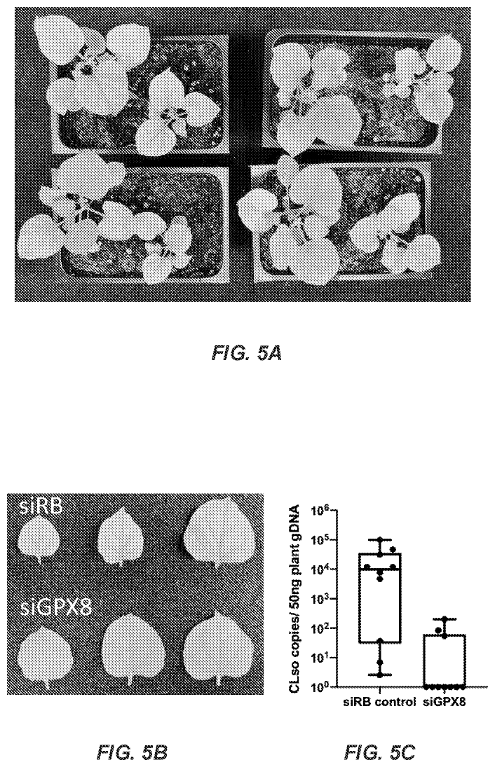

FIG. 5a-c provide data illustrating that GPX8 is a negative regulator in response to Clso infection. GPX8 is a glutathione peroxidase. Reduced GPX expression leads to compromised photoxidative stree tolerance, but increased resistance to virulent bacteria (see, e.g., Chang, et al., Plant Physiol 150: 670-683, 2009), Mutant plants with VIGS-knocked down GPX8 expression displayed decreased CLso bacteria titers, measured by probe-based qPCR in 50 ng of host genomic DNA, compared to control plants in which the RB gene was silenced.

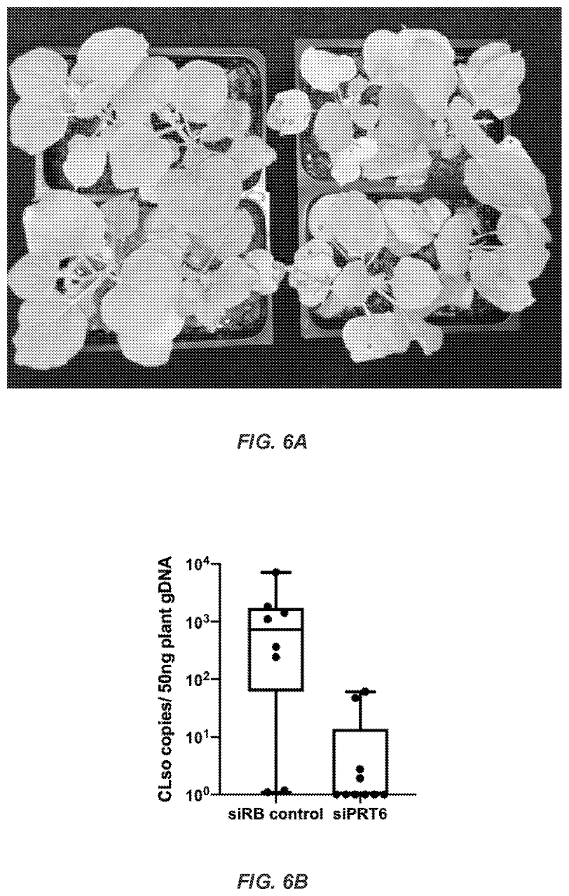

FIG. 6a-b provide data illustrating that PRT6 is a negative regulator in reponse to CLso infection. PRT6 is an E3 ubiquitin-protein ligase. Arabidopsis and barley prt6 mutant plants are resistant to Pst and Ps. japnoica and Blumeria graminis f. sp. hordei (see, e.g., Christopher et al. Plant direct 3:12 e00194, 2019). Mutant plants with VIGS-knocked down PRT6 expression displayed decreased CLso bacteria titers, measured by probe-based qPCR in 50 ng of host genomic DNA, compared to control plants in which the RB gene was silenced.

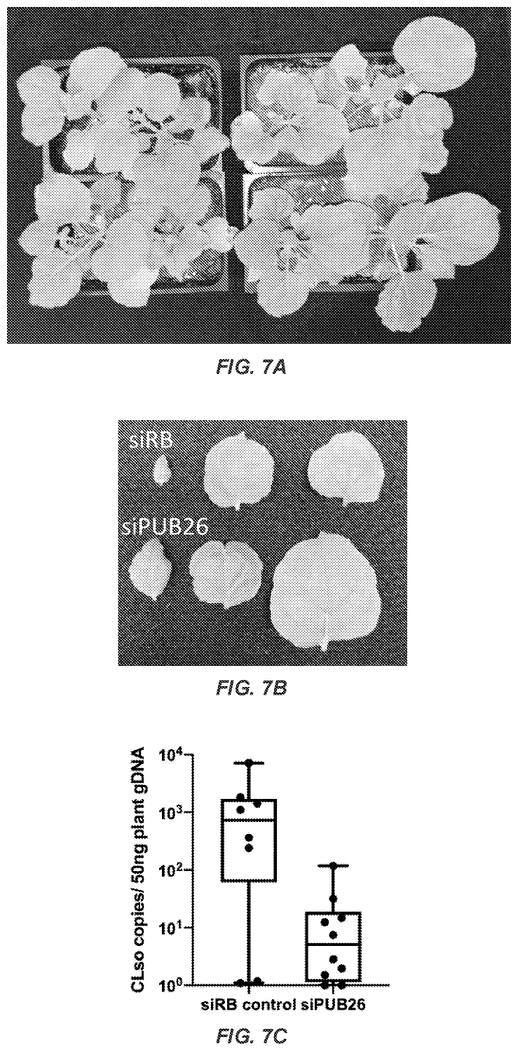

FIG. 7a-b provide data illustrating that PUB25/26 is a negative regulator in response to CLso infection. PUB25/26 is an E3 ligase that targets non-activated immune kinase BIK1 for degradation. Mutant plants with VIGS-knocked down PUB25/26 expression displayed decreased CLso bacteria titers, measured by probe-based qPCR in 50 ng of host genomic DNA, compared to control plants in which the RB gene was silenced.

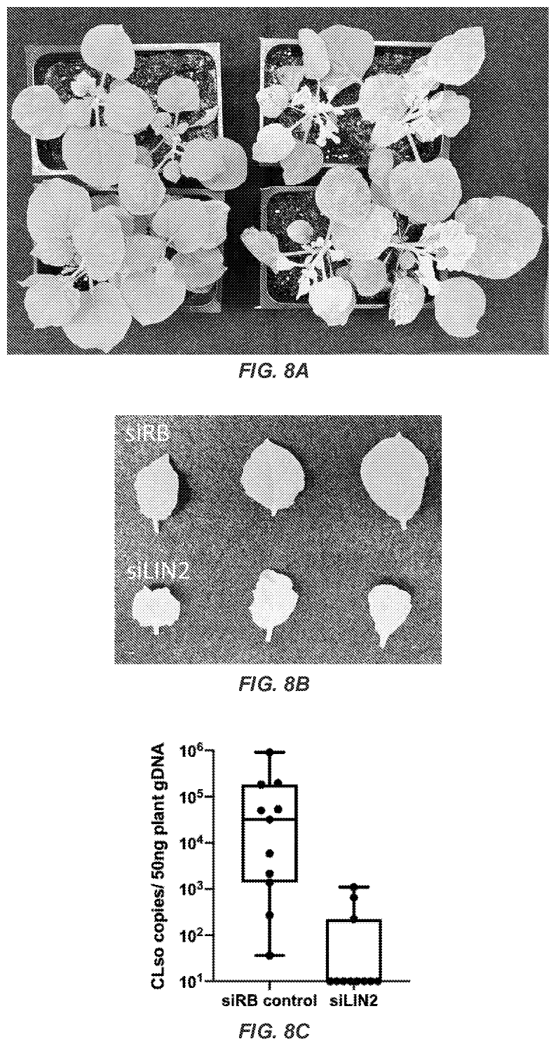

FIG. 8a-c provide data illustrating that LIN2 is a negative regulator in reponse to CLso infection. LIN2 encodes a coproporphyrinogen III oxidase, which is a key enzyme in the biosynthetic pathway of chlorophyll and heme, a tetrapyrrole pathway. LIN2 mutants have higher expression of molecular markers associated with defense responses (see, e.g., Guo, et al., Plant Cell Rep 32:687-702, 2013). Mutant plants with VIGS-knocked down LIN2 expression displayed decreased CLso bacteria titers, measured by probe-based qPCR in 50 ng of host genomnic DNA, compared to control plants in which the RB gene was silenced.

Positive regulators identified in the screen described above were also analyzed as immune response regulators.

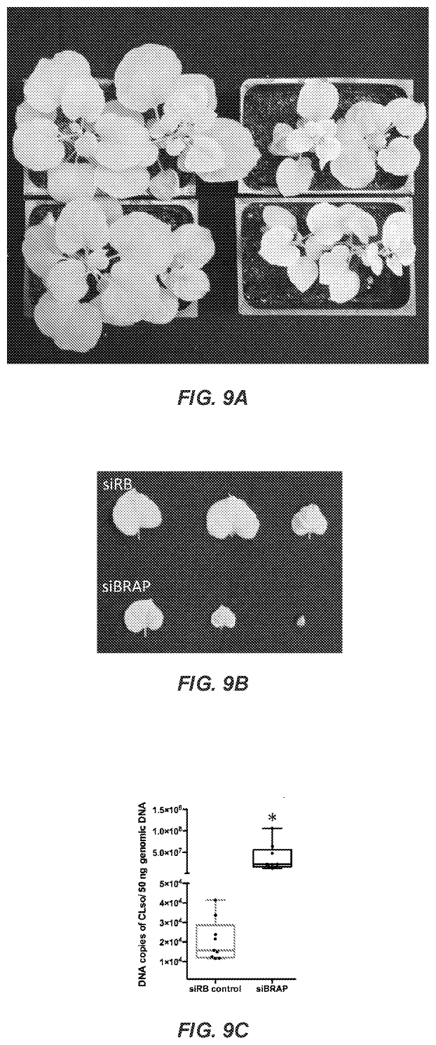

FIG. 9a-c provide data illustrating that BRAP is a positive regulator in response to CLso infection. BRAP is an E3-ligase that positively regulates pathogen-associated molecular patterns triggered in defense responses in plants (see, e.g., Xie, et al., PLoS Pathog 12: 1005529, 2016). Mutant plants with VIGS-knocked down BRAP expression displayed increased CLso bacteria titers, measured by probe-based qPCR in 50 ng of host genomic DNA, compared to control plants in which the RB gene was silenced.

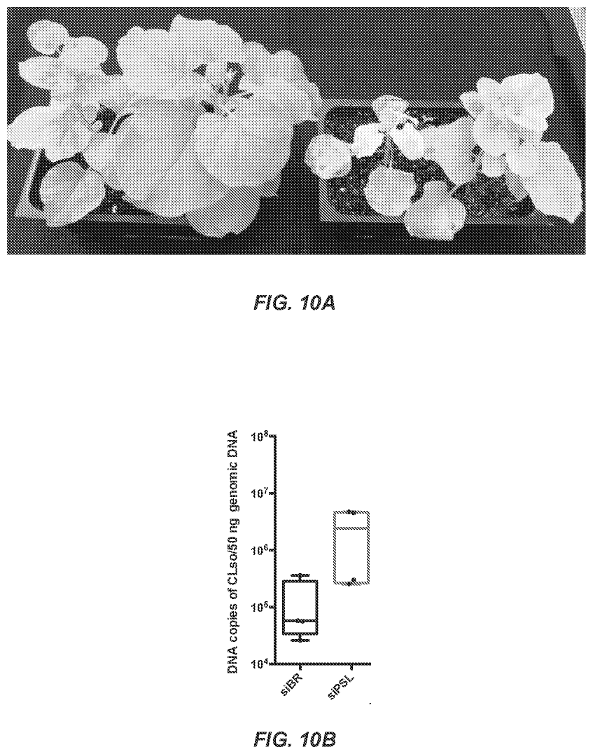

FIG. 10a-b provide data illustrating that PSL4 is a positive regulator in response to CLso infection. PSL4 is essential for stable accumulation and quality control of the elf18 receptor EFR. Mutant plants with VIGS-knocked down PSL4 expression displayed increased CLso bacteria titers, measured by probe-based qPCR in 50 ng of host genomic DNA, compared to control plants in which the RB gene was silenced.

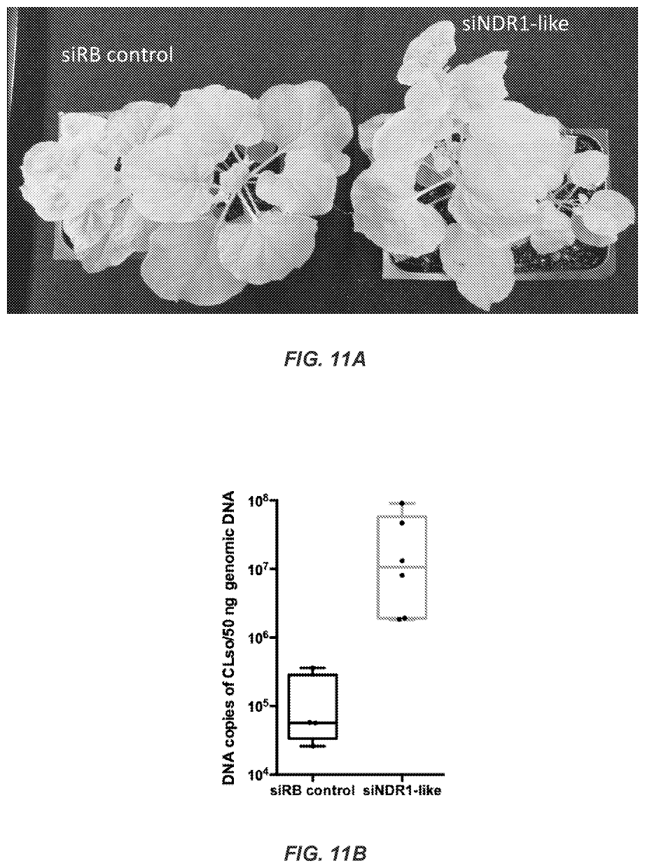

FIG. 11a-b provide data illustrating that NDR1-like is a positive regulator in response to CLso infection. NDR1-like (NON RACE-SPECIFIC DISEASE RESISTANCE 1) is required for non-race specific resistance to bacterial and fungal pathogens. It mediates systemic acquired resistance responses (see, e.g., Day et al., Plant Cell. 18:2782-91, 2006). Mutant plants with VIGS-knocked down NDR1-like expression displayed increased CLso bacteria titers, measured by probe-based qPCR in 50 ng of host genomic DNA, compared to control plants in which the RB gene was silenced.

All references, publications, and accession numbers are incorporated by reference as if each individual accession number were specifically and individually indicated to be incorporated by reference. Although the foregoing disclosure has been described in some detail by way of illustration and example for purposes of clarity of understanding, it will be readily apparent to those of ordinary skill in the art in light of the teachings of this disclosure that certain changes and modifications can be made thereto without departing from the spirit or scope of the invention.

TABLE 3
Polypeptide sequence of citrus plant negative immune
response regulators
VAD1 protein sequences
> CcVAD1_Ciclev10019258m__Citrus_clementina
MALVSASTERINLCGPTDPSSSRSTSEATSSANVSCAADPPDRNVQFSTS
PIPNGDVEVQSSVTLRSEEYRQLFRLPSEEVLVQDFNCAFQESILLQGHM
YLFVHFICFYSNIFGFETKKIIPFYEVTAVRRAKTAGIFPNAIEIFAAGK
KYFFASFLSRDEAFKLITDGWLQHGSGSLASAEQQDSSSETSSPQNGPVV
IEKVNCCSADPIAKSDSIIREEDLSSDSKLPANVEMTPVEMQDDNVEQDF
EPVLDTDSLHPIKTSSWNIENSDAPKIPECYTKVAETNFQMKVEDFYSLF
FSDDTVNFIESFHRKCGDKEFKCTSWHRHYEFGYSRDLSFQHPIKVYFGA
KFGSCKETQKFRVYRNSHLVIETSQEVHDVPYGDYFRVEGLWDVMRDDGG
SKEGCILRVYVNVAFSKKTVWKGKIVQSTLEECRDVYAMWIGMAHDVLKQ
KNLEKPEEGGPAYSTVQNDDVHSERVVNTGETSERLCNADHRIRTLPITD
SLDASQSVGNLLQGNLVDSAAIASLLRESMTKCCSFVKRQSGVSLILVIA
FAVIFLMQVSILVLLNRPQHVHMASPPDYMGAGVGVGLGQRSAESIPWLE
RRMHYLKDEMLMVEARLERMWHEHAVLRAQLKDIEQLHKRE-
>CsVAD1._1_orange1.1g006549m_Citrus_sinesis
MALVSASTERINLCGPTDPSSSRSTSEATSSANVSCAADPPDRNVQFSTS
PIPNGDVEVQSSVTLRSEEYRQLFRLPSEEVLVQDFNCAFQESILLQGHM
YLFVHFICFYSNIFGFETKKIIPFYEVTAVRRAKTAGIFPNAIEIFAAGK
KYFFASFLSRDEAFKLITDGWLQHGSGSLASAEQQDSSSETSSPQNGPVV
IEKVNCCSADPIAKSDSIIREEDLSSDSKLPANVEMTPVEMQDDNVEQDF
EPVLDTDSLHPIKTSSWNIENSDAPKIPECYTKVAETNFQMKVEDFYSLF
FSDDTVNFIESFHRKCGDKEFKCTSWHRHYEFGYSRDLSFQHPIKVYFGA
KFGSCKETQKFRVYRNSHLVIETSQEVHDVPYGDYFRVEGLWDVMRDDGG
SKEGCILRVYVNVAFSKKTVWKGKIVQSTLEECRDVYAMWIGMAHDVLKQ
KNLEKPEEGGPAYSTVQNDDVHSERVVNTGETSERLCNADHRIRTLPITD
SLDASQSVGNLLQGNLVDSAAIASLLRESMTKCCSFVKRQSGVSLILVIA
FAVIFLMQVSILVLLNRPQHVHMASPPDYMGAGVGVGLGQRSAESIPWLE
RRMHYLKDEMLMVEARLERMWHEHAVLRAQLKDIEQLHKRE
>CsVAD1.2_orange1.1g006377m__Citrus_sinesis
MALVSASTERINLCGPTDPSSSRSTSEATSSANVSCAADPPDRNVQFSTS
PIPNGDVEVQSSVTLRSEEYRQLFRLPSEEVLVQDFNCAFQESILLQGHM
YLFVHFICFYSNIFGFETKKIIPFYEVTAVRRAKTAGIFPNAIEIFAAGK
KYFFASFLSRDEAFKLITDGWLQHGSGSLASAEQQDSSSETSSPQNGPVV
IEKVNCCSADPIAKSDSIIREEDLSSDSKLPANVEMTPVEMQDDNVEQDF
EPVLDTDSLHPIKTSSWNIENSDAPKIPECYTKVAETNFQMKVEDFYSLF
FSDDTVNFIESFHRKCGDKEFKCTSWHRHYEFGYSRDLSFQHPIKVYFGA
KFGSCKETQKFRVYRNSHLVIETSQEVHDVPYGDYFRVEGLWDVMRDDGG
SKEGCILRVYVNVAFSKKTVWKGKIVQSTLEECRDVYAMWIGMAHDVLKQ
KNLEKPEGWIVVDSEGGPAYSTVQNDDVHSERVVNTGETSERLCNADHRI
RTLPITDSLDASQSVGNLLQGNLVDSAAIASLLRESMTKCCSFVKRQSGV
SLILVIAFAVIFLMQVSILVLLNRPQHVHMASPPDYMGAGVGVGLGQRSA
ESIPWLERRMHYLKDEMLMVEARLERMWHEHAVLRAQLKDIEQLHKRE
> CsVAD1.3_orange1.1g008222m_Citrus_sinesis
MYLFVHFICFYSNIFGFETKVTSKFQCYVASCNSTLQYQSCFAISNEFML
QKIIPFYEVTAVRRAKTAGIFPNAIEIFAAGKKYFFASFLSRDEAFKLIT
DGWLQHGSGSLASAEQQDSSSETSSPQNGPVVIEKVNCCSADPIAKSDSI
IREEDLSSDSKLPANVEMTPVEMQDDNVEQDFEPVLDTDSLHPIKTSSWN
IENSDAPKIPECYTKVAETNFQMKVEDFYSLFFSDDTVNFIESFARKCGD
KEFKCTSWHRHYEFGYSRDLSFQHPIKVYFGAKFGSCKETQKFRVYRNSH
LVIETSQEVHDVPYGDYFRVEGLWDVMRDDGGSKEGCILRVYVNVAFSKK
TVWKGKIVQSTLEECRDVYAMWIGMAHDVLKQKNLEKPEEGGPAYSTVQN
DDVHSERVVNTGETSERLCNADHRIRTLPITDSLDASQSVGNLLQGNLVD
SAAIASLLRESMTKCCSFVKRQSGVSLILVIAFAVIFLMQVSILVLLNRP
QHVHMASPPDYMGAGVGVGLGQRSAESIPWLERRMHYLKDEMLMVEARLE
RMWHEHAVERAQLKDIEQLHKRE
>Cs VAD1.4_orange1.1g013482m_Citrus_sinesis
MALVSASTERINLCGPTDPSSSRSTSEATSSANVSCAADPPDRNVQFSTS
PIPNGDVEVQSSVTLRSEEYRQLFRLPSEEVLVQDFNCAFQESILLQGHM
YLFVHFICFYSNIFGFETKKIIPFYEVTAVRRAKTAGIFPNAIEIFAAGK
KYFFASFLSRDEAFKLITDGWLQHGSGSLASAEQQDSSSETSSPQNGPVV
IEKVNCCSADPIAKSDSIIREEDLSSDSKLPANVEMTPVEMQDDNVEQDF
EPVLDTDSLHPIKTSSWNIENSDAPKIPECYTKVAETNFQMKVEDFYSLF
FSDDTVNFIESFHRKCGDKEFKCTSWHRHYEFGYSRDLSFQHPIKVYFGA
KFGSCKETQKFRVYRNSHLVIETSQEVHDVPYGDYFRVEGLWDVMRDDGG
SKEGCILRVYVNVAFSKKTVWKGLPLLIHLLISPTCRVLLHV
>AbVAD1_sb18769_Atalantia_buxifolia
MSSATLRSEEYRQLFRLPSEEVIVQDFNCAFQESILLQGHMYLFVHFICF
YSNIFGFETKKIIPFCEVTAVRRAKTAGIFPNAIEIFAAGKKYFFASFLS
RDEAFKLITDGWLQHGSGSLASAEQQDSSSETSSPQNGPVVMEKVNCCSA
DPIAESDSIIREEDLSSDSKLPANVEMTPVEIQDDNVEQDFEPILDTDSS
HPIKTSSWNIENSDAPKIPECYTKVAETKFQMKVEDFYSLFFSDDTVNFI
ESFHRKCGDKEFKCTLWHPHDEFGYSRDLSFQHPIKVYFGAKFGSCKETQ
KFRVYRNSHLVIETSQEVHDVPYGDYFHVEGLWDVMRDDGGSKEGCILRV
YVNVAFSKKTVWKGKIVQSTVEECRDVYAIWIGMAHDVLKQKNLEKPEGW
IVVDSEGGPACSTVQNDDVHSERVVNTGETSERLCNADHQIRTLPITDSL
DASQSIGNLLQGNLVDSAAIASLERESMTKCCSFVKRQSGVSLILVIAFA
VIFLMQVSILVLLNRPQHVHMASPPDYMGAGVGVGVGQRSAESIPWLERR
MHYLKDEMLMVEARLERMWHEHAVLRAQLKDIEQLHKRE-
>CiVAD1_Ci003490_Citrus_ichangensis
MALVSASTERINLCGPTDPSSSRSTSEATSSANVSCAADPPDRNVQFSTS
PIPNGDVEVQSSVALRSEEYRQLFRLPSEEVIVQDFNCAFQESILLQGHM
YLFVHFICFYSNIFGFETKKIIPFYEVTAVRRAKTAGIFPNAIEIFAAGK
KYFFASFLSRDEAFKLITDGWLQHGGGSLASAEQQDSSSETSSPQNGPVV
IEKVNCCSADPIAESDSIIREEDLSSDSKLPANVEMTPVEMQDDNVEQDF
EPVLDTDSSHPIKILSWNIENSDAPKIPECYTKVAETKFQMKVEDFYSLF
FSDDTVNFIESFHRKCGDKEFKCTSWHQHDEFGYSRDLSFQHPIKVYFGA
KFGSCKETQKFQVYRNSHLVIETSQEVHDVPYGDYFRVEGLWDVMRDDGG
SKEGCILRVYVNVAFSKKTVWKGKIVQSTLEECRDVYAMWIGMAHDVLKQ
KNLEKPEEGGPACSTVQNDDVHSERLVNTGETSERLCNADHRIRTLPITD
SLDASQSVGNLLQGNLVDSAAIASWLRESMTKCCSFVKRQSGVSLILVIA
FAVIFLMQVSILVLINRPQHVHMASPPDYMDAGVGLGLGQRSAESIPWLE
RRMHYLKDEMLMVEARLERMWHEHAVLRAQLKDMEQLHKRE-
>CuVAD1.1_GAY41820.1_Citrus_unshiu
MALVSASTERINLCGPTDPSSSRSTSEATSSANVSCAADPPDRNVQFSTSPIPNGDVEVQSSVTLRSEEYRQLFRLPSEE
VIVQDFNCAFQESILLQGHMYLFVHFICFYSNIFGFETKKIIPFYEVTAVRRAKTAGIFPNAIEIFAAGKKYFFASFLSR
DEAFKLITDGWLQHGSGSLASAEQQDSSSETSSPQNGPVVIEKVNCCSADPIAKSDSIIREEDLSSDSKLPANVEMTPVE
MQDDNVEQDFEPVLDTDSLHPIKTSSWNIENSDAPKIPECYTKVAETNFQMKVEDFYSLFFSDDTVNFIESFHRKCGDKG
AKFGSCKETQKFRVYRNSHLVIETSQEVHDVPYGDYFRVEGLWDVMRDDGGSKEGCILRVYVNVAFSKKTVWKGKIVQST
LEECRDVYAMWIGMAHDVLKQKNLEKPEEGGPAYSTVQNDDVHSERVVNTGETSERLCNADHRIRTLPITDSLDASQSVG
NLLQGNLVDSAAIASLLRESMIKCCSFVKRQSGVSLILVIAFAVIFLMQVSILVLLNRPQHVHMASPPDYMGAGVGVGLG
QRSAESIPWLERPMHYLKDEMLMVEARLERMWHEHAVLRAQLKDIEQLHKRE
>CuVAD1.2_GAY41819.1_Citrus_unshiu
MALVSASTERINLCGPTDPSSSRSTSEATSSANVSCAADPPDRNVQFSTSPIPNGDVEVQSSVTLRSEEYRQLFRLPSEE
VLVQDFNCAFQESILLQGHMYLFVHFICFYSNIFGFETKKIIPFYEVTAVRRAKTAGIFPNAIEIFAAGKKYFFASFLSR
DEAFKLITDGWLQHGSGSLASAEQQDSSSETSSPQNGPVVIEKVNCCSADPIAKSDSIIREEDLSSDSKLPANVEMTPVE
MQDDNVEQDFEPVLDTDSLHPIKTSSWNIENSDAPKIPECYTKVAETNFQMKVEDFYSLFFSDDTVNFIESFHRKCGDKG
AKFGSCKETQKERVYRNSHLVIETSQEVHDVPYGDYFRVEGLWDVMRDDGGSKEGCILRVYVNVAFSKKTVWKGKIVQST
LEECRDVYAMWIGMAHDVLKQKNLEKPEGWIVVDSEGGPAYSTVQNDDVHSERVVNTGETSERLCNADHRIRTLPITDSL
DASQSVGNLLQGNLVDSAAIASLLRESMTKCCSFVKRQSGVSLILVIAFAVIELMQVSILVLLNRPQHVHMASPPDYMGA
GVGVGLGQRSAESIPWLERRMHYLKDEMLMVEARLERMWHEHAVLRAQLKDIEQLHKRE
>PtVAD1.1_Ptrif.0003s4973.2_Poncirus_trifoliata
MALVSASTERINLCGPTDPSSSRSTSEATPSANVSCAADPPDRNVQFSTS
PIPNGDVEVQSSVTLRSEEYRQLFRLPSEEVLVQDFNCAFQESILLQGHM
YLFVHFICFYSNIFGFETKKIIPFCEVTAVRRAKTAGIFPNAIEIFAAGK
KYFFASFLSRDEAFKLITDGWLQHGGGSLASAEQQDSSSETSSPQNGPVV
IEKVNCCSADPIAESDSIIREEDLSSDSKLPANVEMTPVEMQDDNVEQDF
EPVLDTDSSHPINISSWNIENSDAPKIPECYTKVAETKFQMKVEDFYSLF
FSDDTVNFIESFHRKCGDKEFKCTSWHRHDEFGYSRDLSFQHPIKVYFGA
KFGSCKETQKFRVYRNSHLVIETSQEVHDVPYGDYFRVEGLWYVMRDDGG
SKEGCILRVYVNVAFSKKTVWKGKILQSTLEECRDVYAMWIGMAHDVLKQ
KNLEKPEEGGPACSTVQNDDVHSERVVNTGETSERLCDADHRIRTLPITD
SLDASQSVGNLLQGNLVDSAAIASWLRESMTKCCSFVKRQSGVSLILVIA
FAVIFLMQVSILVLINRPQHVHMASPPDYMGAGVGVGLGQRSAESIPWLE
RPMHYLKDEMLMVEARLERMWHEHAVERAQLKDMEQLHKRE-
>SIVAD1_Solyc01g090230.2 Tomato Genome protein sequences (ITAG release 2.40)
MAAVVVPEKIMSPSPPPSQHMHLSPPTSRRSTDTSSGTNASPDRRSSLDLPSSSTSSPSRLSDAQNQLALKSEEYRLLFR
LPPDEVLVQDFNCALQENFLLQGHMYLFVHSICFYSNLFGFETKKIIPFHEITAVRRAKAAAIFPTAIEIVAGGKKYFFT
SFLSRDEAFKLIDDGWLQHNGAAKESADLEPQSDLTFLDSGIVEGADSFRQATERVECLERNEDNMVQEDSKPLVNGQFE
IVSNPSRVQDNVEEEVVIVQNTDCSPSEKSYGLKQEDSDAPRVPEGFTLVAEAKFPVTVEKFFELFISDAGVAFQESFRR
NCGDKDFKCTQWRPHEEFGHTRNLSFQHPIKIYLGPKFGGCHEFQKCRRYRNSHLVIESSQEISGVPFADYFRVEAFWDV
ERDGDGPEGGCIMKVYLNLVFTKKTIFRGKIVQSTIDECRALYVKWIALARELLKQKKLEKEKADCLAANVVTSAQPKES
YEHVENIVETSKEIRSQIPPLNQQAADSSTVSLTSSCRDFMLKCSASLKSQSHVSILIVITIAVILILMQMSILVLLGRP
QHVQVISQGDSASSMYRLGETGVDILGFLDKKINHLKDEMFMVETLLGKMQQEHTLLKTQLKEFEHLRKLQKG
>St VAD1_XP_006347965.1 PREDICTED: protein VASCULAR ASSOCIATED DEATH 1,
chloroplastic [Solanum tuberosum]
MAAVVVPEKIMSPSPPPSQHMHTSPSTSRRSMDTASDTNASPDRRSSLDLPSSSTASPSRLSDAQNQLALKSEEYRLLFR
LPPDEVEVQDFNCALQESFLLQGHMYLFGHSICFYSNLFGFETKKIIPFHEITAVRRAKAAAIFPTAIEIVAGGKKYFFT
SFLSRDEAFKLIDDGWLQHNGAAKESADLEPQSDLNFLDSGIVEGADSFRQAKEGVECLERNEDNMVQEDSKPLVNGQFE
IVSNPSGVQDSVEEEAVIVQNTDCSSSEKSYGLKQEDSDAPRVPEGFTLVAEAKFPVKVEKFFEFFISDAGLAFQESFRR
KCGDKDFKCTQWRPHEEFGHTRNLSFQHPIKIYLGPKFGGCHEFQKCRHYRNSHLVIESSQEITGVPYADYFRVEAFWDV
ERDGDGPEGGCCMKVYLNVVFTKKTIFRGKIVQSTIDECRALYVTWIALAHDELLKQKKLEKEKADGQAAIVVTSAQPKK
IYEHVENLDETSNEIRSQIPPLNQQAADSSTVSITSLCRDFMLKCSSSEKSQSHVSILIVITIAVILILMQMSILVLLGR
PQHVQVISQGDSASSMYRLGETGVDILGFLDKKINHLKDEMFMVETLLGKMQQEHTLLKTQLKEFEHLRKLQKG
Proteolysis 6, PRT6 protein sequences
>PtPRT6_Ptrif._0006s0640_Poncirus_trifoliata
MEIDSPPDFSPPKPRDRIVRRLINIGVPEEFLDYSGIVNFAKNDKSRIPE
LVSTILPPDEEVAEVIQDAKAKNKKVSVGPNMKGRFRESMLWLQCLMFER
EPEKVERKLSKIGQRAYRCRTCEHDPTCAICVPCFQNGNHKEHDYSIIYT
GGGCCDCGDVTAWKREGFCSRHKGAEQIQPLPEKYANSATPVLDALFIYW
ENKLSLAESVGQENPRSSDHVAERRKLANELTFAVVEMLLEFCKNSESLL
SFVSKRVISVIGLLDILVRAERFSSDVVVKNLHELLLKLLGEPIFKYEFA
KVFLSYYPVFVKDAIREHSDDTIKKYPLISTFSVQIFTVPTLTPRIVKEM
NLLEMLLGCLREIFDSCAGDDSCIQVAKGANLYETTNRVIGDIRFVMSHA
AVSKYATHEQLNISKAWMKLLTFVQGMNPQKRETGIQIREENEYMHLPLV
LDHSIANIQPLLVDGAFSSAVAEETRYDFSMYKQDIGDGDSLRHAKVGRL
SQESSVCGAMGRSSLSASTLKADDVIFDAVSDVLLPHSVTWLAHECLRAM
ENWLGVDDRSVSVNDILSPNASRISGSNFVALKKTLSKIKKGKSIFSRLA
GTSEVTASIQESGDLDNATSMGKESKITISGERGTASWRSAGFNDSQMEG
ECAAELDNLHVLSLCYWPDITYDVSSQDVSVHIPLHRLLSLIIQKALRRC
YGESAASESADTGAENPLSAVSLDFFGHILGGCHPYGFSAFVMEHPLRIR
VFCAQVHAGMWRRNGDAALSSCEWYRAVRWSEQGLELDLFLLQCCAALAP
ADLYVNRIIERFGLSNYLSINLERRSEYEPILVQEMLTLIIQILQERRFC
GLTTAESLKRELVHRLAIGDATHSQLVKSLPRDLSKEDQLQEILDAVAMY
SHPSGFNQVLLTALHLLALALDVCFQKKKSGDQSCDIGGSTPLLDFASEE
IAEGLNNDNFLEAGNCNLSSVIESLLKKFAEIDSRCMTKLQQLAPEIVSH
LSQSLPRDDTSGSFSASDSEKRKAKARERQAAILEKMKAEQFKFLSSISS
NIEDAPKSAPEVTNYDAEHVSEESVQDVCALCHDPNSRTPVSYLILLQKS
RLLSFVDRGSPSWDQDQWLGKECGTISANNMVNQFGTNTPSSGLGVISSC
QLAQVAEEAVNQFAYNGKPEEVNAVLEFVKAQFPSLRNIPIPFTFSNGRK
CTASSMEMFEQDLYLSICREMRKNMTYPDLMKEDEECSVAEGGFKNRGNS
DSFLLGKYVASISKEMRENASASEVSRGDRIAAESLVYDGFGPIDCDGIH
LSSCGHAVHQGCLDRYVSSLKERQFSLRNAAASINLPAAVMCFVCLYACI
YNRRIIFEGGHIVDPDEGEFLCPVCRQLANSVLPALPWDLQRINEQPTVS
GVGLEENTSLQLQQAVSLLLSASNVVGKADVIESFPLMKNEIMASNVEAV
SRRMCKMYFQNKVDKFFGSARVNPSLIMWDALKYSLMSMEIAARSEKTSM
TPIYDVNALDKELRSSSGFVLSLLLKVVQSMRSKNSLHVLQRFRGIQLFA
ESICSGTSIDNPGGRCKRGGNMLSILKHADVEVSYPDIQFWNRASDPVLA
RDPFSSLMWVLFCLPCQFILCKESLLSLVHVFYAVTLSQAVLSCCGKLQS
KVNELGFSDSLISDISKLLGEFGSAQEYFVSNYIDPSCDIKDMIRRLSFP
YLRRDHVLARSSHGISDMMDSSDDALSDLKEIQEVEKMFKIPSLDVILKD
EVLRSLVLKWFHHFSKEFEVHRFQHVLYSTPAVPFKLMRLPHLYQDLLQR
LCSPRWKPCCRESSCQSHAMACGAGTGVFLLIRRTTILLQRCARQAPWPS
PYLDAFGEEDIEMHRGKPLYLNEERYAALTYMVASHGLDRSSKVLSQTTI
GGFFLV-
>CcPRT6_ESR42326.1_CICLE_v10013610m_Citru_ clementina
MEIDSPPDFSPPKPRDRIVRRLINIGVPEEFLDYSGIVNFAK
NDKSRIPELVSTILPPDEEVAEVIQDAKAKNKKVSVGPNMKGRFRESMLWLQWLMFEREPEKVLRKLSKIGQRGVCGAVW
GNNDIAYRCRTCEHDPTCAICVPCFQNGNHKEHDYSIIYTGGGCCDCGDVTAWKREGFCSRHKGAEQIQPLPEKYANSAA
PVLDALFIYWENKLSLAESVGQENPRASDHVAERRKLANELTFAVVEMLLEFCKNSESLLSFVSKRVISVIGLLDILVRA
ERFSSDVVVRKLHELLLKLIGEPIFKYEFAKVFLSYYPVFVKDAIREHSDDTIKKYPLLSTFSVQIFTVPTLTPRLVKEM
NLLEMLIGCLREIFDSCAGDDSCLQVAKWANLYETTNRVIGDIRFVMSHAAVSKYATHEQLNISKAWMKLLTFVQGMNPQ
KRETGIHIREENEYMHLPLVLDHSIANIQPLLVDGAFSSAVAEETRYDFSMYKQDIGDGDSLRHAKVGRLSQESSVCGAM
GRSSLSASTLKADDVIFDAVSDVLLPHSVTWLAHECLRAMENWLGVDDRSVSVNDILSPNASRISGSNFVALKKELSKIK
KGKSIFSRLAGSSEVTAGIQESGDLDNATSMGKESKITISGERDTASWRSAGFNDSEMEGECATELDNLHVLSLCYWPDI
TYDVSSQDVSVHIPLARLLSLIIQKALRRCYGESAASESADTGAENPLSAVSLDFFGHILGGCHPYGFSAFVMEHPLRIR
VFCAQVHAGMWRRNGDAALSSCEWYRAVRWSEQGLELDLFLLQCCAALAPADLYVNRIIERFGLSNYLSLNLERPSEYEP
ILVQEMLTLIIQILQERRFCGLTTAESLKRELVHRLAIGDATHSQLVKSLPRDLSKFDQLQEILDAVAMYSHPSGFNQLA
ITTCKSKVVLQVIRAVLFYAVFTDNPTDSRAPYGVLLTALHLLALALDVCFQKKKSGDQSCDIGGSTPILDFASEEIAEG
LNNGAGKQSLLSLLVFLMGMYKKDGADNFLEAGNCNLSSVIESLLKKFAEIDSRCMTKLQQLAPEIVSHLSQSLPRDDTS
GSFSASDSEKRKAKARERQAAILEKMKAEQFKFLSSISSNIEDAPKSAPEVINYDAEHVSEESVQDVCALCHDPNSRTPV
SYLILLQKSRLLSFVDRGSPSWDQDQWLGKECGTISANNMVNQFGTNTPSSALGVISSCQLAQVAEEAVNQFAYNGKPEE
VNAVLEFVKAQFPSLRNIPIPFTFSNGRKCTASSMEMFEQDLYLSICREMRKNMTYPDLMKEDEECSVAEGGLKNRGNSD
SFLLGKYVASISKEMRENASASEVSRGDRIAAESLVYDGFGPIDCDGIHLSSCGHAVHQGCLDRYVSSLKERYNRRIIFE
GGHIVDPDQGEFLCPVCRQLANSVLPALPWDLQRINEQPTVSGVGLSLDSNSSFTTREENTSLQLQQAVSLLQSASNVVG
KADVIESFPLLKNEIMASNVEAVSRRMCKMYFQNKLDKFFGSARVNPSLIMWDALKYSLMSMEIAARSEKTSTTPIYDVN
ALDKELKSSSGFVLSLLLKVVQSMRSKNSLHVLQRFRGIQLFAESICSGTSIDNPGGRCKRGGNMLSILKHADVEVSYPD
IQFWNRASDPVLARDPFSSLMWVLFCLPCQFILCKESLLSLVHVFYAVTLSQAVLSCCGKLQSKVNELGFSDSLISDISK
LLGEFGSAQEYFVSNYIDPSCDIKDMIRRLSFPYLRRDHVLARSSHGISDMMDSSDDALSDLKEIQEVEKMFKIPSLDVI
LKDEVLRSLVLKWFHHFSKEFEVHRFQHVLYSTPAVPFKLMCLPHLYQDLLQRLCSPSWKPCCRESSCQSHAVACGAGTG
VFLLIRRTTILLQRCARQAPWPSPYLDAFGEEDIEMHRGKPLYLNEERYAALTYMVASHGLDRSSKVLSQTTIGGFFLV
>CsPRT6 isoform X1_XP_006480821.1_Citrus_sinensis
MEIDSPPDFSPPKPRDRIVRRLINIGVPEEFLDYSGIVNFAKNDKSRIPELVSTILPPDEEVAEVIQDAKAKNKKVSVGP
NMKGRFRESMLWLQWLMFEREPEKVLRKLSKIGQRGVCGAVWGNNDIAYRCRTCEHDPTCAICVPCFQNGNHKEHDYSII
YTGGGCCDCGDVTAWKREGFCSRHKGAEQIQPLPEKYANSAAPVLDALFIYWENKLSLAESVGQENPRASDHVAERRKLA
NELTFAVVEMLLEFCKNSESLLSFVSKRVISVIGLLDILVRAERFSSDVVVRKLHELLLKLLGEPIFKYEFAKVFLSYYP
VFVKDAIREHSDDTIKKYPLLSTFSVQIFTVPTLTPRLVKEMNLLEMLLGCLREIFDSCAGDDSCLQVAKWANLYETTNR
VIGDIRFVMSHAAVSKYATHEQLNISKAWMKLLTFVQGMNPQKRETGIHIREENEYMHLPLVLDHSIANIQPLLVDGAFS
SAVAEETRYDFSMYKQDIGDGDSLRHAKVGRLSQESSVCGAMGRSSLSASTLKADDVIFDAVSDVLLPHSVTWLAHECLR
AMENWLGVDDRSVSVNDILSPNASRISGSNFVALKKTLSKIKKGKSIFSRLAGSSEVTAGIQESGDLDNATSMGKESKIT
ISGERDTASWRSAGFNDSEMEGECATELDNLHVLSLCYWPDITYDVSSQDVSVHIPLHRLLSLIIQKALRRCYGESAASE
SADTGAENPLSAVSLDFFGHILGGCHPYGFSAFVMEHPLRIRVFCAQVHAGMWRRNGDAALSSCEWYRAVRWSEQGLELD
LFLLQCCAALAPADLYVNRIIERFGLSNYLSINLERPSEYEPILVQEMLTLIIQILQERRFCGLTTAESLKRELVHRLAI
GDATHSQLVKSLPRDLSKFDQLQEILDAVAMYSHPSGFNQGMYSLRWSYWKELDIYHPRWSSRDLQVAEERYLRFCSVSA
LTAQLPRWTKIYYPLESIAGIATCKVVLQVIRAVLFYAVFTDNPTDSRAPYGVLLTALHLLALALDVCFQKKKSGDQSCD
IGGSTPILDFASEEIAEGLNNGAGKQSLLSLLVFLMGMYKKDGADNFLEAGNCNISSVIESLLKKFAEIDSRCMTKLQQL
APEIVSHLSQSLPRDDTSGSFSASDSEKRKAKARERQAAILEKMKAEQFKFLSSISSNIEDAPKSAPEVINYDAEHVSEE
SVQDVCALCHDPNSRTPVSYLILLQKSRLLSFVDRGSPSWDQDQWLGKECGTISANNMVNQFGTNTPSSALGVISSCQLA
QVAEEAVNQFAYNGKPEEVNAVLEFVKAQFPSLRNIPIPFTFSNGRKCTASSMEMFEQDLYLSICREMRKNMTYPDLMKE
DEECSVAEGGLKNRGNSDSFLLGKYVASISKEMRENASASEVSRGDRIAAESLVYDGFGPIDCDGIHLSSCGHAVHQGCL
DRYVSSLKERYNRRIIFEGGHIVDPDQGEFLCPVCRQLANSVLPALPWDLQRINEQPTVSGVGLSLDSNSSFTTREENTS
LQLQQAVSLLQSASNVVGKADVIESFPLLKNEIMASNVEAVSRRMCKMYFQNKLDKFFGSARVNPSLIMWDALKYSLMSM
EIAARSEKTSTTPIYDVNALDKELKSSSGFVLSLLLKVVQSMRSKNSLHVLQRFRGIQLFAESICSGTSIDNPGGRCKRG
GNMLSILKHADVEVSYPDIQFWNRASDPVLARDPFSSLMWVLFCLPCQFILCKESLLSLVHVFYAVTLSQAVLSCCGKLQ
SKVNELGFSDSLISDISKLLGEFGSAQEYFVSNYIDPSCDIKDMIRRLSFPYLRRCALLWKLLNSTVPPPFSDRDHVLAR
SSHGISDMMDSSDDALSDLKEIQEVEKMFKIPSLDVILKDEVLRSLVLKWFHHFSKEFEVHRFQHVLYSTPAVPFKLMCL
PHLYQDLLQRYIKQCCSDCKSVLDEPALCLLCGRLCSPSWKPCCRESSCQSHAVACGAGTGVFLLIRRTTILLQRCARQA
PWPSPYLDAFGEEDIEMHRGKPLYLNEERYAALTYMVASHGLDRSSKVLSQTTIGGFFLV
>CsPRT6 isoform X2_XP_006480824.1_Citrus_sinensis
MEIDSPPDFSPPKPRDRIVRRLINIGVPEEFLDYSGIVNFAKNDKSRIPELVSTILPPDEEVAEVIQDAKAKNKKVSVGP
NMKGRFRESMLWLQWLMFEREPEKVLRKLSKIGQRGVCGAVWGNNDIAYRCRTCEHDPTCAICVPCFQNGNHKEHDYSII
YTGGGCCDCGDVTAWKREGFCSRHKGAEQIQPLPEKYANSAAPVIDALFIYWENKLSLAESVGQENPRASDHVAERRKLA
NELTFAVVEMLLEFCKNSESLLSFVSKRVISVIGLLDILVRAERFSSDVVVRKLHELLLKLLGEPIFKYEFAKVFLSYYP
VFVKDAIREHSDDTIKKYPLLSTFSVQIFTVPTLTPRLVKEMNLLEMLLGCLREIFDSCAGDDSCLQVAKWANLYETTNR
VIGDIRFVMSHAAVSKYATHEQLNISKAWMKLLTFVQGMNPQKRETGIHIREENEYMHLPLVLDHSIANIQPLLVDGAFS
SAVAEETRYDFSMYKQDIGDGDSLRHAKVGRLSQESSVCGAMGRSSLSASTLKADDVIFDAVSDVLLPHSVTWLAHECLR
AMENWLGVDDRSVSVNDILSPNASRISGSNFVALKKTLSKIKKGKSIFSRLAGSSEVTAGIQESGDLDNATSMGKESKIT
ISGERDTASWRSAGFNDSEMEGECATELDNLHVLSLCYWPDITYDVSSQDVSVHIPLHRLLSLIIQKALRRCYGESAASE
SADTGAENPLSAVSLDFFGHILGGCHPYGFSAFVMEHPLRIRVFCAQVHAGMWRRNGDAALSSCEWYRAVRWSEQGLELD
LFLLQCCAALAPADLYVNRIIERFGLSNYLSINLERPSEYEPILVQEMLTLIIQILQERRFCGLTTAESLKRELVHRLAI
GDATHSQLVKSLPRDLSKFDQLQEILDAVAMYSHPSGFNQGMYSLRWSYWKELDIYHPRWSSRDLQVAEERYLRFCSVSA
LTAQLPRWTKIYYPLESIAGIATCKVVLQVIRAVLFYAVFTDNPTDSRAPYGVLLTALHLLALALDVCFQKKKSGDQSCD
IGGSTPILDFASEEIAEGLNNGAGKQSLLSLLVFLMGMYKKDGADNFLEAGNCNLSSVIESLLKKFAEIDSRCMTKLQQL
APEIVSHLSQSLPRDDTSGSFSASDSEKRKAKARERQAAILEKMKAEQFKFLSSISSNIEDAPKSAPEVTNYDAEHVSEE
SVQDVCALCHDPNSRTPVSYLILLQKSRLLSFVDRGSPSWDQDQWLGKECGTISANNMVNQFGTNTPSSALGVISSCQLA
QVAEEAVNQFAYNGKPEEVNAVLEFVKAQFPSLRNIPIPFTFSNGRKCTASSMEMFEQDLYLSICREMRKNMTYPDLMKE
DEECSVAEGGLKNRGNSDSFLLGKYVASISKEMRENASASEVSRGDRIAAESLVYDGFGPIDCDGIHLSSCGHAVHQGCL
DRYVSSLKERYNRRIIFEGGHIVDPDQGEFLCPVCRQLANSVLPALPWDLQRINEQPTVSGVGLSLDSNSSFTTREENTS
LQLQQAVSLLQSASNVVGKADVIESFPLLKNEIMASNVEAVSRRMCKMYFQNKLDKFFGSARVNPSLIMWDALKYSLMSM
EIAARSEKTSTTPIYDVNALDKELKSSSGFVLSLLLKVVQSMRSKNSLHVLQRFRGIQLFAESICSGTSIDNPGGRCKRG
GNMLSILKHADVEVSYPDIQFWNRASDPVLARDPFSSLMWVLFCLPCQFILCKESLLSLVHVFYAVTLSQAVLSCCGKLQ
SKVNELGFSDSLISDISKLLGEFGSAQEYFVSNYIDPSCDIKDMIRRLSFPYLRRCALLWKLLNSTVPPPFSDRDHVLAR
SSHGISDMMDSSDDALSDLKEIQEVEKMFKIPSLDVILKDEVLRSLVLKWFHHFSKEFEVHRFQHVLYSTPAVPFKLMCL
PHLYQDLLQRYIKQCCSDCKSVLDEPALCLLCGRLCSPSWKPCCSCQSHAVACGAGTGVFLLIRRTTILLQRCARQAPWP
SPYLDAFGEEDIEMHRGKPLYLNEERYAALTYMVASHGLDRSSKVLSQTTIGGFFLV
>CsPRT6.1_KDO44129.1_CISIN_1g000141mg_Citrus_sinensis
MEIDSPPDFSPPKPRDRIVRRLMNIGVPEEFLDYSGIVNFAKNDKSRIPELVSTILPPDEEVAEVIQDAKAKNKKVSVGP
NMKGRFRESMLWLQWLMFEREPEKVLRKLSKIGQRGVCGAVWGNNDIAYRCRTCEHDPTCAICVPCFQNGNHKEHDYSII
YTGGGCCDCGDVTAWKREGFCSRHKGAEQIQPLPEKYANSAAPVLDALFIYWENKLSLAESVGQENPRASDHVAERRKLA
NELTFAVVEMLLEFCKNSESLLSFVSKRVISVIGLLDILVRAEMFSSDVVVRKLHELLLKLLGEPIFKYEFAKVFLSYYP
VFVKDAIREHSDDTIKKYPLLSTFSVQIFTVPTLTPREVKEMNLLEMLLGCLREIFDSCAGDDSCLQVAKWANLYETTNR
VIGDIRFVMSHAAVSKYATHEQLNISKAWMKLLTFVQGMNPQKRETGIHIREENEYMHLPLVLDHSIANIQPLLVDGAFS
SAVSEETRYDFSMYKQDIGDGDSLRHAKVGRLSQESSVCGAMGRSSLSASTLKADDVIFDAVSDVLLPHSVTWVAHECLR
AMENWLGVDDRSVSVNDILSPNASRISGSNFVALKKTLSKIKKGKSIFSRLAGSSEVTAGIQESGDLDNATSMGKESKIT
ISGERDTASWRSAGFNDSEMEGECATELDNLHVLSLCYWPDITYDVSSQDVSVHIPLHRLLSLIIQKALRRCYGESAASE
SADTGAENPLSAVSLDFFGHILGGCHPYGFSAFVMEHPLRIRVFCAQVHAGMWRRNGDAALSSCEWYRAVRWSEQGLELD
LFLLQCCAALAPADLYVNRIIERFGLSNYLSLNLERPSEYEPILVQEMLTLIIQILQERRFCGLTTAESLKRELVHRLAI
GDATHSQLVKSLPRDLSKFDQLQEILDAVAMYSHPSGFNQGMYSLRWSYWKELDIYHPRWSSRDLQVAEERYLRFCSVSA
LTAQLPRWTKIYYPLESIAGIATCKVVLQVIRAVLFYAVFTDNPTDSRAPYGVLLTALHLLALALDVCFQKKKSGDQSCD
IGGSTPILDFASEEIAEGLNNGAGKQSLLSLLVFLMGMYKKDGADNFLEAGNCNLSSVIESLLKKFAEIDSRCMTKLQQL
APEIVSHLSQSLPRDDTSGSFSASDSEKRKAKARERQAAILEKMKAEQFKFLSSISSNIEDAPKSAPEVINYDAEHVSEE
SVQDVCALCHDPNSRTPVSYLILLQKSRLLSFVDRGSPSWDQDQWLGKECGTISANNMVNQFGTNTPSSGLGVISSCQLA
QVAEEAVNQFAYNGKPEEVNSVLEFVKAQFPSLRNIPIPFTFSNGRKCTASSMEMFEQDLYLSICREMRKNMTYPDLMKE
DEECSVAEGGLKNRGNSDSFLLGKYVASISKEMRENASASEVSRGDRIAAESLVYDGFGPIDCDGIHLSSCGHAVHQGCL
DRYVSSLKERYNRRIIFEGGHIVDPDQGEFLCPVCRQLANSVLPALPWDLQRINEQPTVSGVGLSLDSSSSFTTREENTS
FQLQQAVSLLQSASNVVGKADVIESFPLMKNEIMASNVEAVSRRMCKMYFQNKLDKFFGSARVNPSLIMWDALKYSLMSM
EIAARSEKTSTTPIYDVNALDKELKSSSGFVLSLLLKVVQSMRSKNSLHVLQRFRGIQLFAESICSGTSIDNPGGRCKRG
GNMLSILKHADVEVSYPDIQFWNRASDPVLARDPFSSLMWVLFCLPCQFILCKESLLSLVHVFYAVTLSQAVLSCCGKLQ
SKVNELGFSDSLISDISKLLGEFGSAQEYFVSNYIDPSCDIKDMIRRLSFPYLRRCALLWKLLNSTVPPPFSDRDHVLAR
SSHGISDMMDSSDDALSDLKEIQEVEKMFKIPSLDVILKDEVLRSLVLKWFHHFSKEFEVHRFQHVLYSTPAVPFKLMCL
PHLYQDLLQRYIKQCCSDCKSVLDEPALCLICGRLCSPSWKPCCRESSCQSHAVACGAGTGVFLLIRRTTILLQRCARQA
PWPSPYLDAFGEEDIEMHRGKPLYLNEERYAALTYMVASHGLDRSSKVLSQTTIGGFFLV
> CsPRT6.2_KDO44133.1_CISIN_1g000141mg_Citrus_sinensis
MEIDSPPDFSPPKPRDRIRLMNIGVPEEFLDYSGIVNEAKNDKSRIPELVSTILPPDEEVAEVIQDAKAKNKKVSVGPNM
KGRFRESMLWLQWLMFEREPEKVERKLSKIGQRGVCGAVWGNNDIAYRCRTCEHDPTCAICVPCFQNGNHKEHDYSIIYT
GGGCCDCGDVTAWKREGFCSRHKGAEQIQPLPEKYANSAAPVLDALFIYWENKLSLAESVGQENPRASDHVAERRKLANE
LTFAVVEMLLEFCKNSESLLSFVSKRVISVIGLLDILVRAEMFSSDVVVRKLHELLLKLLGEPIFKYEFAKVFLSYYPVF
VKDAIREHSDDTIKKYPLLSTFSVQIFTVPTLTPRLVKEMNLLEMLLGCLREIFDSCAGDDSCLQVAKWANLYETTNRVI
GDIRFVMSHAAVSKYATHEQLNISKAWMKLLTFVQGMNPQKRETGIHIREENEYMHLPLVLDHSIANIQPLLVDGAFSSA
VSEETRYDFSMYKQDIGDGDSLRHAKVGRLSQESSVCGAMGRSSLSASTLKADDVIFDAVSDVLLPHSVTWVAHECLRAM
ENWLGVDDRSVSVNDILSPNASRISGSNFVALKKTLSKIKKGKSIFSRLAGSSEVTAGIQESGDLDNATSMGKESKITIS
GERDTASWRSAGFNDSEMEGECATELDNLHVLSLCYWPDITYDVSSQDVSVHIPLHRLLSLIIQKALRRCYGESAASESA
DTGAENPLSAVSLDFFGHILGGCHPYGFSAFVMEHPLRIRVFCAQVHAGMWRRNGDAALSSCEWYRAVRWSEQGLELDLF
LLQCCAALAPADLYVNRIIERFGLSNYLSLNLERPSEYEPILVQEMLTLIIQILQERRFCGLTTAESLKRELVHRLAIGD
ATHSQLVKSLPRDLSKFDQLQEILDAVAMYSHPSGFNQGMYSLRWSYWKELDIYHPRWSSRDLQVAEERYLRFCSVSALT
AQLPRWTKIYYPLESIAGIATCKVVLQVIRAVLFYAVFTDNPTDSRAPYGVLLTALHLLALALDVCFQKKKSGDQSCDIG
GSTPILDFASEEIAEGLNNGAGKQSLLSLLVFLMGMYKKDGADNFLEAGNCNLSSVIESLLKKFAEIDSRCMTKLQQLAP
EIVSHLSQSLPRDDTSGSFSASDSEKRKAKARERQAAILEKMKAEQFKFLSSISSNIEDAPKSAPEVTNYDAEHVSEESV
QDVCALCHDPNSRTPVSYLILLQKSRLLSFVDRGSPSWDQDQWLGKECGTISANNMVNQFGTNTPSSGLGVISSCQLAQV
AEEAVNQFAYNGKPEEVNSVLEFVKAQFPSLRNIPIPFTFSNGRKCTASSMEMFEQDLYLSICREMRKNMTYPDLMKEDE
ECSVAEGGLKNRGNSDSFLLGKYVASISKEMRENASASEVSRGDRIAAESLVYDGFGPIDCDGIHLSSCGHAVHQGCLDR
YVSSLKERYNRRIIFEGGHIVDPDQGEFLCPVCRQLANSVLPALPWDLQRINEQPTVSGVGLSLDSSSSFTTREENTSFQ
LQQAVSLLQSASNVVGKADVIESFPLMKNEIMASNVEAVSRRMCKMYFQNKLDKFFGSARVNPSLIMWDALKYSLMSMEI
AARSEKTSTTPIYDVNALDKELKSSSGFVLSLLLKVVQSMRSKNSLHVLQRFRGIQLFAESICSGTSIDNPGGRCKRGGN
MLSILKHADVEVSYPDIQFWNRASDPVLARDPFSSLMWVLFCLPCQFILCKESLLSLVHVFYAVTLSQAVLSCCGKLQSK
VNELGFSDSLISDISKLLGEFGSAQEYFVSNYIDPSCDIKDMIRRLSFPYLRRCALLWKLLNSTVPPPFSDRDHVLARSS
HGISDMMDSSDDALSDLKEIQEVEKMFKIPSLDVILKDEVLRSLVLKWFHHFSKEFEVHRFQHVLYSTPAVPFKLMCLPH
LYQDLLQRYIKQCCSDCKSVLDEPALCLLCGRLCSPSWKPCCRESSCQSHAVACGAGTGVFLLIRRTTILLQRCARQAPW
PSPYLDAFGEEDIEMHRGKPLYLNEERYAALTYMVASHGLDRSSKVLSQTTIGGFGLV
> CsPRT6.3_KDO44131.1_ CISIN_1g000141mg_Citrus_sinensis
MEIDSPPDFSPPKPRDRIVRRLMNIGVPEEFLDYSGIVNFAKNDKSRIPELVSTILPPDEEVAEVIQDAKAKNKKVSVGP
NMKGRFRESMLWLQWLMFEREPEKVLRKLSKIGQRGVCGAVWGNNDIAYRCRTCEHDPTCAICVPCFQNGNHKEHDYSII
YTGGGCCDCGDVTAWKREGFCSRHKGAEQIQPLPEKYANSAAPVLDALFIYWENKLSLAESVGQENPRASDHVAERRKLA
NELTFAVVEMLLEFCKNSESLLSFVSKRVISVIGLLDILVRAEMFSSDVVVRKLHELLLKLLGEPIFKYEFAKVELSYYP
VFVKDAIREHSDDTIKKYPLLSTFSVQIFTVPTLTPRLVKEMNLLEMLLGCLREIFDSCAGDDSCLQVAKWANLYETTNR
VIGDIRFVMSHAAVSKYATHEQLNISKAWMKLLTFVQGMNPQKRETGIHIREENEYMHLPLVLDHSIANIQPLLVDGAFS
SAVSEETRYDFSMYKQDIGDGDSLRHAKVGRLSQESSVCGAMGRSSLSASTLKADDVIFDAVSDVLLPHSVTWVAHECER
AMENWLGVDDRSVSVNDILSPNASRISGSNFVALKKTLSKIKKGKSIFSRLAGSSEVTAGIQESGDLDNATSMGKESKIT
ISGERDTASWRSAGFNDSEMEGECATELDNLHVLSLCYWPDITYDVSSQDVSVHIPLHRLLSLIIQKALRRCYGESAASE
SADTGAENPLSAVSLDFFGHILGGCHPYGFSAFVMEHPLRIRVFCAQVHAGMWRRNGDAALSSCEWYRAVRWSEQGLELD
LFLLQCCAALAPADLYVNRIIERFGLSNYLSLNLERPSEYEPILVQEMETLIIQILQERRFCGLTTAESLKRELVHRLAI
GDATHSQLVKSLPRDLSKFDQLQEILDAVAMYSHPSGFNQGMYSLRWSYWKELDIYHPRWSSRDLQVAEERYLRFCSVSA
LTAQLPRWTKIYYPLESIAGIATCKVVLQVIRAVLFYAVFTDNPTDSRAPYGVLLTALHLLALALDVCFQKKKSGDQSCD
IGGSTPILDFASEEIAEGLNNGAGKQSLLSLLVFLMGMYKKDGADNFLEAGNCNLSSVIESLLKKFAEIDSRCMTKLQQL
APEIVSHLSQSLPRDDTSGSFSASDSEKRKAKARERQAAILEKMKAEQFKFLSSISSNIEDAPKSAPEVINYDAEHVSEE
SVQDVCALCHDPNSRTPVSYLILLQKSRLLSFVDRGSPSWDQDQWLGKECGTISANNMVNQFGTNTPSSGLGVISSCQLA
QVAEEAVNQFAYNGKPEEVNSVLEFVKAQFPSLRNIPIPFTFSNGRKCTASSMEMFEQDLYLSICREMRKNMTYPDLMKE
DEECSVAEGGLKNRGNSDSFLLGKYVASISKEMRENASASEVSRGDRIAAESLVYDGEGPIDCDGIHLSSCGHAVHQGCL
DRYVSSLKERYNRRIIFEGGHIVDPDQGEFLCPVCRQLANSVLPALPWDLQRINEQPTVSGVGLSLDSSSSFTTREENTS
FQLQQAVSLLQSASNVVGKADVIESFPLMKNEIMASNVEAVSRRMCKMYFQNKLDKFFGSARVNPSLIMWDALKYSLMSM
EIAARSEKTSTTPIYDVNALDKELKSSSGFVLSLLLKVVQSMRSKNSLHVLQRFRGIQLFAESICSGTSIDNPGGRCKRG
GNMLSILKHADVEVSYPDIQFWNRASDPVLARDPFSSLMWVLFCLPCQFILCKESLLSLVHVFYAVTLSQAVLSCCGKLQ
SKVNELGFSDSLISDISKLLGEFGSAQEYFVSNYIDPSCDIKDMIRRLSFPYLRRCALLWKLINSTVPPPFSDRDHVLAR
SSHGISDMMDSSDDALSDLKEIQEVEKMFKIPSLDVILKDEVLRSLVLKWFHHFSKEFEVHRFQHVLYSTPAVPFKLMCL
PHLYQDLLQRYIKQCCSDCKSVLDEPALCLLCGRLCSPSWKPCCSCQSHAVACGAGTGVFLLIRRTTILLQRCARQAPWP
SPYLDAFGEEDIEMHRGKPLYLNEERYAALTYMVASHGLDRSSKVLSQTTIGGFFLV
> CsPRT6.4_KDO44132.1_CISIN_1g000141mg_Citrus_sinensis
MEIDSPPDFSPPKPRDRIVRRLMNIGVPEEFLDYSGIVNFAKNDKSRIPELVSTILPPDEEVAEVIQDAKAKNKKVSVGP
NMKGRFRESMLWLQWLMFEREPEKVLRKLSKIGQRGVCGAVWGNNDIAYRCRTCEHDPTCAICVPCFQNGNHKEHDYSII
YTGGGCCDCGDVTAWKREGFCSRHKGAEQIQPLPEKYANSAAPVLDALFIYWENKLSLAESVGQENPRASDHVAERRKLA
NELTFAVVEMLLEFCKNSESLLSFVSKRVISVIGLLDILVRAEMFSSDVVVRKLHELLLKLLGEPIFKYEFAKVFLSYYP
VFVKDAIREHSDDTIKKYPLLSTFSVQIFTVPTLTPRLVKEMNLLEMLLGCLREIFDSCAGDDSCLQVAKWANLYETTNR
VIGDIRFVMSHAAVSKYATHEQLNISKAWMKLLTFVQGMNPQKRETGIHIREENEYMALPLVLDHSIANIQPLLVDGAFS
SAVSEETRYDFSMYKQDIGDGDSLRHAKVGRLSQESSVCGAMGRSSLSASTLKADDVIFDAVSDVLLPHSVTWVAHECLR
AMENWLGVDDRSVSVNDILSPNASRISGSNFVALKKTLSKIKKGKSIFSRLAGSSEVTAGIQESGDLDNATSMGKESKIT
ISGERDTASWRSAGFNDSEMEGECATELDNLHVLSLCYWPDITYDVSSQDVSVHIPLHRLLSLIIQKALRRCYGESAASE
SADTGAENPLSAVSLDFFGHILGGCHPYGFSAFVMEHPLRIRVFCAQVHAGMWRRNGDAALSSCEWYRAVRWSEQGLELD
LFLLQCCAALAPADLYVNRIIERFGLSNYLSLNLERPSEYEPILVQEMLTLIIQILQERRFCGLTTAESLKRELVHRLAI
GDATHSQLVKSLPRDLSKFDQLQEILDAVAMYSHPSGFNQGMYSLRWSYWKELDIYHPRWSSRDLQVAEERYLRFCSVSA
LTAQLPRWTKIYYPLESIAGIATCKVVLQVIRAVLFYAVFTDNPTDSRAPYGVLLTALHLLALALDVCFQKKKSGDQSCD
IGGSTPILDFASEEIAEGLNNGAGKQSLLSLLVGLMGMYKKDGADNFLEAGNCNLSSVIESLLKKFAEIDSRCMTKLQQL
APEIVSHLSQSLPRDDTSGSFSASDSEKRKAKARERQAAILEKMKAEQFKFLSSISSNIEDAPKSAPEVTNYDAEHVSEE
SVQDVCALCHDPNSRTPVSYLILLQKSRLLSFVDRGSPSWDQDQWLGKECGTISANNMVNQFGTNTPSSGLGVISSCQLA
QVAEEAVNQFAYNGKPEEVNSVLEFVKAQFPSLRNIPIPFTFSNGRKCTASSMEMFEQDLYLSICREMRKNMTYPDLMKE
DEECSVAEGGLKNRGNSDSFLLGKYVASISKEMRENASASEVSRGDRIAAESLVYDGFGPIDCDGIHLSSCGHAVHQGCL
DRYVSSLKERYNRRIIFEGGHIVDPDQGEFLCPVCRQLANSVLPALPWDLQRINEQPTVSGVGLSLDSSSSFTTREENTS
FQLQQAVSLLQSASNVVGKADVIESFPLMKNEIMASNVEAVSRRMCKMYFQNKLDKFFGSARVNPSLIMWDALKYSLMSM
EIAARSEKTSTTPIYDVNALDKELKSSSGFVLSLLLKVVQSMRSKNSLHVLQRFRGIQLFAESICSGTSIDNPGGRCKRG
GNMLSILKHADVEVSYPDIQFWNRASDPVLARDPFSSLMWVLFCLPCQFILCKESLLSLVHVFYAVTLSQAVLSCCGKLQ
SKVNELGFSDSLISDISKLLGEFGSAQEYFVSNYIDPSCDIKDMIRRLSFPYLRSDMMDSSDDALSDLKEIQEVEKMFKI
PSLDVILKDEVLRSLVLKWFHHFSKEFEVHRFQHVLYSTPAVPFKLMCLPHLYQDLLQRYIKQCCSDCKSVLDEPALCLL
CGRLCSPSWKPCCRESSCQSHAVACGAGTGVFLLIRRTTILLQRCARQAPWPSPYLDAFGEEDIEMHRGKPLYLNEERYA
ALTYMVASHGLDRSSKVLSQTTIGGFFLV
>CuPRT6_GAY45099.1_CUMW_086920_Citrus_unshiu
MEIDSPPDFSPPKPRDRIVRRLINIGVPEEFLDYSGIVNFAKNDKSRIPELVSTILPPDEEVAEVIQDAKAKNKKVSVGP
NMKGRFRESMLWLQWLMFEREPEKVLRKLSKIGQRGVCGAVWGNNDIAYRCRTCEHDPTCAICVPCFQNGNHKEHDYSII
YTGGGCCDCGDVTAWKREGFCSRAKGAEQIQPLPEKYANSAAPVLDALFIYWENKLSLAESVGQENPRASDHVAERRKLA
NELTFAVVEMLLEFCKNSESLLSFVSKRVISVIGLLDILVRAERFSSDVVVRKLHELLLKLLGEPIFKYEFAKVFLSYYP
VFVKDAIREHSDDTIKKYPLLSTFSVQIFTVPTLTPRLVKEMNLLEMLLGCLREIFDSCAGDDSCLQVAKWANLYETTNR
VIGDIRFVMSHAAVSKYATHEQLNISKAWMKLLTFVQGMNPQKRETGIHIREENEYMHLPLVLDHSIANIQPLLVDGAFS
SAVAEETRYDFSMYKQDIGDGDSLRHAKVGRLSQESSVCGAMGRSSLSASTLKVDDVIFDAVSDVLLPHSVTWLAHECLR
AMENWLGVDDRSVSVNDILSPNASRISGSNFVALKKTLSKIKKGKSIFSRLAGSSEVTAGIQESGDLDNATSMGKESKIT
ISGERDTASWRSAGFNDSEMEGECATELDNLHVLSLCYWPDITYDVSSQDVSVHIPLHRLLSLIIQKALRRCYGESAASE
SADTGAENPLSAVSLDFFRHILGGCHPYGFSAFVMEHPLRIRVFCAQVHAGMWRRNGDAALSSCEWYRAVRWSEQGLELD
LFLLQCCAALAPADLYVNRIIERFGLSNYLSINLERPSEYEPILVQEMLTLIIQILQERRFCGLTTAESLKRELVHRLAI
GDATHSQLVKSLPRDLSKFDQLQEILDAVAMYSHPSGFNQGMYSLRWSYWKELDIYHPRWSSRDLQVAEERYLRFCSVSA
LTAQLPRWTKIYYPLESIAGIATCKVVLQVIRVVLFYAVFTDNPTDSRAPYGVLLTALHLLALALDVCFQKKKSGDQSRD
IGGSTPILDFASEEIAEGLNNGAGKQSLLSLLVFLMGMYKKDGADNFLEAGNCNLSSVIESLLKKFAEIDSRCMTKLQQL
APEIVSHLSQSLPRDDTSGSFSASDSEKRKAKARERQAAILEKMKAEQFKFLSSISSNIEDAPKSAPEVINYDAEHVSEE
SVQDVCALCHDPNSRTPVSYLILLQKSRLLSFVDRGSPSWDQDQWLGKECGTISANNMVNQFGTNTPSSALGVISSCQLA
QVAEEAVNQFAYNGKPEEVNAVLEFVKAQFPSLRNIPIPFTFSNGRKCTASSMEMFEQDLYLSICREMRKNMTYPDLMKE
DEECSVAEGGLKNRGNSDSFLLGKYVASISKEMRENASASEVSRGDRIAAESLVYDGFGPIDCDGIHLSSCGHAVHQGCL
DRYVSSLKERYNRRIIFEGGHIVDPDQGEFLCPVCRQLANSVLPALPWDLQRINEQPTVSGVGLSLDSNSSFTTREENTS
LQLQQAVSLLQSASNVVGKADVIESFPLLKNEIMASNVEAVSRRMCKMYFQNKLDKFFGSARVNPSLIMWDALKYSLMSM
EIAARSEKTSTTPIYDVNALDKELKSSSGFVLSLLLKVVQSMRSKNSLHVLQRFRGIQLFAESICSGTSIDNPGGRCKRG
GNMLSILKHADVEVSYPDIQFWNRASDPVLARDPFSSLMWVLFCLPCQFILCKESLLSLVHVFYAVTLSQAVLSCCGKLQ
SKVNELGFSDSLISDISKLLGEFGSAQEYFVSNYIDPSCDIKDMIRRLSFPYLRRCALLWKLLNSTVPPPFSDRDHVLAR
SSHGISDMMDSSDDALSDLKEIQEVEKMFKIPSLDVILKDEVLRSLVLKWFHHFSKEFEVHRFQHVLYSTPAVPFKLMCL
PHLYQDLLQSVALIASLFLMNLLCACYVVDYVPQAGSHAAGKAVVKAMQWPVVLVLRTTILLQRCARQAPWPSPYLDAFG
EEDIEMHRGKPLYLNEERYAALTYMVASHGLDRSSKVLSQTTIGGFFLGKKTVLRNPKIAKLEVFNFMENKLRILDAGRL
ETNSFVAFWLRRAFALAYIYIYTPKALQIFISLNLNEFGVHRRSAAGKYTSSAPLNGCTVICKA
> CsPRT6.5_KDO44134.1_ CISIN_1g000141mg_Citrus_sinensis
MEIDSPPDFSPPKPRDRIVRRLMNIGVPEEFLDYSGIVNFAKNDKSRIPELVSTILPPDEEVAEVIQDAKAKNKKVSVGP
NMKGRFRESMLWLQWLMFEREPEKVLRKLSKIGQRGVCGAVWGNNDIAYRCRTCEHDPTCAICVPCFQNGNHKEHDYSII
YTGGGCCDCGDVTAWKREGFCSRHKGAEQIQPLPEKYANSAAPVLDALFIYWENKLSLAESVGQENPRASDHVAERRKLA
NELTFAVVEMLLEFCKNSESLLSFVSKRVISVIGLLDILVRAEMFSSDVVVRKLHELLLKLLGEPIFKYEFAKVFLSYYP
VFVKDAIREHSDDTIKKYPLLSTFSVQIFTVPTLTPRLVKEMNLLEMLLGCLREIFDSCAGDDSCLQVAKWANLYETTNR
VIGDIRFVMSHAAVSKYATHEQLNISKAWMKLLTFVQGMNPQKRETGIHIREENEYMHLPLVLDHSIANIQPLLVDGAFS
SAVSEETRYDFSMYKQDIGDGDSLRHAKVGRLSQESSVCGAMGRSSLSASTLKADDVIFDAVSDVLLPHSVTWVAHECLR
AMENWLGVDDRSVSVNDILSPNASRISGSNFVALKKTLSKIKKGKSIFSRLAGSSEVTAGIQESGDLDNATSMGKESKIT
ISGERDTASWRSAGGNDSEMEGECATELDNLHVLSLCYWPDITYDVSSQDVSVHIPLHRLLSLIIQKALRRCYGESAASE
SADTGAENPLSAVSLDFFGHILGGCHPYGFSAFVMEHPLRIRVFCAQVHAGMWRRNGDAALSSCEWYRAVRWSEQGLELD
LFLLQCCAALAPADLYVNRIIERFGLSNYLSLNLERPSEYEPILVQEMLTLIIQILQERRFCGLITAESLKRELVARLAI
GDATHSQLVKSLPRDLSKFDQLQEILDAVAMYSHPSGFNQGMYSLRWSYWKELDIYHPRWSSRDLQVAEERYLRFCSVSA
LTAQLPRWTKIYYPLESIAGIATCKVVLQVIRAVLFYAVFTDNPTDSRAPYGVLLTALHLLALALDVCFQKKKSGDQSCD
IGGSTPILDFASEEIAEGLNNGAGKQSLLSLLVFLMGMYKKDGADNFLEAGNCNESSVIESLLKKFAEIDSRCMTKLQQL
APEIVSHLSQSLPRDDTSGSFSASDSEKRKAKARERQAAILEKMKAEQFKFLSSISSNIEDAPKSAPEVTNYDAEHVSEE
SVQDVCALCHDPNSRTPVSYLILLQKSRLLSFVDRGSPSWDQDQWLGKECGTISANNMVNQFGTNTPSSGLGVISSCQLA
QVAEEAVNQFAYNGKPEEVNSVLEFVKAQFPSIRNIPIPFTFSNGRKCTASSMEMFEQDLYLSICREMRKNMTYPDLMKE
DEECSVAEGGLKNRGNSDSFLLGKYVASISKEMRENASASEVSRGDRIAAESLVYDGFGPIDCDGIHLSSCGHAVHQGCL
DRYVSSLKERYNRRIIFEGGHIVDPDQGEFLCPVCRQLANSVLPALPWDLQRINEQPTVSGVGLSLDSSSSFTTREENTS
FQLQQAVSLLQSASNVVGKADVIESFPLMKNEIMASNVEAVSRRMCKMYFQNKLDKFFGSARVNPSLIMWDALKYSLMSM
EIAARSEKTSTTPIYDVNALDKELKSSSGFVLSLLLKVVQSMRSKNSLHVLQRFRGIQLFAESICSGTSIDNPGGRCKRG
GNMLSILKHADVEVSYPDIQFWNRASDPVLARDPFSSLMWVLFCLPCQFILCKESLLSLVHVFYAVTLSQAVLSCCGKLQ
SKVNELGFSDSLISDISKLLGEFGSAQEYFVSNYIDPSCDIKDMIRRLSFPYLRRCALLWKLLNSTVPPPFSDRDHVLAR
SSHGISDMMDSSDDALSDLKEIQEVEKMFKIPSLDVILKDEVLRSLVLKWFHHFSKEFEVHRFQHVLYSTPAVPFKLMCL
PHLYQDLLQRYIKQCCSDCKSVLDEPALCLLCGRLCSPSWKPCCRESSCQSHAVACGAGTGVFLLIRVFSAPSFNRKINI
IVLCVYCCACCLLA
> CsPRT6.6_KDO44135.1_CISIN_1g000141mg_Citrus_sinensis
MEIDSPPDFSPPKPRDRIVRRLMNIGVPEEFLDYSGIVNFAKNDKSRIPELVSTILPPDEEVAEVIQDAKAKNKKVSVGP
NMKGRFRESMLWLQWLMFEREPEKVLRKLSKIGQRGVCGAVWGNNDIAYRCRTCEHDPTCAICVPCFQNGNHKEHDYSII
YTGGGCCDCGDVTAWKREGFCSRHKGAEQIQPLPEKYANSAAPVLDALFIYWENKLSLAESVGQENPRASDHVAERRKLA
NELTEAVVEMLLEFCKNSESLLSFVSKRVISVIGLLDILVRAEMFSSDVVVRKLHELLLKLLGEPIFKYEFAKVFLSYYP
VFVKDAIREHSDDTIKKYPLLSTFSVQIFTVPTLTPRLVKEMNLLEMLLGCLREIFDSCAGDDSCLQVAKWANLYETTNR
VIGDIRFVMSHAAVSKYATHEQLNISKAWMKLLTFVQGMNPQKRETGIHIREENEYMHLPLVLDHSIANIQPLLVDGAFS
SAVSEETRYDFSMYKQDIGDGDSLRHAKVGRLSQESSVCGAMGRSSLSASTLKADDVIFDAVSDVLLPHSVTWVAHECER
AMENWLGVDDRSVSVNDILSPNASRISGSNFVALKKTLSKIKKGKSIFSRLAGSSEVTAGIQESGDLDNATSMGKESKIT
ISGERDTASWRSAGFNDSEMEGECATELDNLHVLSLCYWPDITYDVSSQDVSVHIPLHRLLSLIIQKALRRCYGESAASE
SADTGAENPLSAVSLDFFGHILGGCHPYGFSAFVMEHPLRIRVFCAQVHAGMWRRNGDAALSSCEWYRAVRWSEQGLELD
LFLLQCCAALAPADLYVNRIIERFGLSNYLSLNLERPSEYEPILVQEMLTLIIQILQERRFCGLTTAESLKRELVHRLAI
GDATHSQLVKSLPRDLSKFDQLQEILDAVAMYSHPSGFNQGMYSLRWSYWKELDIYHPRWSSRDLQVAEERYLRFCSVSA
LTAQLPRWTKIYYPLESIAGIATCKVVLQVIRAVLFYAVFTDNPTDSRAPYGVLLTALHLLALALDVCFQKKKSGDQSCD
IGGSTPILDFASEEIAEGLNNGAGKQSLLSLLVFLMGMYKKDGADNFLEAGNCNESSVIESLLKKFAEIDSRCMTKLQQL
APEIVSHLSQSLPRDDTSGSFSASDSEKRKAKARERQAAILEKMKAEQFKFLSSISSNIEDAPKSAPEVTNYDAEHVSEE
SVQDVCALCHDPNSRTPVSYLILLQKSRLLSFVDRGSPSWDQDQWLGKECGTISANNMVNQFGTNTPSSGLGVISSCQLA
QVAEEAVNQFAYNGKPEEVNSVLEFVKAQFPSLRNIPIPFTFSNGRKCTASSMEMFEQDLYLSICREMRKNMTYPDLMKE
DEECSVAEGGLKNRGNSDSFLLGKYVASISKEMRENASASEVSRGDRIAAESLVYDGFGPIDCDGIHLSSCGHAVHQGCL
DRYVSSLKERYNRRIIFEGGHIVDPDQGEFLCPVCRQLANSVLPALPWDLQRINEQPTVSGVGLSLDSSSSFTTREENTS
FQLQQAVSLLQSASNVVGKADVIESFPLMKNEIMASNVEAVSRRMCKMYFQNKLDKFFGSARVNPSLIMWDALKYSLMSM
EIAARSEKTSTTPIYDVNALDKELKSSSGFVLSLLLKVVQSMRSKNSLHVLQRFRGIQLFAESICSGTSIDNPGGRCKRG
GNMLSILKHADVEVSYPDIQFWNRASDPVLARDPFSSLMWVLFCLPCQFILCKESLLSLVHVFYAVTLSQYYHVVGNFNP
RLMS
> CsPRT6.7_KDO44136.1_CISIN_1g000141mg_Citrus_sinensis
MEIDSPPDFSPPKPRDRIVRRLMNIGVPEEFLDYSGIVNFAKNDKSRIPELVSTILPPDEEVAEVIQDAKAKNKKVSVGP
NMKGRFRESMLWLQWLMFEREPEKVLRKLSKIGQRGVCGAVWGNNDIAYRCRTCEHDPTCAICVPCFQNGNHKEHDYSII
YTGGGCCDCGDVTAWKREGFCSRHKGAEQIQPLPEKYANSAAPVLDALFIYWENKLSLAESVGQENPRASDHVAERRKLA
NELTFAVVEMLLEFCKNSESLLSFVSKRVISVIGLLDILVRAEMFSSDVVVRKLHELLLKLLGEPIFKYEFAKVFLSYYP
VFVKDAIREHSDDTIKKYPLLSTFSVQIFTVPTLTPRLVKEMNLLEMLLGCLREIFDSCAGDDSCLQVAKWANLYETTNR
VIGDIRFVMSHAAVSKYATHEQLNISKAWMKLLTFVQGMNPQKRETGIHIREENEYMHLPLVLDHSIANIQPLLVDGAFS
SAVSEETRYDFSMYKQDIGDGDSLRHAKVGRLSQESSVCGAMGRSSLSASTLKADDVIFDAVSDVLLPHSVTWVAHECLR
AMENWLGVDDRSVSVNDILSPNASRISGSNFVALKKTLSKIKKGKSIFSRLAGSSEVTAGIQESGDLDNATSMGKESKIT
ISGERDTASWRSAGFNDSEMEGECATELDNLHVLSLCYWPDITYDVSSQDVSVHIPLHRLLSLIIQKALRRCYGESAASE
SADTGAENPLSAVSLDFFGHILGGCHPYGFSAFVMEHPLRIRVFCAQVHAGMWRRNGDAALSSCEWYRAVRWSEQGLELD
LFLLQCCAALAPADLYVNRIIERFGLSNYLSLNLERPSEYEPILVQEMLTLIIQILQERRFCGLTTAESLKRELVHRLAI
GDATHSQLVKSLPRDLSKFDQLQEILDAVAMYSHPSGFNQGMYSLRWSYWKELDIYHPRWSSRDLQVAEERYLRFCSVSA
LTAQLPRWTKIYYPLESIAGIATCKVVLQVIRAVLFYAVFTDNPTDSRAPYGVLLTALHLLALALDVCFQKKKSGDQSCD
IGGSTPILDFASEEIAEGLNNGAGKQSLLSLLVFLMGMYKKDGADNFLEAGNCNISSVIESLLKKFAEIDSRCMTKLQQL
APEIVSHLSQSLPRDDTSGSFSASDSEKRKAKARERQAAILEKMKAEQFKFLSSISSNIEDAPKSAPEVTNYDAEHVSEE
SVQDVCALCHDPNSRTPVSYLILLQKSRLLSFVDRGSPSWDQDQWLGKECGTISANNMVNQFGTNTPSSGLGVISSCQLA
QVAEEAVNQFAYNGKPEEVNSVLEFVKAQFPSEMRKNMTYPDLMKEDEECSVAEGGLKNRGNSDSFLLGKYVASISKEMR
ENASASEVSRGDRIAAESLVYDGEGPIDCDGIHLSSCGHAVHQGCLDRYVSSLKERQVLPTTKGNLILLENATDLLITWL
FSISQDDLLENVDKVLEWAILTGFALLCFLFESFHYIMQLHL
>AbPRT6_sb36751.5_Atalantia_buzifolia
MEIDPPPDFSPPKPRDRIVRRLINIGVLEEFLDYSGIVNFAKNDRSRIPE
LVSTILPPDEEVAEVIQDAKAKNKKISVGLNMKGRFRESMLWLQWLMFER
EPEKVLRKLSKIGQRGVCGAVWGNNDIAYRCRTCEHDPTCAICVPCFQNG
NHKEHDYSIIYTGGGCCDCGDVTAWKREGFCSRHKGAEQIQPLPEKYANS
AVPVLDALFIYWENKLSSAESVGQENPRASDHVAERRKLANELTFAVVEM
LLEFCKNSESLLSFVSKRVISVVGLLDILVRAERFLNDVVVRKLHELLLK
LLGEPIFKYEFAKVFLSYYPVFVKDAIREHSDDTIKKYPLLSTFSVQIFT
VPTLTPRLVKEMNLLEMLLECLREIFDSCAGDNSCLQVAKGANLYETTNR
VIGDIRFVMSHAAVSKYATHEQLDISKTWLKLLTFVQGMNPQKRETGIPI
REETEYMHLPLVLDHSIANIQPLLVDGAFSSAVAEETCYDFSMYKQDIGD
GDSLRHAKVGRLSQESSVCGAMGRSSLSASTLKADDVIVDAISDVLLPHS
VTWIAHECLRAMENWLGVNDRSVSVNDIVSPNASRISGSNFVALKKTLSK
IKKGKSIFSRLAGSSEVTAGIQESGDLDNATSMGKESKITISGERDTASW
RSAGFNDSQMEGECATELDNLHVLSLCYWPDIMYDVSSQDVSVHIPLHRL
LSLIIQKALRRCYGESAASESADTGAENPLSAVSLDFFGHVLGGCHPYGF
SAFVMEHPLRIRVFCAQVHAGMWRRNGDAALSSCEWYRAVRWSEQGLELD
LFLLQCCAALAPADQYVNRIIERFGLSNYLSLNLERPSEYEPILVQEMET
LIIQILQERRFCGLTTAESLKRELVHRLAIGDTTHSQLVKSLPRDLSKFD
RLQEILDAVAMYSHPSGFNQGMYSLRWSYWKELDIYHPRWSSRDLQVAEE
RYLRFCSVSALTSQLPRWTKIYFPLESIAGIATCKVVLQVIHAVLFYAVF
TDKPTDSRAPYGALLTALHLLALALDVCFQKKKSGDQSCDIGGSTPILDF
ASDEIAEGLNNGAGKQSLLSLLVFLMGMYKKDGAANFLEAGNCNISSLIE
SLLKKFAEIDSRCMTKLQQLAPEIVSHLSQSLPRDDTSGSFSASDSEKRK
AKARERQAAILEKMKAEQFKFLSSISSNIEDAPKSAPEVTNYDAEHVSEE
SVQDVCALCHDPNSRTPVSYLILLQKSRLLSFVDRGSPSWDQDQWLGKEC
GAISANNMVNQFGTNTPSSGLGVISSCQLAQVAEEAVNQFAYNGKPEEVN
AVLEFVKAQFPSLRNIQIPFTFSNGRKCTASSMEVFEQDLYLSICREMRK
NMTCPDLMKEDEECSVAEGGLKNRGNSDSVLLGKYVASISKEMRENPSAS
EVSHGDRIAAESLVYDGFGPIDCDGIHLSSCGHAVHQGCLDRYVSSLKER
YNRRIIFEGGHIVDPDQGEFLCPVCRQLANSVLPALPWDLQRINEQPTLS
GVGLSLDSNSSFTPREENTSLQLQQAASLLQSASNVVGKADVIESFPLMK
NEIMASNVEGVSRRMCKMYFQNKLDKFFGSARVNPSLIMWDALKYSLMSM
EIAARSEKTSMTPIYDVNALDKELKSSSGFVLSLLLKVVQSMRSKNSLHV
LQRFRGIQLFAESICSGTSIDNPGGRCKRGGNMLSILKHADVEVSYPDIQ
FWNRASDPVLARDPFSSLMWVLFCLPCQFILCKESLLSLVHVFYAVTLSQ
AVLSCCGKLQSKVNELGFSDSLISDISKLLGEFGSAQEYFVSNYIDPSCD
IKDMIRRLSFPYLRRCALLWKLLNSTVPPPFSDRDHVLARSSHGISDMMD
SSDDALSDLKEIQEVEKMFKIPSLDVILKDKVLRSLVLKWFHHFFKEFEV
HRSQRVLYSTPAVPFKLLREPHLYQDLLQRYIKQCCPDCNSVLDEPALCL
LCGRLCSPSWKPCCRESSCQSHAMACGAGTGVFLLIRRTTILLQRCARQA
PWPSPYLDAFGEEDIEMHRGKPLYLNEERYAALTYMVASHGLDRSSKVLS
QTTIGGFFLV-
>SlPRT6_Solyc10g084760.1 sequence match in blast db Tomato Genome protein
sequences (ITAG release 2.40)
MDTGSSPESDTLTPMERILKRLDILGVPAEYLELLQPGLVAYVKNNKSQIAELVPALFPTNEEAVEIIAEQQIQSPRSMV
SSSVNVKDLFQESMEWIQWLMFDGEPSRALEQLEDTGQRGVCGAVWGNNDIAYRCRTCEHDPTCAICVPCFQNGNHKDHD
YSIIYTGGGCCDCGDVTAWKREGFCSKHKGAEQIQPLPEEFANSMGPVLDLLLSCWRKRFLFPDSISGRNPRKNDHSTEL
KMVTDELTSAVVKMLLKFCKHSESLLSFISRRVSSSAGLLDILVRAERFMIIEENVKKIHELLLKLLGEPQFKYEFAKVE
LSYYPTVVNEATSECNDSVYNKYPLLSTFSVQIFTVPTLTPRLVKEMNLLPMLLGCLGDIFASCAGEDGKLQVMKWSNLY
ETTLRVVEDIRFVMSHSVVPRYVTHERRDILRTWMKLLAFVQGANPQKRETGIHVEEENENMHLPFVLGHSIANIHSLLV
SGAFSTSSTEDGADAFFNTHREDFEDQDSQRHAKVGRLSQESSVCSMAGRSPLEHASRVLEVHYDSSPISSSVLCLTFEC
LRAIENWLIVDNTSGPLLHILCPKTSSTPGNNFSVLKKTLSKFRRGREMFKSQSPPSNDVRLVTSAEGYNKQYSNPSLNG
RTILDSGLGSGQEPACLGGHDDSMLEGDNASELGELRLLSLSDWPDIVYKVSLQDISVHNPLQRLLSMVLQKALGKCYGE
NAQPVASSAKLSSSVHYDFFGHILGVYHPQGFSAFIMEHALRIRVFCAQVYAGMWRRNGDSAILSCEWYRSVRWSEQGLE
LDLFLLQCCAALAPADLYISRILERFELSNYLSFNLERPSEYEPALVQEMLTLIIQILKERRFCGLTSSECLQRELVYRL
SIGDATHSQLVKSLPRDLSKIDKFQEVLDKIAIYSNPSGMNQGMYKLRLPYWKELDLYHPRWNSRDLQVAEERYMRFCNA
SALTTQLPGWSKIYPPLGRIAEVATCRTVLQIVRAVVSYAVFSDASNASCAPDGVLLRALHLLSLALDICHAHRESGEHS
CSNGDVIPILALACEEISVGKFGDQSLLSLLVLLMRKHKKENYFVEAGMLNLLSLVESVLKKFAELQPECMKKLQDLAPD
VVNQLSRSFPAGDMNSFKSVSDSDKHKAKARERQAAMLEKMRVQQSKFLASIDSKTDVAADDSKHGKDLCDSDGRPRSEE
ATPVICSLCRDPNSRSPVSYLILLQKSRLLSCTNRGPPSWEQTRRPGKEPTSCAKHVPNISSERSNLSRSSEITSSSCLM
QLIQNKVNEFALEGQPKEVEAFLEYIKEKFPSMKNIQPSCASSTVKKKTSSSFEMLEEHMYSLIWEEMDANSWNWDLLKN
DRKLSALGDNGSAESLLLGRYISALSRECSPSASTNSRKAQLESSMLLPTYNGFGPSDCDGIYLSSCGHAVHQGCLDRYL
SSLKERYTRQIVFEGGHIVDPDQGEFLCPVCRGLANSVLPALPAETKRSTPSLSTDPSDAVGLPTLRFQEVLFLLQSAAD
VAGSREILQSLPVQQFGQMRVNLDYVVRILCEMYFPDKDKISESGRLSHSLILFDTLKYSLISTEIAARSGNTSLAPNYS
LGALYKELKSTNCFILALLLSIVQSTRSKDSLTVLLRLRGIQLFVKSICSDISADEYPDSPIVGGNMQDILEFSETELQY
PDIQFWKRCSDPVLAHDAFSSLTWVLYCLPCQFLSCEKSFLCLVALFYVVTITQIVITYSRKLQSSLSMSGCSDSLVTDI
YRIIAENGVAYKDFDSNHIETHDVKDAIRSLSFPYLRRCALLWKLVRSSVSAPFSGGSNILDGLPYSMGETMECGGNIPV
EFNEIEKLEKLFKIPPLDDVISDETVRFVVPSWLRRFSKQFEARMLNGAMYSSPAVPFKLMLLPHLYQDLLQRYIKQNCP
DCGVVLEEPALCLLCGRLCSPNWKPCCRESGCQTHAMACGAGTGVFLLIKKTTVLLQRSARQASWPSPYLDAFGEEDSGM
NRGKPLYLNEERYAALTHMVASHGLDRSPKVLHQTNIGNFFVL
>StPRT6_XP_006339028.1 PREDICTED: E3 ubiquitin-protein ligase PRT6-like isoform
X3 [Solanum tuberosum]
METDSSPESDTLTPMERILQRLDILGVPAENLEQLQPGLVAYVKNNKSQIAELVPALLPTNEEAMEIITE
QQMESPRSTVSSSVNVKDLFQESMDWIQWLMFDGEPSRALEQLEDTGERGVCGAVWGNNDIAYRCRTCEH
DPTCAICVPCFQNGNHKDHDYSIIYTGGGCCDCGDVTAWKREGFCSKHKGAEQIKPLPEEFANSMGPVLD
LLLSCWRKRLLFPDSISGRNPRRNDHATELKMVTDELTSAVVEMLLKFCKHSESLLSFISRRVSCSAGLL
DILVRAERFMITEENVKKIHELLLKLLGEPQFKYEFAKVFLSYYPTVVNEATRECNDSVFNKYPLLSTFS
VQIFTVPTLTPRLVKEMNLLPMLLGCLGDIFASCAGEDGKLQVMKWSDLYETTLRVVEDIRFVMSHSVVP
RYATHDRRDILRTWIKLLAFVQGTDPQKRETGIHVEEESENMHLPFVLGHSIANIHSLLVGGAFSISTED
AADAFFNTHTEDFEDQDSQRHAKVGRLSQESSVCSMAGRSPLEHASRVPEVTYDSSPISSSVLCETFECL
RAIENWLIVDNTSGALLHILCPKTSSTPGNNFSMLKKTLSKFRRGREMFKSQSPPSNEVRLLTSAEGYNK
QYSNPSINGRTTLDSGQGSGQEAACLGGLDDSMLEGDNASELEALRLESLSDWPDIVYKVSLQDISVHNP
LHRLLSMVLQRALGKCYGESAQPVASSAKLSSSVHYDFFGHILGGYHPQGFSAFIMEHALRIRVFCAQVH
AGMWRRNGDAAILSCEWYRSVRWSEQGLELDLFLLQCCAALAPADLYISRILERFELSNYLLFNLERPSE
YEPTLVQEMLTLIIQILRERRFCGLISSECLQRELVYRLSIGDATHSQLVKSLPRDLSKIDKFQEVLDKI
AIYSNPSGMNQGMYKLRLPYWKELDLYHPRWNSRDVQVAEERYMRFCNASALTTQLPGWSKIYPPLGRIA
EVATCRTVLQIVRAVVSYAVFSDASNASRAPDGVLLRALALLSLALDICHAQRESGEHSCYNGDVIPILA
LACEEISVGKFGDQSLLSLLVLLMRKHKKENYFVEAGMLNLLSLVESVEKKFAELQPECMKKLQDLAPDV
VNQLSRSFPSGDMNSFRSFSDSDKHKAKARERQAAMLEKMRVQQSKFLASIDSTTDVAADDSKHGKDLCD
SDGRPRSEEATPVICSLCRDPNSRSPVSHLVLLQKSRLLSCTNRGPPSWEQTRRPGKEPTSCAKQVPNIS
SERSNLSRSSEITSSSWLMQLIQNKVNEFALEGQPKEVEAFLEYIKEKFPLMKNIQPSCASSTVKKKTSS
SFEMLEEHMYSLIWEEMDANSRNWDLLKNDRKLSALGDNGSAESLLLGRYISALSRECSPSASTNSRKAQ
LESSMLLPTYKGFGPSDCDGIYLSSCGHAVHQGCLDRYLSSLKERYTRQIVFEGGHIVDPDQGEFLCPVC
RGLANSVLPALPAETKRSTPSLSTGPSDAVGLSTLRFQEALFLLQSAADVAGSREILQSLPLQQFGQMRV
NLDYVVRVLCEMYFPDKDKISESGRLSHSLILFDTLKYSLMSTEIAARSGNTSLAPNYSLGALYKELKST
NCFIFALLLSIVQSTRTKDSLTVLLRLRGIQLFVKSICSDISADECPDSPIVGGNMQDILEFSETELQYP
DIQFWKRSSDPVLAHDAFSSLMWVLYCLPCQFLSCEKSFLCLVHLFYVVSITQIVITYSRKRQSSLSMSG
CSDSLVTDIYRIIEENGVAYIYFDSNHIETHDVKDAIRSLSFPYLRRCALLWKLVRSSVSAPFSGGSNIL
DGLPYSMGETMECGGNIPVEFNEIEKLEKLFKIPPLDDVISDEIVRFVVPRWLRHFSKQFEARTINGVMY
STPAVPFKLMLLPHLYQDLLQRYIKQHCPDCGVVLEEPALCLLCGRLCSPNWKPCCRESGCQTHAMACGA
GTGVFLLIKKTTVLLQRSARQASWPSPYLDAFGEEDSGMNRGKPLYLNEERYAALTHMVASHGLDRSPKV
LHQTNIGNFLML
OPT1 protein Sequences
> PtOPT1_Ptrif.0001s1849.1_Poncirus_trifoliata
MTLYDHDGSVPQSEYSDRHLRISGDGEVNDNPIEEVRLTVPITDDPSLPV
LTFRTWVLGILSCGLLAFLNRFFGFRQNQLTVSSVSAQILVLPIGKLMAA
TLPTKKFKCPITNWSFSFNPGPFNIKEHVLITIFASCGASGVYAVHIIAM
LRAFYKRSIHPVAALLLALTTQMLGYGWAGIFRRYLVDSPYMWWPANLVQ
VSLFRALHEKEKRPKGGLTRIQFFFVVFVSSFAYYIVPGYLFPSLTALSF
VCWIWKRSVTAQQIGSGLSGLGIGAIGIDWSTVSSFLGSPLATPLFAIVN
TLVGFALVMYILLPISYWNNVYEAKRFPIFGAATFDAQGRKYNVDRVINK
ETFDLNVEAYNGYSKLYLSVFFAFTYGLSFATLTASISHVALFDGKSIME
MWMKTKDAVGDKFADVHTRMMKSNYDSVPGWWFHAVLVVSVALALYACEG
FGKVLQLPWWGLLLACLIALGFTLPIGIINATTNQQPGLNVITELIIGFL
YPGKPVANVVFKTYGYISMAQALAFLSDFKLGHYMKIPPKSMFIVQLVGT
VVASSVYFVTAWWLLGSIKDICDTAALPEGSPWTCPGDDVFYSASIIWGV
IGPGKMFTKEGIYPEMNWCFLIGFLAPVPVWLLSRKFPKKRWIKQIHMPI
IIGTASSMPTAKAVHFNTWGVVGIFFNYYIYRKYKAWWARHTYILSGALD
AGIAFMGVVIYFALQNYDNFGPNWWGLDSGDHCPLAKCPTAPGVKSKGCP
VQ-
> PtOPT1.2_ Ptrif.0001s1850.2_ Poncirus_trifoliata
MGSRNFEEDGVPQALSLEKPQTEIKIIGDEEVNDSPIEQVRLTVPITDDP
SQPALTFRTWFLGIVSCVVLSFLNRFFGFRQNQLSVGSISAQIIVLPLGK
LMAATLPSKPIRLPFTKWTFSMNPGPFNLKEHVLITIFANCGAGGVYAVY
IITIVKAFYKRKINPLAAMLLAQTTQLLGYGWAGLFRKYLVDSPFMWWPA
NLVQVSLFRALHEKEKRPKGGLTRLQFFFMVFASSFAYYVVPGYLFPTLS
ALSFVCWIWKNSVTAQQIGAGLNGLGIGSFGLDWSTVASFLGSPLASPVF
AIINVLAGFILNLYVLVPIAYWINTYEAKRFPIFSSHTFDSTGQPYNISR
ILNEATFDLDHDAFNSYSKLYLSPFFAFNYGLSFATLTATISHVALFDGS
DIWQMWKRTTSAARDKFADVHTRLMKKHYEAVPQWWFHIILVATVALSIY
ACEGFDKQLQLPWWGILLACAIALFFTLPIGIIQATTNQQPGLNVITELI
IGYMYPGRPLANVAFKTYGYISMSQALSFLADFKLGHYMKIPPKSMFLVQ
LIGTVVASSVYFGTAWWLLTSVEHICDPSALPEGSPWTCPGDDVFYSASI
IWGIVGPGKMFTKEGVYPALNWFFLVGLLAPVPIWFLSRKFPEIKWIGLI
HIPIIFGGTGNMPPARAVHYLSWAAVGIFFNYYVYRRFKGWWARHTYILS
AALDAGVAFMGVFLFLTLQSYDIFGPHWWGLDSTDHCPLATCPTAPGIVI
KGCPVF-
> PtOPT1.3_ Ptrif.0001s1852.1_ Poncirus_trifoliata
MCASHSAMSFTFQFKDRDMGTYVEGGMLQSMSPENSQTDTRTKGDMEEAN
DNPIEEVRLTVPITDDPTIPALTFRTWVLGLTSCCLLAFVNQFFGYRQNQ
LYMSSISAQILVLPIGKLMAATLPSKPIPVPLTPWSFSLNPGPFNLKEHV
LITIFAGCGSSGVYAVSIITIVKAFYKRSLHILPAMMLVQTTQLLGYGWA
GLFRKYLVDSPYMWWPANLVQVSLFRALHEEEKRTKGGLTRLQFFVIVFI
SSFAYYVVPGYLFPSISALSFVCWIWKDSVTAQKLGSGLQGLGMGSFGLD
WATVAGFLGSPLATPFFAIANILVGFFLFLYILIPIAYWCNAFEAQRFPL
FSSHTFDYGGQIYNVSRILNEKEFSFDREGYDNYSRLYLSVLFAFIYGLG
FATLMASISHVALFEGKTIWQMWRKTTAAVKQQFGDVHTRLMKKNYEAVP
QWWFHAILIITFALSLFTCEGFDKQFQLPWWGLLLACAMAFFFTLPVGVI
QATTNLQPGLNIITEMVIGYMYPGKPLANVAFKTYGYISMVQALGFLGDF
KLGHYMKVPPKSMFVVQLVGTIVASTVYFGTAWWLLTSVEHICNPSLLPE
GSPWTCPGDEVFYNASIIWGVVGPLRMFTNYGNYPQMNWFFLIGFLAPFP
VWLLSRKFPEKKWIKNIHMPLLLAGPGGLPSAKAVNYLSWGAVGIFFNYY
VYRRFKGWWARHTYILSAALDAGVAFMGVFLYFTLQSQDIFGPEWWGLAA
TDHCPLAKCPIAPGIKVQGCRVA-
>XP_006452632.2 oligopeptide transporter 1 [Citrus clementina]
MTLYDHDGSVPQSEYSDRHLRISGDGEVNDNPIEEVRLTVPITDDPSLPVLTFRTWVLGILSCGLLAFLNRFFGFRQNQL
TVSSVSAQILVLPIGKLMAATLPTKKFKCPITNWSFSFNPGPFNIKEHVLITIFASCGASGVYAVHIIAMLRAFYKRSIH
PVAALLLALTTQMLGYGWAGLFRRYLVDSPYMWWPANLVQVSLFRALHEKEKRPKGGLTRIQFFFVVFVSSFAYYIVPGY
LEPSLTALSFVCWIWKRSVTAQQIGSGLSGLGIGAIGIDWSTVSSFLGSPLATPLFSIVNTLVGFALVMYILLPIFYWNN
VYEAKRFPIFSAATFDAQGHKYNVDRVLNKETFDLNVEAYNGYSKLYLSVFFAFIYGLSFATLTASISHVALFDGKNIME
MWMKTKDAVGDKFADVHTRMMKRNYDSVPGWWFHAVLVVSVALALYACEGFGKVLQLPWWGLLLACLIALGFTLPIGIIN
ATTNQQPGLNVITELIIGFLYPGKPVANVVFKTYGYISMAQALAFLSDFKLGHYMKIPPKSMFIVQLVGTVVASSVYFGT
AWWLLGSIKDICDPAALPEGSPWTCPGDDVFYSASIIWGVIGPGKMFTKEGIYPEMNWCFLIGFLAPVPVWLLSRKFPKK
RWIKQIHMPIIIGTASSMPTAKAVHFNTWGVVGIFFNYYIYRKYKAWWARHTYILSGALDAGIAFMGVVIYFALQNYDNF
GPNWWGLDSGDHCPLAKCPTAPGVKSKGCPVR
>GAY47750.1 hypothetical protein CUMW_106760 [Citrus unshiu]
MGSRNFEEDGVPQALSLEKPQTEIKIIGDEEVNDSPIEQVRLTVPITDDPSQPALTFRTWFLGIISCVVLSFLNRFFGFR
QNQLSVGSISAQIIVLPLGKLMAATLPSKPIRVPLTKWTFSMNPGPFNLKEHVLITIFANCGAGGVYAVYIITIVKAFYK
RKLNPLAAMLLAQTTQLLGYGWAGLFRKYLVDSPFMALHEKEKRPKGGLTRLQFFFMVFASSFAYYVVPGYLFPTISALS
FVCWIWKNSVTAQQIGAGLNGLGIGSFGLDWSTVASFLGSPLASPVFAIINVLAGFILNLYVLVPIAYWTNTYEAKRFPI
FSSHTFDSTGQPYNISRILNEATFDLDHDAFNSYSKLYLSPFFAFNYGLSFATLTATISHVALFDGSDIWQMWKRTTSAA
RDKFADVHTRLMKKHYEAVPQCYNSHGGEFYWLVLLLYSLPYLLQPGLNVITELIIGYMYPGRPLANVAFKTYGYISMSQ
ALSFLADFKLGHYMKIPPKSMFLVQLIGTVVASSVYFGTAWWLLTSVEHICDPSALPEGSPWTCPGDDVFYSASIIWGIV
GPGKMFTKEGVYPALNWFFLVGLLAPVPIWFLSRKFPEIKWIRLIHIPIIFGGTGNMPPARAVHYLSWAAVGIFFNYYVY
RRFKGWWARHTYILSAALDAGVAFMGVFLFLTLQSYDIFGPHWWGDGEVNDNPIEEVRLTVPITDDPSLPVLTFRTWVLG
ILSCGLLAFLNRFFGFRQNQLTVSSVSAQILVLPIGKLMAATLPTKKFKCPITNWSFSFNPGPFNIKEHVLITIFASCGA
SGVYAVHIIAMLRAFYKRSIHPVAALLLALTTQMLGYGWAGIFRRYLVDSPYMWWPANLVQVSLFRALHEKEKRPKGGLT
RIQFFFVVFVSSFAYYIVPGYLFPSLTALSFVCWIWKRSVTAQQIGSGLSGLGIGAIGIDWSTVSSFLGSPLATPLFAIV
NTLVGFALVMYILLPIFYWNNVYEAKRFPIFGAATFDAQGHKYNVDRVLNKETFDLNVEAYNGYSKLYLSVFFAFTYGLS
FATLTASISHVALFDGKSIMEMWMKTKDAVGDKFADVHTRMMRRNYDSVPGWWFHAVLVVSVALALYACEGFGKVLQLPW
WGLLLACLIALGFTLPIGIINATTNQQPGKNVITELIIGFLYPGKPVANVVFKTYGYISMAQALAFLSDFKLGHYMKIPP
KSMFIVQLVGTVVASSVYFGTAWWLLGSIKDICDTAALPEGSPWTCPGDDVFYSASIIWGVIGPGKMFTKEGIYPEMNWC
FLIGFLAPVPVWLLSRKFPKKRWIKQIHMPIIIGTASSMPTAKAVHFNTWGVVGIFFNYYIYRKYKAWWARHTYILSGAL
DAGIAFMGVVIYFALQNYDNFGPNWWGLDSGDHCPLAKCPTAPGVKSKGCPVQ
>GAY47748.1 hypothetical protein CUMW_106760 [Citrus unshiu]
MGSRNFEEDGVPQALSLEKPQTEIKIIGDEEVNDSPIEQNQLSVGSISAQIIVLPLGKLMAATLPSKPIRVPLTKWTFSM
NPGPFNLKEHVLITIFANCGAGEKHRNLLILFDEIQLLGYGWAGLFRKYLVDSPFMWWPANLVQVSLFRALHEKEKRPKG
GLTRLQFFFMVFASSFAYYVVPGYLFPTLSALSFVCWIWKNSVTAQQIGAGLNGLGIGSFGLDWSTVASFLGSPLASPVF
AIINVLAGFILNLYVLVPIAYWINTYEAKRFPIFSSHTFDSTGQPYNISRILNEATFDLDHDAFNSYSKLYLSPFFAFNY
GLSFATLTATISHVALFDGSDIWQMWKRTTSAARDKFADVHTRLMKKHYEAVPQWWFHIILVATVALSIYACEGFDKQLQ
LPWPLANVAFKTYGYISMSQALSFLADFKLGHYMKIPPKSMFLVQLIGTVVASSVYFGTAWWLLTSVEHICDPSALPEGS
PWTCPGDDVFYSASIIWGIVGTGNMPPARAVHYLSWAAVGIFFNYYVYRRFKGWWARHTYILSAALDAGVAFMGVFLFLT
LQSYDIFGPHWWGDGEVNDNPIEEVRLTVPITDDPSLPVLTFRTWVLGILSCGLLAFLNRFFGFRQNQLTVSSVSAQILV
LPIGKLMAATLPTKKFKCPITNWSFSFNPGPFNIKEHVLITIFASCGASGVYAVHIIAMLRAFYKRSIHPVAALLLALTT
QMLGYGWAGIFRRYLVDSPYMWWPANLVQVSLFRALHEKEKRPKGGLTRIQFFFVVFVSSFAYYIVPGYLFPSLTALSFV
CWIWKRSVTAQQIGSGLSGLGIGAIGIDWSTVSSFLGSPLATPLFAIVNTLVGFALVMYILLPIFYWNNVYEAKRFPIFG
AATFDAQGHKYNVDRVLNKETFDLNVEAYNGYSKLYLSVFFAFTYGLSFATLTASISHVALFDGKSIMEMWMKTKDAVGD
KFADVHTRMMRRNYDSVPGWWFHAVLVVSVALALYACEGFGKVLQLPWWGLLLACLIALGFTLPIGIINATTNQQPGLNV
ITELIIGFLYPGKPVANVVFKTYGYISMAQALAFLSDFKLGHYMKIPPKSMFIVQLVGTVVASSVYFGTAWWLLGSIKDI
CDTAALPEGSPWTCPGDDVFYSASIIWGVIGPGKMFTKEGIYPEMNWCFLIGFLAPVPVWLLSRKFPKKRWIKQIHMPII
IGTASSMPTAKAVHFNTWGVVGIFFNYYIYRKYKAWWARHTYILSGALDAGIAFMGVVIYFALQNYDNFGPNWWGLDSGD
HCPLAKCPTAPGVKSKGCPVQ
>GAY47749.1 hypothetical protein CUMW_106760 [Citrus unshiu]
MGSRNFEEDGVPQALSLEKPQTEIKIIGDEEVNDSPIEQVRLTVPITDDPSQPALTFRTWFLGIISCVVLSFLNRFFGFR
QNQLSVGSISAQIIVLPLGKLMAATLPSKPIRVPLTKWTFSMNPGPFNLKEHVLITIFANCGAGGVYAVYIITIVKAFYK
RKLNPLAAMLLAQTTQLLGYGWAGLFRKYLVDSPFMWWPANLVQVSLFRALHEKEKRPKGGLTRLQFFFMVEASSFAYYV
VPGYLFPTLSALSFVCWIWKNSVTAQQIGAGLNGLGIGSFGLDWSTVASFLGSPLASPVFAIINVLAGFILNLYVLVPIA
YWTNTYEAKRFPIFSSHTFDSTGQPYNISRILNEATFDLDHDAFNSYSKLYLSPFFAFNYGLSFATLTATISHVALFDGS
DIWQMWKRTTSAARDKFADVHTRLMKKHYEAVPQWWFHIILVATVALSIYACEGFDKQLQLPWWGILLACAIALFFTLPI
GIIQATTNQQPGLNVITELIIGYMYPGRPLANVAFKTYGYISMSQALSFLADFKLGHYMKIPPKSMFLVQLIGTVVASSV
YFGTAWWLLTSVEHICDPSALPEGSPWTCPGDDVFYSASIIWGIVGPGKMFTKEGVYPALNWFFLVGLLAPVPIWFLSRK
FPEIKWIRLIHIPIIFGGTGNMPPARAVHYLSWAAVGIFFNYYVYRRFKGWWARHTYILSAALDAGVAFMGVFLFLTLQS
YDIFGPHWWGDGEVNDNPIEEVRLTVPITDDPSLPVLTFRTWVLGILSCGLLAFLNRFFGFRQNQLTVSSVSAQILVLPI
GKLMAATLPTKKFKCPITNWSFSFNPGPFNIKEHVLITIFASCGASGVYAVHIIAMLRAFYKRSIHPVAALLLALTTQML
GYGWAGIFRRYLVDSPYMWWPANLVQVSLFRALHEKEKRPKGGLTRIQFFFVVFVSSFAYYIVPGYLFPSLTALSFVCWI
WKRSVTAQQIGSGLSGLGIGAIGIDWSTVSSFLGSPLATPLFAIVNTLVGFALVMYILLPIFYWNNVYEAKREPIFGAAT
FDAQGHKYNVDRVLNKETFDLNVEAYNGYSKLYLSVFFAFTYGLSFATLTASISHVALFDGKSIMEMWMKTKDAVGDKFA
DVHTRMMRRNYDSVPGWWFHAVLVVSVALALYACEGFGKVLQLPWWGLLLACLIALGFTLPIGIINATTNQQPGLNVITE
LIIGFLYPGKPVANVVFKTYGYISMAQALAFLSDFKLGHYMKIPPKSMFIVQLVGTVVASSVYFGTAWWLLGSIKDICDT
AALPEGSPWTCPGDDVFYSASIIWGVIGPGKMFTKEGIYPEMNWCFLIGFLAPVPVWLLSRKFPKKRWIKQIHMPIIIGT
ASSMPTAKAVHFNTWGVVGIFFNYYIYRKYKAWWARHTYILSGALDAGIAFMGVVIYFALQNYDNFGPNWWGLDSGDHCP
LAKCPTAPGVKSKGCPVQ
>XP_015384626.1 oligopeptide transporter 1-like [Citrus sinensis]
MTLYDHDGSVPQSEYSDRHLRISGDGEVNDNPIEEVRLTVPITDDPSLPVLTFRTWVLGILSCGLLAFLNRFFGFRQNQL
TVSSVSAQILVLPIGKLMAATLPTKKFKCPITNWSFSFNPGPFNIKEHVLITIFASCGASGVYAVHIIAMLRAFYKRSIH
PVAALLLALTTQMLGYGWAGIFRRYLVDSPYMWWPANLVQVSLFRALHEKEKRPKGRLTRIQFFFVVFVSSFAYYIVPGY
LFPSLTALSFVCWIWKRSVTAQQIGSGLSGLGIGAIGIDWSTVSSFLGSPLATPLFAIVNTLVGFALVMYILLPIFYWNN
VYEAKREPIFGAATFDAQGHKYNVDRVINKETFDLNVEAYNGYSKLYLSVFFAFTYGLSFATLTASISHVALFDGKSIME
MWMKTKDAVGDKFADVHTRMMRRNYDSVPGWWFHAVLVVSVALALYACEGFGKVLQLPWWGLLLACLIALGFTLPIGIIN
ATTNQQPGLNVITELIIGFLYPGKPVANVVFKTYGYISMAQALAFLSDFKLGHYMKIPPKSMFIVQLVGTVVASSVYFGT
AWWLLGSIKDICDTAALPEGSPWTCPGDDVFYSASIIWGVIGPGKMFTKEGIYPEMNWCFLIGFLAPVPVWLLSRKFPKK
RWIKQIHMPIIIGTASSMPTAKAVHFNTWGVVGIFFNYYTYRKYKAWWARHTYILSGALDAGIAFMGVVIYFALQNYDNF
GPNWWGLDSGDHCPLAKCPTAPGVKSKGCPVQ
>XP_024952977.1 oligopeptide transporter 1-like [Citrus sinensis]
MGSRNFEEDGVPQALSLEKPQTEIKIIGDEEVNDSPIEQVRLTVPITDDPSQPALTFRTWFLGIISCVVLSFLNRFFGFR
QNQLSVGSISAQIIVLPLGKLMAATLPSKPIRVPLTKWTFSMNPGPFNLKEHVLITIFANCGAGGVYAVYIITIVKAFYK
RKLNPLAAMLLAQTTQLLGYGWAGLFRKYLVDSPFMWWPANLVQVSLFRALHEKEKRPKGGLTRLQFFFMVFASSFAYYV
VPGYLFPTLSALSFVCWIWKNSVTAQQIGAGLNGLGIGSFGLDWSTVASFLGSPLASPVFAIINVLAGFILNLYVLVPIA
YWTNTYEAKRFPIFSSHTFDSTGQPYNISRILNEATFDLDHDAFNSYSKLYLSPFFAFNYGLSFATLTATISHVALFDGS
DIWQMWKRTTSAARDKFADVHTRLMKKHYEAVPQWWFHIILVATVALSIYACEGFDKQLQLPWWGILLACAIALFFTLPI
GIIQATTNQQPGLNVITELIIGYMYPGRPLANVAFKTYGYISMSQALSFLADFKLGHYMKIPPKSMFLVQLIGTVVASSV
YFGTAWWLLTSVEHICDPSALPEGSPWTCPGDDVFYSASIIWGIVGPGKMFTKEGVYPALNWFFLVGLLAPVPIWFLSRK
FPEIKWIRLIHIPIIFGGTGNMPPARAVHYLSWAAVGIFFNYYVYRRFKGWWARHTYILSAALDAGVAFMGVFLFLTLQS
YDIFGPHWWGLDSTDHCPLATCPTAPGIVIEGCPVF
>XP_024033872.1 oligopeptide transporter 1 [Citrus clementina]
MGSRNIEEDGVPQALSLEKPQTEIKIIGDEEVNDSPIEQVRLTVPITDDPSQPALTFRTWFLGIISCVVLSFLNRFFGFR
QNQLSVGSISAQIIVLPLGKLMAATLPSKPIRVPLTKWTFSMNPGPFNLKEHVLITIFANCGAGGVYAVYIITIVKAFYK
RKLNPLAAMLLAQTTQLLGYGWAGLFRKYLVDSPFMWWPANLVQVSLFRALHEKEKRPKGGLTRLQFFFMVFASSFAYYV
VPGYLFPTLSALSFVCWIWKNSVTAQQIGAGLNGLGIGSFGLDWSTVASFLGSPLASPVFAIINVLAGFILNLYVLVPIA
YWINTYEAKRFPIFSSHTFDSTGQPYNISRILNEATFDLDHDAFNSYSKLYLSPFFAFNYGLSFATLTATISHVALFDGS
DIWQMWKRTTSAARDKFADVHTRLMKKHYEAVPQWWFHIILVATVALSIYACEGFDKQLQLPWWGILLACAIALFFTLPI
GIIQATTNQQPGLNVITELIIGYMYPGRPLANVAFKTYGYISMSQALSFLADFKLGHYMKIPPKSMFLVQLIGTVVASSV
YFGTAWWLLTSVEHICDPSALPEGSPWTCPGDDVFYSASIIWGIVGPGKMFTKEGVYPALNWFFLVGLLAPVPIWFLSRK
FPEIKWIGLIHIPIILGGIGNMPPARAVHYLSWAAVGIFFNYYVYRRFKGWWARHTYILSAALDAGVAFMGVFLFITLQS
YDIFGPHWWGLDSTDHCPLATCPTAPGIVIEGCPVF
>XP_024033852.1 oligopeptide transporter 1 [Citrus clementina]
MGSYDEDGVTKTKALEKHQTDIDVNGGEEVNDNPIEEVRLTVPITDDPSQPVLTFRTWILGITSCGLLAFVNQFFGYRQN
QLSVGSVSAQILVLPIGKLMAATLPTKQMRVPFTKWSFSLNPGPFNLKEHVLITIFAGCGASGVYAVNIITIVKAFYNRS
LHPVAAMLLVQTTQLLGYGWAGIFRKYLVDSPYMWWPSNLVQVSLFRALHEKERRPKGGLTRLQFFLLVFVSSFGYYIIP
GYLFPSLSALSFVCLIWKDSITAQKLGSGQHGLGIGSFGLDWSTVAGFLGSPLATPFFAIANILAGYFLFLYVLVPIAYW
SNAFEAKKFPLFSSKTFDSDGQVYNITRILNDKAFDLNEIGYRNYSKLYVSVIFAYIYGLSFATLMASISHVALFEGKTI
WEMWKKTATAVNDKFGDVHTRLMKKNYEAVPQWWFQAILVLTFALSLYACEGFGKQLQLPWWGLLLACGMAFFFTLPVGV
IQATTNLQTGLNVITELVIGYMYPGKPLANVTFKTYGYISMSQALSFLGDFKLGHYMKVPPKSMFIVQLVGTLVASTAYF
GTAWWLLTSIDHICNPSLLPEGSPWTCPGDEVFYNASIIWGVVGPLRMFTNYGNYPQMNWFFLIGFLAPFPGWLLSRKFF
EKKWIKNIHMPILLGGPLNLPSAKAVNYTSWAAVGIFFNYYVFRRYKGWWARHNYILSAALDAGVAFMAIMIYFALQSND
IFGPQWWGLDSTDHCPLAKCPIAPGIKADGCPVL
>XP_006474840.1 oligopeptide transporter 1-like [Citrus sinensis]
MGSFDEDGVTKTKALEKHQTDIDVNGGEEVNDNPIEEVRLTVPITDDPSQPVLTFRTWILGITSCGLLAFVNQFFGYRQN
QLSVGSVSAQILVLPIGKLMAATLPTKQMRVPFTKWSFSLNPGPFNLKEHVLITIFAGCGASGVYAVNIITIVKAFYNRS
LHPVAAMLLVQTTQLLGYGWAGIFRKYLVDSPYMWWPSNLVQVSLFRALHEKERRPKGGLTRLQFFLLVFVSSFGYYIIP
GYLFPSLSALSFVCLIWKDSITAQKLGSGQHGLGIGSFGLDWSTVAGFLGSPLATPFFAIANILAGYFLFLYVLVPIAYW
SNAFEAKKFPLFSSKTFDSDGQVYNITRILNDKAFDLNEIGYRNYSKLYVSVIFAYIYGLSFATLMASISHVALFEGKTI
WEMWKKTAAAVNDKFGDVHTRLMKKNYEAVPQWWFQAILVLTFALSLYACEGFGKQLQLPWWGLLLACGMAFFFTLPVGV
IQATTNLQTGLNVITELVIGYMYPGKPLANVTFKTYGYISMSQALSFLGDFKLGHYMKVPPKSMFIVQLVGTLVASTAYF
GTAWWLLTSIDHICNPSLLPEGSPWTCPGDEVFYNASIIWGVVGPLRMFTNYGNYPQMNWFFLIGFLAPFPGWLLSRKFP
EKKWIKNIHMPILLGGPLNLPSAKAVNYTSWAAVGIFFNYYVFRRYKGWWARHNYILSAALDAGVAFMAIMIYFALQSND
IFGPQWWGLDSTDHCPLAKCPIAPGIKADGCPVL
>GAY47751.1 hypothetical protein CUMW_106770 [Citrus unshiu]
MGSFDEDGVTKTKALEKHQTDIDVNGGEEVNDNPIEEVRLTVPITDDPSQPVLTFRTWILGITSCGLLAFVNQFFGYRQN
QLSVGSVSAQILVLPIGKLMAATLPTKQMRVPFTKWSFSLNPGPFNLKEHVLITIFAGCGASGVYAVNIITIVKAFYNRS
LHPVAAMLLVQTTQLLGYGWAGIFRKYLVDSPYMWWPSNLVQVSLFRALHEKERRPTGGLTRLQFFLLVFVSSFGYYIIP
GYLFPSLSALSFVCLIWKDSITAQKLGSGQHGLGIGSFGLDWSTVAGFLGSPLATPFFAIANILAGYFLFLYVLVPIAYW
SNAFEAKKFPLFSSKTFDSDGQVYNITRILNDKAFDLNEIGYRNYSKLYVSVIFAYIYGLSFATIMASISHVALFEGKTI
WEMWKKTAAAVNDKFGDVHTRLMKKNYEAVPQWWFQAILVLTFALSLYACEGFGKQLQLPWWGLLLACGMAFFFTLPVGV
IQATTNLQTGLNVITELVIGYMYPGKPLANVTFKTYGYISMSQALSFLGDFKLGHYMKVPPKSMFIVQLVGTLVASTAYF
GTAWWLLTSIDHICNPSLLPEGSPWTCPGDEVFYNASIIWGVVGPLRMFTNYGNYPQMNWFFLIGFLAPFPGWLLSRKFP
EKKWIKNIHMPILLGGPLNLPSAKAVNYTSWAAVGIFFNYYVFRRYKGWWARHNYILSAALDAGVAFMAIMIYFALQSND
IFGPQWWGLDSTDHCPLAKCPIAPGIKADGCPVL
>KDO59179.1 hypothetical protein CISIN_19004845mg [Citrus sinensis]
MEEANDNPIEEVRLTVPITDDPTIPALTFRTWVLGLTSCCLLAFVNQFFGYRQNQLYLSSISAQILVLPIGKLMAATLPS
KPIPVPLTPWSFSLNPGPFNLKEHVLITIFAGCGSSGVYAVGIITIVKAFYKRSLHVVPAMMLVQTTQLLGYGWAGLFRK
YLVDSPYMWWPANLVQVSLFRALHEEEKRIKGGLTRLQFFVIVFISSFAYYVVPGYLFPSISALSFVCWIWKDSVTAQKL
GSGLQGLGMGSFGLDWATVAGFLGSPLATPFFAIANILVGFFLFLYILIPIAYWCNAFEAQRFPLFSSHTFDSDGQIYNV
SRILNEKEFSFDREAYDNYSRLYLSVLFAFIYGLGFATLMASISHVALFEGKTIWQMWRKTTAAVKQQFGDVHTRLMKKN
YEAVPQWWFHAILIITFALSLFTCEGFDKQFQLPWWGLLLACAMAFFFTLPVGVIQATTNLQPGLNIITEMVIGYMYPGK
PLANVAFKTYGYISMVQALGFLGDFKLGHYMKVPPKSMFVVQLVGTIVASTVYFGTAWWLLTSVEHICNPSLLPEGSPWT
CPGDDVFYNASIIWGVVGPLRMFTNYGNYPQMNWFFLIGFLAPFPVWLLSRKFPEKKWIKNIHMPLLLAGPGSLPSAKAV
NYLSWGAVGIFFNYYVYRRFKGWWARHTYILSAALDAGVAFMGVFLYFTLQSQDIFGPEWWGLAATDHCPLAKCPIAPGI
KVQGCPVA
>XP_006474839.1 oligopeptide transporter 1-like [Citrus sinensis]
MRVSHSAMSFTFQFKDRDMGTYVEGGMLQSMSPENSQTDTRTKGDMEEANDNPIEEVRLTVPITDDPTIPALTFRTWVLG
LTSCCLLAFVNQFFGYRQNQLYLSSISAQILVLPIGKLMAATLPSKPIPVPLTPWSFSLNPGPFNLKEHVLITIFAGCGS
SGVYAVGIITIVKAFYKRSLHVVPAMMLVQTTQLLGYGWAGLFRKYLVDSPYMWWPANLVQVSLFRALHEEEKRTKGGLT
RLQFFVIVFISSFAYYVVPGYLFPSISALSFVCWIWKDSVTAQKLGSGLQGLGMGSFGLDWATVAGFLGSPLATPFFAIA
NILVGFFLFLYILIPIAYWCNAFEAQRFPLFSSHTFDSDGQIYNVSRILNEKEFSFDREAYDNYSRLYLSVLFAFIYGLG
FATIMASISHVALFEGKTIWQMWRKTTAAVKQQFGDVHTRLMKKNYEAVPQWWFHAILIITFALSLFTCEGFDKQFQLPW
WGLLLACAMAFFFTLPVGVIQATTNLQPGLNIITEMVIGYMYPGKPLANVAFKTYGYISMVQALGFLGDFKLGHYMKVPP
KSMFVVQLVGTIVASTVYFGTAWWLLTSVEHICNPSLLPEGSPWTCPGDDVFYNASIIWGVVGPLRMFTNYGNYPQMNWF
FLIGFLAPFPVWLLSRKFPEKKWIKNIHMPLLLAGPGGLPSAKAVNYLSWGAVGIFFNYYVYRRFKGWWARHTYILSAAL
DAGVAFMGVFLYFTLQSQDIFGPEWWGLAATDHCPLAKCPIAPGIKVQGCPVA
>XP_006452635.2 oligopeptide transporter 1 [Citrus clementina]
MCVSHSAISFTFMFMDRDMGTYVEGGMLQSMSPENSQTDTRTKGDMEEANDNPIEEVRLTVPITDDPTIPALTFRTWVLG
LTSCCLLAFVNQFFGYRQNQLYLSSISAQILVLPIGKLMAATLPSKPIPVPLTPWSFSLNPGPFNLKEHVLITIFAGCGS
SGVYAVSIITIVKAFYKRSLHVVPAMMLVHTTQLLGYGWAGLFRKYLVDSPYMWWPANLVQVSLFRALHEEEKRTKGGET
RLQFFVIVFISSFAYYVVPGYLFPSISALSFVCWIWKDSVTAQKLGSGLQGLGMGSFGLDWATVAGFLGSPLATPFFAIA
NILVGFFLFLYILIPIAYWCNAFEAQRFPLFSSHTFDYDGQIYNVSRILNEKEFSFDREAYDNYSRLYLSVLFAFIYGLG
FATLMASISHVALFEGKTIWQMWRKTTAAVKQQFGDVHTRLMKKNYESVPQWWFHAILILTFALSLFTCEGFDKQFQLPW
WGLLLACAMAFFFTLPVGVIQATTNLQPGLNIITEMVIGYMYPGKPLANVAFKTYGYISMVQALGFLGDFKLGHYMKVPP
KSMFVVQLVGTIVASTVYFGTAWWLLTSVEHICNPSLLPEGSPWTCPGDDVFYNASIIWGVVGPLRMFTNYGNYPQMNWF
FLIGFLAPFPVWLLSRKFPEKKWIKNIHMPLLLAGPGSLPSAKAVNYLSWGAVGIFFNYYVYRRFKGWWARHTYILSAAL
DAGVAFMGVFLYFTLQSQGIFGPDWWGLAATDHCPLAKCPIAPGIKVKGCPVA
>ESR65875.1 hypothetical protein CICLE_v10007550mg [Citrus clementina]
MGTYVEGGMLQSMSPENSQTDTRTKGDMEEANDNPIEEVRLTVPITDDPTIPALTFRTWVLGLTSCCLLAFVNQFFGYRQ
NQLYLSSISAQILVLPIGKLMAATLPSKPIPVPLTPWSFSLNPGPFNLKEHVLITIFAGCGSSGVYAVSIITIVKAFYKR
SLHVVPAMMLVHTTQLLGYGWAGLFRKYLVDSPYMWWPANLVQVSLFRALHEEEKRTKGGLTRLQFFVIVFISSFAYYVV
PGYLFPSISALSFVCWIWKDSVTAQKLGSGLQGLGMGSFGLDWATVAGFLGSPLATPFFAIANILVGFFLFLYILIPIAY
WCNAFEAQRFPLFSSHTFDYDGQIYNVSRILNEKEFSFDREAYDNYSRLYLSVLFAFIYGLGFATLMASISHVALFEGKT
IWQMWRKTTAAVKQQFGDVHTRLMKKNYESVPQWWFHAILILTFALSLFTCEGFDKQFQLPWWGLLLACAMAFFFTLPVG
VIQATTNLQPGLNIITEMVIGYMYPGKPLANVAFKTYGYISMVQALGFLGDFKLGHYMKVPPKSMFVVQLVGTIVASTVY
FGTAWWLLTSVEHICNPSLLPEGSPWTCPGDDVFYNASIIWGVVGPLRMFTNYGNYPQMNWFFLIGFLAPFPVWLLSRKF
PEKKWIKNIHMPLLLAGPGSLPSAKAVNYLSWGAVGIFFNYYVYRRFKGWWARHTYILSAALDAGVAFMGVFLYFTLQSQ
GIFGPDWWGLAATDHCPLAKCPIAPGIKVKGCPVA
>ESR65877.1 hypothetical protein CICLE_v10007550mg [Citrus clementina]
MEEANDNPIEEVRLTVPITDDPTIPALTFRTWVLGLTSCCLLAFVNQFFGYRQNQLYLSSISAQILVLPIGKLMAATLPS
KPIPVPLTPWSFSLNPGPFNLKEHVLITIFAGCGSSGVYAVSIITIVKAFYKRSLHVVPAMMLVHTTQLLGYGWAGLFRK
YLVDSPYMWWPANLVQVSLFRALHEEEKRTKGGLTRLQFFVIVFISSFAYYVVPGYLFPSISALSFVCWIWKDSVTAQKL
GSGLQGLGMGSFGLDWATVAGFLGSPLATPFFAIANILVGFFLFLYILIPIAYWCNAFEAQRFPLFSSHTFDYDGQIYNV
SRILNEKEFSFDREAYDNYSRLYLSVLFAFIYGLGFATLMASISHVALFEGKTIWQMWRKTTAAVKQQFGDVHTRLMKKN
YESVPQWWFHAILILTFALSLFTCEGFDKQFQLPWWGLLLACAMAFFFTLPVGVIQATTNLQPGLNIITEMVIGYMYPGK
PLANVAFKTYGYISMVQALGFLGDFKLGHYMKVPPKSMFVVQLVGTIVASTVYFGTAWWLLTSVEHICNPSLLPEGSPWT
CPGDDVFYNASIIWGVVGPLRMFTNYGNYPQMNWFFLIGFLAPFPVWLLSRKFPEKKWIKNIHMPLLLAGPGSLPSAKAV
NYLSWGAVGIFFNYYVYRRFKGWWARHTYILSAALDAGVAFMGVFLYFTLQSQGIFGPDWWGLAATDHCPLAKCPIAPGI
KVKGCPVA
YSL6 protein Sequences
>XP_006423237.2 probable metal-nicotianamine transporter YSL6 isoform X1 [Citrus
clementina]
METAFSASMGTEVEVSEPLIEKITAEEDQLIPEWKDQITIRGLAVSAIMGTLFCIITHRLNLTVGIIPSLNIAAGLLGFL
SVKSWTSFLSKLGFSTKPFTRQENTVIQTCVVACYGLAASGGFGSSFLAMDKRTYKLIGTEYPGNRAEDVKNPGLGWMIV
FMFVVSFVGLFSIVALRKVMILDGKLTYPSGTATAVLINGFHTNAGAELAGMQVRCIGKYLSISFLCSSFKWFFSGVGDS
CGFDNFPSFGLILFKNTFYFDFSPTYVGCGLICPRIVNCSVLLGAIVSWGFLWPYISQHAGDWYPADLGNSDFKGLYGYK
VFIAISLILGDGLYNLIKIISVTFKELCNKRTKVSKLPIDNEIQDTESSRLLIDQKKRENVFLKDGIPTWFAASGYVGLA
AISTATIPTIFPPLKWYLVLLLYLIAPALAFCNSYGAGLIDCSLSLTYAKIGLFIIASLVGTNGGVIAGLAACGVMMSIV
STAADLMQDFKTGYLTLSSAKSMFVSQLLGTAMGCVFAPLTFWMYWTAFDIGSPDGPYKAPYAVILREMAILGIEGFSEL
PKHCLALCCGFFVAALVINLLRDVIPEKISKFIPVPMAMAIPFFVGAYLAIDMFVGTVILFIWERINRKDSEDYAGAVAS
GLICGDGIWIMPSAVLSIFSINPPICMYFGPTVSS
>ESR36477.1 hypothetical protein CICLE_v10027961mg [Citrus clementina]
MGTEVEVSEPLIEKITAEEDQLIPEWKDQITIRGLAVSAIMGTLFCIITHRLNLTVGIIPSLNIAAGLLGFLSVKSWTSF
LSKLGFSTKPFTRQENTVIQTCVVACYGLAASGGFGSSFLAMDKRTYKLIGTEYPGNRAEDVKNPGLGWMIVFMFVVSFV
GLFSLVALRKVMILDGKLTYPSGTATAVLINGFHTNAGAELAGMQVRCIGKYLSISFLCSSFKWFFSGVGDSCGFDNFPS
FGLILFKNTFYFDFSPTYVGCGLICPRIVNCSVLLGAIVSWGFLWPYISQHAGDWYPADLGNSDFKGLYGYKVFIAISLI
LGDGLYNLIKIISVTFKELCNKRTKVSKLPIDNEIQDTESSRLLIDQKKRENVFLKDGIPTWFAASGYVGLAAISTATIP
TIFPPLKWYLVLLLYLIAPALAFCNSYGAGLTDCSLSLTYAKIGLFIIASLVGTNGGVIAGLAACGVMMSIVSTAADLMQ
DFKTGYLTLSSAKSMFVSQLLGTAMGCVFAPLTFWMYWTAFDIGSPDGPYKAPYAVILREMAILGIEGFSELPKHCLALC
CGFFVAALVINLLRDVIPEKISKFIPVPMAMAIPFFVGAYLAIDMFVGTVILFIWERINRKDSEDYAGAVASGLICGDGI
WTMPSAVLSIFSINPPICMYFGPTVSS
>GAY65240.1 hypothetical protein CUMW_239690 [Citrus unshiu]
METAFSASMGTEVEVSEPLIEKITAEEDQLIPEWKDQITIRGLAVSAIMGTLFCIITHRLNLTVGIIPSLNIAAGLLGFL
SVKSWTSFLSKLGFSTKPFTRQENTVIQTCVVAFMASLLAFDVIINMQLSTGGFGSSFLAMDERTYKLIGTEYPGNRAED
VKNPGLGWMIVFMFVVSFVGLFSLVALRKVMILDGKLTYPSGTATAVLINGFHTNAGAELAGMQVRCIGKYLSISFLCSS
FKWFFSGVGDSCGFDNFPSFGLILFKNTFYFDFSPTYVGCGLICPRIVNCSVLLRAIVSWGFLWPYISQHAGDWYPADLG
NSDFKGLYGYKVFIAISLILGDGLYNLIKIISVTFKELCNKRTKVSKLPIDNEIQDTESSRLLIEQKKRENVFLKDGIPT
WFAASGYVGLAAISTATIPTIFPPLKWYLVLLLYLIAPALAFCNSYGAGLTDCSLSLTYAKIGLFIIASLVGTNGGVIAG
LAACGVMMSIVSTAADLMQDFKTGYLTLSSAKSMFVSQLLGTAMGCVFAPLTFWMYWTAFDIGSPDGPYKAPYAVILREM
AILGIEGFSELPKHCLALCCGFFVAALVINLLRDVIPEKISKFIPVPMAMAIPFFVGAYLAIDMFVGTVILFIWERINRK
DSEDYAGAVASGLICGDGIWIMPSAVLSIFSINPPICMYFGPTVSS
>XP_006447029.1 probable metal-nicotianamine transporter YSL6 [Citrus
clementina]
MGTEVEVSEPLIEKIAAVNDEEEEADQPIPEWKDQITIRGLVASAIMGTLFCIITHKLNLTVGIIPSLNVAAGLLGFFLV
KSWTSFLSKLGFSIKPFTRQENTVIQTCVVACYGLAFSGGFGSSLLAMDERTYQLIGADYPGNRAEDVKNPGLGWMIGFV
VVVSFLGLFSLVPLRKVMILDYKLTYPSGTATAMLINSFHTNTGAELAGKQVRCLGKYLSISFFWSCFKWFFSGVGNSCG
FDNFPSFGLTLFKNTFYFDFSPTYVGCGLICPHIVNCSVLLGAIISWGFLWPFISQHAGDWYPADLGSNDFKGLYGYKVF
IAISLILGDGLYNLIKIITITVKEMWNRSTKDSKLPFVNDIQDTETSKLLLEQKKREIVFLKDGIPTWFAASGYVGLAAI
STATIPTIFPPLKWYLVLCSYLIAPALAFCNSYGTGLTDWNLASTYGKIGLFIIASLVGTDGGVIAGLAACGVMMSIVST
AADLMQDFKTGYLTLSSAKSMFVSQLLGTAMGCVIAPLTFWMYWTAFDIGSPDGPYKAPYAVIFREMAILGIEGFSELPK
HCLALCCGFFVAALVINLLRDATPTKISQFIPVPMAMAVPFYIGAYFAIDMFVGTVILFIWELVNRKDSEDYAGAVASGL
ICGDGIWTIPSAILSIFRVNPPVCMYFGPAVGS
>KDO63888.1 hypothetical protein CISIN_1g005868mg [Citrus sinensis]
MGTEVEVSEPLIEKIAAVNDEEEEADQPIPEWKDQITIRGLVASAIMGTLFCIITHKLNLTVGIIPSINVAAGLLGFFLV
KSWTSFLSKLGFSIKPFTRQENTVIQTCVVACYGLAFSGGFGSSLLAMDERTYQLIGADYPGNRAEDVKNPGLGWMIGFV
VVVSFLGLFSLVPLRKVMILDYKLTYPSGTATAMLINSFHTNTGAELAGKQVRCLGKYLSISFFWSCFKWFFSGVGNSCG
FDNFPSFGLTLFKNTFYFDFSPTYVGCGLICPHIVNCSVLLGAIISWGFLWPFISQHAGDWYPADLGSNDFKGLYGYKVF
IAISLILGDGLYNLIKIITITVKEMWNRSTKDSKLPFVNDIQDTETSKLLLEQKEREIVFLKDGIPTWFAASGYVGLAAI
STATIPTIFPPLKWYLVLCSYLIAPALAFCNSYGTGLTDWNLASTYGKIGLFIIASLVGTDGGVIAGLAACGVMMSIVST
AADLMQDFKTGYLTLSSAKSMFVSQLLGTAMGCVIAPLTFWMYWTAFDIGSPDGPYKAPYAVIFREMAILGIEGFSELPK
HCLALCCGFFVAALVINLLRDATPTKISRFIPVPMAMAVPFYIGAYFAIDMFVGTVILFIWELVNRKDSEDYAGAVASGL
ICGDGIWTIPSAILSIFRVNPPVCMYFGPAVGS
>GAY57997.1 hypothetical protein CUMW_183700 [Citrus unshiu]
MGTEVEVSEPLIEKIAAVNDEEEEADQPIPEWKDQITIRGLVASAIMGTLFCIITHKLNLTVGIIPSLNVAAGLLGFFLV
KSWTSFLSKLGFSIKPFTRQENTVIQTCVVACYGLAFSGGFGSSLLAMDERTYQLIGADYPGNRAEDVKNPGLGWMIGFV
VVVSFLGLFSLVPLRKVMILDYKLTYPSGTATAMLINSFHTNTGAELAGKQVRCLGKYLSISFFWSCFKWFFSGVGNSCG
FDNFPSFGLTLFKNTFYFDFSPTYVGCGLICPHIVNCSVLLGAIISWGFLWPFISQHAGDWYPADLGSNDFKGLYGYKVF
IAISLILGDGLYNLIKIIAITVKEMWNRSTKDSKLPFVNDIQDTETSKLLLEQKKREIVFLKDGIPTWFAASGYVGLAAI
STATIPTIFPPLKWYLVLCSYLIAPALAFCNSYGTGLTDWNLASTYGKIGLFIIASLVGTDGGVIAGLAACGVMMSIVST
AADLMQDFKTGYLTLSSAKSMFVSQLLGTAMGCVIAPLTFWMYWTAFDIGSPDGPYKAPYAVIFREMAILGIEGFSELPK
HCLALCCGFFVAALVINLLRDATPTKISQFIPVPMAMAVPFYIGAYFAIDMFVGTVILFIWELVNQKDSEDYAGAVASGL
ICGDGIWTIPSAILSIFRVNPPVCMYFGPAVGS
>GAY65241.1 hypothetical protein CUMW 239690 [Citrus unshiu]
MGTEVEVSEPLIEKITAEEDQLIPEWKDQITIRGLAFDVIINMQLSTGGFGSSFLAMDERTYKLIGTEYPGNRAEDVKNP
GLGWMIVFMFVVSFVGLFSLVALRKVMILDGKLTYPSGTATAVLINGFHTNAGAELAGMQVRCIGKYLSISFLCSSFKWF
FSGVGDSCGFDNFPSFGLILFKNTFYFDFSPTYVGCGLICPRIVNCSVLLRAIVSWGFLWPYISQHAGDWYPADLGNSDF
KGLYGYKVFIAISLILGDGLYNLIKIISVTFKELCNKRTKVSKLPIDNEIQDTESSRLLIEQKKRENVFLKDGIPTWFAA
SGYVGLAAISTATIPTIFPPLKWYLVLLLYLIAPALAFCNSYGAGLTDCSLSLTYAKIGLFIIASLVGTNGGVIAGLAAC
GVMMSIVSTAADIMQDFKTGYLTLSSAKSMFVSQLLGTAMGCVFAPLTFWMYWTAFDIGSPDGPYKAPYAVILREMAILG
IEGFSELPKHCLALCCGFFVAALVINLERDVIPEKISKFIPVPMAMAIPFFVGAYLAIDMFVGTVILFIWERINRKDSED
YAGAVASGLICGDGIWTMPSAVLSIFSINPPICMYFGPTVSS
>GAY65242.1 hypothetical protein CUMW_239690 [Citrus unshiu]
MQLSTGGFGSSFLAMDERTYKLIGTEYPGNRAEDVKNPGLGWMIVFMFVVSFVGLFSLVALRKVMILDGKLTYPSGTATA
VLINGFHTNAGAELAGMQVRCIGKYLSISFLCSSFKWFFSGVGDSCGFDNFPSFGLILFKNTFYFDFSPTYVGCGLICPR
IVNCSVLLRAIVSWGFLWPYISQHAGDWYPADLGNSDFKGLYGYKVFIAISLILGDGLYNLIKIISVTFKELCNKRTKVS
KLPIDNEIQDTESSRLLIEQKKRENVFLKDGIPTWFAASGYVGLAAISTATIPTIFPPLKWYLVLLLYLIAPALAFCNSY
GAGLTDCSLSLTYAKIGLFIIASLVGINGGVIAGLAACGVMMSIVSTAADLMQDFKTGYLTLSSAKSMFVSQLLGTAMGC
VFAPLTFWMYWTAFDIGSPDGPYKAPYAVILREMAILGIEGFSELPKHCLALCCGFFVAALVINLERDVIPEKISKFIPV
PMAMAIPFFVGAYLAIDMFVGTVILFIWERINRKDSEDYAGAVASGLICGDGIWTMPSAVLSIFSINPPICMYFGPTVSS
>XP_006487169.1 probable metal-nicotianamine transporter YSL6 isoform X2 [Citrus
sinensis]
MDKRTYKLIGTEYPGNRAEDVKNPGLGWMIVFMFVVSFVGLFSLVALRKVMILDGKLTYPSGTATAVLINGFHTNAGAEL
AGMQVRCIGKYLSISFLCSSFKWFFSGVGDSCGFDNFPSFGLILFKNTFYFDFSPTYVGCGLICPRIVNCSVLLGAIVSW
GFLWPYISQHAGDWYPADLGNSDFKGLYGYKVFIAISLILGDGLYNLIKIISVTFKELCNKRTKVSKLPIDNEIQDTESS
RLLIDQKKRENVFLKDGIPTWFAASGYVGLAAISTATIPTIFPPLKWYLVLLLYLIAPALAFCNSYGAGLTDCSLSLTYA
KIGLFIIASLVGTNGGVIAGLAACGVMMSIVSTAADLMQDFKTGYLTLSSAKSMFVSQLLGTAMGCVFAPLTFWMYWTAF
DIGSPDGPYKAPYAVILREMAILGIEGFSELPKHCLALCCGFFVAALVINLLRDVIPEKISKFIPVPMAMAIPFFVGAYL
AIDMFVGTVILFIWERINRKDSEDYAGAVASGLICGDGIWTMPSAVLSIFSINPPICMYFGPTVSS
>ESR60268.1 hypothetical protein CICLE_v10014497mg [Citrus clementina]
MDPMSFCLHFIFLVIELLEFDVIMDTQLPSGGFGSSLLAMDERTYQLIGADYPGNRAEDVKNPGLGWMIGFVVVVSFLGL
FSLVPLRKVMILDYKLTYPSGTATAMLINSFHTNTGAELAGKQVRCLGKYLSISFFWSCFKWFFSGVGNSCGFDNFPSFG
LTLFKNTFYFDFSPTYVGCGLICPHIVNCSVLLGAIISWGFLWPFISQHAGDWYPADLGSNDFKGLYGYKVFIAISLILG
DGLYNLIKIITITVKEMWNRSTKDSKLPFVNDIQDTETSKLLLEQKKREIVELKDGIPTWFAASGYVGLAAISTATIPTI
FPPLKWYLVLCSYLIAPALAFCNSYGTGLTDWNLASTYGKIGLFIIASLVGTDGGVIAGLAACGVMMSIVSTAADLMQDF
KTGYLTLSSAKSMFVSQLLGTAMGCVIAPLTFWMYWTAFDIGSPDGPYKAPYAVIFREMAILGIEGFSELPKACLALCCG
FFVAALVINLLRDATPTKISQFIPVPMAMAVPFYIGAYFAIDMFVGTVILFIWELVNRKDSEDYAGAVASGLICGDGIWT
IPSAILSIFRVNPPVCMYFGPAVGS
>KDO63889.1 hypothetical protein CISIN_1g005868mg [Citrus sinensis]
MIGLFILLGINFSFFSSVLYQVMILDYKLTYPSGTATAMLINSFHTNTGAELAGKQVRCLGKYLSISFFWSCFKWFFSGV
GNSCGFDNFPSFGLTLFKNTFYFDFSPTYVGCGLICPHIVNCSVLLGAIISWGFLWPFISQHAGDWYPADLGSNDFKGLY
GYKVFIAISLILGDGLYNLIKIITITVKEMWNRSTKDSKLPFVNDIQDTETSKLLLEQKEREIVFLKDGIPTWFAASGYV
GLAAISTATIPTIFPPLKWYLVLCSYLIAPALAFCNSYGTGLTDWNLASTYGKIGLFIIASLVGTDGGVIAGLAACGVMM
SIVSTAADLMQDFKTGYLTLSSAKSMFVSQLLGTAMGCVIAPLTFWMYWTAFDIGSPDGPYKAPYAVIFREMAILGIEGF
SELPKHCLALCCGFFVAALVINLLRDATPTKISRFIPVPMAMAVPFYIGAYFAIDMFVGTVILFIWELVNRKDSEDYAGA
VASGLICGDGIWTIPSAILSIFRVNPPVCMYFGPAVGS
> PtYSL6_ Ptrif.0002s1042.2_ Poncirus_trifoliata
MGTEVEVSEPLIENIAAVNDEEEEADQPIPEWKDQITIRGLVASAIMGTL
FCIITHKLNLTVGIIPSLNVAAGLLGFFLVKSWTSFLSKLGFSIKPFTRQ
ENTVIQTCVVACYGLAFSGGFGSSLLAMDERTYQLIGADYPGNRAEDVKN
PGLGWMIGFLVVVSFLGLFSLVPLRKVMILDYKLTYPSGTATAMLINSFH
TNTGAELAGKQVRCLGKYLSISFFWSCFKWFFSGVGNSCGFDNFPSFGLT
LFKNTFYFDFSPTYVGCGLICPHIVNCSVLLGAIISWGLLWPFISQHAGD
WYSADLGSNDFKGLYGYKVFIAISLILGDGLYNLIKIIAITVKEMWNRST
KDSKLPIVNDIQDTETSKLLLEQKKREIVFLKDGIPTWFAASGYVGLAAI
STATIPIIFPPLKWYLVLCSYLIAPALAFCNSYGTGLTDWSLASTYGKIG
LFIIASLVGTDGGVIAGLAACGVMMSIVSTAADLMQDFKTGYLTLSSAKS
MFVSQLLGTAMGCVIAPLTFWMYWTAFDIGSPDGPYKAPYAVIFREMAIL
GIEGFSELPKHCLALCCGFFVAALVINLLRDVTPTKISQFIPVPMAMAVP
FYIGAYFAIDMFVGTVILFIWELVNRKDSEDYAGAVASGLICGDGIWTIP
SAILSIFRVNPPICMYFGPAVGS-
PUB26 protein sequences
>PtPUB26_Ptrif.0008s0466.1_Poncirus_trifoliata
MPGSLEPLDLSVQIPYHFRCPISLELMCDPVTVCTGQTYDRPSIESWVAT
GNTTCPVTRSPLTDFTLIPNHTLRRLIQDWCVANRSFGVQRIPTPKQPAE
PSLVRTLLSQASSESNTYGSRLSALRRLRGLARDSDKNRSLISSHNVRAV
LSQVFFTNINVNTASSPELAHESLALLVMFPLTETECMEIASDADKITSL
SSLLFHSSIEVRVNSAALIEIVLAGMRSQELRTQISNVDEIFEGVIDILK
NLSSYPRGLKVGIKALFALCLVKQTRHKAVAAGAAETLVDRLADFDKCDA
ERALATVELLCRIPAGCAAFAEHALTVPLLVKTILKISDRATEYAAGALA
ALCSASERCQRDAVSAGVLTQLLLLVQSDCTDRAKRKAQLLLKLLRDSWP
QDSIGNSDDFACSEVVPF-
>CcPUB26_XP_006422990.1_Citrus_clementina
MPGSLEPLDLSVQIPYHFRCPISLELMCDPVTVCTGQTYDRPSIESWVATGNTTCPVTRSPLTDFTLIPNHTLRRLIQDW
CVANRSFGVQRIPTPKQPAEPSLVRTLINQASSESNTYGSRMSALRRLRGLARDSDKNRCLISSHNVRAILSQVFFTNIN
VNTASSPELAHESLALLVMFPLTETECMEIASDADKITSLSSLLFHSSIEVRVNSAALIEIVLAGMRSQELRAQISNLDE
IFEGVIDILKNLSSYPRGLKVGIKALFALCLVKQTRHKAVAAGAAETLVDRLADFDKCDAERALATVELLCRIPAGCAEF
AEHALTVPLLVKTILKISDRATEYAAGALAALCSASERCQRDAVSAGVLTQLLLLVQSDCTDRAKRKAQLLLKLLRDSWP
QDSIGNSDDFACSEVVPF
>CsPUB26_XP_006487052.1_Citrus_sinensis
MPGSLEPLDLSVQIPYHFRCPISLELMCDPVTVCTGQTYDRPSIESWVATGNTTCPVTRSPLTDFTLIPNHTLRRLIQDW
CVANRSFGVQRIPTPKQPAEPSLVRTLLNQASSESNTYGSRLSALRRLRGLARDSDKNRSLISSHNVRAILSQVFFTNIN
VKTASSPELAHESLALLVMFPLTETECMEIASDADKITSLSSLLFHSSIEVRVNSAALIEIVLAGMRSQELRAQISNLDE
IFEGVIDILKNLSSYPRGLKVGIKALFALCLVKQTRYKAVAAGAAETLVDRLADFDKCDAERALATVELLCRIPAGCAEF
AEHALTVPLLVKTILKISDRATEYAAGALAALCSASERCQRDAVSAGVLTQLLLLVQSDCTDRAKRKAQLLLKLLRDSWP
QDSIGNSDDFACSEVVPF
>CiPUB26_Ci252440_Citrus_ichangensis
MPGSLEPLDLSVQIPYHFRCPISLELMCDPVTVCTGQTYDRPSIESWVAT
GNTTCPVTRSPLTDFTLIPNHTLRRLIQDWCVANRSFGVQRIPTPKQPAE
PSLVRTLLNQASSESNTYGSRLSALRRLRGLARDSDKNRSLISSHNVRAI
LSQVFFTNINVNTASSPELAHESLALLVMFPLTETECMEIASDADKITSL
SSLLFHSSIEVRVNSAALIEIVLAGMRSQELRAQISNLDEIFEGVIDILK
NLSSYPRGLKVGIKALFALCLVKQTRHKAVAAGAAETLVDRLADFDKCDA
ERALATVELLCRIPAGCAEFAEHALTVPLLVKTILKISDRATEYAAGALA
ALCSASERCQRDAVSAGVLTQLLLLVQSDCTDRAKRKAQLLLKLLRDSWP
QDSIGNSDDFACSEVVPF-
>CrPUB26_MSYJ246190.1_Citrus_reticulata
MPGSLEPLDLSVQIPYHFRCPISLELMCDPVTVCTGQTYDRPSIESWVAT
GNTTCPVTRSPLTDFTLIPNHTERRLIQDWCVANRSFGVQRIPTPKQPAE
PSLVRTLLNQASSESNTYGSRLSALRRLRGLARDSDKNRSLISSHNVRAI
LSRVFFTNINVNTASSPELAHESLALLVMFPLTETECMEIASDADKITSL
SSLLFHSSIEVRVNSAALIEIVLAGMRSQELRAQIMRRGEGAGDGGVTVQ
DPGGLRRVCGARADGAAAGEDDTEDIGQGDGVRGGGAGGAVLGVGEVPEG
RGQRGGVDPAVAVGAERLYGQGQEEGAAAVEATEGEVASGFYWEFR-
>CmaPUB26_Cg8g004360.1_Citrus_maxima
MPGSLEPLDLSVQIPYHFRCPISLELMCDPVTVCTGQTYDRPSIESWVAT
GNTTCPVERSPLTDFTLIPNHTLRRLIQDWCVANRSFGVQRIPTPKQPAE
PSLVRTLLNQASSESNTYGSRLSALRRLRGLARDSDKNRSLISSENVRAI
LSQVFFTNINVKTASSPELAHESLALLVMFPLTETECMEIASDADKITSL
SSLLFHSSIEVRVNSAALIEIVLAGMRSQELRAQISNLDEIFEGVIDILK
NLSSYPRGLKVGIKALFALCLVKQTRYKAVAAGAAETLVDRLADFDKCDA
ERALATVELLCRIPAGCAEFAEHALTVPLLVKTILKISDRATEYAAGALA
ALCSASERCQRDAVSAGVLTQLLLLVQSDCTDRAKRKAQLLLKLLRDSWP
QDSIGNSDDFACSEVVPF-
> CmePUB26_Cm188050.1_Citrus_medica
MPGSLEPLDLSVQIPYHFRCPISLELMCDPVTVCTGQTYDRPSIESWVAT
GNTTCPVTRSPLIDFTLIPNHTERRLIQDWCVANRSFGVQRIPTPKQPAE
PSLVRTLINQASSESNTYGSRLSALRRLRGLARDSDKNRSLISSHNVRAI
LSQVFFTNINVNTASSSPELAHESLALLVMFPLTETECMEIASDADKITS
LSSLLFHSSIEVRVNSAALIEIVLAGMRSQELRAQISNVDEIFEGVIDIL
KNLSSYPRGLKVGIKALFALCLVKQTRQKAVAAGAAETLVDRLADFDKCD
AERALATVELLCRIPAGCAAFAEHALTVPLLVKTILKISDRATEYAAGAL
VALCSASERCQRDAVSAGVLTQLLLLVQSDCTDRAKRKAQLLLKLLRDSW
PQDSIGNSDDFACSEVVPF-
>SlPUB26_Solyc01g107980.2 sequence match in blast db Tomato Genome protein
sequences (ITAG release 2.40)
MPASLDPLDVGVQIPYHFRCPISLELMRDPVTVCTGQTYDRQSIESWVATGNTTCPVTRAPLSDFTLIPNHTLRRLIQEW
CVANRAFGVERIPTPKQPADPSLVRSLLNQAAAQSNHMNSRVAALRRLRGLARDSEKNRSVISANNAREILLAIVFSRMD
SDASELHHESLAILSMFPLSEPECVFVASDPGRVGYLVAMLFHPSIDVRVNSAALIETVVAGMRSPEFRAQISNADDVFE
GVVGILNYPLAYPRALKVGIKALFALCLVKQHRQRAVSAGAVEALIDRLQDFEKCDAERALATIELLSRIPSGCAALASH
ALTVPLLVKIILKISERATEYAAGALLSLCSASEQAQKEAVAAGVLIQLLLLVQSDCTERAKRKAQMLLKQLRDCWPEDS
IANTDDFACSDVVPF
>SlPUB26_KAH0727421.1 hypothetical protein KY284 003286 [Solanum tuberosum]
MPGSLDPLDVGVQIPYHFRCPISLELMRDPVTVCTGQTYDRQSIESWVATGNTTCPVTRAPLSDFTLIPN
HTLRRLIQEWCVANRAFGVERIPTPKQPADPSLVRSLINQAAAQSNHMNSRVAALRRLRGLARDSDKNRS
VILANNAREILLAIVFSRMDSDSSELNHESLAILSMFPLSEPECVFVASDPERVSYLVAMLFHPSIDVRV
NSAALIEIVVAGMRSPELRAQISNADDVFEGIVGILNYPLAYPRALKVGIKALFALCLVKQHRQRAVTAG
AVEALIDRLQDFEKCDAERALATIELLSRIPSGCAALASHALTVPLLVKIILKISERATEYAAGALLSLC
SASEQAQKEAVAAGVLIQLLLLVQSDCTERAKRKAQMLLKQLRDCWPEDSIANSDDFACSDVVPF
DMR6 protein Sequences
>PtDMR6.1_Ptrif.0007s1480.1.v1.3.1_Poncirus_trifoliata_v1.3.1
MAAAAITHIKLLLSDLASTLKNVPSDYIRPISDRPNPTDQAHISDGSIPL
IDLQGLNGPRRSDIIKQIGQACQHGGFFQVKNHGISEAMINNMLSIARTF
FKLPERERLKIYSDDPLKPTRLSTGFNVKTEKASNRRDFLRLHCYPLQDY
VHDWPLNPPSFREDVGDYCASVRGLVERLIEAISESLRLPSDYIDKEALG
KQGQHMALNYYPPCPPPELTYGLPGHTDPNLITIHRAVVSRDKERTLVPI
IAQNLHLIADVDNEGAGNRIYRNTFAVSEDLQSRDIILKKNSY-
> PtDMR6.2_Ptrif.0007s1482.2.v1.3.1_Poncirus_trifoliata_v1.3.1
MSAAATTATKLLLSDLAPTLINVPSDYIRPISDRPSFTDQTHKSDGSIPL
IDLQGLNGPRRSDIIKQIGQACQHCGFFQVKNHGISEAMINNMLSIARTF
FKLPESERLKIYSDDPSKPTRLSTSFNVKTEKLSNWRDFLRLHCYPLQDY
VHDWPLNPPSFREDVGDYCTSVRGLVERLIEAISETLGLPSDYIDKEALG
KHGQHMALNYYPPCPQPELTYGLPGHTDPNLITILLQDDVPGLQVLRDGN
WVPVNPIPSTFIVNIGDQMQVLSNDRYKSVLHRAVVSRDKERISIPTFYC
PSPYAVIGPAKGLVDQDHPAVYRDFTYAEYYKKFWNRGLATECCLDMFKA
SSTV-
> PtDMR6.3_Ptrif.0009s1899.1.v1.3.1_Poncirus_trifoliata_v1.3.1
MQLMHRLCIVMRSDSHRLSVNGDEITILISKYNQALRYRPLDMAAATTKL
LLSDLASTVKSVPSNYIRPISDRPNLTEVQISDGSIPLIDLQVLDGPRRL
DIIKQIGQACQHDGFFQVKNHGIPETIINNMLSIARAFFKLPESERLKSY
SDDPSKSTRLSTSFNVNTEKVANWRDYLRLACYPLQDYTHEWPSTPPSVR
EVAAEYCTSLRGLVLRLLEAISESLGLQRDFIDKALGKHGQHMALNYYPP
CPQPDLTYGLPGHTDPNLITVLLQDDVPGLQVLRNGKWLPVGPIPNTFIV
NIGDQMQVLSNDRYKSVLHRALVNCDKERISIPTFYCPSPDAVIAPAKDL
IDERHPAVYKNFTYAEYYRKFWNRGLDERCLDLFKASTA-
> PtDMR6.4_Ptrif.0009s1896.1.v1.3.1_Poncirus_trifoliata_v1.3.1
MQLVHQFMRPDGHRLSVNSDEITILSSKYNQALRYRPLDIAAATTKLLLS
DLASTVKSVPSNYIRPISDRPNLTEVQISDGSIPLIDLQVLNGPRRLDLI
KQIRQACQHDGFFQVKNHEIPETIINNMLSIARAFFKLPESERLKSYSDD
PSKSTRLSTSFNVNTEKVANWRDYLRLYYYPLQDYMHEWPSNPPSFREVV
TEYYTSVRGLVPKLLEAISESLGVQRDNVDKALGKHGQHMALNYCPPCPQ
PDLTYELPRHTDPNLITVLLQDDVPGLQLLRNGKWLPGSPIPNTFIVNIG
DQMQVLSNDLYKSVLHRALVSCDKERISIPTFYCPSLDAVIAPTKDLIDE
RHLAFYKNFTYAEYYQKF-
>ESR40545.1 hypothetical protein CICLE_v10027096mg [Citrus clementina]
MAAAATTHIKLLLSDLASTLQNVPSDYIRPISDRPNLTDQAHISDGSIPLIDLQGLYGPRRSDIIKQIGQACQHGGFFQV
KNHGISEAMINNMLSIARTFFKLPERERLKNYSDDPLKPTRLSTSFNVKTEKASNRRDFLRLHCYPLQDYVHDWPLNPPS
FREDVGDYCTSVRGLVERLIEAISESLRLPSDYIDKEALGKHGQHMALNYYPPCPPPELTYGLPGHTDPNLITIHRAVVS
RDKERTEVPIIAENLALIADVDNEGLIDQDHPAVYRDFTYAEYYEKFWNRGLAAECRLDMFKAS
>XP_024036895.1 protein DMR6-LIKE QXYGENASE 2 [Citrus clementina]
MAAAATTHIKLLLSDLASTLQNVPSDYIRPISDRPNLTDQAHISDGSIPLIDLQGLYGPRRSDIIKQIGQACQHGGFFQV
KNHGISEAMINNMLSIARTFFKLPERERLKNYSDDPLKPTRLSTSFNVKTEKASNRRDFLRLHCYPLQDYVHDWPLNPPS
FREDVGDYCTSARGFVTGKMWVTTVQVLEGWC
>GAY54543.1 hypothetical protein CUMW_157480 [Citrus unshiu]
MPSMHQHECFFQVKNHGISEAMINNMLSIARTFFNLPERERLKIYSDDPLKPTRLSTSFNVKTEKASNRRDFLRLYCYPL
QDYVHDWPLNPPSFREDVGDYCTSVRGLVERLIEAISESLRLPSDYIDKEALGKHGQHMALNYYPPCPPPELTYGLPGHT
DPNLITIHRAVVSRDKERTLVPIIAENLHLIADVDNEGAGNMIYRNTFAVSEDLQSRDIILKKNSY
>GAY54540.1 hypothetical protein CUMW_157450 [Citrus unshiu]
MSAVATTATKLLLSDLAPTLKNVPSDYIRPISDRPNLTDQAHISDGSIPLIDLQGLYGPRRSDIIKQIGQACQHCGFFQV
KNHGISEAMINNMLSIARTFFKLPESERLKIYSDDPSKPTRLSTSFNVKTEKVSNWRDFLRLHCYPLQDYVHDWPLNPPS
FRYI
>GAY54539.1 hypothetical protein CUMW_157440 [Citrus unshiu]
MAAAATTTTKLLLSDRASTLKNVPSDYIRPISDRPNLTDQAHISEGSIPLIDLQGFYGLRRSDITKQIGQACQHGGFFQD
DVPGLQVLRDGNWVPVNPIPSTFIVNIGDQMQVLSNDRYKSVLHRAVVSRDKERISIPTFYCPSPDAVICPAKGLVDHDH
PAVYRDFTYAEYYEKFWNRGLATECCLDMFKASSTV
>ESR34925. hypothetical protein CICLE_v10006618mg [Citrus clementina]
MAAATTKLLLSDLASTVKSVPSNYIRPISDRPNLTEVQISDGSIPLIDLQVLDGPRRLDIIKQIGQACQHDGFFQVKNHG
IPETIINNMLSIARAFFKLPESERLKSYSDDPSKSTRLSTSFNVNTEKVSNRRDYLRLACYPLQDYIHEWPSNPPSFREV
VAEYCTSARGAWGLQRDYIDKALGKHGQHMALNYYPPCPQPDLTYGLPGHTDPNLITVLLQDDVPGLQVLRKGKWLPFSL
DYDFRIGYRPHTHIHNFESLAICLIPNTFIVNIGDQMQVLSNDRYKSVLHRALVNCDKEHISILTFYCSSPDAVIAHAKD
LIDERHPTVYKNFTYSEYY
>KDO65454.1 hypothetical protein CISIN_1g042145mg, partial [Citrus sinensis]
MAAATTKLLLSDLASTVESVTSNYIRPISDRPNLTEVQISDGSIPLIDLQVLDGPRRLDLIKQIGQACHHDGFFQVKNHG
IPETIINNTLSIAGAFFKLPESERLKSYSDDPSKSKRLSTSFNVNTKKVSNWRDYLRLHCYPLQDCMHEWPSNPPSFEVV
AEYCTSVRGLVLKLLEAISESMGLQRDYIDKALGKHGQQMALNYCPPCPQPDLTYGLPGHTDPNLITVLLQDD
>XP_006465312.1 protein DMR6-LIKE QXYGENASE 2-like [Citrus sinensis]
MSAVATTATKLLLSDLAPTLKNVPSDYIRPISDRPNLTDQAHISDGSIPLIDLQGLYGPRRSDIIKQIGQACQHCGFFQV
KNHGISEAMINNMLSIARTFFKLPESERLKIYSDDPSKPTRLSTSFNVKTEKVSNWRDFLRLHCYPLQDYVHDWPLNPPS
FREDVGDYCTSVRGLVLRLIEAISESLGLPSDYIDKEALGKHGQHMALNYYPPCPQPELTYGLPGHTDPNLITILLQDDV
PGLQVLRDGNWVPVNPIPSTFIVNIGDQMQVLSNDRYKSVLHRAVVSRDKERISIPTFYCPSSDAVIGPAKGLVDQDLPA
VYRDFTYAEYYKKFWNRGLATECCLDMFKASSTV
>XP_006427307.1 protein DMR6-LIKE QXYGENASE 2 [Citrus clementina]
MSAAATTATKLLLSDLAPTLINVPSDYIRPISDRPSLTDQTHISDGSIPLIDLQGLNGPRRSDIIKQIGQACQHCGFFQV
KNHGISEAMINNMLSIARTFFKLPESERLKIYSDDPSKPTRLSTSFNVKTEKVSNWRDFLRLACYPLQDYVHDWPLNPPS
FREDVGDYCTSVRGLVLRLIQAISESLGLPSDYIDKEALGKHGQHMALNYYPPCPQPELTYGLPGHTDPNLITLLLQDDV
PGLQVLRDGNWVPVNPIPSTFIVNIGDQMQVLSNDRYKSVLHRAVVSRDKERISIPTFYCSSPDAVIGPAKGLVDQDHPA
VYRDFTYAEYYKKFWNRGLATECCLEMFKASSTV
>XP_015389438.1 protein DMR6-LIKE QXYGENASE 1-like [Citrus sinensis]
MAAATTKLLLSDLASTVKSVPSNYIRPISDRPNLTEVQISDGSIPLVDLQVLNGPSRLDIIKQIGQACQHDGFFQVKNHG
IPETIINNMLTIARAFFKLPEKSERLKSYSDDPSKSKRLSTSFNVNTKKVSNWRDYLRLHCYPLQDCMHEWPSNPPSFEV
VAEYCTSVRGLVLKLLEAISESMGLQRDYIDKALGKHGQQMALNYCPPCPQPDLTYGLPGHTDPNLITVLLQDDVMKQKQ
TLCP
>CIDMR6_Ci117750.1 _Citrus_ichangensis
MSAVATTATKLLLSDLAPTLKNVPSDYIRPISDRPNLTDQAHISDGSIPL
IDLQGLYGPRRSDIIKQIGQACQHCGFFQVKNHGILEAMINNMLSIARTF
FNLPERERLKIYSDDPLKPTRLSTSFNVKTEKASNRRDFLRLHCYPLQDY
VHDWPLNPPSFREDVGDYCTSVRGLVLRLIEAISESLRLPSDYIDKEALG
KHGQHMTLNYYPPCPPPELTYGLPGHTDPNLITIHQAVVSRDKERTLVPI
IAENLHLIADVDNEGLIDQDHPAVYRDFTYAEYYEKSWNRGLAAQCCLDM
FKAS-
PAO1 protein sequences
>PtPAO1_Ptrif.0004s2552.4.v1.3.1_Poncirus_trifoliata_v1.3.1
MDSTSRSSVIIIGAGISGISAGKILAENGIEDILILEASDRIGGRVRNEK
FGGVSVELGAGWIAGVGGKESNPVWELASKSGLRTCFSDYTNARYNIYDR
SGKIIPSGVAADSYKKAVESAIANLKNLEATNSNIGEVIKAAMELPSSPK
TPLELAIDFILHDFEMAEVEPISTYVDFGEREFLVADERGYAHLLYKMAE
EFLSTSDGKILDNRLKLNKVVRELQHSRNGVTVKTEDGCVYEANYVILSA
SIGVLQSDLISFKPPLPKWKTEAIEKCDVMVYTKIFLKFPCKFWPCSPEK
EFFIYAHERRGYYTFWQHMENAYPGSNILVVTLINGESKRVEAQPDEETL
KEAMEVLRDMFGPDIPNATDILVPRWWNNRFQRGSYSDYPIISDNQLVNN
IRAPVGGIFFTGEHTSERFNGYVAGGYLAGIDTGKAVVEKIRKDNERNNS
ETQNFLLEPLLALTLTQTEAMPSLHKCDIPKQLYLSGKLGIPEAIL-
>XP_006438967.1 polyamine oxidase 1 [Citrus clementina]
MDSTSRSSVIIIGAGVSGISAGKILAENGIEDILILEASDRIGGRVRNEKFGGVSVELGAGWIAGVGGKESNPVWELASK
SGLRTCFSDYTNARYNIYDRSGKIIPSGVAADSYKKAVESAIANLKNLEATNSNIGEVIKAATELPSSPKTPLELAIDFI
LHDFEMAEVEPISTYVDFGEREFLVADERGYAHLLYKMAEEFLSTSDGKILDNRLKLNKVVRELQHSRNGVTVKTEDGCV
YEANYVILSASIGVLQSDLISFKPPLPKWKTEAIEKCDVMVYTKIFLKFPCKFWPCSPEKEFFIYAHERRGYYTFWQHME
NAYPGSNILVVTLTNGESKRVEAQPDEETLKEAMEVLQDMFGPDIPNATDILVPRWWNNRFQRGSYSNYPIISDNQLVNS
IRAPVGGIFFTGEHTSERFNGYVHGGYLAGIDTGKAVVEKIRKDNERNNSETQNFLLEPLLALTLTQTEAMSSLHKCDIP
KQLYLSGKLGIPEAIL
>XP_024956636.1 polyamine oxidase 1 isoform X1 [Citrus sinensis]
MDSTSRSSVIIIGAGVSGISAGKILAENGIEDILILEASDRIGGRVRNEKFGGVSVELGAGWIAGVGGKESNPVWELASK
SGERTCFSDYTNARYNIYDRSGKIIPSGVAADSYKKAVESAIANLKNLEAINSNIGEVIKAATELPSSPKTPLELAIDFI
LHDFEMAEVEPISTYVDFGEREFLVADERGYAHLLYKMAEEFLSTSDGKILDNRLKLNKVVRELQHSRNGVTVKTEDGCV
YEANYVILSASIGVLQSDLISFKPPLPQKWKTEAIEKCDVMVYTKIFLKFPCKFWPCSPEKEFFIYAHERRGYYTFWQHM
ENAYPGSNILVVTLTNGESKRVEAQPDEETLKEAMEVLQDMFGPDIPNATDILVPRWWNNRFQRGSYSNYPIISDNQLVN
SIRAPVGGIFFTGEHTSERFNGYVHGGYLAGIDTGKAVVEKIRKDNERNNSETQNFLLEPLLALTLTQTEAMSSLHKCDI
PKQLYLSGKLGIPEAIL
>AJP16789.1 polyamine oxidase 1 [Citrus trifoliata]
MDSTSRSSVIIIGAGISGISAGKILAENGIEDILILEASDRIGGRVRNEKFGGVSVELGAGWIAGVGGKESNPVWELASK
SGLRTCFSDYTNARYNIYDRSGKIIPSGVAADSYKKAVESAIANLKNLEATNSNIGEVIKAAMELPSSPKTPLELAIDFI
LHDFEMAEVEPISTYVDFGEREFLVADERGYAHLLYKMAEEFLSTSDGKILDNRLKLNKVVRELQHSRNGVTVKTEDGCV
YEANYVILSASIGVLQSDLISFKPPLPKWKTEAIEKCDVMVYTKIFLKFPCKFWPCSPEKEFFIYAHERRGYYTFWQHME
NAYPGSNILVVTLINGESKRVEAQPDEETLKEAMEALRDMFGPDIPNATDILVPRWWNNRFQRGSYSDYPIISDNQLVNN
IRAPVGGIFFTGEHTSERFNGYVHGGYLAGIDTGKAVVEKIRKDNERNNSETQNFLLEPLLALTLTQTEAMPSLHKCDIP
KQLYLSGKLGIPEAIL
>SlPAO1_Solyc01g087590.2 sequence match in blast db Tomato Genome protein
sequences (ITAG release 2.40)
METPRRSSVIIVGAGISGLTAAKVLSENGVDDVVILEAADKIGGRIRKEEFGGVAAELGAGWIAGVGGKQSNPVWELALQ
SNLRTCFSDYSNARYNIYDHSGKIFPSGIAADSYKKAVDSAIQKLRSQEGNHNEDIDDAAETPSTPKTPIELAIDFILHD
FEMAEVEPISTYVDFGEREFLVADERGYEHLLYKMAENFLFTCEGKIMDSRLKLNTVVREVQHSRNGVLVTTEDGSLYEA
NYVILSVSIGVLQSDLISFSPSLPRWKMEAVRNLDVMVYTKIFLKFPNKFWPCEPEKEFFIYAHERRGYYTFWQHMENAY
PGSNMLVVTLTNGESKRVEAQSDQDTLREAMEVLRNMFGPDIPDATDILVPRWWNNRFQRGSYSNYPIYANHQLVHDIKE
PVGRIFFTGEHTSEKFSGYVHGGYLSGIDTSNALLEKMRRDDGRKNESQAFLLEPLLALTGSLTLTQAETVSSLHKCDIP
RQLFLSNSKLGLPEAIL
>StPAO1_XP_006345421.1 PREDICTED: polyamine oxidase 1 [Solanum tuberosum]
MEIPRRSSVIIIGAGISGLTAAKVLSENGVDDVVILEAADKIGGRIRKEEFGGVAAELGAGWIAGVGGKQ
SNPVWELALQANLRTCFSDYSNARYNIYDHSGKIFPSGIAADSYKKAVDSAIQKLRSQEGNHNENTEDAA.
ETPSTPKTPIELAIDFFLHDFEMAEVEPISTYVDFGEREFLVADERGYEHLLYKMAENFLFTSEGKIMDS
RLKLNTVVREVQHSRNGVLVTTEDGPLYEANYVILSVSIGVLQSDLISFSPPLPRWKMEAVRNLDVMIYT
KIFLKFPYKFWPCEPEKEFFIYAHERRGYYTFWQHMENAYPGSNMLVVTLTNGESKRVEAQSDQDTLREA
MEVLRNMFGPDIPNATDILVPRWWNNRFQRGSYSNYPIYANHQLVHDIKEPVGRIFFTGEHTSEKFSGYV
HGGYLSGIDSSNALLEKMRRDDGWKNESQAFLLEPLLALTGSLTLTQAETVSSLHKCDIPRQLFLSNSKL
AEAIL
TPS5 protein sequences
>PtTPS5.1_Ptrif.0005s1029.1.v1.3.1_Poncirus_trifoliata
MVSRSYSNLLDLASGDFPNFSREKKRLPRVATVAGVLSEIDDENSNSVGS
DAPSSVSQERMIIVGNQLPLRAHRSSDGSGGWTFSWDEDSLLLQLKDGLG
EDVEVIYVGCIKEQIDLSEQDEVSQTLLETFKCVPAFIPPELFSKFYHGF
CKQHLWPLFHYMLPLSPDLGGRFDRSLWQAYVSVNKIFADKVMEVISPDD
DFVWVHDYHLMVLPTFLRKRFNRVKLGFFLHSPFPSSEIYRTLPIRDELL
RALLNADLIGFHTFDYARHFLSCCSRMLGVSYQSKRGYIGLEYFGRTVSI
KILPVGIHIGQLQSVLNLPETEAKVAELQDQFKGQIVMLGVDDMDIFKGI
SLKLLAMEQLLSQNPSKRGKIVLVQIANPARGRGRDVQEVQSETHATVRR
INKIFGRPGYQPVVLIDTPLQFYERIAYYVIAECCLVTAVRDGMNLIPYE
YIICRQGNEKLDMTLGLDPSTAKSSMLVVSEFVGCSPSLSGAIRVNPWNI
DAVAEAMDSALGVSDAEKQMRHEKHYRYVSTHDVAYWARSFLQDLERACR
DHMRRRCWGIGFGLGFRVVALDPNFRKLSIDHIVSAYKRTKNRAILLDYD
GTMMVPGSISTSPNAEAVAILDNICRDPKNVVFLVSGKDRDTLAEWFSSC
EGLGIAAEHGYFVRPNYGVDWETCVSVPDFSWKQIAEPVMKLYTETTDGS
TIETKESALVWNFQYADPDFGSCQAKELLDHLESVLANEPVSVKSGPNIV
EVKPQGVNKGLVAQHQLETMHQKGMLPDFVLCIGDDRSDEDMFEVIKSAA
AGPSLSPVAEVFACTVGQKPSKAKYYLDDTAEILRMLLGLAEASAHNAYK
ASQGSQRVVINKE-
> PtTPS5.2_Ptrif.0002s1896.1.v1.3.1_Poncirus_trifoliata
MVSKSYSNLLELASGEAPSFGRMSRRIPRIMTVAGIISDLDDDPADSVCS
DPSSSSVQRDRIIIVANQLPIRAQRKSDNSKGWIFSWDENSLLLQLKDGL
GDDDIEVIYVGCLKEEIHVNEQDEVSQILLETFKCVPTFLPPDLFSRYYH
GFCKQQLWPLFHYMLPLSPDLGGRFNRSLWQAYVSVNKIFADRIMEVINP
EDDFVWVHDYHLMVLPTFLRKRFNRVKLGFFLHSPFPSSEIYKTLPIREE
ILRALLNSDLIGFHTFDYARHFLSCCSRMLGLTYESKRGYIGLEYYGRTV
SIKILPVGIHMGQLQSVLSLPETEAKVSELVKQFHDQGKVMLLGVDDMDI
FKGISLKLLAMEQLLIQHPEWQGKVVLVQIANPARGRGKDVKEVQAETYS
TVERINQTFGKPGYDPVVLIDEPLKFYERIAYYVVAECCLVTAVRDGMNL
IPYEYIISRQGNEKLDKVVGSEPSSPKKSMLVVSEFIGCSPSLSGAIRVN
PWNIDAVADAMDGALEMADQEKQLRHEKHYRYVSTHDVGYWARSFLQDLE
RTCREHVRQRCWGIGFGLSERVVALDPNFKKLSMEHIVSAYKRTTTRAIL
LDYDGTLMPQASIDKSPNSKTIDILNSLCRDKNNMVFLVSAKSRKTLAEW
FSPCENLGIAAEHGYFFRLRRDEEWETCIPVADCGWKQIAEPVMKLYTET
TDGSTIEDKETALVWSYEDADPDFGSCQAKELLDHLESVLANEPVTVKSG
QNLVEVKPQGVNKGLVAKRLLSTMQEWEMLPDFVLCVGDDRSDEDMFEVI
ISSTAGPSIAPRAEVFACTVVQKPSKAKYYLDDTVEIVRLMQGLACVADQ
MVSV-
> PtTPS5.3_Ptrif.0002s2923.1.v1.3.1_Poncirus_trifoliata
MSKSYTNLLDLASGNFPAMGPSREKKRLPRVMTVPGVISELDDDQANSVS
SDVPSSVAQDRVIIVANQLPVKAKRRPDNKGWSFSWDEDSLLLQLKDGLP
EDMEVIYVGSLKVDVDLSEQDDVSQLLLDRFKCVPAFLPPDILTKFYHGF
CKQHLWPLFHYMLPFSATHGGRFDRSLWEAYVSANKIFSQRVIEVINPED
DYVWIHDYHLMVLPTFLRRRFTRLRMGFFLHSPFPSSEIYRTLPVREEIL
KALLNADLIGFHTFDYARHFLSCCSRMLGLEYQSKRGYIGLEYYGRTVGI
KIMPVGIHMGQIESVERLADKDWRVQELKQQFEGKTVLLGVDDMDIFKGV
DLKLLAMEHLLKQHPKWQGRAVLVQIANPARGRGKDLEEIQAEIHATCRR
INETFGRPGYEPVVFIDKPVSLSERAAYYTIAECVVVTAVRDGMNLTPYE
YIVCRQGVSGSESSSESSAPKKSMLVVSEFIGCSPSLSGAIRVNPWNIEA
TAEAMHEAIQMNEAEKQLRHEKHYRYVSTHDVAYWARSFFQDMERTCKDH
FKRRCWGIGLSFGFRVVALDPNFRKLSIDAIVSAYLRSKSRAILFDYDGT
VMPQTSINKAPSQAVISIINTLCNDARNAVFVVSGRGRDSLGKWFSPCKK
LGIAAEHGYFMRWSADEEWQNCGQSVDFGWIQIAEPVMKLYTESTDGSYI
EIKESALVWHHRDADPGFGSSQAKELLDHLESVLANEPAAVKSGQFIVEV
KPQGVSKGVVAEKIFTTMAESGRHADFVLCIGDDRSDEDMFEIIGNATSS
GVLSSNASVFACTVGQKPSKAKYYLDDAAEVVTMLEALAEASAPPSFEVG
ASDSP-
>XP_006432798.1 alpha, alpha-trehalose-phosphate synthase [UDP-forming] 5 [Citrus
clementina]
MVSRSYSNLLDLASGDFPNFSREKKRLPRVATVAGVLSEIDDENSNSVGSDAPSSVSQERMIIVGNQLPLRAHRSSDGSG
GWTFSWDEDSLLLQLKDGLGEDVEVIYVGCIKEQIDLSEQDEVSQTLLETFKCVPAFIPPELFSKFYHGFCKQHLWPLFH
YMLPLSPDLGGRFDRSLWQAYVSVNKIFADKVMEVISPDDDFVWVHDYHLMVLPTFLRKRFNRVKLGFFLHSPFPSSEIY
RTLPIRDELLRALLNADLIGFHTFDYARHFLSCCSRMLGVSYQSKRGYIGLEYFGRTVSIKILPVGIHIGQLQSVINLPE
TEAKVAELQDQFKGQIVMLGVDDMDIFKGISLKLLAMEQLLSQNPSKRGKIVLVQIANPARGRGRDVQEVQSETHATVRR
INKIFGRPGYQPVVLIDTPLQFYERIAYYVIAECCLVTAVRDGMNLIPYEYIICRQGNEKLDMTLGLDPSTAKSSMLVVS
EFVGCSPSLSGAIRVNPWNIDAVAEAMDSALGVSDAEKQMRHEKHYRYVSTHDVAYWARSFLQDLERACRDHMRRRCWGI
GFGLGFRVVALDPNFRKLSIDHIVSAYKRTKNRAILLDYDGTIMVPGSISTSPNAEAVAILDNLCRDPKNVVFLVSGKDR
DTLAEWFSSCEGLGIAAEHGYFVRPNYGVDWETCVSVPDFSWKQIAEPVMKLYTETTDGSTIETKESALVWNFQYADPDF
GSCQAKELLDHLESVLANEPVSVKSGPNIVEVKPQGVNKGLVAQHQLETMHQKGMLPDFVLCIGDDRSDEDMFEVIKSAA
AGPSLSPVAEVEACTVGQKPSKAKYYLDDTAEILRMLLGLAEASAQDACKASLGSQRVVINKE
>GAY56626.1 hypothetical protein CUMW_173340 [Citrus unshiu]
MVLIPTCHLQCCLAEVGVRRASKVLLDSKRSSEYDVSLFEDFCREKKRLPRVATVAGVLSEIDDENSNSVGSDAPSSVSQ
ERMIIVGNQLPLRAHRSSDGSGGWTFSWDEDSLLLQLKDGLGEDVEVIYVGCIKEQIDLSEQDEVSQTLLETFKCVPAFI
PPELFSKFYHGFCKQHLWPLFHYMLPLSPDLGGRFDRSLWQAYVSVNKIFADKVMEVISPDDDFVWVHDYHLMVLPTFLR
KRFNRVKLGFFLHSPFPSSEIYRTLPIRDELLRALLNADLIGFHTFDYARHFLSCCSRMLGVSYQSKRGYIGLEYFGRTV
SIKILPVGIHIGQLQSVINLPETEAKVAELQDQFKGQIVMLGVDDMDIFKGISLKLLAMEQLLSQNPSKRGKIVLVQIAN
PARGRGRDVQEVQSETHATVRRINKIFGRPGYQPVVLIDTPLQFYERIAYYVIAECCLVTAVRDGMNLIPYEYIICRQGN
EKLDMTLGLDPSTAKSSMLVVSEFVGCSPSLSGAIRVNPWNIDAVAEAMDSALGVSDAEKQMRHEKHYRYVSTHDVAYWA
RSFLQDLERACRDHMRRRCWGIGFGLGFRVVALDPNFRKLSIDHIVSAYKRTKNRAILLDYDGTIMVPGSISTSPNAEAV
AILDNICRDPKNVVFLVSGKDRDTLAEWFSSCEGLGIAAEHGYFVRPNYGVDWETCVSVPDFSWKQIAEPVMKLYTETTD
GSTIETKESALVWNFQYADPDFGSCQAKELLDHLESVLANEPVSVKSGPNIVEVKPQGVNKGLVAQHQLETMHQKGMLPD
FVLCIGDDRSDEDMFEVIKSAAAGPSLSPVAEVFACTVGQKPSKAKYYLDDTAEILRMLLGLAEASAHDACKASQGSQRV
VINKE
>KDO53157.1 hypothetical protein CISIN_1g002958mg [Citrus sinensis]
MIIVGNQLPLRAHRSSDGSGGWTFSWDEDSLLLQLKDGLGEDVEVIYVGCIKEQIDLSEQDEVSQTLLETFKCVPAFIPP
ELFSKFYHGFCKQHLWPLFHYMLPLSPDLGGRFDRSLWQAYVSVNKIFADKVMEVISPDDDFVWVHDYHLMVLPTFLRKR
FNRVKLGFFLHSPFPSSEIYRTLPIRDELLRALLNADLIGFHTFDYARHFLSCCSRMLGVSYQSKRGYIGLEYFGRTVSI
KILPVGIHIGQLQSVINLPETEAKVAELQDQFKGQIVMLGVDDMDIFKGISLKLLAMEQLLSQNPSKRGKIVIVQIANPA
RGRGRDVQEVQSETHATVRRINKIFGRPGYQPVVLIDTPLQFYERIAYYVIAECCLVTAVRDGMNLIPYEYIICRQGNEK
LDMTLGLDPSTAKSSMLVVSEFVGCSPSLSGAIRVNPWNIDAVAEAMDSALGVSDAEKQMRHEKHYRYVSTHDVAYWARS
FLQDLERACRDHMRRRCWGIGFGLGFRVVALDPNFRKLSIDHIVSAYKRTKNRAILLDYDGTIMVPGSISTSPNAEAVAI
LDNLCRDPKNVVFLVSGKDRDTLAEWFSSCEGLGIAAEHGYFVRPNYGVDWETCVSVPDFSWKQIAEPVMKLYTETTDGS
TIETKESALVWNFQYADPDFGSCQAKELLDHLESVLANEPVSVKSGPNIVEVKPQGVNKGLVAQHQLETMHQKGMLPDFV
LCIGDDRSDEDMFEVIKSAAAGPSLSPVAEVFACTVGQKPSKAKYYLDDTAEILRMLLGLAEASAQDACKASLGSQRVVI
NKE
>XP_006448141.1 alpha, alpha-trehalose-phosphate synthase [UDP-forming] 6 [Citrus
clementina]
MVSKSYSNLLELASGEAPSFGRMRRRIPRIMTVAGIISDLDDDPADSVCSDPSSSSVQRDRIIIVANQLPIRAQRKSDNS
KGWIFSWDENSLLLQLKDGLGDDDIEVIYVGCLKEEIHVNEQDEVSQILLDTFKCVPTFLPPDLFSRYYHGFCKQQLWPL
FHYMLPLSPDLGGRFNRSLWQAYVSVNKIFADRIMEVINPEDDFVWVHDYHLMVLPTFLRKRFNRVKLGFFLHSPFPSSE
IYKTLPIREEILRALLNSDLIGFHTFDYARHFLSCCSRMLGLTYESKRGYIGLEYYGRTVSIKILPVGIHMGQLQSVLSL
PGTEAKVSELIKQFHDQGKVMLLGVDDMDIFKGISLKLLAMEQLLIQHPEWQGKVVLVQIANPARGRGKDVKEVQAETYS
TVERINQTFGKPGYDPVVLIDEPLKFYERIAYYVVAECCLVTAVRDGMNLIPYEYIISRQGNEKLDKVLGSEPSSPKKSM
LVVSEFIGCSPSLSGAIRVNPWNIDAVSDAMDSALEMADQEKQLRHEKHYRYVSTHDVGYWARSFLQDLERTCREHVRQR
CWGIGFGLSFRVVALDPNFKKLSMEHIVSAYKRTTTRAILLDYDGTLMPQASIDKSPNSKTIDILNSLCRDKNNMVFLVS
AKSRKTLAEWFSPCENLGIAAEHGYFFRLRRDEEWETCIPVADCGWKQIAEPVMKLYTETTDGSTIEDKETALVWSYEDA
DPDFGSCQAKELLDHLESVLANEPVTVKSGQNLVEVKPQGVNKGLVAKRLLSTMQEREMLPDFVLCVGDDRSDEDMFEVI
ISSMAGPSIAPRAEVEACTVGRKPSKAKYYLDDTVEIVRLMQGLACVADQMVPV
>KDO60795.1 hypothetical protein CISIN_19044635mg [Citrus sinensis]
MVSKSYSNLLELASGEAPSFGRMRRRIPRIMTVAGIISDLDDDPADSVCSDPSSSSVQRDRIIIVANQLPIRAQRKSDNS
KGWIFSWDENSLLLQLKDGEGDDDIEVIYVGCLKEEIHVNEQDEVSQILLDTFKCVPTFLPPDLFSRYYHGFCKQQLWPL
FHYMLPLSPDLGGRFNRSLWQAYVSVNKIFADRIMEVINPEDDFVWVHDYHLMVLPTFLRKRFNRVKLGFFLASPFPSSE
IYKTLPIREEILRALLNSDLIGFHTFDYARHFLSCCSRMLGLTYESKRGYIGLEYYGRTVSIKILPVGIHMGQLQSVLSL
PGTEAKVSELIKQFHDQGKVMLLGVDDMDIFKGISLKLLAMEQLLIQHPEWQGKVVLVQIANPARGRGKDVKEVQAETYS
TVERINQTFGKPGYDPVVLIDEPLKFYERIAYYVVAECCLVTAVRDGSEPSSPKKSMLVVSEFIGCSPSLSGAIRVNPWN
IDAVSDAMDSALEMADQEKQLRHEKHYRYVSTHDVGYWARSFLQDLERTCREHVRQRCWGIGFGLSFRVVALDPNFKKLS
MEHIVSAYKRTTTRAILLDYDGTLMPQASIDKSPNSKTIDILNSLCRDKNNMVFLVSAKSRKTLAEWFSPCENLGIAAEH
GYFFRLRRDEEWETCIPVADCGWKQIAEPVMKLYTETTDGSTIEDKETALVWSYEDADPDFGSCQAKELLDHLESVLANE
PVTVKSGQNLVEVKPQGVNKGLVAKRLLSTMQEREMLPDFVLCVGDDRSDEDMFEVIISSMAGPSIAPRAEVEACTVGRK
PSKAKYYLDDTVEIVRLMQGLACVADQMVPV
>XP_006449549.1 probable alpha, alpha-trehalose-phosphate synthase [UDP-forming]
7 [Citrus clementina]
MMSKSYTNLLDLASGNFPAMGPSREKKRLPRVMTVPGVISELDDDQANSVSSDVPSSVAQDRVIIVANQLPVKAKRRPDN
KGWSFSWDEDSLLLQLKDGLPEDMEVIYVGSLKVDVDLSEQDDVSQLLLDRFKCVPAFLPPDILTKFYHGFCKQHLWPLF
HYMLPFSATHGGRFDRSLWEAYVSANKIFSQRVIEVINPEDDYVWIHDYHLMVLPTFLRRRFTRLRMGFFLHSPFPSSEI
YRTLPVREEILKALLNADLIGFHTFDYARHFLSCCSRMLGLEYQSKRGYIGLEYYGRTVGIKIMPVGIHMGQIESVERLA
DKDWRVQELKQQFEGKTVLLGVDDMDIFKGVDLKLLAMEHLLKQHPKWQGRAVLVQIANPARGRGKDLEEIQAETHATCK
RINETFGRPGYEPVVFIDKPVTLSERAAYYTIAECVVVTAVRDGMNLTPYEYIVCRQGVSGSESSSESSAPKKSMLVVSE
FIGCSPSLSGAIRVNPWNIEATAEAMHEAIQMNEAEKQLRHEKHYRYVSTHDVAYWARSFFQDMERTCKDHFKRRCWGIG
LSFGFRVVALDPNFRKLSIDAIVSAYLRSKSRAILFDYDGTVMPQTSINKAPSQAVISIINTLCNDARNTVFVVSGRGRD
SLGKWFSPCKKLGIAAEHGYFMRWSADEEWQNCGQSVDFGWIQIAEPVMKLYTESTDGSYIEIKESALVWHHRDADPGFG
SSQAKELLDHLESVLANEPAAVKSGQFIVEVKPQGVSKGVVAEKIFTTMAESGRHADFVLCIGDDRSDEDMFEIIGNATS
SGVLSSNASVFACTVGQKPSKAKYYLDDAAEVVTMLEALAEASAPPSFEVGASDSP
>XP_006467609.1 probable alpha, alpha-trehalose-phosphate synthase [UDP-forming]
7 [Citrus sinensis]
MMSKSYTNLLDLASGNFPAMGPSREKKRLPRVMTVPGVISELDDDQANSVSSDVPSSVAQDRVIIVANQLPVKAKRRPDN
KGWSFSWDEDSLLLQLKDGLPEDMEVIYVGSLKVDVDLSEQDDVSQLLLDRFKCVPAFLPPDILTKFYHGFCKQHLWPLF
HYMLPFSATHGGRFDRSLWEAYVSANKIFSQRVIEVINPEDDYVWIHDYHLMVLPTFLRRRFTRLRMGFFLHSPFPSSEI
YRTLPVREEILKALLNADLIGFHTFDYARHFLSCCSRMLGLEYQSKRGYIGLEYYGRTVGIKIMPVGIHMGQIESVLRLA
DKDWRVQELKQQFEGKTVLLGVDDMDIFKGVDLKLLAMEHLLKQHPKWQGRAVLVQIANPARGRGKDLEEIQAEIHATCK
RINETFGRPGYEPVVFIDKPVTLSERAAYYTIAECVVVTAVRDGMNLTPYEYIVCRQGVSGSESSSESSAPKKSMLVVSE
FIGCSPSLSGAIRVNPWNIEATAEAMHEAIQMNEAEKQLRHEKHYRYVSTHDVAYWARSFFQDMERTCKDHFKRRCWGIG
LSFGFRVVALDPNFRKLSIDAIVSAYLRSKSRAILFDYDGTVMPQTSINKAPSQAVISIINTLCNDARNTVFVVSGRGRD
CLGKWFSPCKKLGIAAEHGYFMRWSADEEWQNCGQSVDFGWIQIAEPVMKLYTESTDGSYIEIKESALVWHHRDADPGFG
SSQAKELLDHLESVLANEPAAVKSGQFIVEVKPQGVSKGVVAEKIFTTMAESGRHADFVLCIGDDRSDEDMFEIIGNATS
SGVLSSNASVFACTVGQKPSKAKYYLDDAAEVVTMLEALAEASAPPSFEVGASDSP
>KDO77921.1 hypothetical protein CISIN_19003025mg [Citrus sinensis]
MMSKSYTNLLDLASGNFPAMGPSREKKRLPRVMTVPGVISELDDDQANSVSSDVPSSVAQDRVIIVANQLPVKAKRRPDN
KGWSFSWDEDSLLLQLKDGLPEDMEVIYVGSLKVDVDLSEQDDVSQLLLDRFKCVPAFLPPDILTKFYHGFCKQHLWPLF
HYMLPFSATHGGRFDRSLWEAYVSANKIFSQRVIEVINPEDDYVWIHDYHLMVLPTFLRRRFTRLRMGFFLHSPFPSSEI
YRTLPVREEILKALLNADLIGFHTFDYARHFLSCCSRMLGLEYQSKRGYIGLEYYGRTVGIKIMPVGIHMGQIESVLRLA
DKDWRVQELKQQFEGKTVLLGVDDMDIFKGVDLKLLAMEHLLKQHPKWQGRAVLVQIANPARGRGKDLEEIQAEIHATCK
RINETFGRPGYEPVVFIDKPVTLSERAAYYTIAECVVVTAVRDGMNLTPYEYIVCRQGVSGSESSSESSAPKKSMLVVSE
FIGCSPSLSGAIRVNPWNIEATAEAMHEAIQMNEAEKQLRHEKHYRYVSTHDVAYWARSFFQDMERTCKDHFKRRCWGIG
LSFGFRVVALDPNFRKLSIDAIVSAYERSKSRAILFDYDGTVMPQTSINKAPSQAVISIINTLCNDARNTVFVVSGRGRD
CLGKWFSPCKKLGIAAERGYFMRWSADEEWQNCGQSVDFGWIQIAEPVMKLYTESTDGSYIEIKESALVWHHRDADPGFG
SSQAKELLDHLESVLANEPAAVKSGQFIVEVKPQVYIQLRI
ACA11 protein sequences
>PtACA11.1_Ptrif.0004s2523.1.v1.3.1_Poncirus_trifoliata
MENYLKKNFDVDPKRPSEEALMRWRSAVRVVKNPRRRFRMVADLAKRAEA
ERKRKKLQEKLRVALYVQKAALHFIDAGSRPIEYKLSQETLLAGYGIEPD
ELESIVRSHNSKAVESHGGVEGLAREVSVSLPDGVASEEVSNRQNVYGFN
RYAEKPARSFWMFVWEALHDLTLIILMICAAVSIGVGIPTEGWPDGMYDG
LGIVLSILLVVIVTAVSDYKQSLQFKALDKEKKNLIVQVTRDGYRKKLSI
YDLVVGDIVHLSIGDQVPADGILISGYNLTIDESSLSGETEPVHINRDRP
FLLSGTKVQDGSGKMLVTSVGMRTEWGRLMVTLSEGGEDETPLQVKLNGV
ATVIGKIGLVFAVLTFLVLTLRFLVEKAQHHQIKNWSSIDAMKLLNYFAI
AVTIVVVAVPEGLPLAVTLSLAFAMKKLMNDKALVRHLSACETMGSASCI
CTDKTGTLTTNHMVVTKLWICNEAKTIKRGDNEKLLKPSVSDAVFNIFLQ
SIFQNTGSEVVKDKDGRTNILGTPTERAILEFGLILGGDSKFHREESAIV
KVEPFNSVKKRMSVLVSLPNNGGFRAFCKGASEIILNMCNKIINADRKAV
PISEEQRKNLTNVINGFSSEALRTLCLAFQDIKGNHKAESIPENNYTLIA
VVGIKDPVRPGVREAVETCLAAGITVRMVTGDNIHTAKAIAKECGILTDG
GLAIEGTDFRSKNPQEMQELIPKLQVMARSSPTDKYILVTQLRNVFKEVV
AVTGDGTNDAPALHEADIGLAMGIAGTEVAKENADVIIMDDNFTTIVTVA
RWGRSVYINIQKFVQFQLTVNIVALVINFVAACITGSAPLTAVQLLWVNM
IMDTLGALALATEPPHEGLMQRPPIGRNVHFITVIMWRNIIGQSIYQIIV
LGVLTFCGKKILKLSGPNATLILNTFIFNSFVFCQVFNEINSRDMEKINV
FRGIFSSWVFIAVLVATVGFQVIIVELLGTFATTVPLNWKLWLASVVIGA
ISMPFGVLLKCIPAGTCTSAANSKHHDGYEPLPTGPDLA-
> PtACA11.2_Ptrif.0009s1528.2.v1.3.1_Poncirus_trifoliata
MDKFLNWKDEDVEHKNPSEEALRRWRSAVSIVKNRRRRERMVADLVKRSE
GEKKKLKIQEKIRVALYVQKAALTFIDAAGRPEYKLSEETRDAGFLIDPD
DLAAIVRGRDIKGLKSNDGVEGVAQKLSVSLNEGVCKRDLPIRQKIYGVN
RYTEKPPRSFLMFVWDALQDLTLIILIVCAVLSIGVGLATEGWPEGMYDG
LGIILSILLVVMVTAISDYKQSLQFRDLDREKKKIFIQVTRDGQRQKVSI
YDLVVGDIVHLSIGDQVAADGIFISGYSLLIDESSLSGESEPMYICEENP
FLLAGTKVQDGSGKMLVTTVGMRTEWGKLMETLNEGGEDETPLQVKLNGV
ATIIGKIGLFFSVLTFLVLAGRFLGEKAIHNEFTVWSSADALTLIDYFAV
AVTIIVVAVPEGLPLAVTLSLAFAMKKLMNDRALVRHLSACETMGSASCI
CTDKTGTLTTNHMVVDKIWICNTISKVEGNNKEDILQLEISERVEDITLQ
AIFQNTGSEVVKDKDGKNSILGTPTESAILEFGLRLGGDFEAQRREFKIV
KVEPFNSVRKKMSVLIALPAGGMRAFCKGASEIVLSMCDKVVSDNGEPVP
LSEEQFRNITDVINGFASEALRTLCLAFKDLNDSSNENNIPDSGYTLIAV
VGIKDPVRPGVKEAVQTCLEAGITVRMVTGDNINTARAIAKECGILTSDG
EAVEGPEFRNMSPADMKRIIPKLQVMARSLPLDKHTLVTQLRKTFGEVVA
VTGDGTNDAPALHEADIGLSMGIAGTEVAKGNADVIILDDNFSTIVNVAK
WGRAVYINIQKFVQFQLTVNVVALVINFVSACASGSAPLTAVQLLWVNMI
MDTLGALALATEPPHEGLMKRPPVAKGESFITKVMWRNIIGQSIYQLIIL
VALNFDGKQILGLSGSDATAVLNTVIFNSFVFCQVFNEINSREMEKINVF
KGMFDSWMFVGILVLTVAFQIIIIEFLGAFASTVPLSWHLWLLCILIGAV
SMPIAVVIKCIPVKKSEPKLQHHDGYEEIPSGPESA-
> PtACA11.3_Ptrif.0007s0578.1.v1.3.1_Poncirus_trifoliata
MESYLQENFGVKPKHSSTEALEKWRNLCGVVKNPKRRFRFTANLPKRYEA
AAMRKTNQEKLRIAVLVSKAAIQFLLGVTPSDYNVPEEVKAAGFQVCAEE
LGSITEGHDVKKLKFHGGVTGIAEKLSTSISDGLTSNTDLFNRRQEIYGL
NQFAESTPRSFWVFVWEALQDMTLMILGACAFVSLIVGIVMEGWPHGAHD
GLGIVASILLVVFVTATSDYRQSLQFKDLDKEKKKIFVQVTRNGFRQKLS
IYDLLPGDIVHLGIGDQVPADGLFVSGFSVLIDESSLTGESEPVMVNEEN
PFMLSGTKLQDGSCKMMVTTVGMRTQWGKLMATLSEGGDDETPLQVKLNG
VATIIGKGGLFFAVVTFAVLVQGLLSHKLGEGSIWSWSGDDALKLLEYFA
VAVTIVVVAVPEGLPLAVTLSLAFAMKKMMNDKALVRHLAACETMGSASS
ICSDKTGTLTTNHMTVVKSCICMNVKEVSKTDSASSLCSEIPDSAVQLLL
QSIFTNTGGEVVVNKDGKREILGTPTETALLEFGLSLGGDFQAERQTSKI
VKVEPFNSSKKRMGVVLELPGGGLRVHSKGASEIVLSGCDKVVNSTGEVV
PLDEESLNHLKLTIDQFANEALRTLCLAFMELETGFSPENPIPVSGYTLI
AIVGIKDPVRPGVKESVAVCRSAGITVRMVTGDNINTAKAIARECGILTD
DGIAIEGPVFREKTTEELMELIPKIQVMARSSPLDKHTLVKHLRTTFDEV
VAVTGDGTNDAPALHEADIGLAMGIAGTEVAKESADVIILDDNFSTIATV
AKWGRSVYINIQKFVQFQLTVNIVALIVNFSSACLTGSAPLTAVQLLWVN
MIMDTLGALALATEPPTDELMKRPPVGKRGNFISNVMWRNILGQSLYQFM
VISLLQAKGKAIFWLDGPDSTLVINTLIFNSFVFCQIFNEISSREMEEIN
VFKGILDNYVFASVLGVTVFFQIIIVEFLGTFANTTPLTLTQWFASIVIG
FIGMPIAAGLKTIQV-
> PtACA11.4_Ptrif.0005s1708.2.v1.3.1_Poncirus_trifoliata
MENYLNENFSDVKAKNTSEEALQRWRKLCGFVKNKKRRFRFTANLSKRFE
AEAIRRSNQEKFRVAVLVSQAALQFIHGLNLSSEYTVPEEVAASGFQICP
DELGSIVEGHDIKKLKVHGGVEGIAEKLSTSITDGISTSEQLLNRRKEIY
GINKFTESPARGFWVYVWEALHDMTLMILAVCALVSLVVGIATEGWPKGA
HDGLGIVMSILLVVFVTATSDYKQSLQFKDLDKEKKKITVQVARNGFRRK
ISIYDLLPGDIVHLCMGDQVPADGLFVSGFSVLINESSLTGESEPVNVNA
LNPFLLSGTKVQNGSCKMLVTTVGMRTQWGKLMATLSEGGDDETPLQVKL
NGVATIIGKIGLFFAVVTFAVMVQGLFTRKLQEGTHWTWSGDDALEILEF
FAIAVTIVVVAVPEGLPLAVTLSLAFAMKKMMNDKALVRHLAACETMGSA
TSICSDKTGILTTNHMTVLKACICEEIKEVDNYKGTPAFGSSIPASASKL
LLQSIFNNTGGEVVIGEGNKTEILGTPTETAILEFGLLLGGDFQAERQAS
KIVKVEPFNSVKKQMGVVIELPEGGFRVHCKGASEIILAACDKFLNANGE
VVPLNEAAVNHLNKTIEKFASEALRTLCLAYMEIGNEFSADAPIPTQGYT
CIGIVGIKDPMRPGVKESVAICRSAGITVRMVTGDNINTAKAIARECGIL
TDNGIAIEGPEFREKSDEELSKLIPKIQVMARSSPMDKHTLVKHLRTTLG
EVVAVTGDGTNDAPALHEADIGLAMGIAGTEVAKESADVIILDDNFSTIV
TVAKWGRSVYINIQKFVQFQLTVNVVALIVNFSSACLTGNAPLTAVQLLW
VNMIMDTLGALALATEPPNGDLMKRSPVGRKGNFISNVMWRNILGQSLYQ
FLIIWYLQTRGKAVFHLDGPDPDLILNTLIFNTFVFCQVFNEISSREMEK
INVLKGILKNYVEVAVLTCTVLFQIIIIELLGTFANTTPLNLQQWFVSIL
LGFLGMPIAAVLKLIHVG-
> PtACA11.5_Ptrif.0009s1531.2.v1.3.1_Poncirus_trifoliata
MDKFFNWKDFDVEHKNPSEEALRRWRSAACIVKNRRRRFRMVADLDKRSD
AEKKKLEIKQKIQVAIDVQREALRLTDAAGRAEYKLSEETRQAGFGTDPD
DLAAIVCGHDTEGLKSNEGVEGVAQKLSVSLNEGVRKRDVPIRQNIYGVN
RYTEKPPRSFFMFVWEALQDLTLIILMVCAGLSIGVGLAREGWPEGIYDG
LGIILSKFLVVMVTAISDYKQSLQFRDLDREKKKIFIQVTRDGQRQKVCI
YDLVVGDIVALSIGDQVPADGIFISGHSLLIDESSLSGQSEPRYMYEENP
FLLAGTEVQGGSGKMLVTTVGMRTEWGKLMETLNEGGEDETPLQVKLNGV
ATIIGKIELFFSVLEFLVLIGRFLGEKVIHNEFTDWSSADALTLIDYFAV
VVTIIDVAVPEGLPLAVTLSLAFAVKKLMNDGALVRHLSACETMGSASCI
CTDKTGTLTTNHMVVDKIWICNTISKVEGNNREDILQLEISERVLDITLQ
AIFQNTGSEVVEDKDGKNSILGTPTESAILEFGLRLGGDFEAQRREFKLV
KVEPFNSVRKKMSVLIALPAGGMRAFCKGASEIVLSMCDKVVSDNGEPVP
LSEEQFRNITDVINGFASEALRTLCLAFKDLNDSSNENNIPDSGYTLIAV
VGIKDPVRPGVKEAVQTCLEAGITVRMVTGDNINTARAIAKECGILTSDG
EAVEGPEFRNMSPADIIPKLQVMARSLPSDKHTLVTQLRNTFGEVVAVTG
DGTNDAAALHEADIGLAMGIAGTECKISAEQNKFIKK-
>XP_006438912.1 putative calcium-transporting ATPase 11, plasma membrane-type
[Citrus clementina]
MENYLKKNFDVDPKRPSEEALMRWRSAVRVVKNPRRRFRMVADLAKRAEAERKRKKLQEKLRVALYVQKAALHFIDAGSR
PIEYKLSQETLLAGYGIEPDELESIVRSHNSKAVESHGGVEGLAREVSVSLPDGVASEEVSNRQNVYGFNRYAEKPARSF
WMFVWEALHDLTLIILMICAAVSIGVGIPTEGWPDGVYDGLGIVLSILLVVIVTAVSDYKQSLQFKALDKEKKNLIVQVT
RDGYRKKLSIYDLVVGDIVHLSIGDQVPADGILISGYSLTIDESSLSGETEPVHINRDRPFLLSGTKVQDGSGKMLVTSV
GMRTEWGRLMVTLSEGGEDETPLQVKLNGVATVIGKIGLVFAVLTFLVLALRFLVEKAQHHQIKNWSSIDAMKLLNYFAI
AVTIVVVAVPEGLPLAVTLSLAFAMKKLMNDKALVRHLSACETMGSASCICTDKTGTLTTNHMVVTKLWICNEAKTIKSG
DNEKLLKPSVSDAVFNIFLQSIFQNTGSEVVKDKDGRTNILGTPTERAILEFGLILGGDSTFHREESAIVKVEPFNSVKK
RMSVLVSLPNNGGFRVFCKGASEIILNMCDKIINADGKAVPISEEQRKNLINVINGFSSEALRTLCLAFQDIKGNHKAES
IPENNYTLIAVVGIKDPVRPGVREAVETCLAAGITVRMVTGDNIHTAKAIAKECGILTDGGLAIEGTDFRSKNPQEMQEL
IPKLQVMARSSPTDKYILVTQLRNVFKEVVAVTGDGTNDAPALHEADIGLAMGIAGTEVAKENADVIIMDDNFTTIVIVA
RWGRSVYINIQKFVQFQLTVNIVALVINFVAACITGSAPLTAVQLLWVNMIMDTLGALALATEPPHEGLMQRPPIGRNVH
FITVTMWRNIIGQSIYQIIVLGVLTFCGKKILKLSGPNATLILNTFIFNSFVFCQVFNEINSRDMEKINVFRGIFSSWVF
VAVLVATVGFQVIIVELLGTFATTVPLNWKLWLASVVIGAISMPFGVLLKCIPVGTCTSAANSKHHDGYEPLPTGPDLA
>KDO83263.1 hypothetical protein CISIN_1g0016382mg, partial [Citrus sinensis]
EKLRVALYVQKAALHFIDAGSRPIEYKLSQETLLAGYGIEPDELESIVRSHNSKAVESRGGVEGLAREVSVSLPDGVASE
EVSNRQNVYGFNRYAEKPARSFWMFVWEALHDLTLIILMICAAVSIGVGIPTEGWPDGVYDGLGIVLSILLVVIVTAVSD
YKQSLQFKALDKEKKNLIVQVTRDGYRKKLSIYDLVVGDIVHLSIGDQVPADGILISGYSLTIDESSLSGETEPVHINRD
RPFLLSGTKVQDGSGKMLVTSVGMRTEWGRLMVTLSEGGEDETPLQVKLNGVATVIGKIGLVFAVLTFLVLALRFLVEKA
QHHQIKHWSSIDAMKLLNYFAIAVTIVVVAVPEGLPLAVTLSLAFAMKKLMNDKALVRHLSACETMGSASCICTDKTGTL
TTNHMVVTKLWICNEAKTIKSGDNEKLLKPSVSDAVFNIFLQSIFQNTGSEVVKDKDGRTNILGTPTERAILEFGLILGG
DSTFHREESAIVKVEPFNSVKKPMSVLVSLPNNGGFRVFCKGASEIILNMCDKIINADGKAVPISEEQRKNLTNVINGFS
SEALRTLCLAFQDIKGNHKAESIPENNYTLIAVVGIKDPVRPGVREAVETCLAAGITVRMVTGDNIHTAKAIAKECGILT
DGGLAIEGTDFRSKNPQEMQELIPKLQVMARSSPTDKYILVTQLRNVFKEVVAVTGNGTNDAPALHEADIGLAMGIAGTE
VAKENADVIIMDDNFTTIVTVARWGRSVYINIQKFVQFQLTVNIVALVINFVAACITGSAPLTAVQLLWVNMIMDTLGAL
ALATEPPHEGLMQRPPIGRNVHFITVTMWRNIIGQSIYQIIVLGVLTFCGKKILKLSGPNATLILNTFIFNSFVFCQVFN
EINSRDMEKINVFRGIFSSWVFIAVLVATVGFQVIIVELLGTFATTVPLNWKLWLASVVIGAISMPFGVLLKCIPVGTCT
SAANSKHHDGYEPLPTGPDLA
>XP_006492951.1 putative calcium-transporting ATPase 11, plasma membrane-type
[Citrus sinensis]
MDKFLNWKDFDVEHKNPSEEALRRWRSAVSIVKNRRRRFRMVADLVKRSEGEKKKLKIQEKIRVALYVQKAALTFIDAAG
RPEYKLSEETREVGFRIEPDDLAVIVRGRDIKGLKSNDGVEGVAQKLSVSLNEGVCKRDLPIRQKTYGVNRYTEKPPRSF
LMFVWDALQDLTLIILIVCAVLSIGVGLATEGWPEGMYDGLGIILSILLVVMVTAISDYKQSLQFRDLDREKKKIFIQVT
RDGQRQKVSIYDLVVGDIVHLSIGDQVAADGIFISGYSLLIDESSLSGESEPMYICEENPFLLAGTKVQDGSGKMLVTTV
GMRTEWGKLMETLNEGGEDETPLQVKLNGVATIIGKIGLFFSVLTFLVLAGRFLGEKAIHNEFTVWSSADALTLIDYFAV
AVTIIVVAVPEGLPLAVTLSLAFAMKKLMNDRALVRHLSACETMGSASCICTDKTGTLTTNHMVVDKIWICNTISKVEGN
NREDILQLEISERVLDVTLQAIFQNTGSEVVKDKDGKNSILGTPTESAILEFGLHLGGDFEAQRREFKIVKVEPFNSVRK
KMSVLIALPAGGMRAFCKGASEIVLSMCDKVVSDNGEPVPLSEEQFRNITDVINGFASEALRTLCLAFKDLNDSSNENNI
PDSGYTLIAVVGIKDPVRPGVKEAVQTCLEAGITVRMVTGDNINTARAIAKECGILTSDGEAVEGPEFRNMSPADMKRII
PKLQVMARSLPLDKHTLVTQLRKTFGEVVAVTGDGTNDAPALHEADIGLSMGIAGTEVAKGNADVIILDDNFSTIVNVAK
WGRAVYINIQKFVQFQLTVNVVALVINFVSACASGSAPLTAVQLLWVNMIMDTLGALALATEPPHEGLMKRPPVAKGESF
ITKVMWRNIIGQSIYQLIILVALNFDGKQILGLSGSDATAVINTVIFNSFVFCQVFNEINSREMEKINVFKGMFDSWLFV
GILVLTVAFQIIIVEFLGALASTVPLSWHLWLLCILIGAVSMPIAVVIKCIPVKKSEPKLQHHDGYEEIPSGPESA
>XP_006421285.2 putative calcium-transporting ATPase 11, plasma membrane-type
[Citrus clementina]
MDKFLNWKDFDVEHKNPSEEALRRWRSAVSIVKNRRRRFRMVADLVKRSEGEKKKLKIQEKIRVALYVQKAALQFIDAAG
RPEYKLSEETREAGFRIEPDDLAAIVRGRDIKGLKSNDGVEGVAQKLSVSLNEGVCKRDLPIRQKIYGVNRYTEKPPRSF
LMFVWDALQDLTLIILIVCAVLSIGVGLATEGWPEGMYDGLGIIVSILLVVMVTAISDYKQSLQFRDLDREKKKIFIQVT
RDGQRQKVSIYDLVVGDIVHLSIGDQVAADGIFISGYSLLIDESSLSGESEPMYICEENPFLLAGTKVQDGSGKMLVTTV
GMRTEWGKLMETLNEGGEDETPLQVKLNGVATIIGKIGLFFSVLTFLVLAGRFLGVKAIHNEFTVWSSADALTLIDYFAV
AVTIIVVAVPEGLPLAVTLSLAFAMKKLMNDRALVRHLSACETMGSASCICTDKTGTLTTNHMVVDKIWICNTISKVEGN
NREAILQLEISERVLDITLQAIFQNTGSEVVKDKDGKNSILGTPTESAILEFGLRLGGDFEAQRREFKIVKVEPFNSVRK
KMSVLIALPAGGMRAFCKGASEIVLSMCDKVVSDNGEPVPLSEEQFRNITDVINGFASEALRTLCLAFKDLNDSSNENNI
PDSGYTLIAVVGIKDPVRPGVKEAVQTCLEAGITVRMVTGDNINTARAIAKECGILTSDGEAVEGPEFRNMSPADMKRII
PKLQVMARSLPLDKHTLVTQLRKTFGEVVAVTGDGTNDAPALHEADIGLSMGIAGTEVAKGNADVIILDDNFSTIVNVAK
WGRAVYINIQKFVQFQLTVNVVALVINFVSACASGSAPLTAVQLLWVNMIMDTLGALALATEPPHEGLMKRPPVAKGESF
ITKVMWRNIIGQSIYQLIILVALNFDGKQILGLSGSDATAVINTVIFNSFVFCQVFNEINSREMEKINVFKGMFDSWMFV
GILVLTVAFQIIIIEFLGAFASTVPLSWHQWLLCILIGAVSMPIAVVIKCIPVKKSEPKIQHHDGYEEIPSGPESA
>XP_024037041.1 calcium-transporting ATPase 4, plasma membrane-type [Citrus
clementina]
MDKFFNWKDFDVEHKNPSEEALRRWRSAASIVKNRRRRFRMVADLDKRSEAEKKKLEIKQKIQVAIDVQRAALQLTDAAG
RAEYKLSEETRQAGFGIDPDDLAAIVCGHDIEGLKSNEGVEGVAQKLSVSLNEGVHKRDVPIRQNIYGVNRYTEKPPRSF
FMFVWEALQDLTLIILMVCAGLSIGVGLAREGWPEGIYDGIFLVVLGIILSKFLVVMVTAISDYKQSLQFRDLDREKKKI
FIQVTRDGQRQKVCTYDLVVGDIVHLSIGDQVPAYGIFISGHSLLIDESSLSGQSEPRYMYEENPFLLAGTKVQGGSGKM
LVTTVGMRTEWGKLMETLNEGGEDETPLQVKLNGVATIIGKIELFFSVLEFLVLIGRFLGEKVIHNEFTDWSSADALTLI
DYFAVVVTIIDVAVPEGLPLAVTLSLAFAMKKLMNDRALVRHLSACETMGSASCICTDKTRMLTTNHMVVDKIWIANTIS
NVEGNNRKDILQSEISERVLDITLQAIFQNTGSEVVKDKDGKNSILGTPTESAILEFGLRLGGDFEAQRREFKIVKVEPF
NSVRKKMSVLIALPAGGMRAFCKGASEIVLSMCDKVVSDNGEPVPLSEEQFRNITDVINGFASEALRTLCLAFKDLNDSS
NENNIPDSGYTLIAVVGIKDPVRPGVKEAVQTCLEAGITVRMVTGNNINTARAIAKECGILTSDGEAVEGPEFRNMSPAD
IIPKLQVMARSLPSDKHTLVTQLRNTFGEVVAVTGDGTNDASALHEADIGLAMGIAGTEVAKGNADVIILDDNFSTIVNV
AKWGRAVYINIQKFVQFQLTVNVVALVINFVSACASGSAPLTAVQLLWVNMIMDTLGALALATEPPHEGLMKRPPVAKGE
SFITKVMWRNIIGQSIYQLIILVVLNFDGKQILRLSGSDASAVINTVIFNSFVFFQVFNEINSRDMEKINVFKGMFDSWM
FVGILVETVAFQIIIVEFLGAFASTVPLSWQLWLLCILIGAGSMPIAAVIKCVPVKKCEPKLQRHD
>ESR34525.1 hypothetical protein CICLE v10004282mg [Citrus clementina]
MFVWDALQDLTLIILIVCAVLSIGVGLATEGWPEGMYDGLGIIVSILLVVMVTAISDYKQSLQFRDLDREKKKIFIQVTR
DGQRQKVSIYDLVVGDIVHLSIGDQVAADGIFISGYSLLIDESSLSGESEPMYICEENPFLLAGTKVQDGSGKMLVTTVG
MRTEWGKLMETLNEGGEDETPLQVKLNGVATIIGKIGLFFSVLTFLVLAGRFLGVKAIHNEFTVWSSADALTLIDYFAVA
VTIIVVAVPEGLPLAVTLSLAFAMKKLMNDRALVRHLSACETMGSASCICTDKTGTLTTNHMVVDKIWICNTISKVEGNN
REAILQLEISERVLDITLQAIFQNTGSEVVKDKDGKNSILGTPTESAILEFGLRLGGDFEAQRREFKIVKVEPFNSVRKK
MSVLIALPAGGMRAFCKGASEIVLSMCDKVVSDNGEPVPLSEEQFRNITDVINGFASEALRTLCLAFKDLNDSSNENNIP
DSGYTLIAVVGIKDPVRPGVKEAVQTCLEAGITVRMVTGDNINTARAIAKECGILTSDGEAVEGPEFRNMSPADMKRIIP
KLQVMARSLPLDKHTLVTQLRKTFGEVVAVTGDGINDAPALHEADIGLSMGIAGTEVAKGNADVIILDDNFSTIVNVAKW
GRAVYINIQKFVQFQLTVNVVALVINFVSACASGSAPLTAVQLLWVNMIMDTLGALALATEPPHEGLMKRPPVAKGESFI
TKVMWRNIIGQSIYQLIILVALNFDGKQILGLSGSDATAVLNTVIFNSFVFCQVFNEINSREMEKINVFKGMFDSWMFVG
ILVLIVAFQIIIIEFLGAFASTVPLSWHQWLLCILIGAVSMPIAVVIKCIPVKKSEPKIQHHDGYEEIPSGPESA
>XP_006472295.1 calcium-transporting ATPase 1 [Citrus sinensis]
MENYLNENFSDVKAKNTSEEALQRWRKLCGFVKNKKRRFRFTANLSKRFEAEAIRRSNQEKFRVAVLVSQAALQFIHGLN
LSSEYTVPEEVAASGFQICPDELGSIVEGHDIKKLKVHGGVEGIAEKLSTSITDGISTSEHLLNRRKEIYGINKFTESPA
RGFWVYVWEALHDMTLMILAVCALVSLVVGIATEGWPKGAHDGLGIVMSILLVVFVTATSDYKQSLQFKDLDREKKKITV
QVARNGFRRKISIYDLLPGDIVHLCMGDQVPADGLFVSGFSVLINESSLTGESEPVNVNALNPFLLSGTKVQNGSCKMLV
TTVGMRTQWGKLMATLSEGGDDETPLQVKLNGVATIIGKIGLFFAVVTFAVMVQGLFTRKLQEGTHWTWSGDDALEILEF
FAIAVTIVVVAVPEGLPLAVTLSLAFAMKKMMNDKALVRHLAACETMGSATSICSDKTGTLTTNHMTVLKACICEEIKEV
DNSKGTPAFGSSIPASASKLLLQSIFNNTGGEVVIGEGNKTEILGTPTETAILEFGLLLGGDFQAERQASKIVKVEPFNS
VKKQMGVVIELPEGGFRVHCKGASEIILAACDKFLNSNGEVVPLNEAAVNHLNETIEKFASEALRTLCLAYMEIGNEFSA
DAPIPTEGYTCIGIVGIKDPMRPGVKESVAICRSAGITVRMVTGDNINTAKAIARECGILTDNGIAIEGPEFREKSDEEL
SKLIPKIQVMARSSPMDKHTLVKHLRTTLGEVVAVTGDGTNDAPALHEADIGLAMGIAGTEVAKESADVIILDDNFSTIV
TVAKWGRSVYINIQKFVQFQLTVNVVALIVNFSSACLTGNAPLTAVQLLWVNMIMDTLGALALATEPPNGDLMKRSPVGR
KGNFISNVMWRNILGQSLYQFLIIWYLQTRGKAVFRLDGPDPDLILNTLIFNTFVFCQVFNEISSREMEKINVFKGILKN
YVFVAVLTCTVLFQIIIIELLGTFANTTPLNLQQWFVSILLGFLGMPIAAVLKLIQVG
>GAY47979.1 hypothetical protein CUMW_108500 [Citrus unshiu]
MENYLNENFSDVKAKNTSEEALQRWRKLCGFVKNRKRRFRFTANLSKRFEAEAIRRSNQEKFRVAVLVSQAALQFIHGLN
LSSEYTVPEEVAASGFQICPDELGSIVEGHDIKKLKVHGGVEGIAEKLSTSITDGISTSEHLLNRRKEIYGINKFTESPA
RGFWVYVWEALHDMTLMILAVCALVSLVVGIATEGWPKGAHDGLGIVMSILLVVFVTATSDYKQSLQFKDLDREKKKITV
QVARNGFRRKISIYDLLPGDIVHLCMGDQVPADGLFVSGFSVLINESSLTGESEPVNVNALNPFLLSGTKVQNGSCKMLV
TTVGMRTQWGKLMATLSEGGDDETPLQVKINGVATIIGKIGLFFAVVTFAVMVQGLFTRKLQEGTHWTWSGDDALEILEF
FAIAVTIVVVAVPEGLPLAVTLSLAFAMKKMMNDKALVRHLAACETMGSATSICSDKTGTLTTNHMTVLKACICEEIKEV
DNSKGTPAFGSSIPASASKLLLQSIFNNTGGEVVIGEGNKTEILGTPTETAILEFGLLLGGDFQAERQASKIVKVEPFNS
VKKQMGVVIELPEGGFRVHCKGASEIILAACDKFLNSNGEVVPLNEAAVNHLNETIEKFASEALRTLCLAYMEIGNEFSA
DAPIPTEGYTCIGIVGIKDPMRPGVKESVAICRSAGITVRMVTGDNINTAKAIARECGILTDNGIAIEGPEFREKSDEEL
SKLIPKIQVMARSSPMDKHTLVKHLRTTLGEVVAVTGDGTNDAPALHEADIGLAMGIAGTEVAKESADVIILDDNFSTIV
TVAKWGRSVYINIQKFVQFQLTVNVVALIVNFSSACLTGNAPLTAVQLLWVNMIMDTLGALALATEPPNGDLMKRSPVGR
KGNFISNVMWRNILGQSLYQFLIIWYLQTRGKAVFRLDGPDPDLILNTLIFNTFVFCQVFNEISSREMEKINVFKGILKN
YVFVAVLTCTVLFQIIIIELLGTFANTTPLNLQQWFVSILLGFLGMPIAAVLKLIQVG
>KDO78876.1 hypothetical protein CISIN_19001775mg [Citrus sinensis]
MESYLQENFGVKPKHSSTEALEKWRNLCGVVKNPKRRFRFTANLSKRYEAAAMRKTNQEKLRIAVLVSKAAIQFLLGVTP
SDYNVPEEVKAAGFQVCAEELGSITEGHDVKKLKFHGGVTGIAEKLSTSISDGLTSNTDLFNRRQEIYGLNQFAESTPRS
FWVFVWEALQDMTLMILGACAFVSLIVGIVMEGWPHGAHDGLGIVASILLVVFVTATSDYRQSLQFKDLDKEKKKIYVQV
TRNGFRQKLSIYDLLPGDIVHLGIGDQVPADGLFVSGFSVLIDESSLTGESEPVMVNEENPFMLSGTKLQDGSCKMMVTT
VGMRTQWGKLMATLSEGGDDETPLQVKLNGVATIIGKGGLFFAVVTFAVLVQGLLSHKLGEGSIWSWSGDDALKLLEYFA
VAVTIVVVAVPEGLPLAVTLSLAFAMKKMMNDKALVRHLAACETMGSASSICSDKTGTLTTNHMTVVKSCICMNVKEVSK
TDSASSLCSEIPDSAVQLLLQSIFTNTGGEVVVNKDGKREILGTPTETALLEFGLSLGGDFQAERQTSKIVKVEPFNSSK
KRMGVVLELPGGGLRAHSKGASEIVLSGCDKVVNSTGEVVPLDEESLNHLKLTIDQFANEALRTLCLAFMELETGFSPEN
PIPVSGYTLIAIVGIKDPVRPGVKESVAVCRSAGITVRMVTGDNINTAKAIARECGILTDDGIAIEGPVFREKTTEELME
LIPKIQVMARSSPLDKHTLVKHLRTTFDEVVAVTGDGTNDAPALHEADIGLAMGIAGTEVAKESADVIILDDNFSTIATV
AKWGRSVYINIQKFVQFQLTVNIVALIVNFSSACLTGSAPLTAVQLLWVNMIMDTLGALALATEPPTDELMKRPPVGKRG
NFISNVMWRNILGQSLYQFMVISLLQAKGKAIFWLDGPDSTLVLNTLIFNSFVFCQIFNEISSREMEEINVFKGILDNYV
FASVLGVTVFFQIIIVEFLGTFANTTPLTLTQWFASIVIGFIGMPIAAGLKTIQV
>XP_006433631.1 calcium-transporting ATPase 1 [Citrus clementina]
MENYLNENFSDVKAKNTSEEALQRWRKLCGFVKNRKRRFRFTANLSKRFEAEAIRRSNQEKFRVAVLVSQAALQFIHGLN
LSSEYTVPEEVAASGFQICPDELGSIVEGHDIKKLKVHGGVEGIAEKLSTSITDGISTSEHLLNRRKEIYGINKFTESPA
RGFWVYVWEALHDMTLMILAVCALVSLVVGIATEGWPKGAHDGLGIVMSILLVVFVTATSDYKQSLQFKDLDREKKKITV
QVARNGFRRKISIYDLLPGDIVHLCMGDQVPADGLFVSGFSVLINESSLTGESEPVNVNALNPFLLSGTKVQNGSCKMLV
TTVGMRTQWGKLMATLSEGGDDETPLQVKINGVATIIGKIGLFFAVVTFAVMVQGLFTRKLQEGTHWTWSGDDALEILEF
FAIAVTIVVVAVPEGLPLAVTLSLAFAMKKMMNDKALVRHLAACETMGSATSICSDKTGTLTTNHMTVLKACICEEIKEV
DNSKGTPAFGSSIPASASKLLLQSIFNNTGGEVVIGEGNKTEILGTPTETAILEFGLLLGGDFQAERQASKIVKVEPFNS
VKKQMGVVIELPEGGFRVHCKGASEIILAACDKFLNSNGEVVPLNEAAVNHLNETIEKFASEALRTLCLACMEIGNEFSA
DAPIPTEGYTCIGIVGIKDPMRPGVKESVAICRSAGITVRMVTGDNINTAKAIARECGILTDNGIAIEGPEFREKSDEEL
SKLIPKIQVMARSSPMDKHTLVKHLRTTLGEVVAVTGDGTNDAPALHEADIGLAMGIAGTEVAKESADVIILDDNFSTIV
TVAKWGRSVYINIQKFVQFQLTVNVVALIVNFSSACLTGNAPLTAVQLLWVNMIMDTLGALALATEPPNGDLMKRSPVGR
KGNFISNVMWRNILGQSLYQFLIIWYLQTRGKAVFRLDGPDPDLILNTLIFNTFVFCQVFNEISSREMEKINVFKGILKN
YVFVAVLTCTVLFQIIIIELLGTFANTTPLNLQQWFVSILLGFLGMPIAAVLKLIQVG
>XP_006466431.1 calcium-transporting ATPase 2, plasma membrane-type-like [Citrus
sinensis]
MESYLQENFGVKPKHSSTEALEKWRNLCGVVKNPKRRFRFTANLSKRYEAAAMRKTNQEKLRIAVLVSKAAIQFLLGVTP
SDYNVPEEVKAAGFQVCAEELGSITEGHDVKKLKFHGGVTGIAEKLSTSISDGLTSNTDLFNRRQEIYGLNQFAESTPRS
FWVFVWEALQDMILMILGACAFVSLIVGIVMEGWPHGAHDGLGIVASILLVVFVTATSDYRQSLQFKDLDKEKKKIYVQV
TRNGFRQKLSIYDLLPGDIVALGIGDQVPADGLFVSGFSVLIDESSLTGESEPVMVNEENPFMLSGTKLQDGSCKMMVTT
VGMRTQWGKLMATLSEGGDDETPLQVKLNGVATIIGKGGLFFAVVTFAVLVQGLLSHKLGEGSIWSWSGDDALKLLEYFA
VAVTIVVVAVPEGLPLAVTLSLAFAMKKMMNDKALVRHLAACETMGSASSICSDKTGTLTTNHMTVVKSCICMNIKEVSK
TDSASSLCSEIPDSAVQLLLQSIFTNTGGEVVVNKDGKREILGTPTETALLEFGLSLGGDFQAERQTSKIVKVEPFNSSK
KRMGVVLELPGGGLRAHSKGASEIVLSGCDKVVNSTGEVVPLDEESLNHLKLTIDQFANEALRTLCLAFMELETGFSPEN
PIPVSGYTLIAIVGIKDPVRPGVKESVAVCRSAGITVRMVTGDNINTAKAIARECGILTDDGIAIEGPVFREKTTEELME
LIPKIQVMARSSPLDKHTLVKHLRTTFDEVVAVTGDGTNDAPALHEADIGLAMGIAGTEVAKESADVIILDDNFSTIATV
AKWGRSVYINIQKFVQFQLTVNIVALIVNFSSACLTGSAPLTAVQLLWVNMIMDTLGALALATEPPTDELMKRPPVGKRG
NFISNVMWRNILGQSLYQFMVISLLQAKGKAIFWLDGPDSTLVLNTLIFNSFVFCQIFNEISSREMEEINVFKGILDNYV
FASVLGVTVFFQIIIVEFLGTFANTTPLTLTQWFASIVIGFIGMPIAAGLKTIQV
>XP_006426128.1 calcium-transporting ATPase 2, plasma membrane-type [Citrus
clementina]
MESYLQENFGVKPKHSSTEALEKWRNLCGVVKNPKRRFRFTANLSKRYEAAAMRKTNQEKLRIAVLVSKAAIQFLLGVTP
SDYNVPEEVKAAGFQVCAEELGSITEGHDVKKLKFHGGVTGIAEKLSTSISDGLTSNTDLFNRRQEIYGLNQFAESTPRS
FWVFVWEALQDMTLMILGACAFVSLIVGIVMEGWPHGAHDGLGIVASILLVVFVTATSDYRQSLQFKDLDKEKKKIYVQV
TRNGFRQKLSIYDLLPGDIVHLGIGDQVPADGLFVSGFSVLIDESSLTGESEPVMVNEENPFMLSGTKLQDGSCKMMVTT
VGMRTQWGKLMATLSEGGDDETPLQVKLNGVATIIGKGGLFFAVVTFAVLVQGLLSHKLGEGSIWSWSGDDALKLLEYFA
VAVTIVVVAVPEGLPLAVTLSLAFAMKKMMNDKALVRHLAACETMGSASSICSDKTGTLTTNHMTVVKSCICMNVKEVSK
TDSASSLCSEIPDSAVQLLLQSIFTNTGGEVVVNKDGKREILGTPTETALLEFGLSLGGDFQAERQTSKIVKVEPFNSSK
KRMGVVLELPGGGLRAHSKGASEIVLSGCDKVVNSTGEVVPLDEESLNHLKLTIDQFANEALRTLCLAFMELETGFLPEN
PIPVSGYTLIAIVGIKDPVRPGVKESVAVCRSAGITVRMVTGDNINTAKAIARECGILTDDGIAIEGPVFREKTTEELME
LIPKIQVMARSSPLDKHTLVKHLRTTFDEVVAVTGDGTNDAPALHEADIGLAMGIAGTEVAKESADVIILDDNFSTIATV
AKWGRSVYINIQKFVQFQLTVNIVALIVNFSSACLTGSAPLTAVQLLWVNMIMDTLGALALATEPPTDELMKRPPVGKRG
NFISNVMWRNILGQSLYQFMVISLLQAKGKAIFWLDGPDSTLELNTLIFNSFVFCQIFNEISSREMEEINVFKGILDNYV
FASVLGVTVFFQIIIVEFLGTFANTTPLTLTQWFASIVIGFIGMPIAAGLKTIQV
>XP_024949070.1 putative calcium-transporting ATPase 11, plasma membrane-type
[Citrus sinensis]
MDKFLNWKDFDVEHKNPSEEALRRWRSAVSIVKNPRRRFRMVADLDKRSEAEKKKLEIKVISDKDKSQATNMVACTAMAR
GFPTSQKDISPQKNLTLIILMVCAGLSIGVGLAREGWPEGIYDGLGIILSKFLVVMVTAISDYKQSLQFRDLDREKKKIF
IQVTRDGQRQKVSIYDLVVGDIVHLSIGDQVAADGIFISGHSLLIDESSLSGESEPMYIYVEKFKDGSGKMLVTTVGMRT
EWGKLMETLNEGSEDETPLQVKINGVATIIGKIELFFSVLEFLVLVGRFLGEKVIHNEFTDWSSADALTLIDYLAVVVTL
IDVAVPEGLPLAVILSLAFAVKKLMNDGALVRHLSACEAMGSSNCICTDKTGMLTTNHMVVDKIWGHGNTISNVEGNNRE
EILQSEISERVLDITLQAIFQNTGSEVVKDKDGKNSILRTPTESTVLEFGLRLGGNFEAQRREFKIIKVEPFNSVGKRMS
VITALPEGGMRAFCKGASEIVLSMCDKVVSDNGEPVPLSEEQFRNITDVINGFASEALRTLCLAFKDLNDSSDENNIPDS
GYTLIAVVGIKDPVRPGVKEAVQTCLEAGITVRMVTGDNINTARAIAKECGILTSDGEAVEGPELRNMSPADMKRIIPKL
QVMARSLPLDKHTLVTQLRNTFGEFVAVTGDGTNDAPALHEADIGLSMGIAGTEVAKQNADVIILDDNFSTIVNVAKWGR
AVYINIQKFLQFQLTINVVINFVSACASGSAPLTAVQVLWVNMIMDTLGALALATEPPHEGLMKRPPVAKGESLITKVMW
RNIIGQCIYQLIILVVLNFDGKQLLGLSGSGATAVLNTVIFNSFVFCQLFNEINSREMEKINVFKGMFNSWMFVGILVLT
VAFQIIIVEFLGAFASTVPLRWQLWLLSILIGAVSMPIAAVIKCIPVKKCEPKLQRHD
>KDO81510.1 hypothetical protein CISIN_1g001743mg [Citrus sinensis]
MENYLNENFSDVKAKNTSEEALQRWRKLCGFVKNRKRRFRFTANLSKRFEAEAIRRSNQEKFRVAVLVSQAALQFIHGLN
LSSEYTVPEEVAASGFQICPDELGSIVEGHDIKKLKVHGGVEGIAEKLSTSITDGISTSEHLLNRRKEIYGINKFTESPA
RGFWVYVWEALHDMTLMILAVCALVSLVVGIATEGWPKGAHDGLGIVMSILLVVFVTATSDYKQSLQFKDLDREKKKITV
QVARNGFRRKISIYDLLPGDIVHLCMGDQVPADGLFVSGFSVLINESSLTGESEPVNVNALNPFLLSGTKVQNGSCKMLV
TTVGMRTQWGKLMATLSEGGDDETPLQVKLNGVATIIGKIGLFFAVVTFAVMVQGLFTRKLQEGTHWTWSGDDALEILEF
FAIAVTIVVVAVPEGLPLAVTLSLAFAMKKMMNDKALVRHLAACETMGSATSICSDKTGTLTTNHMTVLKACICEEIKEV
DNSKGTPAFGSSIPASASKLLLQSIFNNTGGEVVIGEGNKTEILGTPTETAILEFGLLLGGDFQAERQASKIVKVEPFNS
VKKQMGVVIELPEGGFRVHCKGASEIILAACDKFLNSNGEVVPLNEAAVNHLNETIEKFASEALRTLCLACMEIGNEFSA
DAPIPTEGYTCIGIVGIKDPMRPGVKESVAICRSAGITVRMVTGDNINTAKAIARECGILTDNGIAIEGPEFREKSDEEL
SKLIPKIQVMARSSPMDKHTLVKHLRTTLGEVVAVTGDGTNDAPALHEADIGLAMGIAGTEVAKESADVIILDDNFSTIV
TVAKWGRSVYINIQKFVQFQLTVNVVALIVNESSACLTGNAPLTAVQLLWVNMIMDTLGALALATEPPNGDLMKRSPVGR
KGNFISNVMWRNILGQSLYQFLIIWYLQTRGKAVFRLDGPDPDLILNTLIFNTFVFCQVCLSTCIRSTEP
>KDO81509.1 hypothetical protein CISIN_1g001743mg [Citrus sinensis]
MENYLNENFSDVKAKNTSEEALQRWRKLCGFVKNRKRRFRFTANLSKRFEAEAIRRSNQEKFRVAVLVSQAALQFIHGLN
LSSEYTVPEEVAASGFQICPDELGSIVEGHDIKKLKVHGGVEGIAEKLSTSITDGISTSEHLLNRRKEIYGINKFTESPA
RGFWVYVWEALHDMTLMILAVCALVSLVVGIATEGWPKGAHDGLGIVMSILLVVFVTATSDYKQSLQFKDLDREKKKITV
QVARNGFRRKISIYDLLPGDIVALCMGDQVPADGLFVSGFSVLINESSLTGESEPVNVNALNPFLLSGTKVQNGSCKMLV
TTVGMRTQWGKLMATLSEGGDDETPLQVKLNGVATIIGKIGLFFAVVTFAVMVQGLFTRKLQEGTHWTWSGDDALEILEF
FAIAVTIVVVAVPEGLPLAVTLSLAFAMKKMMNDKALVRHLAACETMGSATSICSDKTGTLTTNHMTVLKACICEEIKEV
DNSKGTPAFGSSIPASASKLLLQSIFNNTGGEVVIGEGNKTEILGTPTETAILEFGLLLGGDFQAERQASKIVKVEPFNS
VKKQMGVVIELPEGGFRVHCKGASEIILAACDKFLNSNGEVVPLNEAAVNHLNETIEKFASEALRTLCLACMEIGNEFSA
DAPIPTEGYTCIGIVGIKDPMRPGVKESVAICRSAGITVRMVTGDNINTAKAIARECGILTDNGIAIEGPEFREKSDEEL
SKLIPKIQVMARSSPMDKHTLVKHLRTTLGEVVAVTGDGTNDAPALHEADIGLAMGIAGTEVAKESADVIILDDNFSTIV
TVAKWGRSVYINIQKFVQFQLTVNVVALIVNFSSACLTGNAPLTAVQLLWVNMIMDTLGALALATEPPNGDLMKRSPVGR
KGNFISNVMWRNILGQSLYQFLIIWYLQTRGKAVFRLDGPDPDLILNTLIFNTFVFCQLQRDGKDKRLQGYTEELCLCSC
AHLHRSFSNNNHRAPGYICKYNSSQFATVVC
>KDO81514.1 hypothetical protein CISIN_19001743mg [Citrus sinensis]
MTLMILAVCALVSLVVGIATEGWPKGAHDGLGIVMSILLVVFVTATSDYKQSLQFKDLDREKKKITVQVARNGFRRKISI
YDLLPGDIVHLCMGDQVPADGLFVSGFSVLINESSLTGESEPVNVNALNPFLLSGTKVQNGSCKMLVTTVGMRTQWGKLM
ATLSEGGDDETPLQVKLNGVATIIGKIGLFFAVVTFAVMVQGLFTRKLQEGTHWTWSGDDALEILEFFAIAVTIVVVAVP
EGLPLAVTLSLAFAMKKMMNDKALVRHLAACETMGSATSICSDKTGTLTTNHMTVLKACICEEIKEVDNSKGTPAFGSSI
PASASKLLLQSIFNNTGGEVVIGEGNKTEILGTPTETAILEFGLLLGGDFQAERQASKIVKVEPFNSVKKQMGVVIELPE
GGFRVHCKGASEIILAACDKFLNSNGEVVPLNEAAVNHLNETIEKFASEALRTLCLACMEIGNEFSADAPIPTEGYTCIG
IVGIKDPMRPGVKESVAICRSAGITVRMVTGDNINTAKAIARECGILTDNGIAIEGPEFREKSDEELSKLIPKIQVMARS
SPMDKHTLVKHLRTTLGEVVAVTGDGTNDAPALHEADIGLAMGIAGTEVAKESADVIILDDNFSTIVTVAKWGRSVYINI
QKFVQFQLTVNVVALIVNFSSACLTGNAPLTAVQLLWVNMIMDTLGALALATEPPNGDLMKRSPVGRKGNFISNVMWRNI
LGQSLYQFLIIWYLQTRGKAVFRLDGPDPDLILNTLIFNTFVFCQVFNEISSREMEKINVFKGILKNYVFVAVLTCTVLF
QIIIIELLGTFANTTPLNLQQWFVSILLGFLGMPIAAVLKLIQVG
>GAY36889.1 hypothetical protein CUMW_025210 [Citrus unshiu]
MENYLKKNFDVDPKRPSEEALMRWRSAVRVVKNPRRRFRMVADLAKRAEAERKRKKLQEKLRVALYVQKAALHFIDVSNR
QNVYGFNRYAEKPARSFWMFVWEALHDLTLIILMICAAVSIGVGIPTEGWPDGVYDGLGIVESILLVVIVTAVSDYKQSL
QFKALDKEKKNLIVQVTRDGYRKKLSIYDLVVGDIVHLSIGDQVPADGILISGYSLTIDESSLSGETEPVHINRDRPFLL
SGTKVQDGSGKMLVTSVGMRTEWGRLMVTLSEGGEDETPLQVKLNGVATVIGKIGLVFAVLTFLVLALRFLVEKAQHHQI
KNWSPIDAMKLLNYFAIAVTIVVVAVPEGLPLAVTLSLAFAMKKLMNDKALVRHLSACETMGSASCICTDKTGTLTTNHM
VVTKLWICNEAKTIKSGDNEKLLKPSVSDANTGSEVVKDKDGRTNILGTPTERAILEFGLILGGDSTFHREESAIVKVEP
FNSVKKRMSVLVSLPNNGGFRVECKGASEIILNMCDKIINADGKAVPISEEQRKNLTNVINGFSSEALRTLCLAFQDIKG
NHKAESIPENNYTLIAVVGIKDPVRPGVREAFQLTVNIVALVINFVAACITGSAPLTAVQLLWVNMIMDTLGALALATEP
PHEGLMQRPPIGRNVHFITVTMWRNIIGQSIYQIIVLGVLTFCGKKILKLSGPNATLILNTFIFNSFVFCQVFNEINSRD
MEKINVFRGIFSSWVFVAVLVATVGFQVIIVELLGTFATTVPLNWKLWLASVVIGAISMPFGVLLKCIPVGTCTSAANSK
HHDGYEPLPTGPDLA
>KDO81511.1 hypothetical protein CISIN_19001743mg [Citrus sinensis]
MENYINENFSDVKAKNTSEEALQRWRKLCGFVKNRKRRFRFTANLSKREEAEAIRRSNQEKFRVAVLVSQAALQFIHGLN
LSSEYTVPEEVAASGFQICPDELGSIVEGHDIKKLKVHGGVEGIAEKLSTSITDGISTSEHLLNRRKEIYGINKFTESPA
RGFWVYVWEALHDMTLMILAVCALVSLVVGIATEGWPKGAHDGLGIVMSILLVVFVTATSDYKQSLQFKDLDREKKKITV
QVARNGFRRKISIYDLLPGDIVHLCMGDQVPADGLFVSGFSVLINESSLTGESEPVNVNALNPFLLSGTKVQNGSCKMLV
TTVGMRTQWGKLMATLSEGGDDETPLQVKLNGVATIIGKIGLFFAVVTFAVMVQGLFTRKLQEGTHWTWSGDDALEILEF
FAIAVTIVVVAVPEGLPLAVTLSLAFAMKKMMNDKALVRHLAACETMGSATSICSDKTGTLTTNHMTVLKACICEEIKEV
DNSKGTPAFGSSIPASASKLLLQSIFNNTGGEVVIGEGNKTEILGTPTETAILEFGLLLGGDFQAERQASKIVKVEPFNS
VKKQMGVVIELPEGGFRVHCKGASEIILAACDKFLNSNGEVVPLNEAAVNHLNETIEKFASEALRTLCLACMEIGNEFSA
DAPIPTEGYTCIGIVGIKDPMRPGVKESVAICRSAGITVRMVTGDNINTAKAIARECGILTDNGIAIEGPEFREKSDEEL
SKLIPKIQVMARSSPMDKHTLVKHLRTTLGEVVAVTGDGTNDAPALHEADIGLAMGIAGTEVAKESADVIILDDNFSTIV
TVAKWGRSVYINIQKFVQFQLTVNVVALIVNFSSACLTGK
>GAY50112.1 hypothetical protein CUMW_124220 [Citrus unshiu]
MESYLQENFGVKPKHSSTEALEKWRNLCGVVKNPKRRFRFTANLSKRYEAAAMRKTNQEKLRIAVLVSKAAIQFLLGVTP
SDYNVPEEVKAAGFQVCAEELGSITEGHDVKKLKFHGGVTGIAEKLSTSISDGLTSNTDLFNRRQEIYGLNQFAESTPRS
FWVFVWEALQDMTLMILGACAFVSLIVGIVMEGWPHGAHDGLGIVASILLVVFVTATSDYRQSLQFKDLDKEKKKIYVQV
TRNGFRQKLSIYDLLPGDIVHLGIGDQVPADGLFVSGFSVLIDESSLTGESEPVMVNEENPFMLSGTKLQDGSCKMMVTT
VGMRTQWGKLMATLSEGGDDETPLQVKLNGVATIIGKGGLFFAVVTFAVLVQGLLSHKLGEGSIWSWSGDDALKLLEYFA
VAVTIVVVAVPEGLPLAVTLSLAFAMKKMMNDKALVRHLAACETMGSASSICSDKTGTLTINHMTVVKSCICMNVKEVSK
TDSASSLCSEIPDSAVQLLLQSIFTNTGGEVVVNKDGKREILGTPTETALLEFGLSLGGDFQAERQTSKIVKVEPFNSSK
KRMGVVLELPGGGLRAHSKGASEIVLSGCDKVVNSTGEVVPLDEESLNHLKLTIDQFANEALRTLCLAFMELETGFLPEN
PIPVSGYTLIAIVGIKDPVRPGVKESVAVCRSAGITVRMVTGDNINTAKAIARECGILTDDGIAIEGPVFREKTTEELME
LIPKIQVMARSSPLDKHTLVKHLRTTFDEVVAVTGDGTNDAPALHEADIGLAMGIAGTEVSTLQMIS
>KDO81512.1 hypothetical protein CISIN_19001743mg [Citrus sinensis]
MENYLNENFSDVKAKNTSEEALQRWRKLCGFVKNRKRRFRFTANLSKRFEAEAIRRSNQEKFRVAVLVSQAALQFINGLN
LSSEYTVPEEVAASGFQICPDELGSIVEGHDIKKLKVHGGVEGIAEKLSTSITDGISTSEHLLNRRKEIYGINKFTESPA
RGFWVYVWEALHDMTLMILAVCALVSLVVGIATEGWPKGAHDGLGIVMSILLVVFVTATSDYKQSLQFKDLDREKKKITV
QVARNGFRRKISIYDLLPGDIVHLCMGDQVPADGLFVSGFSVLINESSLTGESEPVNVNALNPFLLSGTKVQNGSCKMLV
TTVGMRTQWGKLMATLSEGGDDETPLQVKLNGVATIIGKIGLFFAVVTFAVMVQGLFTRKLQEGTHWTWSGDDALEILEF
FAIAVTIVVVAVPEGLPLAVTLSLAFAMKKMMNDKALVRHLAACETMGSATSICSDKTGTLTINHMTVLKACICEEIKEV
DNSKGTPAFGSSIPASASKLLLQSIFNNTGGEVVIGEGNKTEILGTPTETAILEFGLLLGGDFQAERQASKIVKVEPFNS
VKKQMGVVIELPEGGFRVHCKGASEIILAACDKFLNSNGEVVPLNEAAVNHLNETIEKFASEALRTLCLACMEIGNEFSA
DAPIPTEGYTCIGIVGIKDPMRPGVKESVAICRSAGITVRMVTGDNINTAKAIARECGILTDNGIAIEGPEFREKSDEEL
SKLIPKIQVMARSSPMDKHTLVKHLRTTLGEVVAVTGDGTNDAPALHEADIGLAMGIAGTEVELECCCFNFSSRKTYIL
MPK1 protein sequences
>PtMPK1_Ptrif.0004s0435.1.v1.3.1_Poncirus_trifoliata
MLEKEDDLGNPRGSCQLPGSRKMFWRSASWSSSRTASQNPETEERDLADP
SGTNIVNSNGRRFPVPLTPRSQQNSKARSCLPPLQPLSIARRSLDEWPKA
SSDDVGEWHQPPTPSGNKSGERLKLDLSSIQRNSDKNGGLVKRDKIAFFD
KECSKVAEHIYLGGDAVARDRDILKQHGITHILNCVGFVCPEYFKADFVY
RTLWLQDSPSEDITSILYDVFDYFEDVREKGGRVFVHCCQGVSRSTSLVI
AYLMWREGQSFDDAFQYVKAARGIADPNMGFACQLLQCQKRVHAFPLSPS
SLLRMYRIAPHSPYDPLHLVPKMLNDPTPSALDSRGAFIVHIPAAIYIWI
GKHCESIMERDARGAVCQLVRYERAQGRIVIIKEGEEPGYFWDAFSNFLP
LMDKSRNGVEIRESTIKMVPGERKVNSYDVDYEIFRKAIMGGFVPPFSSS
ENEHETHLPARESSWSALRRKFASGDMKEFVSVPKISLCRVYSESMMLVH
SSSPSSSTSSLLSSSSSPPYLSPDSLCSDSSTSSKCSSESSMDSPSAASC
SLPVSSTLSNFSNLSLHSFKNSSEDIPNKPETCGSQPPLSPVKRISPSLA
ERRGSLSKSLKLPVMTSNVRANSSLDLLASQEDGASRSDNTYTLCNSDSI
DIVFKSKSAIRNGEEDATQMCKLKISPSSVDTAELCHKVSSSANNCVDSG
RNYSWREGLKANRLDESVPDHCNQMQPLIYRWPTFERVGKEDLSALNSKS
AFAIFSPSRDSGKSAARVLYFWVGRSFCHGKSQIQLDNNKELGNIEGSDQ
NQFGYDILTRMGLPKDTPIKIVKEDEEPREFLALLSAP-
>XP_006436100.1 protein-tyrosine-phosphatase MKP1 [Citrus clementina]
MLEKEDDLGNPRGSCQLPGSRKMFWRSASWSSSRTASQNPETEERDLADPSGSNIVNSNGRRFPVPLTPRSQQNSKARSC
LPPLQPLSIARRSLDEWPKASSDDVGEWHQPPTPSGNKSGERLKLDLSSIQRNSDKNGGLVKRDKIAFFDKECSKVAEHI
YLGGDAVARDRDILKQHGITHILNCVGFVCPEYFKADFVYRTLWLQDSPSEDITSILYDVFDYFEDVREKGGRVFVHCCQ
GVSRSTSLVIAYLMWREGQSFDDAFQYVKAARGIADPNMGFACQLLQCQKRVHAFPLSPSSLLRMYRIAPHSPYDPLHLV
PKMLNDPTPLALDSRGAFIVHIPAAIYIWIGKHCESIMERDARGAVCQLVRYERAQGRIVIIKEGEEPGYFWDAFSNFLP
LMDKSRNGVEIRESTIKMVPGERKVNSYDVDYEIFRKAIMGGFVPPFSSSENEHETHLPARESSWSALRRKFASGDMKEF
VSVPKISLCRVYSESMMLVHSSSPSSSTSSLLSSSSSPPYLSPDSVCSDSSTSSKCSSESSMDSPSAASCSLPVSSTLSI
FSNLSLHSFKNSSEDNKPETCGSQPPLSPVKRISPSLAERRGSLSKSLKLPVMTSNVRANSSLDLLASQEDVASRSDNTY
TLCNSDSIDIVFKSKSAIRNGEEDATQMCKLKISPSSVDTAELCHKVSSSANNCVDSGRNYSWREGLKANRLDESVPDHC
NQMQPLIYRWPTFERVGKFDSSALNSKSAFAIFSPSRDSGKSAARVLYFWVGRSFCHGKSPIQLDNNKELGNIEGSDQNQ
FGYDILTRMGLPKDTPIKIIKEDEEPREFLALLSTP
>XP_006486024.1 protein-tyrosine-phosphatase MKP1 [Citrus sinensis]
MLEKEDDIGNPRGSCQLPGSRKMFWRSASWSSSRTASQNPETEERDLADPSGSNIVNSNGRRFPVPLTPRSQQNSKARSC
LPPLQPLSIARRSLDEWPKASSDDVGEWHQPPTPSGNKSGERLKLDLSSIQRNSDKNGGLVKRDKIAFFDKECSKVAEHI
YLGGDAVARDRDILKQHGITHILNCVGFVCPEYFKADFVYRTLWLQDSPSEDITSILYDVFDYFEDVREKGGRVFVHCCQ
GVSRSTSLVIAYLMWREGQSFDDAFQYVKAARGIADPNMGFACQLLQCQKRVHAFPLSPSSLLRMYRIAPHSPYDPLHLV
PKMLNDPTPLALDSRGAFIVHIPAAIYIWIGKHCESIMERDARGAVCQLVRYERAQGRIVIIKEGEEPGYFWDAFSNFLP
LMDKSRNGVEIRESTIKMVPGERKVNSYDVDYEIFRKAIMGGFVPPFSSSENEHETHLPARESSWSALRRKFASGDMKEF
VSVPKISLCRVYSESMMLVHSSSPSSSTSSLLSSSSSPPYLSPDSVCSDSSTSSKCSSESSMDSPSAASCSLPVSSTLSI
FSNLSLHSFKNSSEDNKPETCGSQPPLSPVKRISPSLAERRGSLSKSLKLPVMTSNVRANSSLDLLASQEDVASRSDNTY
TLCNSDSIDIVFKSKSAIRNGEEDATQMCKLKISPSSVDTAELCHKVSSSANNCVDSGRNYSWREGLKANRLDESVPDHC
NQMQPLIYRWPTFERVGKFDSSALNSKSAFAIFSPSRDSGKSAARVLYFWVGRSFCHGESPIQLDNNKELGNIEGSDQNQ
FGYDILTRMGLPKDTPIKIIKEDEEPREFLALLSTP
>KDO67766.1 hypothetical protein CISIN_190032231mg, partial [Citrus sinensis]
MLEKEDDLGNPRGSCQLPGSRKMFWRSASWSSSRTASQNPETEERDLVDPSGSNIVNSNGRRFPVPLTPRSQQNSKARSC
LPPLQPLSIARRSLDEWPKASSDDVGEWHQPPTPSGNKSGERLKLDLSSIQRNSDKNGGLVKRDKIAFFDKECSKVAEHI
YLGGDAVARDRDILKQHGITHILNCVGFVCPEYFKADFVYRTLWLQDSPSEDITSILYDVFDYFEDVREKGGRVFVHCCQ
GVSRSTSLVIAYLMWREGQSFDDAFQYVKAARGIADPNMGFACQLLQCQKRVHAFPLSPSSLLRMYRIAPHSPYDPLHLV
PKMLNDPTPSALDSRGAFIVHIPAAIYIWIGKHCESIMERDARGAVCQLVRYERAQGRIVIIKEGEEPGYFWDAFSNFLP
LMDKSRNGVEIRESTIKMVPGERKVNSYDVDYEIFRKAIMGGFVPPFSSSENEHETHLPARESSWSALRRKFASGDMKEF
VSVPKISLCRVYSESMMLVHSSSPSSSTSSLLSSSSSPPYLSPDSVCSDSSTSSKCSSESSMDSPSAASCSLPVSSTLSN
FSNLSLRSFKNSSEDIPNKPETCGSQPPLSPVKRISPSLAERRGSLSKSLKLPVMTSNVRANSSLDLLASQEDVASRSDN
TYTLCNSDSIDIVFKSKSAIRNGEEDATQMCKLKISPSSVDTAELCHKVSSSANNCVDSGRNYSWREGLKANRLDESVPD
HCNQMQPLIYRWPTFERVGKEDSSALNSKSAFAIFSPSRDSGKSAARVLYFWVGRSFCHGKSRIQLDNNKELGNIEGSDQ
NQFGYDILTRMGLPKDTPIK
>XP_006483970.1 protein-tyrosine-phosphatase MKP1-like isoform X2 [Citrus
sinensis]
MVGEEDNNKHVDRLSGGSGNRRAYLRSVSWTDRSPNKPNPIPRPQPNSKARSLLPPLQPLSINRRPVEQWPRAGSDDLGV
WPNPQTPRGSVQLNPLESSSSELQPVKEFEFKKDKLAFFDKECSRIADHIYLGSDAVAKNRGILRQNGITHVLNCVGFVC
PEYFKGDLVYKTLWLQDSPSEDITSILYDVFDYFEDVREQGGRVFVHCCQGVSRSTSLVIAYLMWREGQSFEDAFQDVKA
ARGVTNPNMGFACQLLLCQKRVHAMPASPNSMLRIYRIAPHSSYDPLHLVPKLLNYPVAQGFDTRGAFIVLVPSAIYVWI
GKNCSVMMSNRAREAANQVIRYEKAQGQITSIKEGEEPLEFWDALVRGQFFADGCNKEEVKNEQVSFSGSNKIATLMQDG
AGEIDEYDLDFELFHKALDGGVVPPFSVSNAGSETCVPARESGWCRLRRKFVNGLMREFVASKINCATSAVNDESNMIID
TGKASEDAVSLAGFASPSSPPADVCGSPDSFDCFPNVSPNRISPQLSSKSPTLSPSTSDYSSSFTFSPSSCNWSDLSRQP
SPSGLEATDSSHSLCEETAFSLSKVFSPNHTSGVANSCFPCKGNFPSIAERRGSNPPPRLLPSAGKPSIVPRNLVRSWSF
SLTDLENDEVKDMDNNQIVHEGDREELMLNADLACASNDSHDKIKDKKEYDRVHFSLGTIDKRMGVANPVLYQWPALSKV
ESSSFQVLDSRSVYILLAPDTSIGTNESGILYVWLGCEVLCEKGQSQLVSNNCTCKHGHLQLETIGHNIINQMGLPADAS
VQIVREGEEPEQFLNHLNCFSFQKASNSANH
>XP_006483969.1 protein-tyrosine-phosphatase MKP1-like isoform X1 [Citrus
sinensis]
MVGEEDNNKHVDRLSGGSGNRRAYLRSVSWTDRSPNKPNPIPRPQPNSKARSLLPPLQPLSINRRPVEQWPRAGSDDLGV
WPNPQTPRGSVQLNPLESSSSELQPVKEFEFKKDKLAFFDKECSRIADHIYLGSDAVAKNRGILRQNGITHVLNCVGFVC
PEYFKGDLVYKTIWLQDSPSEDITSILYDVFDYFEDVREQGGRVFVHCCQGVSRSTSLVIAYLMWREGQSFEDAFQDVKA
ARGVTNPNMGFACQLLLCQKRVHAMPASPNSMLRIYRIAPHSSYDPLHLVPKLLNYPVAQGFDTRGAFIVLVPSAIYVWI
GKNCSVMMSNRAREAANQVIRYEKAQGQITSIKEGEEPLEFWDALVRGQFFADGCNKEEVKNEQVSFSGSNKIATLMQDG
AGEIDEYDLDFELFHKALDGGVVPPFSVSNAGSETCVPARESGWCRLRRKFVNGLMREFVASKLNCATSAVNDESNMIID
TGKASEDAVSLAGFASPSSPPADVCGSPDSFDCFPNVSPNRISSPQLSSKSPTLSPSTSDYSSSFTFSPSSCNWSDLSRQ
PSPSGLEATDSSHSLCEETAFSLSKVFSPNHTSGVANSCFPCKGNFPSIAERRGSNPPPRLLPSAGKPSIVPRNLVRSWS
FSLTDLENDEVKDMDNNQIVHEGDREELMLNADLACASNDSHDKIKDKKEYDRVHFSLGTIDKRMGVANPVLYQWPALSK
VESSSFQVLDSRSVYILLAPDTSLGTNESGILYVWLGCEVLCEKGQSQLVSNNCTCKHGHLQLETIGHNIINQMGLPADA
SVQIVREGEEPEQFLNHLNCESFQKASNSANH
CRT1 protein sequences
>PtCRT1_Ptrif.0005s1608.1.v1.3.1_Poncirus_trifoliata
MAKLNPSFLSLTLLTIFLTIASAHVFFEEREDDGWESRWVKSDWKTDENT
AGEWNYTAGKWNGDPNDKGIQTSEDYRFYAISAEFPEFSNKDKTLVFQFS
VKHEQKLDCGGGYMKLLSGEVDQKKFGGDTPYSIMFGPDICGYSTKKVHA
ILTYNGINKLIKKEVPCETDQLTHVYTFILRPDASYSILIDNVEKQSGSL
YSDWDLLPPKTIKDPDAKKPEDWDDKEYIPDPEDKKPEGYDDIPKEITDP
DAKKPDDWDDEEDGEWTAPTIPNPEYKGPWKPKKIKNPNYKGKWKAPMID
NPDFKDDPDLYVYPNLKYVGIELWQVKSGTMFDNVLVSDDPEYAKKLAEE
TWGKHKDAEKAAFDEAEKKREEEESKDAPDSDAEDNDDDDTEDDDDADAD
ADAETKSDSSSGDSDKDVHDEL-
>XP_006433523.1 calreticulin [Citrus clementina]
MAKLNPSFLSLTLLTIFLTIASAHVFFEERFDDGWESRWVTSDWKKDENTAGEWNYTAGKWNGDPNDKGIQTSEDYRFYA
ISAEFPEFSNKDKTLVFQFSVKHEQKLDCGGGYMKLLSGEVDQKKFGGDTPYSIMFGPDICGYSTKKVHAILTYNGTNKL
IKKEVPCETDQLTHVYTFILRPDATYSILIDNVEKQSGSLYSDWDLLPPKTIKDPDAKKPEDWDDKEYIPDPEDKKPEGY
DDIPKEITDPDAKKPDDWDDEEDGEWTAPTIPNPEYKGPWKPKKIKNPNYKGKWKAPMIDNPDFKDDPDLYVYPNLKYVG
IELWQVKSGTMFDNVLVSDDPEYANKLAEETWGKHKDAEKAAFDEAEKKREEEESKDAPDSDAEDNDDDDTEDDDDADTE
TKSDSSSGDADKDVHDEL
>GAY54380.1 hypothetical protein CUMW_156290, partial [Citrus unshiu]
YIQSSFTPHSTEHFLSLALKMAKLNPSFLSLTLLTIFLTIASAHVFFEERFDDGWESQWVKSDWKKDENTAGEWNYTAGK
WNGDPNDKGIQTSEDYRFYAISAEFPEFSNKDKTLVFQFSVKHEQKLDCGGGYMKLLSGEVDQKKFGGDTPYSIMFGPDI
CGYSTKKVHAILTYNGTNKLIKKEVPCETDQLTHVYTFILRPDATYSILIDNVEKQSGSLYSDWDLLPPKTIKDPDAKKP
EDWDDKEYIPDPEDKKPEGYDDIPKEITDPDAKKPDDWDDEEDGEWTAPTIPNPEYKGPWKPKKIKNPNYKGKWKAPMID
NPDFKDDPDLYVYPNLKYVGIELWQVKSGTMEDNVLVSDDPEYANKLAEETWGKHKDAEKAAFDEAEKKREEENEQIVKK
PEPPPAPSPVRSINALKIGAYATTRSAAGEPVPYSSNVVKKHVPFSVANNGSYRCSESLLLRRPDEFLCPLD
>ESR46762.1 hypothetical protein CICLE_v10001298mg [Citrus clementina]
MAKLNPSFLSLTLLTIFLTIASAHVFFEERFDDGWESRWVTSDWKKDENTAGEWNYTAGKWNGDPNDKGIQTSEDYRFYA
ISAEFPEFSNKDKTLVFQFSVKHEQKLDCGGGYMKLLSGEVDQKKFGGDTPYSIMFGPDICGYSTKKVHAILTYNGTNKL
IKKEVPCETDQLTHVYTFILRPDATYSILIDNVEKQSGSLYSDWDLLPPKTIKDPDAKKPEDWDDKEYIPDPEDKKPEGY
DDIPKEITDPDAKKPDDWDDEEDGEWTAPTIPNPEYKGPWKPKKIKNPNYKGKWKAPMIDNPDFKDDPDLYVYPNLKYVG
IELWQVKSGTMFDNVLVSDDPEYANKLAEETWGKHKDV
>GAY54381.1 hypothetical protein CUMW_156280, partial [Citrus unshiu]
YIQSSFTPHSTEHFLSLALKMAKLNPSFLSLTLLTIFLTIASAHVFFEERFDDGWESQWVKSDWKKDENTAGEWNYTAGK
WNGDPNDKGIQTSEDYRFYAISAEFPEFSNKDKTLVFQFSVKHEQKLDCGGGYMKLLSGEVDQKKFGGDTPYSIMFGPDI
CGYSTKKVHAILTYNGTNKLIKKEVPCETDQLTHVYTFILRPDATYSILIDNVEKQSGSLYSDWDLLPPKTIKDPDAKKP
EDWDDKEYIPDPEDKKPEGYDDIPKEITDPDAKKPDDWDDEEDGEWTAPTIPNPEYKGPWKPKKIKNPNYKGKWKAPMID
NPDFKDDPDLYVYPNLKYVGIELWQVKSGTMFDNVLVSDDPEYANKLAEETWGKHKDAEKAAFDEAEKKREEEVLFCSIT
LLHILFFLWNWVFSILS
>GAY54382.1 hypothetical protein CUMW_156290, partial [Citrus unshiu]
YIQSSFTPHSTEHFLSLALKMAKLNPSFLSLTLLTIFLTIASAHVFFEERFDDGWESQWVKSDWKKDENTAGEWNYTAGK
WNGDPNDKGIQTSEDYRFYAISAEFPEFSNKDKTLVFQFSVKHEQKLDCGGGYMKLLSGEVDQKKFGGDTPYSIMFGPDI
CGYSTKKVHAILTYNGTNKLIKKEVPCETDQLTHVYTFILRPDATYSILIDNVEKQSGSLYSDWDLLPPKTIKDPDAKKP
EDWDDKEYIPDPEDKKPEGYDDIPKEITDPDAKKPDDWDDEEDGEWTAPTIPNPEYKGPWKPKKIKNPNYKGKWKAPMID
NPDFKDDPDLYVYPNLKYVGIELWQVKSGTMFDNVLVSDDPEYANKLAEETWGKHKDV
>XP_006472186.1 calreticulin [Citrus sinensis]
MAKLNPSSLSLILLIIFLTIASAHVFFEERFDDGWESQWVKSDWKKDENTAGEWNYTAGKWNGDPNDKGIQTSEDYRFYA
ISAEFPEFSNKDKTLVFQFSVKHEQKLDCGGGYMKLLSGEVDQKKFGGDTPYSIMFGPDICGYSTKKVHAILTYNGTNKL
IKKEVPCETDQLTHVYTFILRPDATYSILIDNAEKQSGSLYSDWDLLPPKTIKDPDAKKPEDWDDKEYIPDPEDKKPEGY
DDIPKEITDPDAKKPDDWDDEEDGEWTAPTIPNPEYKGPWKPKKIKNPNYKGKWKAPMIDNPDFKDDPDLYVYPNLKYVG
IELWQVKSGTMFDNVLVSDDPEYAKKLAEETWGKHKDAEKAAFDEAEKKREEEESKDAPDSDAEDNDDDDTEDDDDADAE
TKSDSSSGDADKDVHDEL
>KDO81693.1 hypothetical protein CISIN_1g0453962mg, partial [Citrus sinensis]
MAKLNPSFLSLTLLTIFLTIASAHVFFEERFDDGWESRWVTSDWKKDENTAGEWNYTAGKWNGDPNDKGIQTSEDYRFYA
ISAEFPEFSNKDKTLVFQFSVKHEQKLDCGGGYMKLLSGEVDQKKFGGDTPYSIMFGPDICGYSTKKVHAILTYNGTNKL
IKKEVPCETDQLTHVYTFILRPDATYSILIDNAEKQSGSLYSDWDLLPPKTIKDPDAKKPEDWDDKEYIPDPEDKKPEGY
DDIPKEITDPDAKKPDDWDDEEDGEWTAPTIPNPEYKGPWKPK
LIN2 (HEMF1) protein sequences
>PtLIN2_Ptrif.0006s1200.1.v1.3.1_Poncirus_trifoliata
MPPTTTVSASSSFTLFRVPSSSSTKLKPTTTYIQIPNRFFPKHPTFKMTT
TTIRAAVSIEKETPETERPPTFLRESDDKESSSSSASSVRARFEKMIRDA
QDSVCQAIEKTDGGGKFKEDVWSRPGGGGGISRVLQDGAIWEKAGVNVSV
VYGVMPPEAYRAAKAAASDEKPGPIPFFAAGISSVLAPKNPFAPTLHFNY
RYFETDAPKDTPGAPRQWWFGGGTDLTPAYIFEEDVKHFHSTQKSACDKF
DPTFYPRFKKWCDDYFYIKHRGERRGLGGIFFDDLNDYDQEMLLSFATEC
ANSVIPAYIPIIEKRKDTPFTDQHKAWQQLRRGRYVEFNLVYDRGTTFGL
KIGGRIESILVSLPLTARWEYDHNPEEGSEEWKLLDACINPKEWI-
>XP_006429803.1 oxygen-dependent coproporphyrinogen-III oxidase, chloroplastic
[Citrus clementina]
MPPTTAVSASSSFTLFRVPSSSSTKLKPTTTYIQIPNRFFPKHPTFKMTTTTIRAAVSIEKETPETERPPTFLRESDDKE
SSSSSASSVRARFEKMIRDAQDSVCQAIEKTDGGGKFKEDVWSRPGGGGGISRVLQDGAIWEKAGVNVSVVYGVMPPEAY
RAAKAAASDEKPGPIPFFAAGISSVLHPKNPFAPTLHFNYRYFETDAPKDTPGAPRQWWFGGGTDLTPAYIFEEDVKHFH
STQKSACDKFDPTFYPRFKKWCDDYFYIKHRGERRGLGGLFFDDLNDYDQEMLLSFATECANSVIPAYIPIIEKRKDTPF
TDQHKAWQQLRRGRYVEFNEVYDRGTTFGLKTGGRIESILVSLPLTARWEYDHNPKEGSEEWKLLDACINPKEWI
>XP_006492904.1 oxygen-dependent coproporphyrinogen-III oxidase, chloroplastic
isoform X2 [Citrus sinensis]
MPPTTAVSASSSFTLFRVPSSWSTKLKPTTTYIQIPNRFFPKHPTFKMTTTTIRAAVSIEKETPETERPPTFLRESDDKE
SSSSSASSVRARFEKMIRDAQDSVCQAIEKTDGGGKFKEDVWSRPGGGGGISRVLQDGAIWEKAGVNVSVVYGVMPPEAY
RAAKAAASDEKPGPIPFFAAGISSVLHPKNPFAPTLHFNYRYFETDAPKDTPGAPRQWWFGGGTDLTPAYIFEEDVKHEH
STQKSACDKFDPTFYPRFKKWCDDYFYIKHRGERRGLGGLFFDDLNDYDQEMLLSFATECANSVIPAYIPIIEKRKDTPF
TDQHKAWQQLRRGRYVEFNLVYDRGTTFGLKTGGRIESILVSLPLTARWEYDHNPKEGSEEWKLLDACINPKEWI
>KDO50201.1 hypothetical protein CISIN_1g014082mg [Citrussinensis]
MPPTTAVSASSSFTLFRVPSSWSTKLKPTTTYIQIPNRFFPKHPTFKMTTTTIRAAVSIEKETPETERPPTFLRESDDKE
SSSSSASSVRARFEKMIRDAQDSVCQAIEKTDGGGKFKEDVWSRPGGGGGISRVLQDGAIWEKAGVNVSVVYGVMPPEAY
RAAKAAASDEKPGPIPFFAAGISSVLHPKNPFAPTLHFNYRYFETDAPKDTPGAPRQWWFGGGTDLTPAYIFEEDVKHFH
STQKSACDKFDPTFYPRFKKWCDDYFYIKHRGERRGLGGLFFDDLNDYDQEMLLSFATECANSVIPAYIPIIEKRKDTPF
TDQHKAWQQLRRGRYVEFNLVYDRGTTFGLKTGGRIESILVSLPLTARWEYDHVSFLEHSGEYASDVTKSLKSWTDEGSF
FFFSLFSMQNPKEGSEEWKLLDACINPKEWI
>XP_024949038.1 oxygen-dependent coproporphyrinogen-III oxidase, chloroplastic
isoform X1 (Citrus sinensis]
MPPTTAVSASSSFTLFRVPSSWSTKLKPTTTYIQIPNRFFPKHPTFKMTTTTIRAAVSIEKETPETERPPTFLRESDDKE
SSSSSASSVRARFEKMIRDAQDSVCQAIEKTDGGGKFKEDVWSRPGGGGGISRVLQDGAIWEKAGVNVSVVYGVMPPEAY
RAAKAAASDEKPGPIPFFAAGISSVLHPKNPFAPTLHFNYRYFETDAPKDTPGAPRQWWFGGGTDLTPAYIFEEDVKHFH
STQKSACDKFDPTFYPRFKKWCDDYFYIKHRGERRGLGGLFFDDLNDYDQEMLLSFATECANSVIPAYIPIIEKRKDTPF
TDQHKAWQQLRRGRYVEFNLVYDRGTTFGLKTGGRIESILVSLPLTARWEYDHVSFLEHSGEYASDVTKSLKSWTDEGSF
FFFFLVFYAEPERGK
>KDO50203.1 hypothetical protein CISIN_1g014082mg [Citrus sinensis]
MPPTTAVSASSSFTLFRVPSSWSTKLKPTTTYIQIPNRFFPKHPTFKMTTTTIRAAVSIEKETPETERPPTFLRESDDKE
SSSSSASSVRARFEKMIRDAQDSVCQAIEKTDGGGKFKEDVWSRPGGGGGISRVLQDGAIWEKAGVNVSVVYGVMPPEAY
RAAKAAASDEKPGPIPFFAAGISSVLHPKNPFAPTLHFNYRYFETDAPKDTPGAPRQWWFGGGTDLTPAYIFEEDVKHFH
STQKSACDKFDPTFYPRFKKWCDDYFYIKARGERRGLGGLFFDDLNDYDQEMLLSFATECANSVIPAYIPIIEKRKDTPF
TDQHKAWQQLRRGRYVEFNLVSNSPED
CRWN (LINC4) protein sequences
>PtCRWN_Ptrif.0007s0608.1.v1.3.1_Poncirus_trifoliata
MASPTSGRLAITPSSRVLQSPLSDESIWKRLKEAGFDEESIKRRDKAALI
AYIAKLETEIFEHQHHMGLLILEKKELASKYEQIKASAEAAELLQKHDQA
SHLSAIAEARKREESLKKTIGVEKECIASLEKAVHEIRAESAETKVAADS
KFAEARCMVENAQKKFAEAEAKLHAAEPLQAEANRYHRSAERKLQEVVAR
EDDLSRRIASFKADCEEKEREIIRERQSLSDRKKILQQEHERLLDAQTLL
NEREDHILSKLQELSRKEKELEASRANVEEKFKALNEEKSNLDLTLVSLS
KREEAVIEREASLQKKEQKLLVSQETLASKESNEIQKIIANHESALRVKQ
SEFEAELAIKYKLAEDEIEKKRRAWELRDLDLSQREESLLEREHDLEVQS
RALVDKEKDLVERSHLLEEKENKLIAFEKEADLKKSLLQKEKEEVNIIKS
DLQKSLSSLDEKKKQLNCAKDKLEAMKSEAGELSVLEIKLKEELDVVRAQ
KLELMVETDKLELEKAKFEAEWEMIDEKREELRKEAERVAVERVVVSKSL
KDERDSLRQERDAMRDQHKRDVDSLNREREEFMNKMVHEHSEWFTKIQQE
RADFLLGIEMQKRDLENCIEKRREELESSFREREKAFEEEKMRELQQISS
LKEKAEKELEQVTLEIKRLDLERMEINMDRQRRDREWAELNNSIEELKVQ
RQKLKEQRQLLHADREEIQAESERLKKLEDLKIAVDYMAVSEMQRSRLEH
SQKKISAKRHLNQQTSVAHADFGSDQKFDVTNNGDRFNTPSVQKTASASP
PSLARFSWIKRFADLVFKHSGENSVENDEEKSPTSDHEDASLTINSRKRQ
PVRYSFGEPKVILEVPSENEVVKRTVDLESENNQNAAQKCKQSVSEDGIH
AARKRRVDVDCVDPSELLMQNNKRRKQQEDFPRNSSEEAINHGAVAEQSN
LPEDQHTLTSKNKSNVPEGLHTLTSNNHIQGGNEEASILIVDKIIKISEV
TCEMTDADNFINQEKIDGSQNSVAESVQDIVKVGGINDHSTSAHTDDVIL
PYVSEIDGMGQEKQMGNVKDLTECGQAQNE
>ESR39398.1 hypothetical protein CICLE_v10024751mg [Citrus clementina]
MASPSSGRESITPSSRVLQSPLSDESIWKRLKEAGLDEESIKRRDKAALIAYIAKLETEIFEHQHHMGLLILEKKELASK
YEQIKASAEAAELLQKHDQASHLSAIAEARKREESLKKTLGVEKECIASLEKAVHEIRAESAETKVAADSKFAEARCMVE
NAQKKFAEAEAKLHASESLQAEANRYHRSAERKLQDVVAREDDLSRRIASFKADCEEKEREIIRERQSLSDRKKILQQEH
ERLLDAQTLLNEREDHILSKLQELSRKEKELEASRANVEEKFKALNEEKSNLDLTLVSLLKREEAVIEREASLQKKEQKL
LVSQETLASKESNEIQKIIANHESALRVKQSEFEAELAIKYKLAEDEIEKKRRAWELRDLDLGQREESLLEREHDLEVQS
RALVDKEKDLVERSHLLEEKENKLIAFEKEADLKKSLLQKEKEEVNIIKSDLQKSLSSLDEKKKQVNCAKDKLEAMKSEA
GELSVLEIKLKEELDVVRAQKLELMVETDKLQLEKAKFEAEWEMIDEKREELRKEAESVAVERVVVSKSLKDERDSLRQE
RDAMRDQHKRDVDSLNREREEFMNKMVHEHSEWFTKIQQERADFLLGIEMQKRDLENCIEKRREELESSFREREKAFEEE
KMRELQQISSLKEKAEKELEQVTLEIKRLDLERMEINMDRQRRDREWAELNNSIEELKVQRQKLEEQRQLLHADREEIQA
ESERLKKLEDLKIAVDYMAVSEMQRSRLEHSQKKISAKRHLNQQTSLAHADLGSDQKFDVTNNGDRFNTPSVQKTASASP
PSLARFSWIKRFADLVFKHSGENSIENDEEKSPTSDHEDASLTINSRKRQPVRYSFGEPKVILEVPSENEVVKRTVDLES
ENNQNAAQKCKQSVSEDGIHAARKRRVDVDCVDPSELLMQNNKRRKQQEDFPRNSSEEAINHGAVAEQSNLPEDQHTLTS
KNKSNVPEGLHTLTSNNHTQGGNEEASILIVDKIIKISEVTCEMPDADNFINQEKIDGSQNSVAESVQDIVKVGGTNDHS
TPAHTDDVVLPYVSEIDGMVQEKQMGNVKDLTECGQAQVLMFLHTSFLYIILAYDSCSLFLHDLLVCLYDGISCFC
>KDO78822.1 hypothetical protein CISIN_19001119mg [Citrus sinensis]
MASPSSGRLAITPSSRVLQSPLSDESIWKRLKEAGLDEVSIKRRDKAALIAYIAKLETEIFEHQHHMGLLILEKKELASK
YEQIKASAEAAELLQKADRASHLSAIAEARKREESLKKTLGVEKECIASLEKAVHEIRAESAETKVAADSKFAEARCMVE
NAQKKFAEAEAKLHAAESLQAEANRYHRSAERKLQEVVAREDDLSRRIASFKADCEEKEREIIRERQSLSDRKKILQQEH
ERLLDAQTLLNEREDHILSKLQELSRKEKELEASRANVEEKFKALNEEKSNLDLTLVSLLKREEAVIEREASLQKKEQKL
LVSQETLASKESNEIQKIIANHESALRVKQSEFEAELAIKYKLAEDEIEKKRRAWELRDLDLGQREESLLEREHDLEVQS
RALVDKEKDLVERSHLLEEKENKLIAFEKEADLKKSLLQKEKEEVNIIKSDLQKSLSSLDEKKKQVNCAKDKLEAMKSEA
GELSVLEIKLKEELDVVRAQKLELMVETDKLQLEKAKFEAEWEMIDEKREELRKEAERVAVERVVVSKSLKDERDSLRQE
RDAMRDQHKRDVDSLNREREEFMNKMVHEHSEWFTKIQQERADFLLGIEMQKRDLENCIEKRREELESSFREREKAFEEE
KMREFQQISSLKEKAEKELEQVTLEIKRLDLERMEINMDRQRRDREWAELNNSIEELMVQRQKLEEQRQLLHADREEIQA
ESERLKKLEDLKIAVDYMAVSEMQRSRLEHSQKKISAKRHLNQQTSLAHADLGSDQKFDVTNNGDRFNTPSVQKTASASP
PSLARFSWIKRFADLVFKHSGENSVENDEEKSPTSDHEDASLTINSRKRQPVRYSFGEPKVILEVPSENEVVKRTVDLES
ENNQNAAQKCKQSVSEDGIHAARKRRVDVDCVDPSELLMQNNKRRKQQEDFPRNSSEEAINHGAVAEQSNLPEDQHTLTS
KNKSNVPEGLHTLTSNNHTQGGNEEASILIVDKIIKISEVTCEMTDADNFINQEKIDGSQNSVAESVQDIVKVGGTNDHS
TPAHTDDVVLPYISEIDGMVQEKQMGNVKDLTECGQAQVLMFLHTSFLYIILAYDSCSLFLHDLLVCLYDGISYFC
>XP_006426157.1 protein CROWDED NUCLEI4 isoform X2 [Citrus clementina]
MASPSSGRLSITPSSRVLQSPLSDESIWKRLKEAGLDEESIKRRDKAALIAYIAKLETEIFEHQHHMGLLILEKKELASK
YEQIKASAEAAELLQKHDQASHLSAIAEARKREESLKKTLGVEKECIASLEKAVHEIRAESAETKVAADSKFAEARCMVE
NAQKKFAEAEAKLHASESLQAEANRYHRSAERKLQDVVAREDDLSRRIASFKADCEEKEREIIRERQSLSDRKKILQQEH
ERLLDAQTLLNEREDHILSKLQELSRKEKELEASRANVEEKEKALNEEKSNLDLTLVSLLKREEAVIEREASLQKKEQKL
LVSQETLASKESNEIQKIIANHESALRVKQSEFEAELAIKYKLAEDEIEKKRRAWELRDLDLGQREESLLEREHDLEVQS
RALVDKEKDLVERSHLLEEKENKLIAFEKEADLKKSLLQKEKEEVNIIKSDLQKSLSSLDEKKKQVNCAKDKLEAMKSEA
GELSVLEIKLKEELDVVRAQKLELMVETDKLQLEKAKFEAEWEMIDEKREELRKEAESVAVERVVVSKSLKDERDSLRQE
RDAMRDQHKRDVDSINREREEFMNKMVHEHSEWFTKIQQERADFLLGIEMQKRDLENCIEKRREELESSFREREKAFEEE
KMRELQQISSLKEKAEKELEQVTLEIKRLDLERMEINMDRQRRDREWAELNNSIEELKVQRQKLEEQRQLLHADREEIQA
ESERLKKLEDLKIAVDYMAVSEMQRSRLEHSQKKISAKRHLNQQTSLAHADLGSDQKFDVENNGDRFNTPSVQKTASASP
PSLARFSWIKRFADLVFKHSGENSIENDEEKSPTSDHEDASLTINSRKRQPVRYSFGEPKVILEVPSENEVVKRTVDLES
ENNQNAAQKCKQSVSEDGIHAARKRRVDVDCVDPSELLMQNNKRRKQQEDFPRNSSEEAINHGAVAEQSNLPEDQHTLTS
KNKSNVPEGEHTLTSNNHTQGGNEEASILIVDKIIKISEVTCEMPDADNFINQEKIDGSQNSVAESVQDIVKVGGTNDHS
TPAHTDDVVLPYVSEIDGMVQEKQMGNVKDLTECGQAQNEIGEHKLECELVQSDNSKKNKELIAYRTRSKQKK
>KDO78816.1 hypothetical protein CISIN_19001119mg [Citrus sinensis]
MASPSSGRLAITPSSRVLQSPLSDESIWKRLKEAGLDEVSIKRRDKAALIAYIAKLETEIFEHQHHMGLLILEKKELASK
YEQIKASAEAAELLQKHDRASHLSAIAEARKREESLKKTLGVEKECIASLEKAVHEIRAESAETKVAADSKFAEARCMVE
NAQKKFAEAEAKLHAAESLQAEANRYHRSAERKLQEVVAREDDLSRRIASFKADCEEKEREIIRERQSLSDRKKILQQEH
ERLLDAQTLLNEREDHILSKLQELSRKEKELEASRANVEEKFKALNEEKSNLDLTLVSLLKREEAVIEREASLQKKEQKL
LVSQETLASKESNEIQKIIANHESALRVKQSEFEAELAIKYKLAEDEIEKKRRAWELRDLDLGQREESLLEREHDLEVQS
RALVDKEKDLVERSHLLEEKENKLIAFEKEADLKKSLLQKEKEEVNIIKSDLQKSLSSLDEKKKQVNCAKDKLEAMKSEA
GELSVLEIKLKEELDVVRAQKLELMVETDKLQLEKAKFEAEWEMIDEKREELRKEAERVAVERVVVSKSLKDERDSLRQE
RDAMRDQHKRDVDSLNREREEFMNKMVHEHSEWFTKIQQERADFLLGIEMQKRDLENCIEKRREELESSFREREKAFEEE
KMREFQQISSLKEKAEKELEQVTLEIKRLDLERMEINMDRQRRDREWAELNNSIEELMVQRQKLEEQRQLLHADREEIQA
ESERLKKLEDLKIAVDYMAVSEMQRSRLEHSQKKISAKRHLNQQTSLAHADLGSDQKFDVTNNGDRFNTPSVQKTASASP
PSLARFSWIKRFADLVFKHSGENSVENDEEKSPTSDHEDASLTINSRKRQPVRYSFGEPKVILEVPSENEVVKRTVDLES
ENNQNAAQKCKQSVSEDGIHAARKRRVDVDCVDPSELLMQNNKRRKQQEDFPRNSSEEAINHGAVAEQSNLPEDQHTLTS
KNKSNVPEGLHTLTSNNHTQGGNEEASILIVDKIIKISEVTCEMTDADNFINQEKIDGSQNSVAESVQDIVKVGGTNDHS
TAHTDDVVLPYISEIDGMVQEKQMGNVKDLTECGQAQNEMGEHKLECELVQSDNSKKNKELIAYRTRSKQKK
>XP_024035967.1 protein CROWDED NUCLEI4 isoform X1 [Citrus clementina]
MASPSSGRISITPSSRVLQSPLSDESIWKRLKEAGLDEESIKRRDKAALIAYIAKLETEIFEHQHHMGLLILEKKELASK
YEQIKASAEAAELLQKHDQASHLSAIAEARKREESLKKTLGVEKECIASLEKAVHEIRAESAETKVAADSKFAEARCMVE
NAQKKEAEAEAKLHASESLQAEANRYHRSAERKLQDVVAREDDLSRRIASFKADCEEKEREIIRERQSLSDRKKILQQEH
ERLLDAQTLLNEREDHILSKLQELSRKEKELEASRANVEEKFKALNEEKSNLDLTLVSLLKREEVYTISFPFLFLNLVLI
CFHVLFTGNYIKYDSSIECTQAVIEREASLQKKEQKLLVSQETLASKESNEIQKIIANHESALRVKQSEFEAELAIKYKL
AEDEIEKKRRAWELRDLDLGQREESLLEREHDLEVQSRALVDKEKDLVERSHLLEEKENKLIAFEKEADLKKSLLQKEKE
EVNIIKSDLQKSLSSLDEKKKQVNCAKDKLEAMKSEAGELSVLEIKLKEELDVVRAQKLELMVETDKLQLEKAKFEAEWE
MIDEKREELRKEAESVAVERVVVSKSLKDERDSLRQERDAMRDQHKRDVDSLNREREEFMNKMVHEHSEWFTKIQQERAD
FLLGIEMQKRDLENCIEKRREELESSFREREKAFEEEKMRELQQISSLKEKAEKELEQVTLEIKRLDLERMEINMDRQRR
DREWAELNNSIEELKVQRQKLEEQRQLLHADREEIQAESERLKKLEDLKIAVDYMAVSEMQRSRLEHSQKKISAKRHINQ
QTSLAHADLGSDQKFDVTNNGDRFNTPSVQKTASASPPSLARFSWIKRFADLVFKHSGENSIENDEEKSPTSDHEDASLT
INSRKRQPVRYSFGEPKVILEVPSENEVVKRTVDLESENNQNAAQKCKQSVSEDGIHAARKRRVDVDCVDPSELLMQNNK
RRKQQEDFPRNSSEEAINHGAVAEQSNLPEDQHTLTSKNKSNVPEGLHTLTSNNHTQGGNEEASILIVDKIIKISEVTCE
MPDADNFINQEKIDGSQNSVAESVQDIVKVGGTNDHSTPAHTDDVVLPYVSEIDGMVQEKQMGNVKDLTECGQAQNEIGE
HKLECELVQSDNSKKNKELIAYRTRSKQKK
>KDO78814.1 hypothetical protein CISIN_19001119mg [Citrus sinensis]
MASPSSGRLAITPSSRVLQSPLSDESIWKRLKEAGLDEVSIKRRDKAALIAYIAKLETECYILKIFEHQHHMGLLILEKK
ELASKYEQIKASAEAAELLQKHDRASHLSAIAEARKREESLKKTLGVEKECIASLEKAVHEIRAESAETKVAADSKFAEA
RCMVENAQKKFAEAEAKLHAAESLQAEANRYHRSAERKLQEVVAREDDLSRRIASFKADCEEKERETIRERQSLSDRKKI
LQQEHERLLDAQTLLNEREDHILSKLQELSRKEKELEASRANVEEKFKALNEEKSNLDLTLVSLLKREEAVIEREASLQK
KEQKLLVSQETLASKESNEIQKIIANHESALRVKQSEFEAELAIKYKLAEDEIEKKRRAWELRDLDLGQREESLLEREHD
LEVQSRALVDKEKDLVERSHLLEEKENKLIAFEKEADLKKSLLQKEKEEVNIIKSDLQKSLSSLDEKKKQVNCAKDKLEA
MKSEAGELSVLEIKLKEELDVVRAQKLELMVETDKLQLEKAKFEAEWEMIDEKREELRKEAERVAVERVVVSKSLKDERD
SLRQERDAMRDQHKRDVDSLNREREEFMNKMVHEHSEWFTKIQQERADFLLGIEMQKRDLENCIEKRREELESSFREREK
AFEEEKMREFQQISSLKEKAEKELEQVTLEIKRLDLERMEINMDRQRRDREWAELNNSIEELMVQRQKLEEQRQLLHADR
EEIQAESERLKKLEDLKIAVDYMAVSEMQRSRLEHSQKKISAKRHLNQQTSLAHADLGSDQKFDVINNGDRFNTPSVQKT
ASASPPSLARFSWIKRFADLVFKHSGENSVENDEEKSPTSDHEDASLTINSRKRQPVRYSFGEPKVILEVPSENEVVKRT
VDLESENNQNAAQKCKQSVSEDGIHAARKRRVDVDCVDPSELLMQNNKRRKQQEDFPRNSSEEAINHGAVAEQSNLPEDQ
HTLTSKNKSNVPEGLHTLTSNNHTQGGNEEASILIVDKIIKISEVICEMTDADNFINQEKIDGSQNSVAESVQDIVKVGG
TNDHSTPAHTDDVVLPYISEIDGMVQEKQMGNVKDLTECGQAQNEMGEHKLECELVQSDNSKKNKELIAYRTRSKQKK
>KDO78821.1 hypothetical protein CISIN_1g001119mg [Citrus sinensis]
MASPSSGRLAITPSSRVLQSPLSDESIWKRLKEAGLDEVSIKRRDKAALIAYIAKLETEIFEHQHHMGLLILEKKELASK
YEQIKASAEAAELLQKHDRASHLSAIAEARKREESLKKTLGVEKECIASLEKAVHEIRAESAETKVAADSKFAEARCMVE
NAQKKFAEAEAKLHAAESLQAEANRYHRSAERKLQEVVAREDDLSRRIASFKADCEEKEREIIRERQSLSDRKKILQQEH
ERLLDAQTLLNEREDHILSKLQELSRKEKELEASRANVEEKFKALNEEKSNLDLTLVSLLKREEAVIEREASLQKKEQKL
LVSQETLASKESNEIQKIIANHESALRVKQSEFEAELAIKYKLAEDEIEKKRRAWELRDLDLGQREESLLEREHDLEVQS
RALVDKEKDLVERSHLLEEKENKLIAFEKEADLKKSLLQKEKEEVNIIKSDLQKSLSSLDEKKKQVNCAKDKLEAMKSEA
GELSVLEIKLKEELDVVRAQKLELMVETDKLQLEKAKFEAEWEMIDEKREELRKEAERVAVERVVVSKSLKDERDSLRQE
RDAMRDQHKRDVDSLNREREEFMNKMVHEHSEWFTKIQQERADFLLGIEMQKRDLENCIEKRREELESSFREREKAFEEE
KMREFQQISSLKEKAEKELEQVTLEIKRLDLERMEINMDRQRRDREWAELNNSIEELMVQRQKLEEQRQLLHADREEIQA
ESERLKKLEDLKIAVDYMAVSEMQRSRLEHSQKKISAKRHINQQTSLAHADLGSDQKFDVINNGDRFNTPSVQKTASASP
PSLARFSWIKRFADLVFKHSGENSVENDEEKSPTSDHEDASLTINSRKRQPVRYSFGEPKVILEVPSENEVVKRTVDLES
ENNQNAAQKCKQSVSEDGIHAARKRRVDVDCVDPSELLMQNNKRRKQQEDFPRNSSEEAINHGAVAEQSNLPEDQHTLTS
KNKSNVPEGLHTLTSNNHTQGGNEEASILIVDKIIKISEVTCEMTDADNFINQEKIDGSQNSVAESVQDIVKVGGTNDHS
TPAHTDDVVLPYISEIDGMVQEKQMGNVKDLTECE
>XP_006466411.1 protein CROWDED NUCLEI4 isoform X2 [Citrus sinensis]
MASPSSGRLAITPSSRVLQSPLSDESIWKRLKEAGLDEVSIKRRDKAALIAYIAKLETEIFEHQHHMGLLILEKKELASK
YEQIKASAEAAELLQKHDRASHLSAIAEARKREESLKKTLGVEKECIASLEKAVHEIRAESAETKVAADSKFAEARCMVE
NAQKKFAEAEAKLHAAESLQAEANRYHRSAERKLQEVVAREDDLSRRIASFKADCEEKEREIIRERQSLSDRKKILQQEH
ERLLDAQTLLNEREDHILSKLQELSRKEKELEASRANVEEKFKALNEEKSNLDLTLVSLLKREEAVIEREASLQKKEQKL
LVSQETLASKESNEIQKIIANHESALRVKQSEFEAELAIKYKLAEDEIEKKRRAWELRDLDLSQREESLLEREHDLEVQS
RALVDKEKDIVERSHLLEEKENKLIAFEKEADLKKSLLQKEKEEVNIIKSDLQKSLSSLDEKKKQVNCAKDKLEAMKSEA
GELSVLEIKLKEELDVVRAQKLELMVETDKLQLEKAKFEAEWEMIDEKREELRKEAERVAVERVVVSKSLKDERDSLRQE
RDAMRDQHKRDVDSINREREEFMNKMVHEHSEWFTKIQQERADFLLGIEMQKRDLENCIEKRREELESSFREREKAFEEE
KMREFQQISSLKEKAEKELEQVTLEIKRLDLERMEINMDRQRRDREWAELNNSIEELMVQRQKLEEQRQLLHADREEIQA
ESERLKKLEDLKIAVDYMAVSEMQRSRLEHSQKKISAKRHLNQQTSLAHADFGSDQKFDVTNNGDRFNTPVQKTASASPP
SLARFSWIKRFADLVFKHSGENSVENDEEKSPTSDHEDASLTINSRKRQPVRYSFGEPKVILEVPSENEVVKRTVDLESE
NNQNAAQKCKQSVSEDGIHAARKRRVDVDCVDPSELLMQNNKRRKQQEDFPRDSSEEAINHGAVAEQSNLPEDQHTLTSK
NKSNVPEGLHTLTSNNHTQGGNEEASILIVDKIIKISEVTCEMTDADNFINQEKIDGSQNSVAESVQDIVKVGGTNDHST
PAHTDDVVLPYISEIDGMVQEKQMGNVKDLTECGQAQNEMGEHKLECELVQSDNSKKNKELIAYRTRSKQKK
>KDO78820.1 hypothetical protein CISIN_1g001119mg [Citrus sinensis]
MASPSSGRLAITPSSRVLQSPLSDESIWKRLKEAGLDEVSIKRRDKAALIAYIAKLETEIFEHQHHMGLLILEKKELASK
YEQIKASAEAAELLQKHDRASHLSAIAEARKREESLKKTLGVEKECIASLEKAVHEIRAESAETKVAADSKFAEARCMVE
NAQKKFAEAEAKLHAAESLQAEANRYHRSAERKLQEVVAREDDLSRRIASFKADCEEKEREIIRERQSLSDRKKILQQEH
ERLLDAQTLLNEREDHILSKLQELSRKEKELEASRANVEEKFKALNEEKSNLDLTLVSLLKREEAVIEREASLQKKEQKL
LVSQETLASKESNEIQKIIANHESALRVKQSEFEAELAIKYKLAEDEIEKKRRAWELRDLDLGQREESLLEREHDLEVQS
RALVDKEKDLVERSHLLEEKENKLIAFEKEADLKKSLLQKEKEEVNIIKSDLQKSLSSLDEKKKQVNCAKDKLEAMKSEA
GELSVLEIKLKEELDVVRAQKLELMVETDKLQLEKAKFEAEWEMIDEKREELRKEAERVAVERVVVSKSLKDERDSLRQE
RDAMRDQHKRDVDSINREREEFMNKMVHEHSEWFTKIQQERADFLLGIEMQKRDLENCIEKRREELESSFREREKAFEEE
KMREFQQISSLKEKAEKELEQVTLEIKRLDLERMEINMDRQRRDREWAELNNSIEELMVQRQLLHADREEIQAESERLKK
LEDLKIAVDYMAVSEMQRSRLEHSQKKISAKRHLNQQTSLAHADLGSDQKEDVTNNGDRFNTPSVQKTASASPPSLARFS
WIKRFADLVFKHSGENSVENDEEKSPTSDHEDASLTINSRKRQPVRYSFGEPKVILEVPSENEVVKRTVDLESENNQNAA
QKCKQSVSEDGIHAARKRRVDVDCVDPSELLMQNNKRRKQQEDFPRNSSEEAINHGAVAEQSNLPEDQHTLTSKNKSNVP
EGLHTLTSNNHTQGGNEEASILIVDKIIKISEVTCEMTDADNFINQEKIDGSQNSVAESVQDIVKVGGTNDHSTPAHTDD
VVLPYISEIDGMVQEKQMGNVKDLTECGQAQNEMGEHKLECELVQSDNSKKNKELIAYRTRSKQKK
>GAY50146.1 hypothetical protein CUMW_124490 [Citrus unshiu]
MASPSSGRLSITPSSRVLQSPLSDESIWKRLKEAGLDEESIKRRDKAALIAYIAKLETECYILKIFEHQHHMGLLILEKK
ELASKYEQIKASAEAAELLQKHDQASHLSAIAEARKREESLKKTLGVEKECIASLEKAVHEIRAESAETKVAADSKFAEA
RCMVENAQKKFAEAEAKLHASESLQAEANRYHRSAERKLQEVVAREDDLSRRIASFKAHCEEKEREIIRERQSLSDRKKI
LQQEHERLLDAQTLLNEREDHILSKLQELSRKEKELEASRANVEEKFKALNEEKSNLDLTLVSLLKREEAVIEREASLQK
KEQKLLVSQETLASKESNEIQKIIANHESALRVKQSEFEAELAIKYKLAEDEIEKKRRAWELRDLDLGQREESLLEREHD
LEVQSRALVDREKDIVERSHLLEEKENKLIAFEKEADLKKSLLQKEKEEVNIIKSDLQKSLSSLDEKKKQVNCAKDKLEA
MKSEAGELSVLEIKLKEELDVVRAQKLELMVETDKLQLEKAKFEAEWEMIDEKREELRKEAERVAVERVVVSKSLKDERD
SLRQERDAMRDQHKRDVDSLNREREEFMNKMVHEHSEWFTKIQQERADFLLGIEMQKRDLENCIEKRREELESSFREREK
GFEEEKMREFQQISSLKEKAEKELEQVTLEIKRLDLERMEINMDRQRRDREWAELNNSIEELKVQRQKLEEQRQLLHADR
EEIQAESERLKKLEDLKIAVDYMAVSEMQRSRLEHSQKKISAKRHLNQQTSLAHADEGSDQKEDVINNGDRFNTPVQKTA
SASPPSLARFSWIKRFADLVFKHSGENSVENDEEKSPTSDHEDASLTINSRKRQPVRYSFGEPKVILEVPSENEVVKRTV
DLESENNQNAAQKCKQSVSEDGIHAARKRRVDVDCVDPSELPMQNNKRRKQQEDFPRDSSEEAINHGAVAEQSNLPEDQH
TLTSKNKSNVPEGLHTLTSNNHTQGGNEEASILIVDKIIKISEVTCEMPDADNFINQEKIDGSQNSVAESVQDIVKVGGT
NDHSTPAHTDDVVLPYVSEIDGMVQEKQMGNVKDLTECGQAQYAGLFVTSLGGDCLKSMYADPGTFDLFFEVNVVDPLGG
DGKTALFGGGGEANGKCEGDAYNHYVREGDKDRLGYFAERPAITCAAAALPYLLLVECWVPGIFLYLLSPCVDSCSLFLH
DLLNEMGEHKLECELVQSDNSKKNKELIAYRTRSKQKK
>KDO78815.1 hypothetical protein CISIN_1g001119mg [Citrus_sinensis]
MASPSSGRLAITPSSRVLQSPLSDESIWKRLKEAGLDEVSIKRRDKAALIAYIAKLETEIFEHQHHMGLLILEKKELASK
YEQIKASAEAAELLQKHDRASHLSAIAEARKREESLKKTLGVEKECIASLEKAVHEIRAESAETKVAADSKFAEARCMVE
NAQKKFAEAEAKLHAAESLQAEANRYHRSAERKLQEVVAREDDLSRRIASFKADCEEKEREIIRERQSLSDRKKILQQEH
ERLLDAQTLLNEREDHILSKLQELSRKEKELEASRANVEEKFKALNEEKSNLDLTLVSLLKREEVYTISFPFLFLNLVLI
CFHVLFTGNYIKYDSSIECTQAVIEREASLQKKEQKLLVSQETLASKESNEIQKIIANHESALRVKQSEFEAELAIKYKL
AEDEIEKKRRAWELRDLDLGQREESLLEREHDLEVQSRALVDKEKDLVERSHLLEEKENKLIAFEKEADLKKSLLQKEKE
EVNIIKSDLQKSLSSLDEKKKQVNCAKDKLEAMKSEAGELSVLEIKLKEELDVVRAQKLELMVETDKLQLEKAKFEAEWE
MIDEKREELRKEAERVAVERVVVSKSLKDERDSLRQERDAMRDQHKRDVDSINREREEFMNKMVHEHSEWFTKIQQERAD
FLLGIEMQKRDLENCIEKRREELESSFREREKAFEEEKMREFQQISSLKEKAEKELEQVTLEIKRLDLERMEINMDRQRR
DREWAELNNSIEELMVQRQKLEEQRQLLHADREEIQAESERLKKLEDLKIAVDYMAVSEMQRSRLEHSQKKISAKRHINQ
QTSLAHADLGSDQKFDVTNNGDRFNTPSVQKTASASPPSLARFSWIKRFADLVFKHSGENSVENDEEKSPTSDHEDASLT
INSRKRQPVRYSFGEPKVILEVPSENEVVKRTVDLESENNQNAAQKCKQSVSEDGIHAARKRRVDVDCVDPSELLMQNNK
RRKQQEDFPRNSSEEAINHGAVAEQSNLPEDQHTLTSKNKSNVPEGLHTLTSNNHTQGGNEEASILIVDKIIKISEVTCE
MTDADNFINQEKIDGSQNSVAESVQDIVKVGGTNDHSTPAHTDDVVLPYISEIDGMVQEKQMGNVKDLTECGQAQNEMGE
HKLECELVQSDNSKKNKELIAYRTRSKQKK
>GAY50145.1 hypothetical protein CUMW 124490 [Citrus_unshiu]
MASPSSGRLSITPSSRVLQSPLSDESIWKRLKEAGLDEESIKRRDKAALIAYIAKLETECYILKIFEHQHHMGLLILEKK
ELASKYEQIKASAEAAELLQKHDQASHLSAIAEARKREESLKKTLGVEKECIASVRYHQDHCLVELEKAVHEIRAESAET
KVAADSKFAEARCMVENAQKKFAEAEAKLHASESLQAEANRYHRSAERKLQEVVAREDDLSRRIASFKAHCEEKEREIIR
ERQSLSDRKKILQQEHERLLDAQTLLNEREDHILSKLQELSRKEKELEASRANVEEKFKALNEEKSNLDLTLVSLLKREE
AVIEREASLQKKEQKLLVSQETLASKESNEIQKIIANHESALRVKQSEFEAELAIKYKLAEDEIEKKRRAWELRDLDLGQ
REESLLEREHDLEVQSRALVDREKDLVERSHLLEEKENKLIAFEKEADLKKSLLQKEKEEVNIIKSDLQKSLSSLDEKKK
QVNCAKDKLEAMKSEAGELSVLEIKLKEELDVVRAQKLELMVETDKLQLEKAKFEAEWEMIDEKREELRKEAERVAVERV
VVSKSLKDERDSLRQERDAMRDQHKRDVDSLNREREEFMNKMVHEHSEWFTKIQQERADFLLGIEMQKRDLENCIEKRRE
ELESSFREREKGFEEEKMREFQQISSLKEKAEKELEQVTLEIKRLDLERMEINMDRQRRDREWAELNNSIEELKVQRQKL
EEQRQLLHADREEIQAESERLKKLEDLKIAVDYMAVSEMQRSRLEHSQKKISAKRHLNQQTSLAHADFGSDQKFDVTNNG
DRFNTPVQKTASASPPSLARFSWIKRFADLVFKHSGENSVENDEEKSPTSDHEDASLTINSRKRQPVRYSFGEPKVILEV
PSENEVVKRTVDLESENNQNAAQKCKQSVSEDGIHAARKRRVDVDCVDPSELPMQNNKRRKQQEDFPRDSSEEAINHGAV
AEQSNLPEDQHTLTSKNKSNVPEGLHTLTSNNHTQGGNEEASILIVDKIIKISEVTCEMPDADNFINQEKIDGSQNSVAE
SVQDIVKVGGTNDHSTPAHTDDVVLPYVSEIDGMVQEKQMGNVKDLTECGQAQYAGLFVTSLGGDCLKSMYADPGTFDLF
FEVNVVDPLGGDGKTALFGGGGEANGKCEGDAYNHYVREGDKDRLGYFAERPAITCAAAALPYLLLVECWVPGIFLYLLS
PCVDSCSLFLHDLLNEMGEHKLECELVQSDNSKKNKELIAYRTRSKQKK
>XP_006466410.1 protein CROWDED NUCLEI4 isoform X1 [Citrus sinensis]
MASPSSGRLAITPSSRVLQSPLSDESIWKRLKEAGLDEVSIKRRDKAALIAYIAKLETEIFEHQHHMGLLILEKKELASK
YEQIKASAEAAELLQKHDRASHLSAIAEARKREESLKKTLGVEKECIASLEKAVHEIRAESAETKVAADSKFAEARCMVE
NAQKKEAEAEAKLHAAESLQAEANRYHRSAERKLQEVVAREDDLSRRIASFKADCEEKEREIIRERQSLSDRKKILQQEH
ERLLDAQTLLNEREDHILSKLQELSRKEKELEASRANVEEKFKALNEEKSNLDLTLVSLLKREEVYMISFPFLFLNLVLI
CFHVFFTGNYIKYDSSIECTQAVIEREASLQKKEQKLLVSQETLASKESNEIQKIIANHESALRVKQSEFEAELAIKYKL
AEDEIEKKRRAWELRDLDLSQREESLLEREHDLEVQSRALVDKEKDLVERSHLLEEKENKLIAFEKEADLKKSLLQKEKE
EVNIIKSDLQKSLSSLDEKKKQVNCAKDKLEAMKSEAGELSVLEIKLKEELDVVRAQKLELMVETDKLQLEKAKFEAEWE
MIDEKREELRKEAERVAVERVVVSKSLKDERDSLRQERDAMRDQHKRDVDSLNREREEFMNKMVHEHSEWFTKIQQERAD
FLLGIEMQKRDLENCIEKRREELESSFREREKAFEEEKMREFQQISSLKEKAEKELEQVTLEIKRLDLERMEINMDRQRR
DREWAELNNSIEELMVQRQKLEEQRQLLHADREEIQAESERLKKLEDLKIAVDYMAVSEMQRSRLEHSQKKISAKRHLNQ
QTSLAHADFGSDQKFDVTNNGDRFNTPVQKTASASPPSLARFSWIKREADLVFKHSGENSVENDEEKSPTSDHEDASLTI
NSRKRQPVRYSFGEPKVILEVPSENEVVKRTVDLESENNQNAAQKCKQSVSEDGIHAARKRRVDVDCVDPSELLMQNNKR
RKQQEDFPRDSSEEAINHGAVAEQSNLPEDQHTLTSKNKSNVPEGLHTLTSNNHTQGGNEEASILIVDKIIKISEVTCEM
TDADNFINQEKIDGSQNSVAESVQDIVKVGGTNDHSTPAHTDDVVLPYISEIDGMVQEKQMGNVKDLTECGQAQNEMGEH
KLECELVQSDNSKKNKELIAYRTRSKQKK
>GAY50148.1 hypothetical protein CUMW_124490 [Citrus unshiu]
MASPSSGRLSITPSSRVLQSPLSDESIWKRLKEAGLDEESIKRRDKAALIAYIAKLETECYILKIFEHQHHMGLLILEKK
ELASKYEQIKASAEAAELLQKHDQASHLSAIAEARKREESLKKTLGVEKECIASLEKAVHEIRAESAETKVAADSKFAEA
RCMVENAQKKFAEAEAKLHASESLQAEANRYHRSAERKLQEVVAREDDLSRRIASFKAHCEEKEREIIRERQSLSDRKKI
LQQEHERLLDAQTLLNEREDHILSKLQELSRKEKELEASRANVEEKFKALNEEKSNLDLTLVSLLKREEVYMISFPFLFL
NLVLICFHVFFTGNYIKYDSSIECTQAVIEREASLQKKEQKLLVSQETLASKESNEIQKIIANHESALRVKQSEFEAELA
IKYKLAEDEIEKKRRAWELRDLDLGQREESLLEREHDLEVQSRALVDREKDLVERSHLLEEKENKLIAFEKEADLKKSLL
QKEKEEVNIIKSDLQKSLSSLDEKKKQVNCAKDKLEAMKSEAGELSVLEIKLKEELDVVRAQKLELMVETDKLQLEKAKF
EAEWEMIDEKREELRKEAERVAVERVVVSKSLKDERDSLRQERDAMRDQHKRDVDSINREREEFMNKMVHEHSEWFTKIQ
QERADFLLGIEMQKRDLENCIEKRREELESSFREREKGFEEEKMREFQQISSLKEKAEKELEQVTLEIKRLDLERMEINM
DRQRRDREWAELNNSIEELKVQRQKLEEQRQLLHADREEIQAESERLKKLEDLKIAVDYMAVSEMQRSRLEHSQKKISAK
RHLNQQTSLAHADFGSDQKFDVTNNGDRFNTPVQKTASASPPSLARFSWIKRFADLVFKHSGENSVENDEEKSPTSDHED
ASLTINSRKRQPVRYSFGEPKVILEVPSENEVVKRTVDLESENNQNAAQKCKQSVSEDGIHAARKRRVDVDCVDPSELPM
QNNKRRKQQEDFPRDSSEEAINHGAVAEQSNLPEDQHTLTSKNKSNVPEGLHTLTSNNHTQGGNEEASILIVDKIIKISE
VTCEMPDADNFINQEKIDGSQNSVAESVQDIVKVGGTNDHSTPAHTDDVVLPYVSEIDGMVQEKQMGNVKDETECGQAQY
AGLFVTSLGGDCLKSMYADPGTFDLFFEVNVVDPLGGDGKTALFGGGGEANGKCEGDAYNHYVREGDKDRLGYFAERPAI
TCAAAALPYLLLVECWVPGIFLYLLSPCVDSCSLFLHDLLNEMGEHKLECELVQSDNSKKNKELIAYRTRSKQKK
>KDO78818.1 hypothetical protein CISIN_1g001119mg [Citrus sinensis]
MASPSSGRLAITPSSRVLQSPLSDESIWKRLKEAGLDEVSIKRRDKAALIAYIAKLETEIFEHQHHMGLLILEKKELASK
YEQIKASAEAAELLQKHDRASHLSAIAEARKREESLKKTLGVEKECIASLEKAVHEIRAESAETKVAADSKFAEARCMVE
NAQKKFAEAEAKLHAAESLQAEANRYHRSAERKLQEVVAREDDLSRRIASFKADCEEKEREIIRERQSESDRKKILQQEH
ERLLDAQTLLNEREDHILSKLQELSRKEKELEASRANVEEKFKALNEEKSNLDLTLVSLLKREEAVIEREASLQKKEQKL
LVSQETLASKESNEIQKIIANHESALRVKQSEFEAELAIKYKLAEDEIEKKRRAWELRDLDLGQREESLLEREHDLEVQS
RALVDKEKDIVERSHLLEEKENKLIAFEKEADLKKSLLQKEKEEVNIIKSDLQKSLSSLDEKKKQVNCAKDKLEAMKSEA
GELSVLEIKLKEELDVVRAQKLELMVETDKLQLEKAKFEAEWEMIDEKREELRKEAERVAVERVVVSKSLKDERDSLRQE
RDAMRDQHKRDVDSLNREREEFMNKMVHEHSEWFTKIQQERADFLLGIEMQKRDLENCIEKRREELESSFREREKAFEEE
KMREFQQISSLKEKAEKELEQVTLEIKRLDLERMEINMDRQRRDREWAELNNSIEELMVQRQKLEEQRQLLHADREEIQA
ESERLKKLEDLKIAVDYMAVSEMQRSRLEHSQKKISAKRHLNQQTSLAHADLGSDQKFDVTNNGDRFNTPSVQKTASASP
PSLARFSWIKRFADLVFKHSGENSVENDEEKSPTSDHEDASLTINSRKRQPVRYSFGEPKVILEVPSENEVVKRTVDLES
ENNQNAAQKCKQSVSEDGIHAARKRRVDVDCVDPSELLMQNNKRRKQQEDFPRNSSEEAINHGAVAEQSNLPEDQHTLTS
KNKSNVPEGLHTLTSNNHTQGEKIDGSQNSVAESVQDIVKVGGTNDHSTPAHTDDVVLPYISEIDGMVQEKQMGNVKDLT
ECGQAQNEMGEHKLECELVQSDNSKKNKELIAYRTRSKQKK
>KDO78819.1 hypothetical protein CISIN_19001119mg [Citrus sinensis]
MASPSSGRLAITPSSRVLQSPLSDESIWKRLKEAGLDEVSIKRRDKAALIAYIAKLETEIFEHQHHMGLLILEKKELASK
YEQIKASAEAAELLQKHDRASHLSAIAEARKREESLKKTLGVEKECIASLEKAVHEIRAESAETKVAADSKFAEARCMVE
NAQKKFAEAEAKLHAAESLQAEANRYHRSAERKLQEVVAREDDLSRRIASFKADCEEKEREIIRERQSLSDRKKILQQEH
ERLLDAQTLLNEREDHILSKLQELSRKEKELEASRANVEEKFKALNEEKSNLDLTLVSLLKREEAVIEREASLQKKEQKL
LVSQETLASKESNEIQKIIANHESALRVKQSEFEAELAIKYKLAEDEIEKKRRAWELRDLDLGQREESLLEREHDLEVQS
RALVDKEKDLVERSHLLEEKENKLIAFEKEADLKKSLLQKEKEEVNIIKSDLQKSLSSLDEKKKQVNCAKDKLEAMKSEA
GELSVLEIKLKEELDVVRAQKLELMVETDKLQLEKAKFEAEWEMIDEKREELRKEAERVAVERVVVSKSLKDERDSLRQE
RDAMRDQHKRDVDSLNREREEFMNKMVHEHSEWFTKIQQERADFLLGIEMQKRDLENCIEKRREELESSFREREKAFEEE
KMREFQQISSLKEKAEKELEQVTLEIKRLDLERMEINMDRQRRDREWAELNNSIEELMVQRQKLEEQRQLLHADREEIQA
ESERLKKLEDLKIAVDYMAVSEMQRSRLEHSQKKISAKRHINQQTSLAHADLGSDQKFDVTNNGDRFNTPSVQKTASASP
PSLARFSWIKRFADLVFKHSGENSVENDEEKSPTSDHEDASLTINSRKRQPVRYSFGEPKVILEVPSENEVVKRTVDLES
ENNQNAAQKCKQSVSEDGIHAARKRRVDVDCVDPSELLMQNNKRRKQQEDFPRNSSEEAINHGAVAEQSNLPEDQHTLTS
KNKSNVPEGLHTLTSNNHTQGDGSQNSVAESVQDIVKVGGINDHSTPAHTDDVVLPYISEIDGMVQEKQMGNVKDLTECG
QAQNEMGEHKLECELVQSDNSKKNKELIAYRTRSKQKK
>KDO78824.1 hypothetical protein CISIN_1g001119mg [Citrus sinensis]
MGLLILEKKELASKYEQIKASAEAAELLQKHDRASHLSAIAEARKREESLKKTLGVEKECIASLEKAVHEIRAESAETKV
AADSKFAEARCMVENAQKKFAEAEAKLHAAESLQAEANRYHRSAERKLQEVVAREDDLSRRIASFKADCEEKEREIIRER
QSLSDRKKILQQEHERLLDAQTLLNEREDHILSKLQELSRKEKELEASRANVEEKFKALNEEKSNLDLTLVSLLKREEAV
IEREASLQKKEQKLLVSQETLASKESNEIQKIIANHESALRVKQSEFEAELAIKYKLAEDEIEKKRRAWELRDLDLGQRE
ESLLEREHDLEVQSRALVDKEKDLVERSHLLEEKENKLIAFEKEADLKKSLLQKEKEEVNIIKSDLQKSLSSLDEKKKQV
NCAKDKLEAMKSEAGELSVLEIKLKEELDVVRAQKLELMVETDKLQLEKAKFEAEWEMIDEKREELRKEAERVAVERVVV
SKSLKDERDSLRQERDAMRDQHKRDVDSLNREREEFMNKMVHEHSEWFTKIQQERADFLLGIEMQKRDLENCIEKRREEL
ESSFREREKAFEEEKMREFQQISSLKEKAEKELEQVTLEIKRLDLERMEINMDRQRRDREWAELNNSIEELMVQRQKLEE
QRQLLHADREEIQAESERLKKLEDLKIAVDYMAVSEMQRSRLEHSQKKISAKRHENQQTSLAHADLGSDQKFDVTNNGDR
FNTPSVQKTASASPPSLARFSWIKRFADLVFKHSGENSVENDEEKSPTSDHEDASLTINSRKRQPVRYSFGEPKVILEVP
SENEVVKRTVDLESENNQNAAQKCKQSVSEDGIHAARKRRVDVDCVDPSELLMQNNKRRKQQEDFPRNSSEEAINHGAVA
EQSNLPEDQHTLTSKNKSNVPEGLHTLTSNNHTQGGNEEASILIVDKIIKISEVICEMTDADNFINQEKIDGSQNSVAES
VQDIVKVGGTNDHSTPARTDDVVLPYISEIDGMVQEKQMGNVKDLTECGQAQNEMGEHKLECELVQSDNSKKNKELIAYR
TRSKQKK
>KDO78823.1 hypothetical protein CISIN_1g001119mg [Citrus sinensis]
MASPSSGRLAITPSSRVLQSPLSDESIWKRLKEAGLDEVSIKRRDKAALIAYIAKLETEIFEHQHHMGLLILEKKELASK
YEQIKASAEAAELLQKHDRASHLSAIAEARKREESLKKTLGVEKECIASLEKAVHEIRAESAETKVAADSKFAEARCMVE
NAQKKFAEAEAKLHAAESLQAEANRYHRSAERKLQEVVAREDDLSRRIASFKADCEEKEREIIRERQSLSDRKKILQQEH
ERLLDAQTLLNEREDHILSKLQELSRKEKELEASRANVEEKFKALNEEKSNLDLTEVSLLKREEAVIEREASLQKKEQKL
LVSQETLASKESNEIQKIIANHESALRVKQSEFEAELAIKYKLAEDEIEKKRRAWELRDLDLGQREESLLEREHDLEVQS
RALVDKEKDLVERSHLLEEKENKLIAFEKEADLKKSLLQKEKEEVNIIKSDLQKSLSSLDEKKKQVNCAKDKLEAMKSEA
GELSVLEIKLKEELDVVRAQKLELMVETDKLQLEKAKFEAEWEMIDEKREELRKEAERVAVERVVVSKSLKDERDSLRQE
RDAMRDQHKRDVDSINREREEFMNKMVHEHSEWFTKIQQERADFLLGIEMQKRDLENCIEKRREELESSFREREKAFEEE
KMREFQQISSLKEKAEKELEQVTLEIKRLDLERMEINMDRQRRDREWAELNNSIEELMVQRQKLEEQRQLLHADREEIQA
ESERLKKLEDLKIAVDYMAVSEMQRSRLEHSQKKISAKRHINQQTSLAHADLGSDQKFDVINNGDRFNTPSVQKTASASP
PSLARFSWIKREADLVFKHSGENSVENDEEKSPTSDHEDASLTINSRKRQPVRYSFGEPKVILEVPSENEVVKRTVDLES
ENNQNAAQKCKQSVSEDGIHAARKRRVDVDCVDPSELLMQNNKRRKQQEDFPRNSSEEAINHGC
>XP_024035968.1 protein CROWDED NUCLEI4 isoform X3 [Citrus clementina]
MASPSSGRLSITPSSRVLQSPLSDESIWKRLKEAGLDEESIKRRDKAALIAYIAKLETEIFEHQHHMGLLILEKKELASK
YEQIKASAEAAELLQKHDQASHLSAIAEARKREESLKKTLGVEKECIASLEKAVHEIRAESAETKVAADSKFAEARCMVE
NAQKKFAEAEAKLHASESLQAEANRYHRSAERKLQDVVAREDDLSRRIASFKADCEEKEREIIRERQSLSDRKKILQQEH
ERLLDAQTLLNEREDHILSKLQELSRKEKELEASRANVEEKFKALNEEKSNLDLTLVSLLKREEVYTISFPELFLNLVLI
CFHVLFTGNYIKYDSSIECTQAVIEREASLQKKEQKLLVSQETLASKESNEIQKIIANHESALRVKQSEFEAELAIKYKL
AEDEIEKKRRAWELRDLDLGQREESLLEREHDLEVQSRALVDKEKDLVERSHLLEEKENKLIAFEKEADLKKSLLQKEKE
EVNIIKSDLQKSLSSLDEKKKQVNCAKDKLEAMKSEAGELSVLEIKLKEELDVVRAQKLELMVETDKLQLEKAKFEAEWE
MIDEKREELRKEAESVAVERVVVSKSLKDERDSLRQERDAMRDQHKRDVDSLNREREEFMNKMVHEHSEWFTKIQQERAD
FLLGIEMQKRDLENCIEKRREELESSFREREKAFEEEKMRELQQISSLKEKAEKELEQVTLEIKRLDLERMEINMDRQRR
DREWAELNNSIEELKVQRQKLEEQRQLLHADREEIQAESERLKKLEDLKIAVDYMAVSEMQRSRLEHSQKKISAKRHLNQ
QTSLAHADLGSDQKFDVINNGDRFNTPSVQKTASASPPSLARFSWIKRFADLVFKHSGENSIENDEEKSPTSDHEDASLT
INSRKRQPVRYSFGEPKVILEVPSENEVVKRTVDLESENNQNAAQKCKQSVSEDGIHAARKRRVDVDCVDPSELLMQNNK
RRKQQEDFPRNSSEEAINHGC
>KDO78825.1 hypothetical protein CISIN_1g001119mg [Citrus sinensis]
MVENAQKKFAEAEAKLHAAESLQAEANRYHRSAERKLQEVVAREDDLSRRIASFKADCEEKEREIIRERQSLSDRKKILQ
QEHERLLDAQTLLNEREDHILSKLQELSRKEKELEASRANVEEKFKALNEEKSNLDLTLVSLLKREEAVIEREASLQKKE
QKLLVSQETLASKESNEIQKIIANHESALRVKQSEFEAELAIKYKLAEDEIEKKRRAWELRDLDLGQREESLLEREHDLE
VQSRALVDKEKDEVERSHLLEEKENKLIAFEKEADLKKSLLQKEKEEVNIIKSDLQKSLSSLDEKKKQVNCAKDKLEAMK
SEAGELSVLEIKLKEELDVVRAQKLELMVETDKLQLEKAKFEAEWEMIDEKREELRKEAERVAVERVVVSKSLKDERDSL
RQERDAMRDQHKRDVDSLNREREEFMNKMVHEHSEWFTKIQQERADFLLGIEMQKRDLENCIEKRREELESSFREREKAF
EEEKMREFQQISSLKEKAEKELEQVTLEIKRLDLERMEINMDRQRRDREWAELNNSIEELMVQRQKLEEQRQLLHADREE
IQAESERLKKLEDLKIAVDYMAVSEMQRSRLEHSQKKISAKRHLNQQTSLAHADLGSDQKFDVTNNGDRFNTPSVQKTAS
ASPPSLARFSWIKRFADLVEKHSGENSVENDEEKSPTSDHEDASITINSRKRQPVRYSFGEPKVILEVPSENEVVKRTVD
LESENNQNAAQKCKQSVSEDGIHAARKRRVDVDCVDPSELLMQNNKRRKQQEDFPRNSSEEAINHGAVAEQSNLPEDQHT
LTSKNKSNVPEGLHTLTSNNHTQGGNEEASILIVDKIIKISEVTCEMTDADNFINQEKIDGSQNSVAESVQDIVKVGGTN
DHSTPAHTDDVVLPYISEIDGMVQEKQMGNVKDLTECGQAQNEMGEHKLECELVQSDNSKKNKELIAYRTRSKQKK
>XP_006466412.1 protein CROWDED NUCLEI4 isoform X3 [Citrus sinensis]
MASPSSGRLAITPSSRVLQSPLSDESIWKRLKEAGLDEVSIKRRDKAALIAYIAKLETEIFEHQHHMGLLILEKKELASK
YEQIKASAEAAELLQKHDRASHLSAIAEARKREESLKKTLGVEKECIASLEKAVHEIRAESAETKVAADSKFAEARCMVE
NAQKKFAEAEAKLHAAESLQAEANRYHRSAERKLQEVVAREDDLSRRIASFKADCEEKEREIIRERQSLSDRKKILQQEH
ERLLDAQTLLNEREDHILSKLQELSRKEKELEASRANVEEKEKALNEEKSNLDLTLVSLLKREEVYMISEPFLFINLVLI
CFHVFFTGNYIKYDSSIECTQAVIEREASLQKKEQKLLVSQETLASKESNEIQKIIANHESALRVKQSEFEAELAIKYKL
AEDEIEKKRRAWELRDLDLSQREESLLEREHDLEVQSRALVDKEKDLVERSHLLEEKENKLIAFEKEADLKKSLLQKEKE
EVNIIKSDLQKSLSSLDEKKKQVNCAKDKLEAMKSEAGELSVLEIKLKEELDVVRAQKLELMVETDKLQLEKAKFEAEWE
MIDEKREELRKEAERVAVERVVVSKSLKDERDSLRQERDAMRDQHKRDVDSLNREREEFMNKMVHEHSEWFTKIQQERAD
FLLGIEMQKRDLENCIEKRREELESSFREREKAFEEEKMREFQQISSLKEKAEKELEQVTLEIKRLDLERMEINMDRQRR
DREWAELNNSIEELMVQRQKLEEQRQLLHADREEIQAESERLKKLEDLKIAVDYMAVSEMQRSRLEHSQKKISAKRHINQ
QTSLAHADFGSDQKFDVTNNGDRFNTPVQKTASASPPSLARFSWIKRFADLVFKHSGENSVENDEEKSPTSDHEDASLTI
NSRKRQPVRYSFGEPKVILEVPSENEVVKRTVDLESENNQNAAQKCKQSVSEDGIHAARKRRVDVDCVDPSELLMQNNKR
RKQQEDFPRDSSEEAINHGC
>SlCRWN_Solyc02g091960.2 sequence match in blast db Tomato Genome protein
sequences (ITAG release 2.40)
MASPGSGRLALTPVNPTPISGLGRVSKTPLTDEVIWKRLREAGFDEDSIKRRDKAALIAYIAKLETELYDHQYQMGLLIL
ERKEWVSKNEQSKAASESAELLYKREQAARLSDTAEAKKLEANLKKALGIEKECVANIEKALHEMRAECAEAKVASENKL
AEAQSMMEDAQKKYTDVEEKLRKAESLEAEASLFHRTAERKLREVESREDDLRRQTLLFKSECEAKEKEIQLERQSLSER
QKTLQRSQEELLDGQALLNKREEFIFSRSQELNRHEKDLEDEKSNFENDIKSLNEEKRNLEVKLKSLSAREEGIIRREHE
LYEKEKELLLLQGKIQSKEIDGSKQVMVNQEATLVTKISSIERCADTLLDRTPSNKRRREDGDFISQLTENGASCPLPPT
PDAPDVENLEVLPNQTHIAAEETTVYIDKIVTVHEVTEIDVRKVTEGSPGTLSGDSGRKVGNNGSLESDQNGKPEGRARR
TRATRK
>StCRWN_PGSC0003DMP400037089 sequence match in blast db Potato PGSC DM v3.4
protein sequences
MASPGSGRLALTPVNPTPISGLGRVSKTPLTDEVIWKRLREAGFDEDSIKRRDKAALIAYIAKLETELYDHQYQMGLLIL
ERKEWVSKNEQFKAASVSAELLYKREQAARLSDMAEAKKLEANLKKALGIEKECVANIEKALHEMRAECAEAKVASENKL
TEAQSMMEDAQKKYADVEEKLRKAESLEAEASLFHRTAERKLREVESREDDLRRQTLLFKSDCEAKEKEIQLERQSLSER
LKTLQRSQEELLDAQALLNKREEFIFSRSQELNRHEKDLEDEKSNLENDIKSLNEKKRNLEVKLKSLSAREEGIIKREHK
LNEKEEELLLLQGKMQSKEIDDSKQVMVNQEATLVTKISSIEAELETKRKLVEDEIQTKRRAWELKDMDIKSREDLITDK
EYDLERQSRTLAEKEKELEDKVHVIEEKERNLQAAEKEVELQRTVLQQEREGISKMRNDLEKSLKMLDEKRKCVDHEEEK
VEAMKNETQELLILETRLKLEIDMIRAEKEEIEMEADRLKAEKAKFETEWEVIDEKREELQKEAERVAEEKLAISKLLKD
SRDSLKAEKNAIQEEYKQNLESLSRDRETFMYEIESERAEWFNKIQKERENFLLDVEMQKKELENRIEKRREEIETDLKE
KEKAFEELKKRELQDIASLRETVEKELEHVGLELNKLDAERKEINLDRERRDKEWAELNNAIEELKVQRLKLEKQRELLH
ADRKEILAQIEQLKKLEDVKIIPDRIATPKKLHSGLPSNELKPSAKRLLKHASVLGSGLDGNGNNGVRQDTPSIMKENGN
SSSTLSTPFSWLKRCADTLLDRTPSNKRRREDGHFISQLTEYGASGTLSSSPDAPDVEHLEVLPNHTPIAAEETTVYIDK
IVTVHEVTEIDVRKVTEGSLETLSGESGRKVGNNGSLQSDKNGKPEGRSRRTKATRK
GPX8 protein sequences
>PtGPX8.1_Ptrif.0003s0313.1.v1.3.1_Poncirus_trifoliata
MTSQFIQNPESIFDLSVKDARGHEVDLSTYKGKVLLIVNVASKCGMTNSN
YIELSQLYDKYKDQGLEILAFPCNQFGEEEPGSNDQIADFVCTREKSEFP
IFEKIDVNGEHASPLYKLLKSGKWGIFGDDIQWNFAKFLVDKNGQVVDRY
YPTTSPLSLEHDIKKLLGLS-
>PPtGPX8.2_trif.0003s0292.1.v1.3.1_Poncirus_trifoliata
MLRCYHLKRNLGGIATSLILTRHFTSNYKQTLLRPSKSNPISLVSRPCFF
ASRSDHTMASQSKTSVHDFTVKDAKGQDVDLSIYKGKLLLIVNVASQCGL
TNSNYTELSQLYDKYKNQGLEILAFPCNQFGAQEPGDNEQIQEFACTRFK
AEFPIFDKVDVNGDNAAPLYKHLKSSKGGLFGDSIKWNFSKFLVDKEGNV
VERYAPTTSPLSIEKDIKKLLETA-
>XP_006439619.1 probable glutathione peroxidase 8 [Citrus clementina]
MTSQFIQNPESIFDLSVKDARGHEVDLSTYKGKVLLIVNVASKCGMTNSNYIELSQLYDKYKDQGLEILAFPCNQFGEEE
PGSNDQIADFVCTRFKSEFPIFEKIDVNGEHASPLYKLLKLGKWGIFGDDIQWNFAKFLVDKNGEVVDRYYPTTSPLSLE
RDIKKLLGLS
>ESR52856.1 hypothetical protein CICLE_v10022566mg [Citrus clementina]
MTNSNYIELSQLYDKYKDQGLEILAFPCNQFGEEEPGSNDQIADFVCTRFKSEFPIFEKIDVNGEHASPLYKLLKLGKWG
IFGDDIQWNFAKFLVDKNGEVVDRYYPTTSPLSLERDIKKLLGLS
>GAY39049.1 hypothetical protein CUMW_041400 [Citrus unshiu]
MTSQFIQNPEMPEAMSGMTNSNYIELSQLYDKYKDQGLEILAFPCNQFGEEEPGSNDQIADFVCTRFKSEFPIFEKIDVN
GEHASPLYKLLKSGKWGIFGDDIQWNFAKFLVDKNGEVVDRYYPTTSPLSLERDIKKLLGLS
>GAY39048.1 hypothetical protein CUMW_041400 [Citrus unshiu]
MTSQFIQNPESIFDLSVKDARGHEVDLSTYKGKVLLIVNVASKCGMTNSNYIELSQLYDKYKDQGLEILAFPCNQFGEEE
PGSNDQIADFVCTRFKSEFPIFEKIDVNGEHASPLYKLLKSGKWGIFGDDIQWNFAKFLVDKNGEVVDRYYPTTSPLSLE
RDIKKLLGLS
>XP_006476628.1 probable glutathione peroxidase 8 isoform X2 [Citrus sinensis]
MTSQFIQNPESIFDLSVKDARGHEVDLSTYKGKVLLIVNVASKCGMTNSNYIELSQLYDKYKDQGLEILAFPCNQFGEEE
PGSNDQIADFVCTRFKSEFPIFEKIDVNGEHASPLYKLLKSGKWGIFGDDIQWNFAKFLVDKNGQVVDRYYPTTSLLSLE
HDIKKLLGLS
>XP_006476629.1 probable glutathione peroxidase 8 isoform X1 [Citrus sinensis]
MFLGVLFIYLIIIIGHSQDARGHEVDLSTYKGKVLLIVNVASKCGMTNSNYIELSQLYDKYKDQGLEILAFPCNQFGEEE
PGSNDQIADFVCTRFKSEFPIFEKIDVNGEHASPLYKLLKSGKWGIFGDDIQWNFAKFLVDKNGQVVDRYYPTTSLLSLE
HDIKKLLGLS
>GAY39050.1 hypothetical protein CUMW_041410 [Citrus unshiu]
MTSQFIQNPESIFDLSVKDARGHEVDLSTYKGKVLLIVNVASKCGMTNSNYIELSQLYDKYKDQGLEILAFPCNQFGEEE
PGSNDQIADEVCTRFKSEFPIFEKIDVNGEHASPLYKLLKSGKWGIFGDDIQWNFAKFLVDKNGEVVDRYYPTTSPLSLE
LHQILTSQRDIKKLLGLS
>KDO76095.1 hypothetical protein CISIN_1g030881mg [Citrus sinensis]
MTNSNYIELSQLYDKYKDQGLEILAFPCNQFGEEEPGSNDQIADFVCTRFKSEFPIFEKIDVNGEHASPLYKLLKSGKWG
IFGDDIQWNFAKFLVDKNGQVVDRYYPTTSLLSLEHDIKKLLGLS
>KDO76099.1 hypothetical protein CISIN_1g030881mg [Citrus sinensis]
MTSQFIQNPESIFDLSVKDARGHEVDLSTYKGKVLLIVNVASKCGMTNSNYIELSQLYDKYKDQGLEILAFPCNQFGEEE
PGSNDQIADFVCTRFKSEFPIFEKIDVNGEHASPLYKLLKSGKWGIFGDDIQWNFAKFLVDKNGQVVDRYYPTTSLLSLE
VIL
>ESR52860.1 hypothetical protein CICLE_v10022566mq [Citrus clementina]
MTSQFIQNPESIFDLSVKDARGHEVDLSTYKGLEILAFPCNQFGEEEPGSNDQIADFVCTRFKSEFPIFEKIDVNGEHAS
PLYKLLKLGKWGIFGDDIQWNFAKFLVDKNGEVVDRYYPTTSPLSLERDIKKLLGLS
>GAY39047.1 hypothetical protein CUMW_041400 [Citrus unshiu]
MTSQFIQNPESIFDLSVKDARGHEILAFPCNQFGEEEPGSNDQIADFVCTRFKSEFPIFEKIDVNGEHASPLYKLLKSGK
WGIFGDDIQWNFAKFLVDKNGEVVDRYYPTTSPLSLERDIKKLLGLS
>Q06652.1 RecName: Full = Probable phospholipid hydroperoxide glutathione
peroxidase; Short = PHGPx; AltName: Full = Salt-associated protein [Citrus sinensis]
MASQSKTSVHDFTVKDAKGQDVDLSIYKGKLLLIVNVASQCGLTNSNYTELSQLYDKYKNQGLEILAFPCNQFGAQEPGD
NEQIQEFACTRFKAEFPIFDKVDVNGDNAAPLYKHLKSSKGGLFGDSIKWNFSKFLVDKEGNVVERYAPTTSPLSIEKDI
KKLLETA
>CAE46896.1 phospholipid hydroperoxide glutathione peroxidase [Citrus sinensis]
MASQSKTSVHDFSVKDAKGQDVDLSIYKGKLLLIVNVASQCGLTNSNYTELSQLYDKYKNQGLEILAFPCNQFGAQEPGD
NEQIQEFACTRFKAEFPIFDKVDVNGDNAAPLYKHLKSSKGGLFGDSIKWNFSKFLVDKEGNVVERYAPTTSPLSIEKDI
KKLLETA
>GAY39080.1 hypothetical protein CUMW_041630, partial [Citrus unshiu]
DAKGQDVDLSIYKGKLLLIVNVASQCGLTNSNYTELSQLYDKYKNQGLEILAFPCNQFGAQEPGDNEQIQEFACTRFKAE
FPIFDKVDVNGDNAAPLYKHLKSSKGGLFGDSIKWNFSKFLVDKEGNVVERYAPTTSPLSIEKDIKKLLETA
>XP_006439586.1 probable phospholipid hydroperoxide glutathione peroxidase
[Citrus clementina]
MLRCYLLKRNLGIATSHILTRHFTSNYKQTLLRPSKSNPISLVSRPCFFASRSDHTMASQSKTSVHDFTVKDAKGQDVDL
SIYKGKLLLIVNVASQCGLINSNYTELSQLYDKYKNQGLEILAFPCNQFGAQEPGDNEQIQEFACTRFKAEFPIFDKVDV
NGDNAAPLYKHLKSSKGGLFGDSIKWNFSKFLVDKEGNVVERYAPTTSPLSIEKDIKKLLETA
>XP_006476598.1 probable phospholipid hydroperoxide glutathione peroxidase
[Citrus sinensis]
MLRCCASRYLLKRNLGIATSLILTRHFTSNCKQTLLRPSKSNPISLVSRPCFFASRSDHTMASQSKTSVHDFSVKDAKGQ
DVDLSIYKGKLLLIVNVASQCGLTNSNYTELSQLYDKYKNQGLEILAFPCNQFGAQEPGDNEQIQEFACTRFKAEFPIFD
KVDVNGDNAAPLYKHLKSSKGGLFGDSIKWNFSKFLVDKEGNVVERYAPTTSPLSIEKDIKKLLETA
>ESR52825.1 hypothetical protein CICLE_v10022130mg [Citrus clementina]
MISPRDSLILAQCRRQFIFYFLFFIFSFIRFIHLPDFERSGLTNSNYTELSQLYDKYKNQGLEILAFPCNQFGAQEPGDN
EQIQEFACTRFKAEFPIFDKVDVNGDNAAPLYKHLKSSKGGLFGDSIKWNFSKFLVDKEGNVVERYAPTTSPLSIEKDIK
KLLETA
>KDO76161.1 hypothetical protein CISIN_1g027134mg [Citrus sinensis]
MLRCCASRYLLKRNLGIATSLILTRHFTSNCKQTLLRPSKSNPISLVSRPCFFASRSDHTMASQSKTSVHDFSVKDAKGQ
DVDLSIYKGKLLLIVNVASQCGLTNSNYTELSQLYDKYKNQGLEILAFPCNQFGAQEPGDNEQIQEFACTRFKAEFPIFD
KVDVNGDNAAPLYKHLKSSKGGLFGDSIKWNFSKFLVDKEGNVVERYAPTTSPLSIEVVLECLCC
>SlGPX8_Solyc12g056240.1 sequence match in blast db Tomato Genome protein
sequences (ITAG release 2.40)
MAGQPEKKPQSVYDFSLKDATGNDVDLSIFKGKVLLIVNVASKCGMTNSNYTELNQLYEKYKDQGLEILAFPCNQFGEEE
PGTNDQILNFVCTRFKSDFPIFDKIEVNGENASPLYKFLKSGKWGIFGDDIQWNFAKFLVDKNGQVVDRYYPTTSPLTIE
RDMKKLLETI
>StGPX8_XP_006360688.1 PREDICTED: probable glutathione peroxidase 8 [Solanum
tuberosum]
MAGQPEKKPQSVYDFTLKDAIGNDVDLSIYKGKVLLIVNVASKCGMTNSNYTELNQLYEKYKDQDLEILA
FPCNQFGEEEPGTNDQILDFVCTRFKSDFPIFDKIEVNGENASPLYKFLKSGKWGIFGDDIQWNFAKFLV
DKNGQVVDRYYPTTSPLTIERDMKKLLEII
LQX2 protein sequences
>PtLOX2.1_Ptrif.0002s0213.1.v1.3.1_Poncirus_trifoliata
MFNPVLVNQTRSIRTILPLSKPFLHGNGNVFRQIQSSPSFKKGPKIRLGS
VSSNSVKAMADTAVSNGVTAVVTVRPPINPLTAGGQVIDDVEDLFSKSLQ
LELVSAKDENKPTISGNAKIKGVVVKDCEVQYEAEFQVPVDFGEIGAILV
ANEHALEMYLKDIVLDGLPSGLVTITCESWVQPNTSKDPRIFFTNKSYLP
SKTPNGLQKLRYAELVNLRGNGEGERQKADRIYDYDVYNDLGDPDKDEAL
KRPVLGGKQHPYPRRCRTGRPHCKTDEASEERVPSKSLINISPYVPRDEE
FSAIKETTFVIRTLFGLFRSLIPNLKAEFVDTDGFPNFTEIDKLFREGVK
IKDAEFWKSLLPGFVEEIKDIGEFFLKFTSPETFKRDRFFWFRDEEFARE
TLAGINPCSIRLITEWPLKSSLDRKIYGPSESAITTEMIESEIKGCTTVK
EALNQKKLFILDYHDLFLPHVEQVRELGDRTLYGSRTVFYLNPDGTLRPL
AIELTRPPMDGKPQWKQAFTPSSDSTKSWLWKLAKAHVLAHDSGYHQLIS
HWLRTHCSVEPYAIAAHRQLSALHPINRLLKPHFRYTMEINSLARQSLIN
AGGVIESTFLPGKYSMLLSSIIYDKQWRFDHQALPQDLISRGMAVKDPSS
PHGLKLTIEDYPFAQDGLDLWDIIKQWVTDYVSHYYPDPSLVESDEELQA
WWTEIRTVGHGDKQDETWWPVLNSPKDLIDTITNIVWVASGLHAAVNFGQ
YEYAAYFPNRPTIARANMPNEDPSDDEWKIFFERPEAALLTTFPNQKQAT
AVISVLDVLSAHSPDEDYLGKYMEPAWGEDKIIKGAFEKFQGRLMELKGT
INLRNADKNLKNRNGAGSLPYELLMPLADKSGVTGKGVPYSISI-
> PtLOX2.2_Ptrif.0002s0215.1.v1.3.1_Poncirus_trifoliata
MLKPQVHQPQSIKPLFPLSKPFLHGNYGYAFRPVPSTSSLIKGSPKLRIG
SVPRNTIKAIATSTEKSVKVKAVVTVKPTVGGFLSNISLERGLDDIGDLF
GKSLLLELVSAELDPKTGLDKSTIQDYARKIGADGDGNMQYESEFEVPSG
FGEIGAILVENEHHKEMYLKDIVLDGLPNGPVNVTCNSWLHSKHDNKQKR
VFFTNKLYLPSQTPDGLKRYRAEELAILRGNGQGERKTYDRIYDYDVYND
LGDPDKKPELARPVLGGKQNPYPRRCRTGRPRCDTDQSSEKREGNFYVPR
DEAFSEVKQVTFSAKTVYSVLHALVPSLETAIVDPDLGFPYFSAIDALFN
EGVNLPPLKQEGFWNTLLPRLVKAIEDTGDNILLFETPETMDRDKFFWFR
DEEFSRQTLAGLNPYSIRLITEWPLKSTLDPEIYGPPESAITTELIEKEI
GGMISVEEAIKQKKLFILDYHDLFLPYVEKVRQLKATTLYGSRTIFFLTP
GGTLRPIAIELTRPPMNGKPQWKQVFLPSWHSTECWLWKLAKAHVLAHDA
GYHQLVSHWLRTHCCTEPYVIATNRQLSVMAPIYRLLDPHFRYTMEINGL
ARQALVNADGIIESSFSPGKYSMEFSSVAYDKQWRFDHEALPKDLISRGL
AVEDPSAPHGLKLTIEDYPFANDGLDLWDAIKQWVTDYVNHYYPDKSLVE
SDEELQAWWTEIRTVGHGDKKDEPWWPVLKTPQDLIEIITTIVWVTSGHH
AAVNFGQYIYGGYFPNRPTTARCNIATEDPTDEQWKFFLEKPENALLNTF
PSQIQATKVMAILDVLSTHSPDEEYLGKEIEPAWREEPVINAAFEKFRGR
LMELEGIIDARNADPKERNRNGAGMVPYELLKPFSEPGVTGKGVPYSISI
> PELOX2.3_Ptrif.0002s0208.1.v1.3.1_Poncirus_trifoliata
MLKPQVHQSHQSLKPLVPLSKPFLQGNVHAFRALQSSRSIKNIPKIRIGI
SPSVNIKAITTFTEKSTEVKAFVTIIPSVGGLVSGFVDDVKDMFGKSLLL
ELVSAELDPKTGAEKPTIKGFAHRAGEDKDGHIIYESKFEVPPSFGEVGA
ILVENEHHKEMYLNDIVLDGPRNGPVNITCGSWVQSKHVNKQKRIFFTNK
SYLPSQTPNGLTRLRAEELINLRGDGQGERKTHDRIYDYDVYNDLGVPDF
CSELARPVLGGKEHPYPRRCRTGRPPCETDPASESRTLINYVPRDEAFSE
IKQLQFSAKTLYSVLHGLVPSLETAIIDTDLGFPYFTAIDKLFNEGVNVP
MPETFKEKALWRTILPRLVKGIEDTGKEVERFETPETMDRDKFFWFRDEE
FGRQTLAGLNPYSIRLVTEWPLRSTSDPEIYGPPESAITKELIEKEIGGI
MTVEEAIKQKKLFILDYHDLLLPYVEKVRELKGTTLYGSRTLFFSYPSGT
LRPLAIELTRPPMDGKPQWKQVFTPSWHSTECWLWRLAKAHVLAHDSGYH
QLVSHWLRTHCCTEPYIIATNRQLSAMHPINRLLQPHFRYTMEINALARE
ALINAGGIIESTFSPGKYSMEISSVAYDKHWRFDHEALPKDLISRGMAVE
DPSASHGIKLTIEDYPFAKDGLDLWDALKQWVTDYVNHYYPNPISVESDE
ELQSWWTEIRTVGHADKKDEPWWPVLKTPEDLIDIITTIVWVASGHHAAV
NFGQYTFGGYFPNRPTVARTKMPIEDPSDEDWKFFLEKPEDVLLQCFPSQ
IQATIVMAILDTLSSHSPDEEYLGKQMEQAWGDDPVIKAAFERFSGRLKE
LEGIIDERNANENLKNRTGAGMVPYELMKPFSEPGVTGQGVPYSISI
>XP_006445968.1 linoleate 13S-lipoxygenase 2-1, chloroplastic [Citrus
clementina]
MFNPVLVHQTRSIRTILPLSKPFLHGNGNVFRQIQSSPSFKKGPKIRLGSVPSNSVKAMADTAVSNGVTAVVTVRPPINP
LTAGGQVIDDVEDLFSKSLQLELVSAKDENKPTISGNAKIKGVVVKDCEVQYEAEFQVPVDFGEIGAILVVNEHALEMYL
KDIVLDGLPSGLVTITCESWVQPNTSKDPRIFFTNKSYLPSKTPNGLQKLRYAELVNLRGNGEGERQKADRIYDYDVYND
LGDPDEDEELKRPVLGGKQHPYPRRCRTGRPHCKTDEASEERVPSKSLIPISPYVPRDEEFSAIKETTFVIRGLFGQLRS
LIPNLKAEFVDTDGFPNFTEIDKLFREGVKIKDAEFWKSLLPGFVEEIKDIGDFFLRFTSPETFKRDRFFWFRDEEFARE
TLAGLNPCSIRLITEWPLKSSLDPKIYGPSESAITTEMIESEIKGCTTVKEALNQKKLFILDYHDLFLPYVEQVRELGDR
TLYGSRTVFYLNPDGTLRPLAIELTRPPMDGKPQWKQAFTPSSDSTKSWLWKLAKAHVLAHDSGYHQLISHWLRTHCSVE
PYAIAAHRQLSAMHPINRLLKPHFRYTMEINSLARQSLINAGGVIESTFLPGKYSMLLSSIIYDKQWRFDHQALPQDLIS
RGMAAKDPSSPHGLKLTIEDYPFAQDGLDLWDIIKQWVIDYVSHYYPDPSEVESDEELQAWWTEIRTVGHGDKQDETWWP
VINSPKDLIDTITSIVWVASGLHAAVNFGQYEYAAYFPNRPTIARANMPNEDPSDDEWQIFFERPEAALLTTFPNQRQAT
AVISVLDVLSAHSPDEDYLGKYMEPAWGEDKIIKGAFEKFQGRLMELKGIINLRNADKNLKNRHGAGSLPYELLMPLADK
SGVTGKGVPYSISI
>AHG99489.1 lipoxygenase [Citrus suavissima]
MFNPVLVHQTRSIRTILPLSKPFLHGNGNVFRQIQSSPSFKKGPKIRLGSVPSNSVKAMADTAVSNGVTAVVTVRPPINP
LTAGGQVIDDVEDLFSKSLQLELVSAKDENKPTISGNAKIKGVVVKDCEVQYEAEFQVPVDFGEIGAILVVNEHALEMYL
KDIVLDGLPSGLVTITCESWVQPNTSKDPRIFFTNKSYLPSKTPNGLQKLRYAELVNLRGNGEGERQKADRIYDYDVYND
LGDPDEDEELKRPVLGGKQHPYPRRCRTGRPHCKTDEASEERVPSKSLIPISPYVPRDEESRRLKETTFVIKGIVSDSCV
SLNTKLESRICRHRWVSQTSQKIDKLFREGVKIKDAEFWKSLLPGFVEEIKDIGDFFLRFTSPETFKRDRFFWFRDEEFS
RQTLAGLNPYSIRLIAEWPLKSTLDPEIYGPPESAITTELIEKEIGGMISVEEAIKQKKLFILDYHDLFLPYVEKVRQLK
STTLYGSRTIFFLTPAGTLRPIAIELTRPPMNGKPQWKQVFLPSWHSTECWLWKLAKAHVLAHDAGYHQLVSHWLNTHCC
TEPYVIATNRQLSVMHPIYRLLDPHFRYTMEINGLARQALVNADGIIESSFSPGKYSMEFSSVAYDKQWRFDHEALPKDL
ISRGLAVEDPSAPHGLKLTIEDYPFANDGLDLWDAIKQWVTDYVNHYYPDKSLVESDEELQAWWTEIRTVGHGDKKHEPW
WPVLKTPKDLIEIITTIVWVTSGHHAAVNFGQYTYGGYFPNRPTTARCNIATEDPSDEQWKFFLEKPENALLNTFPSQIQ
ATKVMAILDVLSTHSPDEEYLGKEIEPAWREDPVINAAFEKFRGKLMELEGIIDARNADPKLRNRNGAGMVPYELLKPFS
EPGVTGKGVPYSISI
>BAB84352.1 lipoxygenase [Citrus jambhiri]
MFNPVLVHQTRSIRTILPLSKPFLHGNGNVFRQIQSSPSFKKGPKIRLGSVPSNSVKAMADTAVSNGVTAVVTVRPPINP
LTAGGQVIDDVEDLFSKSLQLELVSAKDENKPTISGNAKIKGVVVKDCEVQYEAEFQVPVDFGEIGAILVVNEHALEMYL
KDIVLDGLPSGLVTITCESWVQPNTSKDPRIFFTNKSYLPSKTPNGLQKLRYAELVNLRGNGEGERQKADRIYDYDVYND
LGDPDEDEELKRPVLGGKQHPYPRRCRTGRPHCKTDEASEERVPSKSLIPISPYVPRDEESRRLKETTFVIKGIVSDSCV
SLNTKLESRICRHRWVSQTSQKIDKLFREGVKIKDAEFWKSLLPGFVEEIKDIGDFFLRFTSPETFKRDRFFWFRDEEFS
RQTLAGLNPYSIRLIAEWPLKSTLDPEIYGPPESAITTELIEKEIGGMISVEEAIKQKKLFILDYHDLFLPYVEKVRQLK
STTLYGSRTIFFLTPAGTLRPIAIELTRPPMNGKPQWKQVFLPSWHSTECWLWKLAKAHVLAHDAGYHQLVSHWLNTHCC
TEPYVIATNRQLSVMHPIYRLLDPHFRYTMEINGLARQALVNADGIIESSFSPGKYSMEFSSVAYDKQWRFDHEALPKDL
ISRGLAVEDPSAPHGLKLTIEDYPFANDGLDLWDAIKQWVTDYVNHYYPDKSLVESDEELQAWWTEIRTVGHGDKKDEPW
WPALKTPQDLIEIITTIVWVISGHHAAVNFGQYIYGGYFPNRPTTARCNIATEDPSDEQWKFFLEKPENALLNTFPSQIQ
ATKVMAILDVLSTHSPDEEYLGKEIEPAWREDPVINAAFEKFRGKLMELEGIIDARNADPKLRNRNGAGMVPYELLKPFS
EPGVTGKGVPYSISI
>ESR59207.1 hypothetical protein CICLE_v10014207mg [Citrus clementina]
MFNPVLVHQTRSIRTILPLSKPFLHGNGNVFRQIQSSPSFKKGPKIRLGSVPSNSVKAMADTAVSNGVTAVVTVRPPINP
LTAGGQVIDDVEDLFSKSLQLELVSAKDENKPTISGNAKIKGVVVKDCEVQYEAEFQVPVDFGEIGAILVVNEHALEMYL
KDIVLDGLPSGLVTITCESWVQPNTSKDPRIFFTNKSYLPSKTPNGLQKLRYAELVNLRGNGEGERQKADRIYDYDVYND
LGDPDEDEELKRPVLGGKQHPYPRRCRTGRPHCKTDEASEERVPSKSLIPISPYVPRDEEFSAIKETTFVIRGLFGQLRS
LIPNLKAEFVDTDGFPNFTEIDKLFREGVKIKDAEFWKSLLPGFVEEIKDIGDFFLRFTSPETFKRDRFFWFRDEEFARE
TLAGLNPCSIRLITEWPLKSSLDPKIYGPSESAITTEMIESEIKGCTTVKEALNQKKLFILDYHDLFLPYVEQVRELGDR
TLYGSRTVFYLNPDGTLRPLAIELTRPPMDGKPQWKQAFTPSSDSTKSWLWKLAKAHVLAHDSGYHQLISHWLRTHCSVE
PYAIAAHRQLSAMHPINRLLKPHFRYTMEINSLARQSLINAGGVIESTFLPGKYSMLLSSIIYDKQWRFDHQALPQDLIS
R
>XP_006494720.1 linoleate 13S-lipoxygenase 2-1, chloroplastic [Citrus sinensis]
MLKPQVHQPQSIKPLFPLSKPFLHGNYGHAFRPVQSTSTLFKGSPKLRIGSVPRNTIKAIATSTEKSIKVKAVVTVKPTV
GGFLSNISLDQGLDDLGDLFGKSLLLELVSAELDPKTGLDKSTIQDYARKIGADGDGNMQYESEFEVPSGFGEIGAILVE
NEHHKEMYLKDIVLDGLPNGPVNVTCNSWLHSKHDNKQKRVFFTNKLYLPSQTPDGLKRYRAEELTILRGNGQGERKTYD
RIYDYDVYNDLGDPDKKPELARPVLGGKQNPYPRRCRTGRPRCDTDQFSEKREGNFYVPRDEAFSEVKQLTFSAKTVYSV
LHALVPSLETAFVDPDLGFPYFSAIDALFNEGVNLPPLKQEGFWNTLLPRLVKAIEDTGDNILLFETPETMDRDKFFWFR
DEEFSRQTLAGLNPYSIRLITEWPLKSTLDPEIYGPPESAITTELIEKEIGGMISVEEAIKQKKLFILDYHDLFLPYVEK
VRQLKSTTLYGSRTIFFLTPAGTLRPIAIELTRPPMNGKPQWKQVFLPSWHSTECWLWKLAKAHVLAHDAGYHQLVSHWL
RTHCCTEPYVIATNRQLSVMHPIYRLLDPHFRYTMEINGLARQALVNADGIIESSESPGKYSMEFSSVAYDKQWRFDHEA
LPKDLISRGLAVEDPSAPHGLKLTIEDYPFANDGLDLWDAIKQWVTDYVNHYYPDKSLVESDEELQAWWTEIRTVGHGDK
KHEPWWPVLKTPKDLIEIITTIVWVTSGHHAAVNFGQYTYGGYFPNRPTTARCNIATEDPSDEQWKFFLEKPENALLNTF
PSQIQATKVMAILDVLSTHSPDEEYLGKEIEPAWREDPVINAAFEKERGKIMELEGIIDARNADPKLRNRNGAGMVPYEL
LKPFSEPGVTGKGVPYSISI
>GAY49879.1 hypothetical protein CUMW_122450 [Citrus unshiu]
MLKPQVHQPQSIKPLFPLSKPFLHGNYGHAFRPVQSTSTLFKGSPKLRIGSVPRNTIKAIATSTEKSIKVKAVVTVKPTV
GGFLSNISLDQGLDDLGDLFGKSLLLELVSAELDPKTGLDKSTIQDYARKIGADGDGNMQYESEFEVPSGFGEIGAILVE
NEHHKEMYLKDIVLDGLPNGPVNVTCNSWLHSKHDNKQKRVFFTNKLYLPSQTPDGLKRYRAEELTILRGNGQGERKTYD
RIYDYDVYNDLGDPDKKPELARPVLGGKQNPYPRRCRTGRPRCDTDQFSEKREGNFYVPRDEAFSEVKQLTFSAKTVYSV
LHALVPSLETAFVDPDLGFPYFSAIDALFNEGVNLPPLKQEGFWNTLLPRLVKAIEDTGDNILLFETPETMDRDKFFWFR
DEEFSRQTLAGLNPYSIRLITQEDKKLHEIAQEWPLKSTLDPEIYGPPESAITTELIEKEIGGMISVEEAIKQKKLFILD
YHDLFLPYVEKVRQLKSTTLYGSRTIFFLTPAGTLRPIAIELTRPPMNGKPQWKQVFLPSWHSTECWLWKLAKAHVLAHD
AGYHQLVSHWLRTHCCTEPYVIATNRQLSVMHPIYRLLDPHFRYTMEINGLARQALVNADGIIESSFSPGKYSMEFSSVA
YDKQWRFDHEALPKDLISRGLAVEDPSAPHGLKLTIEDYPFANDGLDLWDAIKQWVTDYVNHYYPDKSLVESDEELQAWW
TEIRTVGHGDKKHEPWWPVLKTPKDLIEIITTIVWVTSGHHAAVNFGQYTYGGYFPNRPTTARCNIATEDPSDEQWKFFL
EKPENALLNTFPSQIQATKVMAILDVLSTHSPDEEYLGKEIEPAWREDPVINAAFEKFRGKLMELEGIIDARNADPKLRN
RNGAGMVPYELLKPFSEPGVTGKGVPYSISI
>XP_006445963.1 linoleate 13S-lipoxygenase 2-1, chloroplastic [Citrus
clementina]
MLKPQVHQSHQSLKPLVPLSKPFLRGNFHAFRALQSSSSIKNIPKIRIGISPSVNIKAITTFTQKSTQVKAFVTIKPSVG
GLVSGFVDDVKDMFGKSLLLELVSAELDPKTGAEKPTIKGFAHRAGEDKDGHIIYESKFEVPPSFGEVGAILVENEHHKE
MYINDIVIDGPRNGPVNITCGSWVQSKHVNKQKRIFFTNKSYLPSQTPNGLTRLRAEELLNERGDGQGERKTHDRIYDYD
VYNDLGVPDFCSELARPVLGGKEHPYPRRCRTGRPPCETDPASESRTLINYVPRDEAFSEIKQLQFSAKTLYSVLHGLVP
SLETAIIDTDLGFPYFTTIDKLFNEGVNVPMPETFKEKALWRTILPRLVKGIEDTGKEVLRFETPETMDRDKFFWFRDEE
FGRQTLAGLNPYSIRLVTEWPLRSTLDPEIYGPPESAITKELIEKEIGGIMTVEEAIKQKKLFILDYHDLLLPYVEKVRE
LKGTTLYGSRTLFFSYPSGTLRPLAIELTRPPMDGKPQWKQVFTPSWHSTECWLWRLAKAHVLAHDSGYHQLVSHWLRTH
CCTEPYIIATNRQLSAMHPINRLLQPHFRYTMEINALAREALVNAGGIIESTFSPGKYSMELSSVAYDKHWRFDHEALPK
DLISRGMAVEDPSAPRGIKLTIEDYPFAKDGLDLWDALKQWVTDFVNHYYPNPSSVESDEELRSWWTEIRTVGHADKKDE
PWWPVLKTPEDLIDIITTIAWVASGHHAAVNFGQYTFGGYFPNRPTVARTKMPIEDPSDEDWKLFLEKPEDVLLQCFPSQ
IQATTVMAILDTLSSHSPDEEYLGKQMEQAWGDDPVIKAAFERFSGRLKELEGIIDERNANENLKNRTGAGMVPYELMKP
FSEPGVTGQGVPYSISI
>XP_006445970.1 linoleate 13S-lipoxygenase 2-1, chloroplastic [Citrus
clementina]
MLKPQVHQPQSIKPLFPLSNPFLAGNYGHAFLPVPSTSSLFKGSPKLRIGSVPRNTIKAIATSTEKSIKVKAVVTVKPTV
GSFLSNISLDRGLDDLGDLFGKSLLLELVSAELDPKTGLDKSTIQDYARKIGADGDGNMQYESEFEVPSGFGEIGAILVE
NEHHKEMYLKDIVLDGLPNGPVNVTCNSWLHSKHDNKQKRVFFTNKYLPSQTPDGLKRYRAEELTILRGNGQGERKTYDR
IYDYDVYNDLGDPDKKPELARPVLGGKQNPYPRRCRTGRPRCDTDQFSEKREGNFYVPRDEAFSEVKQLTFSAKTVYSVL
HALVPSLETAFVDPDLGFPYFSAIDALFNEGVNLPPLKQEGFWNTLLPRLVKAIEDTGDNILLFETPETMDRDKFFWFRD
EEFSRQTLAGLNPYSIRLITEWPLKSTLDPEIYGPPESAITTELIEKEIGGMISVEEAIKQKKLFILDYHDLFLPYVEKV
RQLKSTTLYGSRTIFFLTPAGTLRPIAIELTRPPMNGKPQWKQVFLPSWHSTECWLWKLAKAHVLAHDAGYHQLVSHWLR
THCCTEPYVIATNRQLSVMHPIYRLLDPHFRYTMEINGLARQALVNADGIIESSFSPGKYSMEFSSVAYDKQWRFDHEAL
PKDLISRGLAVEDPSAPHGLKLTIEDYPFANDGLDLWDAIKQWVTDYVNHYYPDKSLVESDEELQAWWTEIRTVGHGDKK
HEPWWPVLKTPKDLIEIITTIVWVTSGHHAAVNFGQYIYGGYFPNRPTTARCNIATEDPSDEQWKFFLEKPENALINTFP
SQIQATKVMAILDVLSTHSPDEEYLGKEIEPAWREDPVINAAFEKFRGKLMELEGIIDARNADPKLRNRNGAGMVPYELL
KPFSEPGVTGKGVPYSISI
>GAY49897.1 hypothetical protein CUMW_122560 [Citrus unshiu]
MSYSPSQRSLTCERMLNPQVHHQSQSIRTLCPLPKPFLRGNGQAFRPAQINPSFKKASKIGVGFSPANNSIKAIFNLTEK
STKVKAVITVKPIIPDPLALSSLVGALGLELVSAELDPKTGEEKPTIKGLALGVLGKDDDGNIKYKAELKIPASFGDVGA
ILVESDQLTEMYLQDIVLDGLPNGPVNLTCDSWIQPKIVDKQKRIFFTNKSYLPSQTPNGLKRLRAEELNNLQGDGQGER
KRHERIYDYDVYNDLALPEIKELARPVLGGEEHPYPRRCRTGRPKSFADPASESRSVSIYVERDEAFADIKLGQFSASSL
YSGLHALVPFLEAILIDKDLGFSSLSDIDKVFNEGIELPPELKDQPLWQKILPILFKTVSNTGKEVFRFDTPETVDRDKF
FWIRNEEFGRETLAGLNPYSIKLLSQLPLKSTLDPEIYGPPESAITTELIEQEIGGLMTVNEAIKQKKLFIIDYHDALLP
YVGKLRQIEGSTLYGSRALFFLNPDGTLRPLAIELTRPPLDGKPQWKQVLRTHCCVEPYIIAANRKLSAMHPINRLLKPH
FRYTMEINAKARLILVNAGGIVETTFFPGKYSMEFSSVIYDKQWRFDHEALPKDLISRGMAVEDPNAPHGLKLTIEDYPF
ANDGLDLWDALKQWVTEYVNHYYTDPSLVESDEELQAWWTEIRTVGHADKKDEPWWPVLKTPQDLIEIVTTIAWVASGQH
AAVNFGQYLYGGYFPNRPTMSRTNMPTEDQSEADWKSFLANPEDTLLQCFPSKMQAMQDMVILDTLSTHSPDEEYLGKEM
EPAWGDDPVIKAAFEKFNRKMQELEGIIDDRNSNENLRNRTGAGIVPYELLKPFSGPGATGKGVPMLKPQVHQSHQSLKP
LVPLSKPFLRGNFHAFRALQSSSSIKNIPKIRIGISPSVNIKAITTFTQKSTQVKAFVTIKPSVGGLVSGFVDDVKDMFG
KSLLLELVSAELDPKTGAEKPTIKGFAHRAGEDKDGHIIYESKFEVPPSFGEVGAILVENEHHKEMYLNDIVLDGPRNGP
VNITCGSWVQSKHVNKQKRIFFTNKSYLPSQTPNGLTRLRAEELLNLRGDGQGERKTHDRIYDYDVYNDLGVPDFCSELA
RPVLGGKEHPYPRRCRTGRPPCETDPASESRTLINYVPRDEAFSEIKQLQFSAKTLYSVLHGLVPSLETAIIDTDLGFPY
FTTIDKLFNEGVNVPMPETFKEKALWRTILPRLVKGIEDTGKEVLRFETPETMDRDKFFWFRDEEFGRQTLAGLNPYSIR
LVTEWPLRSTLDPEIYGPPESAITKELIEKEIGGIMTVEEAIKQKKLFILDYHDLLLPYVEKVRELKGTTLYGSRTLFFS
YPSGTLRPLAIELTRPPMDGKPQWKQVFTPSWHSTECWLWRLAKAHVLAHDSGYHQLVKPYIIATNRQLSAMHPINRLLQ
PHFRYTMEINALAREALVNAGGIIESTFSPGKYSMELSSVAYDKHWRFDHEALPKDLISRGMAVEDPSAPRGIKLTIEDY
PFAKDGLDLWDALKQWVTDFVNHYYPNPSSVESDEELRSWWTEIRTVGHADKKDEPWWPVLKTPEDLIDIITTIAWVASG
HHAAVNFGQYTFGGYFPNRPTVARTKMPIEDPSDEDWKLFLEKPEDVLLQCFPSQIQATTVMAILDTLSSHSPDEEYLGK
QMEQAWGDDPVIKAAFERFSGRLKELEGIIDERNANENLKNRTGAGMVPYELMKPFSEPGVTGQGVPYSISI
>XP_006445964.1 linoleate 13S-lipoxygenase 2-1, chloroplastic [Citrus
clementina]
MLNPQVHHQSQSIRTLCPLPKPFLRGNGQAFRPAQLNPSFKKASKIGVGFSPSNNSIKAIFNLTEKSTKVKAVITVKPII
PDPLALSSLVGALGLELVSAELDPKTGEEKPTIKGLALGVLGKDDDGNIKYKAELKIPASFGDVGAILVESDQLTEMYLQ
DIVLDGLPNGPVNLTCDSWIQPKIVDKQKRIFFTNKSYLPSQTPNGLKRLRAEELNNLQGDGQGERKRHERIYDYDVYND
LALPEIKELARPVLGGEEHPYPRRCRTGRPKSFADPASESRSVSIYVPRDEAFADIKLGQFSASSLYSGLHALVPFLEAI
LIDKDLGFSSLSDIDKVFNEGIELPPELKDQPLWQKILPILFKTVSNTGKEVFRFDTPETVDRDKFFWIRNEEFGRETLA
GLNPYSIKLLSQLPLKSTLDPEIYGPPESAITTELIEQEIGGLMTVNEAIKQKKLFIIDYHDALLPYVGKLRQIEGSTLY
GSRALFFLNPDGTLRPLAIELTRPPLDGKPQWKQVFTPSRHSTDSWLWTLAKAHFLVHDTGYHQLISHWLRTHCCVEPYI
IAANRKLSAMHPINRLLKPHFRYTMEINAKARLILVNAGGLVETTFFPGKYSMEFSSVIYDKQWRFDHEALPKDLISRGM
AVEDPNAPHGLKLTIEDYPFANDGLDLWDALKQWVTEYVNHYYTDPSLVESDEELQAWWTEIRTVGHADKKDEPWWPVLK
TPQDLIEIVITIAWVASGQHAAVNFGQYLYGGYFPNRPTMSRTNMPTEDQSEADWKSFLANPEDTLLQCFPSKMQAMQDM
VILDTLSTHSPDEEYLGKEMEPAWGDDPVIKAAFEKFNRKMQELEGIIDDRNSNENLRNRTGAGIVPYELLKPFSGPGAT
GKGVPYSISI
>KDO56506.1 hypothetical protein CISIN_19002644mg [Citrus sinensis]
MTANTYEVQINIYSETGAEKPTIKGFAHRAGEDKDGHIIYESKFEVPPSFGEVGAILVENEHHKEMYINDIVIDGPRNGP
VNITCGSWVQSKHVNKQKRIFFTNKSYLPSQTPNGLTRLRAEELLNLRGDGQGERKTHDRIYDYDVYNDLGVPDFCSELA
RPVLGGKEHPYPRRCRTGRPPCETDPASESRTLINYVPRDEAFSEIKQLQFSAKTLYSVLHGLVPSLETAIIDTDLGFPY
FTTIDKLFNEGVNVPMPETFKEKALWRTILPRLVKGIEDTGKEVLRFETPETMDRDKFFWFRDEEFGRQTLAGLNPYSIR
LVTEWPLRSTLDPEIYGPPESAITKELIEKEIGGIMTVEEAIKQKKLFILDYHDLLLPYVEKVRELKGTTLYGSRTLFFS
YPSGTLRPLATELTRPPMDGKPQWKQVFTPSWHSTECWLWRLAKAHVLAHDSGYHQLVSHWIRTHCCTEPYIIATNRQLS
AMHPINRLLQPHFRYTMEINALAREALVNAGGIIESTFSPGKYSMELSSVAYDKHWRFDHEALPKDLISRGMAVEDPSAP
RGIKLTIEDYPFAKDGLDLWDALKQWVTDFVNHYYPNPSSVESDEELRSWWTEIRTVGHADKKDEPWWPVLKTPEDLIDI
ITTIAWVASGHHAAVNFGQYTFGGYFPNRPTVARTKMPIEDPSDEDWKLFLEKPEDVLLQCFPSQIQATTVMAILDTLSS
HSPDEEYLGKQMEQAWGDDPVIKAAFERFSGRLKELEGIIDERNANENLKNRTGAGMVPYELMKPFSEPGVTGQGVPYSI
SI
>KDO64920.1 hypothetical protein CISIN_19002617mg [Citrus sinensis]
MQYESEFEVPSGFGEIGAILVENEHHKEMYLKDIVLDGLPNGPVNVTCNSWLHSKHDNKQKRVFFTNKLYLPSQTPDGLK
RYRAEELTILRGNGQGERKTYDRIYDYDVYNDLGDPDKKPELARPVLGGKQNPYPRRCRTGRPRCDTDQFSEKREGNFYV
PRDEAFSEVKQLTFSAKTVYSVLHALVPSLETAFVDPDLGFPYFSAIDALFNEGVNLPPLKQEGFWNTLLPRLVKAIEDT
GDNILLFETPETMDRDKFFWFRDEEFSRQTLAGLNPYSIRLITEWPLKSTLDPEIYGPPESAITTELIEKEIGGMISVEE
AIKQKKLFILDYHDLFLPYVEKVRQLKSTTLYGSRTIFFLTPAGTLRPIAIELTRPPMNGKPQWKQVFLPSWHSTECWLW
KLAKAHVLAHDAGYHQLVSHWLRTHCCTEPYVIATNRQLSVMHPTYRLLDPHFRYTMEINGLARQALVNADGIIESSFSP
GKYSMEFSSVAYDKQWRFDHEALPKDLISRGLAVEDPSAPHGLKLTIEDYPFANDGLDLWDAIKQWVTDYVNHYYPDKSL
VESDEELQAWWTEIRTVGHGDKKHEPWWPVLKTPKDLIEIITTIVWVTSGHHAAVNFGQYTYGGYFPNRPTTARCNIATE
DPSDEQWKFFLEKPENALLNTFPSQIQATKVMAILDVLSTHSPDEEYLGKEIEPAWREDPVINAAFEKFRGKLMELEGII
DARNADPKLRNRNGAGMVPYELLKPFSEPGVTGKGVPYSISI
>XP_006445965.1 linoleate 13S-lipoxygenase 2-1, chloroplastic [Citrus
clementina]
MLNLQFHNQSQSIRTLSPLPNPFLHGNGPAFRPAQLRPSFKKAPKIGVGFSPSINSIKAIFNLTAEKSTKVKAVITVKPI
VSDPLAVEKLIGTLVLELVSAELDPETGKEKPTIKSRAHRSLFTDDDGNLKYKTEFDVPSNFGEVGAILVEADQLTETFL
KDIALDGLRNGPVNIACDSWIQPKIVDKQKRIFFINKSYLPSQTPNGLTRERAEELNNLQGDGQGERKIHERTYDYDVYN
DLGMPDSILKSDLVRPVLGGKEHPYPRRCRTGHPKSSKDPASESWSLSVYVPRDEAFSLLKTAQFSATGVYSALHAVIPF
VESILRIGKDKGFPSLEAIDKLFNEGVELPPEIEKLPSWLKILPNFFKSIANTGKDILRFETPETLKRDKLFWLRDEEFG
RETLAGLNPYGISLVADWPLKSTLDPETYGPTESAITKELIEKEIGGSMTVEEAIKQKKLFIIDYHDALLPYVEKVRQIK
DTTLYGSRTVFFLNPDGTLRPLAIELTRPPMDGKPQWKQVFTPSTGNSTESWLWRLAKAHVLAHDSGYHQLISHWLRTHC
CVEPYVIATNRRLSAMHPINRLLKPHFRYTMEINALARKVLINADGIFETNFFPGKYCMEFSSVIYDKHWRFDNEGLPKD
LIRRGIAVEDPKAPHGLKLNIEDYPYANDGLDLWDALKQWVTNYVNHYYPDPSLVESDEELQAWWTEIRTVGHAEKKDEP
WWPVLKTPQDLIEIITTIAWVASGHHAAVNFGQYLYGGYFPNRPTVARTNLPNEDQTKEEWKSFLEKPEAALLRCFPAQF
QALTVMEVIDLLSTHSPDEEYLGKEMEPAWGDDPVIKAAFEEFNLKMQELERIIDDRNSNENLKNRTGAAIVPYELLKPF
SEPGATGKGVPYSISI
>XP_006494272.1 linoleate 13S-lipoxygenase 2-1, chloroplastic [Citrus sinensis]
MLNLQFHNQSQSIRTLSPLPNPFLHGNGPAFRPAQLRPSFKKAPKIGVGFSPSINSIKAIFNLTAEKSTKVKAVITVKPI
VSDPLAVEKLIGTLVLELVSAELDPETGKEKPTIKSRAHRSLFTDDDGNLKYKTEFDVPSNFGEVGAILVEADQLTETFL
KDIALDGLRNGPVNIACDSWIQPKIVDKQKRIFFTNKSYLPSQTPNGLARLRAEELNNLQGDGQGERKIHERIYDYDVYN
DLGMPDSILKSDEVRPVLGGKEHPYPRRCRTGHPKSSKDPASESWSLSVYVPRDEAFSLLKTAQFSATGVYSALHAVIPF
VESILRIGKDKGFPSLEAIDKLFNEGVELPPEIEKLPSWLKILPNFFKSIANTGKDILRFETPETLKRDKLFWLRDEEFG
RETLAGLNPYGISLVADWPLKSTLDPETYGPTESAITKELIEKEIGGSMTVEEAIKQKKLFIIDYHDALLPYVEKVRQIK
DTTLYGSRTVFFLNPDGTLRPLAIELTRPPMDGKPQWKQVFTPSTGNSTESWLWRLAKAHVLAHDSGYHQLISHWLRTHC
CVEPYVIATNRRLSAMHPINRLLKPHFRYTMEINALARKVLINADGIFETNFFPGKYCMEFSSVIYDKHWRFDNEGLPKD
LIRRGIAVEDPKAPHGLKLNIEDYPYANDGLDLWDALKQWVTNYVNHYYPDPSLVESDEELQAWWTEIRTVGHAEKKDEP
WWPVLKTPQDLIEIITTIAWVASGHHAAVNFGQYLYGGYFPNRPTVARTNLPNEDQTKEEWKSFLEKPEAALLRCFPAQF
QALTVMLVIDLLSTHSPDEEYLGKEMEPAWGDDPVIKAAFEEFNLKMQELERIIDDRNSNENLKNRTGAAIVPYELLKPF
SEPGATGKGVPYSISI
>XP_006445969.1 linoleate 13S-lipoxygenase 2-1, chloroplastic [Citrus
clementina]
MLKPQVHRYSTPTTVLFPFSKPFLHGNCHVFRQVQPSPSLKIGSKVRVSCHSKRHNVSSKNIEAIATSTEKSVSVINAVV
TVKASWKDDYEDYFLGRTLRLELVSADLDHTTGSEKSTIYAHARKAGKDKHGNELYESTFNVPSDFGEVGAMSVENEHHV
EIYLMNIVLDGFSNGDPVNITCNSWIQPKNKNELKRIFFANKSYLPSQTPDGLKRFRIEELYHLRGNGQGVRQPSDRIYD
YDVYNDLGNPDKRRPVLGGKKFPYPRRCRTGRPHYESDPLKEKRDKYIYVPRDETFSDVKQEAFDNMKDYKSMCHAALPY
IEKFFDGDKKFEYFTEIDELFNEDGFSLPEAEPGFLNSLARFAKTLKEMGEEVFQFDAPEAMLRDKFFWFRDEEFARQTL
AGLNPCSIQLITEWPLKSSLDPKIYGPQESAITKDIIEKELGEMISVEEAIEQKKLFMLDYHDLFLPYVEKVRKLEPTTL
YGSRTVFFLTPDNTLRPLAIELTRPPMDDKPHWRKVYTPGSWNSTKTWLWRLAKAHVLAHDSGYHQLVSHWLRSHCVVEP
YIIATNRQLSVMHPIYRLLHPHLRYTLELNAIGRDILISAGGVIENTFSPGEYCMEMSSVIYDKQWRFDEQALPKDLMKR
GMAVEDPNARHGLKLTIDDYPFAKDGLDLWGILKQWVTDYVNHYYPDQSLVESDDELQAWWTEIRTVGHADKKDEPWWPV
LKTPQNLIEILTTIIWVASGHHAAVNFGQYTYAAYFPNRPTIARVNMPDEDPTEKIWKTFIEKPEDALLYTFPNQDQAIL
VIATLDLLSTHSPDEEFLGKDKEPAWGEDPVINAAFEKFSGRLMELEGIIDERNGDSTLANRNGAGVVPYNLLKPYWKDG
DKEKGVPYSISI
>XP_006494271.1 linoleate 13S-lipoxygenase 2-1, chloroplastic [Citrus sinensis]
MLKPQVHRYSTPTTVLFPFSKPFLHGNCHVFRQVQPSPSLKIGSKVRVSCHSKRHNVSSKNIEAIATSTEKSVSVINAVV
TVKATWKDDYEDYLFGRTLRLELVSAELDHTTGSEKSTIYAHASKAGKDKHGNELYEATFNVPSDFGEVGAMSVENEHHV
EIYLMNIVLDGFSNGDPVNITCNSWIQPKNKNEPKRIFFTNKSYLPSQTPDGLKRFRIEELYHLRGHGQGVRQPSDRIYD
YDVYNDLGNPDKRRPVLGGKKFPYPRRCRTGRQHYESDPLKEKRDKYIYVPRDETFSDVKQEAFDNMKDYKSMCHAALPY
IEKFFDGDKKFEYFTEIDELFNEDGFSLPEAEPGFLNSLARFAKTLKEMGEEVFQFDAPEAMLRDKFFWFRDEEFARQTL
AGLNPCSIQLITEWPLKSSLDPKIYGPRLQESEITKDIIEKELGAMISVEEAIEQKKLFMLDYHDLFLPYVEKVRKLEHT
TLYGSRTVFFLTPQNTLRPLAIELTRPPMDDKPLWRKVYTPGSWNSTKTWLWRLAKAHVLAHDSGYHQLVSHWLRSHCVV
EPYIIATNRQLSVMHPIYRLLHPHLRYTLELNAIGRDILISAGGVIENTFSPGEYCMEMSSVIYDKQWRFDEQALPKDLM
KRGMAVEDPNARHGLKLTIDDYPFAKDGLDLWGILKQWVTDYVNHYYPDQSLVESDDELQAWWTEIRTVGHADKKDEPWW
PVLKTPQNLIEILTTIIWVASGHHAAVNFGQYTYAAYFPNRPTIARVNMPDEDPTEKFWKTFIEKPEDALLYTFPNQDQA
ILVIATLDLLSTHSPDEEFLGKGKEPAWGEDPVINAAFEKFSGRLMELEGIIDERNGDSTLVNRNGAGVVPYNLLKPYWK
DGDKEKGVPYSISI
>ESR59202.1 hypothetical protein CICLE_v10018178mg, partial [Citrus clementina]
QKKILNQQVHRSRSIKTLIPFSKPFLHGNGRAFLPVHSSPSFQKSLKIRVGFSASNNIKAIAGATAPSVVSVKVKAVVTV
KHGSEKPTINVYASVAGVDLFNIRYEAEFEVPSSFGDVGGITVQHENQKEMYLKDVVLDGFLDGPMNITCDSWVQPLAID
AQKRVFFTNKSYLPSQTPNGLTRLRDEELISLRGSGQGERQPYDRIYDYDVVARPVLGGQEHPYPRRCRTGRPHCTTDPE
SETRSDSNYVPRDEAFSRIKQATFSAKTLYSLLHGLIPAIKAAFGVNKDLGFPYFTAVDTLFNQGIALPPQEQEEFWGRN
LPELIQLAKHILKFATMERHQFFWFRDEEFGRQTLAGLNPCAIQLVTKWPLESTLDPAIYGPPESAITTERVEKLMGGDI
TVAEAIQQKKLFILDYNDLLLPYVEMVRQLEGTTLYGSRTLFFLTSEGTLRPLVIELTRPPMNGQPQWKQAFQPSWQSTE
SWLWRLAKAHVLAHDSGYHQLVSHWLRTHACTEPYVIATNRHLSAMHPICTLLKPHLRYTMEINTLARESLINAELSSAV
YDQLWRFDYEALPKDLIKRGMAVDDPTAPNGLKLTIEDYPYANDGLNLWFALKKWVTDYSDKELQAWWTEIRTVGHADKK
DEPWWPALKTSEDLIEIITTIVWVASGHHAAVNFGQYAYAGYVPNRPSIARTNMPTEEPISEKDMKFFLENPQAVLLRSF
PTQLQAIQVMAVLDVLSTHSPDEEYLGNQMEPAWGKDPTIFAAFERFSGRIMELEGIIDERNADMKLKNRNGAGVVPYEL
LKPFSGRGVTEKGVPYSISI
>XP_024949679.1 linoleate 13S-lipoxygenase 2-1, chloroplastic-like [Citrus
sinensis]
MYLKDIVLKSESRNDDHGVKITCNSWLQPKEENTPTRIFFANKSYLPSATPDGLKRLRRQELRHLQGDGRGVRQLSDRIY
DYDVYNDLGNPEKDPKLKRPVLGGKEYPYPRRCRTGRPRSELGDENRADDIYVPRDEAFSDIKLAAFDSKKTYSFVSTLP
TLIETKEDGDKKFEYFTEIDELFDEDGFSIPPNENESIWNIIPRWIRKIKETGEQYLQFETPEALHRDKFFWFRDEEFAR
QTLARLNPCSIQLITELPRDSSITEEIIEKKLEEILLLHRYKHYAIQQKKLFILDYHDLFLPYVEKVRHIEDEDEALKTT
LYGSRTIFFLNPDDTLRPVAIELTRPPMDGKPFWRKVYTPSWNSTDSWLWRLAKAHVLAHDAGYHQLVSHWLSTHCVVEP
YVIATNRQLSVIHPIYRLLHPHFRYTVEINAFARKDLVNAGGIIESTFSPGKYSMELSSVAYDKQWRFDHEALPKNLISR
RMAAEDPCSPHGLKLTIEDYPYAKDGLDLWDILKKWVTDYVNHYYPNQSLVESDEELQAWWTEIRTVGHGDKEDEPWWPV
LKTPQDLIETITTIIWVTSGQHAAVNFGQYTYAGYFPNRPAITRLNMPDEDKSNEIWKIFNEKPDNALLHTFPNPTQATK
VMLILSLLSCHSPDEEFLGKDMEPAWGEDPEIKVAFEEFRGRLMELEGTINERNGDINLKNRNGAGVVPYNLLKPFWKDG
DKEKGVPYSISI
>KDO64921.1 hypothetical protein CISIN_19002617mg [Citrus sinensis]
MCVLMHADQFSEKREGNFYVPRDEAFSEVKQLTFSAKTVYSVLHALVESLETAFVDPDLGFPYFSAIDALFNEGVNLPPL
KQEGFWNTLLPRLVKAIEDTGDNILLFETPETMDRDKFFWFRDEEFSRQTLAGLNPYSIRLITEWPLKSTLDPEIYGPPE
SAITTELIEKEIGGMISVEEAIKQKKLFILDYHDLFLPYVEKVRQLKSTTLYGSRTIFFLTPAGTLRPIAIELTRPPMNG
KPQWKQVFLPSWHSTECWLWKLAKAHVLAHDAGYHQLVSHWLRTHCCTEPYVIATNRQLSVMHPIYRLLDPHFRYTMEIN
GLARQALVNADGIIESSFSPGKYSMEFSSVAYDKQWRFDHEALPKDLISRGLAVEDPSAPHGLKLTIEDYPFANDGLDLW
DAIKQWVTDYVNHYYPDKSLVESDEELQAWWTEIRTVGHGDKKHEPWWPVLKTPKDLIEIITTIVWVTSGHHAAVNFGQY
TYGGYFPNRPTTARCNIATEDPSDEQWKFFLEKPENALLNTFPSQIQATKVMAILDVLSTHSPDEEYLGKEIEPAWREDP
VINAAFEKERGKLMELEGIIDARNADPKLRNRNGAGMVPYELLKPFSEPGVTGKGVPYSISI
>KDO56507.1 hypothetical protein CISIN_19002644mg [Citrus sinensis]
MSIFRNQHDDYLSPIITNKKRLITSIKQYFPFSSFDHNVDLSLCTFPIADPASESRTLINYVPRDEAFSEIKQLQFSAKT
LYSVLHGLVPSLETAIIDTDLGFPYFTTIDKLFNEGVNVPMPETFKEKALWRTILPRLVKGIEDTGKEVLRFETPETMDR
DKFFWFRDEEFGRQTLAGLNPYSIRLVTEWPLRSTLDPEIYGPPESAITKELIEKEIGGIMTVEEAIKQKKLFILDYHDL
LLPYVEKVRELKGTTLYGSRTLFFSYPSGTLRPLAIELTRPPMDGKPQWKQVFTPSWHSTECWLWRLAKAHVLAHDSGYH
QLVSHWLRTHCCTEPYIIATNRQLSAMHPINRLLQPHFRYTMEINALAREALVNAGGIIESTFSPGKYSMELSSVAYDKH
WRFDHEALPKDLISRGMAVEDPSAPRGIKLTIEDYPFAKDGLDLWDALKQWVTDFVNHYYPNPSSVESDEELRSWWTEIR
TVGHADKKDEPWWPVLKTPEDLIDIITTIAWVASGHHAAVNFGQYTFGGYFPNRPTVARTKMPIEDPSDEDWKLFLEKPE
DVLLQCFPSQIQATTVMAILDTLSSHSPDEEYLGKQMEQAWGDDPVIKAAFERFSGRLKELEGIIDERNANENLKNRTGA
GMVPYELMKPFSEPGVTGQGVPYSISI
>GAY49883.1 hypothetical protein CUMW_122450 [Citrus unshiu]
MCVLMHADQFSEKREGNFYVPRDEAFSEVKQLTFSAKTVYSVLHALVPSLETAFVDPDLGFPYESAIDALFNEGVNLPPL
KQEGFWNTLLPRLVKAIEDTGDNILLFETPETMDRDKFFWFRDEEFSRQTLAGLNPYSIRLITQEDKKLHEIAQEWPLKS
TLDPEIYGPPESAITTELIEKEIGGMISVEEAIKQKKLFILDYHDLFLPYVEKVRQLKSTTLYGSRTIFFLTPAGTLRPI
AIELTRPPMNGKPQWKQVFLPSWHSTECWLWKLAKAHVLAHDAGYHQLVSHWLRTHCCTEPYVIATNRQLSVMHPIYRLL
DPHFRYTMEINGLARQALVNADGIIESSFSPGKYSMEFSSVAYDKQWRFDHEALPKDLISRGLAVEDPSAPHGLKLTIED
YPFANDGLDLWDAIKQWVTDYVNHYYPDKSLVESDEELQAWWTEIRTVGHGDKKHEPWWPVLKTPKDLIEIITTIVWVTS
GHHAAVNFGQYTYGGYFPNRPTTARCNIATEDPSDEQWKFFLEKPENALLNTFPSQIQATKVMAILDVLSTHSPDEEYLG
KEIEPAWREDPVINAAFEKERGKLMELEGIIDARNADPKLRNRNGAGMVPYELLKPFSEPGVTGKGVPYSISI
>GAY49899.1 hypothetical protein CUMW_122570 [Citrus unshiu]
MYLKDVVLDGFLDGPMNITCDSWVQPLAIDAQKRVFFTNKSYLPSQTPNGLTRLRDEELISLRGSGQGERQPYDRIYDYD
VVARPVLGGQEHPYPRRCRTGRPHCTTDPESETRSDSNYVPRDEAFSRIKQATFSAKTLYSLLHGLIPAIKAAFGENKDL
GFPYFTAIDTLFNQGIALPPQEQEEFWGRNLPELIQLAKHILKFATMERHQFFWFRDEEFGRQTLAGLNPCAIQLVTKWP
LESTLDPAIYGPPESAITTERVEKLMGGDITVAEAIEQKKLFILDYNDLLLPYVEKVRQLEGTTLYRSRTLFFLTSEGTL
RPLVIELTRPPMNGQPQWKQAFQPSWQSTESWLWRLAKAHVLAHDSGYHQLVSHWLRTHACTEPYVTATTRHLSAMHPIC
TLLKPHLRYTMETNTLARESLINAELSSAVYDQLWRFDYEALPKDLIKRGMAVDDPTAPNGLKLTIEDYPYANDGLNLWF
ALKKWVTEYSDKELQAWWTEIRTVGHADKKDEPWWPALKTSEDLIEIITTIVWVASGHHAAVNFGQYAYAGYVPNRPSIA
RTNMPTEEPISEKDMKFFLENPQVVLERSFPTQLQAIQVMAVLDVLSTHSPDEEYLGNQMEPTWGKDPTIFAAFERFSGR
MMELEGIIDERNADMKLKNRNGARVVPYELLKPFSGRGVTEKGVPYSISI
>GAY49886.1 hypothetical protein CUMW_122470 [Citrus unshiu]
MADPLKEKRDKYIYVPRDETFSDVKQEAFDNMKDYKSMCHAALPYIEKFFDGDKKFEYFTEIDELFNEDGFSLPEAEPGF
LNSLARFAKTLKEMGEEVFQFDAPEAMLRDKFFWFRDEEFARQTLAGENPCSIQLITEWPLKSSLDPKIYGPRLQESEIT
KDIIEKELGAMISVEEAIEQKKLFMLDYHDLFLPYVEKVRKLEHTTLYGSRTVFFLTPDNTLRPLAIELTRPPMDDKPLW
RKVYTPGSWNSTKTWLWRLAKAHVLAHDSGYHQLVSHWLRSHCVVEPYIIATNRQLSVMHPIYRLLHPHLRYTLELNAIG
RDILISAGGVIENTFSPGEYCMEMSSVIYDKQWRFDEQALPKDLMKRGMAVEDPNARHGLKLTIDDYPFAKDGLDLWGIL
KQWVTDYVNHYYPDQSIVESDDELQAWWTEIRTVGHADKKDEPWWPVLKTPQNLIEILTTIIWVASGHHAAVNFGQYTYA
AYFPNRPTIARVNMPDEDPTEKFWKTFIEKPEDALLYTFPNQDQAILVIATLDLLSTHSPDEEFLGKGKEPAWGEDPVIN
AAFEKFSGRLMELEGIIDERNGDSTLVNRNGAGVVPYNLLKPYWKDGDKEKGVPYSISI
PI4K ALPHA protein sequences
>Pt PI4K ALPHA_Ptrif.0008s0676.1.v1.3.1_Poncirus_trifoliata
MEALFELCDLIAQNPKQFSEKLAWICNRCPQPELLLSGSPRVSRSHLNAV
LAVARFLSKCGDSADSRPKSVILEFIRAIPSSFSRSFWPQAFSTSDSISS
CFTEFLGYVSKSCDDSPDFAAEVAGLTGEVIISAVCCYGAEDSGITRAFL
LASSKNFPPILPSDANKLVTVLLEQLALPIPASPREHIPINSGTSSSQSS
PLSANHLQPSQSNGSESSPGNEGASIVSGSSVSMNGGASIFGGFTMNDGQ
QFRQQVASFEEESVESLEKQEIAFKLITHVLDKVQIDTKPLEQIRFLAKR
QLQSMSAFLKIRKRDWTEQGPLLKARINAKLSVYQSVARLKIKSLASLDM
EGKTSKRIVLETLALLVDAAESCLLSVWRKLRVCEELFSSLLAGIAQIAV
IRGGQPLRVLLIRLKPLVLTACAQGDTWGSSKGAMFETVMKTSCEIIESG
WTKDRAPVDTFIMGLATSIRERNDYDEQVEKEKQAVPAVQLNVIRLLADL
TVAVNKSEVVDMILPLFIESLEEGDASTPSLLRERLLDAVSHMASLGFEK
SYRETVVLMTRSYLSKLSIVGSAESKTMAAEATTERVETLPAGFLLIAGG
LRNAKLRSDYRQRLLSLCSDVGLAAESKSGRSGADFLGPLLPAVAEICSD
FDPTVDVEPSLLKLERNLWFYIALFGLAPPIQKTQPPVKSVSSTINSVGS
MGTIPLQAVTGPYMWNTQWSSAVQHIAQGTPPLVVSSVKWLEDELELNAL
HNPGSRRGSGNEKAAGTQRAALSAALGGRVEVAAMSTISGVKATYLLAVA
FLEIIRFSSNGGILNGGTSLTAARSAFSCVFEYLKTPNLMPSVFQCLNAI
VLRAFETAVSWLEERTAETGKEAEIKESTLFAHACFLIKSMSQREEHLRD
TAVNLLTHLRDKFPQVLWHSSCLDSLLFSFDSDASSAVINDPAWVATVRS
LYQRLVREWVLTSLSYAPCTTQGLLQDKLCKANNWQRVQPTTDMVSLLSE
IRIGTCKNDCWPGIRTANIPAVTAAAAAASGATLKPAEALEVLSTGIVSA
TVKCNHAGEIAGMRRLYNSIGGFQSGTMPTGSFGFGGGFQRLISGAFSQQ
PQTEDDSFNEMLLSKFVHLLQQFVNVAEKGGEVDKGQFRETCSQATALLL
SNLDSNSKSNVEGFSQLLRLLCWCPAYISTPDAMETGVFIWTWLVSAAPQ
LGSLVLAELVDAWLWTIDTKRGLFATDVRYSGPAAKLRPHLAPGEPEPQP
EIDPVQQIIAHRLWLGFFIDRFEVVRHNSVEQLLLLGRMLQGTTNFPWKF
SRHPAAAGTFFTLMLLGLKFCSCQSQGYLQNFKSGLQLLEDRIYRASLGW
FAYEPEWYDINCVNFAQSEAQSLSLFLHYLLNERADAVHHDAKGRGHENG
SALVDVNDQFHPIWGQIENYDVGREKRKQLLLMLCQHEADRLDVWAHPII
SKESVSSRPRISSEKLVEYARTAFQVDPRIALSLASRFPANASLKAEVTQ
LVQLHILDIRCIPEALPYFVTPKAVDEDSALLQQLPHWAACSITQALEFL
TPAYKGHPRVMAYILRVLESYPPERVTFFMPQLVQALRYDDERLVEGYLL
RATQRSDIFAHILIWHLQGETFVPESGKEKDANSVKNSSFQTLLPMVRHR
IIDGFNPKARDLFQREFDFFDKVTNISGALYPLPKEERRAGIRRELEKIE
MEGEDLYLPTAPNKLVRGIRVDSGIPLQSAAKVPIMITFNVVDRDGDQSN
VMPQACIFKVGDDCRQDVLALQVISLLRDIFEAVGINLYLFPYGVLPTGP
EKGIIEVVPNTRSRSQMGETTDGGLYEIFQQDFGPVGSTSFEAARENFII
SSAGYAVASLLLQPKDRHNGNLLFDNMGRLVHIDFGFILETSPGRNMRFE
SAHFKLSHEMTQLLDPSGGMKSDTWNQFVSLCIKGYLAARRFMDGIINTV
LLMLDSGLPCFSRGDPIGNLRKRFHPEMSDREAAIFMRNVCTDAYNKWTT
AGYDLIQYLQQGIEK-
>XP_006423217.1 phosphatidylinositol 4-kinase alpha 1 isoform X1 [Citrus
clementina]
MEALFELCDLIAQNPKQFSEKLAWICNRCPQPELLLSGSPRVSRSHLNAVLAVARFLSKCGDSADSRPKSVILEFIRAIP
SSFNRSFWPQAFSTSDSISSFFTEFLGYVSKSCDDSPDFAAEVAGLIGEVIISAVCCYGAEDSGITRAFLLASSKNFPPI
LPSDANKLVTVLLEQLALPIPASPREHIPINSGTSSSQSSPLSANHLQPSQSNGSESSPGNEGASIVSGSSVSMNGGASI
FGGFTMNDGQQFGQQFRQQVASFEEESVESLEKQEIAFKLITHVLDKVQIDTKLLEQIRFLAKRQLQSMSAFLKIRKRDW
TEQGPLLKARINAKLSVYQSVARLKIKSLASLDMEGKTSKRLVLETLALLVDAAESCLLSVWRKLRVCEELFSSLLAGIA
QIAVIRGGQPLRVLLIRLKPLVLTACAQGDTWGSSKGAMFETVMKTSCEIIESGWTKDRAPVDTFIMGLATSIRERNDYD
EQVEKEKQAVPAVQLNVIRLLADLTVAVNKSEVVDMILPLFIESLEEGDASTPSLLRLRLLDAVSHMASLGFEKSYRETV
VLMTRSYLSKLSIVGSAESKTMAAEATTERVETLPAGFLLIAGGLRNAKLRSDYRHRLLSLCSDVGLAAESKSGRSGADF
LGPLLPAVAEICSDFDPTVDVEPSLLKLFRNLWFYIALFGLAPPIQKTQPPVKSVSSTLNSVGSMGTIPLQAVTGPYMWN
TQWSSAVQHIAQGTPPLVVSSVKWLEDELELNALHNPGSRRGSGNEKAAGTQRAALSAALGGRVEVAAMSTISGVKATYL
LAVAFLEIIRFSSNGGILNGGTSLTAARSAFSCVFEYLKTPNLMPSVFQCLNAIVLRAFETAVSWLEERTAETGKEAEIK
ESTLEAHACFLIKSMSQREEHLRDTAVNLLTQLRDKFPQVLWHSSCLDSLLFSFDSDASSAVINDPAWVATVRSLYQRLV
REWVLTSLSYAPCTTQGLLQDKLCKANNWQRAQPTTDMVSLLSEIRIGTCKNDCWPGIRTANIPAVTAAAAAASGATLKP
AEALEVLSTGIVSATVKCNHAGEIAGMRRLYNSIGGFQSGTMPTGSFGFGGGFQRLISGAFSQQPQTEDDSFNEMLLSKF
VHLLQQFVNVAEKGGEVDKGQFRETCSQATALLLSNLDSNSKSNVEGFSQLLRLLCWCPAYISTPDAMETGVFIWTWLVS
AAPQLGSLVLAELVDAWLWTIDTKRGLFATDVRYSGPAAKLRPHLAPGEPEPQPEIDPVQQIIAHRLWLGFFIDRFEVVR
HNSVEQLLLLGRMLQGTTNFPWKFSRHPAAAGTFFTLMLLGLKFCSCQSQGYLQNFKSGLQLLEDRIYRASLGWFAYEPE
WYDINCVNFAQSEAQSLSLFLHYLLNERADAFQHDAKGRGHENGSALVDVNDQFHPIWGQIENYDVGREKRKQLLLMLCQ
HEADRLDVWAHPIISKESVSSRPRISSEKLVEYARTAFQVDPRIALSLASRFPANASLKAEVTQLVQLHILDIRCIPEAL
PYFVTPKAVDEDSALLQQLPHWAACSITQALEFLTPAYKGHPRVMAYILRVLESYPPERVTFFMPQLVQALRYDDERLVE
GYLLRATQRSDIFAHILIWHLQGETFVPESGKEKDANSVKNGSFQTLLPMVRQRIIDGFNPKALDLFQREFDFFDKVTNI
SGALYPLPKEERRAGIRRELEKIEMAGEDLYLPTAPNKLVRGIRVDSGIPLQSAAKVPIMITFNVVDRDGDQSNVMPQAC
IFKVGDDCRQDVLALQVISLLRDIFEAVGLNLYLFPYGVLPTGPERGIIEVVPNTRSRSQMGEITDGGLYEIFQQDFGPV
GSTSFEAARENFIISSAGYAVASLLLQPKDRANGNLLFDNIGRLVHIDFGFILETSPGRNMRFESAHFKLSHEMTQLLDP
SGVMKSDTWNQFVSLCIKGYLAARRYMDGIINTVLLMLDSGLPCFSRGDPIGNLRKRFHPEMSDREAAIFMRNVCTDAYN
KWTTAGYDLIQYLQQGIEK
>KDO46183.1 hypothetical protein CISIN_19000157mg [Citrussinensis]
MEALFELCDLIAQNPKQFSEKLAWICNRCPQPELLLSGSPRVSRSHLNAVLAVARFLSKCGDSADSRPKSVILEFIRAIP
SSFNRSFWPQAFSTSDSISSFFTEFLGYVSKSCDDSPDFAAEVAGLTGEVIISAVCCYGAEDSGITRAFLLASSKNFPPI
LPSDANKLVTVLLEQLALPIPASPREHIPINSGTSSSQSSPLSANHLQPSQSNGSESSPGNEGASIVSGSSVSMNGGASI
FGGFTMNDGQQFRQQVASFEEESVESLEKQEIAFKLITHVLDKVQIDTKLLEQIRFLAKRQLQSMSAFLKIRKRDWTEQG
PLLKARINAKLSVYQSVARLKIKSLSSLDMEGKTSKRLVLETLALLVDAAESCLLSVWRKLRVCEELFSSLLAGIAQIAV
IRGGQPLRVLLIRLKPLVLTACAQGDTWGSSKGAMFETVMKTSCEIIESGWTKDRAPVDTFIMGLATSIRERNDYDEQVE
KEKQAVPAVQLNVIRLLADETVAVNKSEVVDMILPLFIESLEEGDASTPSLERLRLLDAVSHMASLGFEKSYRETVVLMT
RSYLSKLSIVGSAESKTMAAEATTERVETLPAGFLLIAGGLRNAKLRSDYRHRLLSLCSDVGLAAESKSGRSGADFLGPL
LPAVAAICSDFDPTVDVEPSLLKLFRNLWFYIALFGLAPPIQKTQPPVKSVSSTLNSVGSMGTIPLQAVTGPYMWNTQWS
SAVQHIAQGTPPLVVSSVKWLEDELELNALHNPGSRRGSGNEKAAGTQRAALSAALGGRVEVAAMSTISGVKATYLLAVA
FLEIIRFSSNGGILNGGTSLTAARSAFSCVFEYLKTPNLMPSVFQCLNAIVLRAFETAVSWLEERTAETGKEAEIKESTL
FAHACFLIKSMSQREEHLRDTAVNLLTQLRDKFPQVLWHSSCLDSLLFSFDSDASSAVINDPAWVATVRSLYQRLVREWV
LTSLSYAPCTTQGLLQDKLCKANNWQRAQPTTDMVSLLSEIRIGTCKNDCWPGIRTANIPAVTAAAAAASGATLKPAEAL
EVLSTGIVSATVKCNHAGEIAGMRRLYNSIGGFQSGTMPTGSFGFGGGFQRLISGAFSQQPQTEDDSFNEMLLSKFVHLL
QQFVNVAEKGGEVDKGQFRETCSQATALLLSNLDSNSKSNVEGFSQLLRLLCWCPAYISTPDAMETGVFIWTWLVSAAPQ
LGSLVLAELVDAWLWTIDTKRGLFATDVRYSGPAAKLRPHLAPGEPEPQPEIDPVQQIIAHRLWLGFFIDRFEVVRHNSV
EQLLLLGRMLQGTTNFPWKFSRHPAAAGTFFTLMLLGLKFCSCQSQGYLQNFKSGLQLLEDRIYRASLGWFAYEPEWYDI
NCVNFAQSEAQSLSLFLHYLLNERADAFQHDAKGRGHENGSALVDVNDQFHPIWGQIENYDVGREKRKQLLLMLCQHEAD
RLDVWAHPIISKESVSSRPRISSEKLVEYARTAFQVDPRIALSLASRFPANASLKAEVTQLVQLHILDIRCIPEALPYFV
TPKAVDEDSALLQQLPHWAACSITQALEFLTPAYKGHPRVMAYILRVLESYPPERVTFFMPQLVQALRYDDERLVEGYLL
RATQRSDIFAHILIWHLQGETFVPESGKEKDANSVKNGSFQTLLPMVRQRIIDGFNPKALDLFQREFDFFDKVTNISGAL
YPLPKEERRAGIRRELEKIEMAGEDLYLPTAPNKLVRGIRVDSGIPLQSAAKVPIMITFNVVDRDGDQSNVMPQACIFKV
GDDCRQDVLALQVISLLRDIFEAVGINLYLFPYGVLPTGPEKGIIEVVPNTRSRSQMGETTDGGLYEIFQQDFGPVGSTS
FEAARENFIISSAGYAVASLLLQPKDRHNGNLLFDNIGRLVHIDFGFILETSPGRNMRFESAHFKLSHEMTQLLDPSGVM
KSDTWNQFVSLCIKGYLAARRYMDGIINTVLLMLDSGLPCFSRGDPIGNLRKRFHPEMSDREAAIFMRNVCTDAYNKWTT
AGYDLIQYLQQGIEK
>GAY65440.1 hypothetical protein CUMW_241100 [Citrus unshiu]
MEALFELCDLIAQNPKQFSEKLAWICNRCPQPELLLSGSPRVSRSHLNAVLAVARFLSKCGDSADSRPKSVILEFIRAIP
SSFNRSFWPQAFSTSDSISSFFTEFLGYVSKSCDESPDFAAEVAGLTGEVIISAVCCYGAEDSGITRAFLLASSKNFPPI
LPSDANKLVTVLLEQLALPIPASPREHIPINSGTSSSQSSPLSANHLQPSQSNGSESSPGNEGASIVSGSSVSMNGGASI
FGGFTMNDGQQFRQQVASFEEESVESLEKQEIAFKLITHVLDKVQIDTKLLEQIRFLAKRQLQSMSAFLKIRKRDWTEQG
PLLKARINAKLSVYQSVARLKIKSLASLDMEGKTSKRLVLETLALLVDAAESCLLSVWRKLRVCEELFSSLLAGIAQIAV
IRGGQPLRVLLIRLKPLVLTACAQGDTWGSSKGAMFETVMKTSCEIIESGWTKDRAPVDTFIMGLATSIRERNDYDEQVE
KEKQAVPAVQLNVIRLLADLTVAVNKSEVVDMILPLFIESLEEGDASTPSLLRLRLLDAVSHMASLGFEKSYRETVVLMT
RSYLSKLSIVGSAESKTMAAEATTERVETLPAGFLLIAGGLRNAKLRSDYRHRLLSLCSDVGLAAESKSGRSGADFLGPL
LPAVAAICSDFDPTVDVEPSLLKLFRNLWFYIALFGLAPPIQKTQPPVKSVSSTLNSVGSMGTIPLQAVTGPYMWNTQWS
SAVQHIAQGTPPLVVSSVKWLEDELELNALHNPGSRRGSGNEKAAGTQRAALSAALGGRVEVAAMSTISGVKATYLLAVA
FLEIIRFSSNGGILNGGTSLTAARSAFSCVFEYLKTPNLMPSVFQCLNAIVLRAFETAVSWLLKCKYCAFYLEACTSGGA
LLVLFLHLSLPDRAIDFCGNIALLEERTAETGKEAEIKESTLFAHACFLIKSMSQREEHLRDTAVNLLTQLRDKFPQVLW
HSSCLDSLLFSFDSDASSAVINDPAWVATVRSLYQREVREWVLTSLSYAPCTTQGLLQDKLCKANNWQRAQPTTDMVSLL
SEIRIGTCKNDCWPGIRTANIPAVTAAAAAASGATLKPAEALEVLSTGIVSATVKCNHAGEIAGMRRLYNSIGGFQSGTM
PTGSFGFGGGFQRLISGAFSQQPQTEDDSFNEMLLSKFVHLLQQFVNVAEKGGEVDKGQFRETCSQATALLLSNLVTIYF
SSSFLHILGIENYSLRKCFILIYVRVHVFFFSFLCQDSNSKSNVEGFSQLLRLLCWCPAYISTPDAMETGVFIWTWLVSA
APQLGSLVLAELVDAWLWTIDTKRGLFATDVRYSGPAAKLRPHLAPGEPEPQPEIDPVQQIIAHRLWLGFFIDRFEVVRH
NSVEQLLLLGRMLQGTTNFPWKFSRHPAAAGTFFTLMLLGLKFCSCQSQGYLQNFKSGLQLLEDRIYRASLGWFAYEPEW
YDINCVNFAQSEAQSLSLFLHYLLNERADAFQHDAKGRGHENGSALVDVNDQFHPIWGQIENYDVGREKRKQLLLMLCQH
EADRLDVWAHPIISKESVSSRPRISSEKLVEYARTAFQVDPRIALSLASRFPANASLKAEVTQLVQASWSGEMAVALHIL
DIRCIPEALPYFVTPKAVDEDSALLQQLPHWAACSITQALEFLTPAYKGHPRVMAYILRVLESYPPERVTFFMPQLVQAL
RYDDEVSNRLVEGYLLRATQRSDIFAHILIWHLQGETFVPESGKEKDANSVKNGSFQTLLPMVRQRIIDGFNPKALDLFQ
REFDFFDKVTNISGALYPLPKEERRAGIRRELEKIEMVGEDLYLPTAPNKLVRGIRVDSGIPLQSAAKVPIMITFNVVDR
DGDQSNVMPQACIFKVGDDCRQDVLALQVISLLRDIFEAVGLNLYLFPYGVLPTGPERGIIEVVPNTRSRSQMGETTDGG
LYEIFQQDFGPVGSTSFEAARENFIISSAGYAVASLLLQPKDRHNGNLLFDNIGRLVHIDFGFILETSPGRNMRFESAHF
KLSHEMTQLLDPSGVMKSDTWNQFVSLCIKGYLAARRYMDGIINTVLLMLDSGLPCFSRGDPIGNLRKRFHPEMSDREAA
IFMRNVCTDAYNKWTTAGYDLIQYLQQGIEK
>XP_024035329.1 phosphatidylinositol 4-kinase alpha 1 isoform X2 [Citrus
clementina]
MEALFELCDLIAQNPKQFSEKLAWICNRCPQPELLLSGSPRVSRSHLNAVLAVARFLSKCGDSADSRPKSVILEFIRAIP
SSFNRSFWPQAFSTSDSISSFFTEFLGYVSKSCDDSPDFAAEVAGLTGEVIISAVCCYGAEDSGITRAFLLASSKNFPPI
LPSDANKLVTVLLEQLALPIPASPREHIPINSGTSSSQSSPLSANHLQPSQSNGSESSPGNEGASIVSGSSVSMNGGASI
FGGFTMNDGQQFGQQFRQQVASFEEESVESLEKQEIAFKLITHVLDKVQIDTKLLEQIRFLAKRQLQSMSAFLKIRKRDW
TEQGPLLKARINAKLSVYQSVARLKIKSLASLDMEGKTSKRLVLETLALLVDAAESCLLSVWRKLRVCEELFSSLLAGIA
QIAVIRGGQPLRVLLIRLKPLVLTACAQGDTWGSSKGAMFETVMKTSCEIIESGWTKDRAPVDTFIMGLATSIRERNDYD
EQVEKEKQAVPAVQLNVIRLLADLTVAVNKSEVVDMILPLFIESLEEGDASTPSLLRLRLLDAVSHMASLGFEKSYRETV
VLMTRSYLSKLSIVGSAESKTMAAEATTERVETLPAGFLLIAGGLRNAKLRSDYRHRLLSLCSDVGLAAESKSGRSGADF
LGPLLPAVAEICSDFDPTVDVEPSLLKLFRNLWFYIALFGLAPPIQKTQPPVKSVSSTINSVGSMGTIPLQAVTGPYMWN
TQWSSAVQHIAQGTPPLVVSSVKWLEDELELNALANPGSRRGSGNEKAAGTQRAALSAALGGRVEVAAMSTISGVKATYL
LAVAFLEIIRFSSNGGILNGGTSLTAARSAFSCVFEYLKTPNLMPSVFQCLNAIVLRAFETAVSWLEERTAETGKEAEIK
ESTLFAHACFLIKSMSQREEHLRDTAVNLLTQLRDKFPQVLWHSSCLDSLLFSFDSDASSAVINDPAWVATVRSLYQRLV
REWVLTSLSYAPCTTQGLLQDKLCKANNWQRAQPTTDMVSLLSEIRIGTCKNDCWPGIRTANIPAVTAAAAAASGATLKP
AEALEVLSTGIVSATVKCNHAGEIAGMRRLYNSIGGFQSGTMPTGSFGFGGGFQRLISGAFSQQPQTEDDSFNEMLLSKF
VHLLQQFVNVAEKGGEVDKGQFRETCSQATALLLSNLDSNSKSNVEGFSQLLRLLCWCPAYISTPDAMETGVFIWTWLVS
AAPQLGSLVLAELVDAWLWTIDTKRGLFATDVRYSGPAAKLRPHLAPGEPEPQPEIDPVQQIIAHRLWLGFFIDRFEVVR
HNSVEQLLLLGRMLQGTTNFPWKFSRHPAAAGTFFTLMLLGLKFCSCQSQGYLQNFKSGLQLLEDRIYRASLGWFAYEPE
WYDINCVNFAQSEAQSLSLFLHYLLNERADAFQHDAKGRGHENGSALVDVNDQFHPIWGQIENYDVGREKRKQLLLMLCQ
HEADRLDVWAHPIISKESVSSRPRISSEKLVEYARTAFQVDPRIALSLASRFPANASLKAEVTQLVQLHILDIRCIPEAL
PYFVTPKAVDEDSALLQQLPHWAACSITQALEFLTPAYKGHPRVMAYILRVLESYPPERVTFFMPQLVQALRYDDELLCR
VWTRWPSNDKKYPILGKLKLYKESQKKCWDGQQKYPSTTVKAEQPK

TABLE 4
Polypeptide sequence of citrus plant positive defense
regulators
BRAP2 protein sequences
>PtBRAP2 Ptrif. 0001s0545.2. v1. 3.1_Poncirus_trifoliata
MFVLRLHSVDDNHPITIEEAGESTVSSTATRSSANPNPKESERRGLVHLF
RGTSQSYQQNPNSRSTCIFVVAVPNYLSSDEFVRFCGEHIDHVEELIFIR
NDAMEDRYSVLIKLVDQLTADEFYSNINGKRFSPAEAEVCHMLFMLSVEY
TELAEIASTPPAGFTELPTCPICLERLDPDTSGILSTICDHSFQCSCTAK
WTVLSCQVCRECHQQDERPTCSVCGTVENLWVCLICGFVGCGRYKEGHAV
RHWKDTQHWYSLDLRTQQIWDYVGDNYVHRLNQSKADGKLVEMNSPCMSH
EAHCGTCECSEDSGISGALENSKVEAIVDEYNRLLATQLETQRQYYESLL
AEAKSKRESLIPETVEKAVASKMQDIQNELEICEEAKKAVADVNSKLIKN
QEIMRKKEKEIEEREKTSLRLRDATILDLEEQIRDLTVYIEAQKTLENMT
DSDGIKGGTVLPVSYQQSSPTNTRRHKKSSRRKN-
>ESR64115.1 hypothetical protein CICLE_v10008137mg [Citrus clementina]
MFVLRVHSVDDNHPITIEEAGFCTVSSTATRSSANPNPKFSERRGLVHLERGTSQSYQQNPNSRSTCIFVVAVPNYLSSD
EFVRFCGSHIDHVEELIFIRNDAMEDRYSVLIKLVDqLTADEFYSNINGKRFSPAEAEVCHMLFMLSVEYTELAEIASTP
PAGFTELPTCPICLERLDPDTSGILSTICDHSFQCSCTAKWTVLSCqVCRFCHQQDERPTCSVCGTVENLWVCLICGEVG
CGRYKEGHAVRHWKDTQHWYSLDLRTQQIWDYVGDNYVHRLNQSKADGKLVEMNSPCMSHEAHCGTCECSEDSGISGALE
NSKVEAIVDEYNRLLATQLETQRQYYESLLAEAKSKRESLIPETVEKAVASKMQDIQNELDICEEAKKAVADVNPLTTHE
RSVILFFFWGVGGCYLMLLIETE
>KDO80178.1 hypothetical protein CISIN_1g011525mg [Citrus sinensis]
MFVLRVHSVDDNHPITIEEAGFCTVSSTATRSRANPNPKESERRGLVHLERGTSQSYQQNPNSRSTCIFVVAVPNYLSSD
EFVRECGSHIDHVEELIFIRNDAMEDRYSVLIKLVDQLTADEFYSNINGKRESPAEAEVCHMLEMLSVEYTELAEIASTP
PAGFTELPTCPICLERLDPDTSGILSTICDHSFQCSCTAKWTVLSCQVCRFCHQQDERPTCSVCGTVENLWVCLICGFVG
CGRYKEGHAVRHWKDTQHWYSLDLRTQQIWDYVGDNYVHRLNQSKADGKLVEMNSPCMSHEAHCGTCECSEDSGISGALE
NSKVEAIVDEYNRLLATQLETQRQYYESLLAEAKSKRESLIPETVEKAVASKMQDIQNELDICEEAKKAVADVNPLTTHE
RSVILFFFGGVGGCYLMLLIETE
>XP_006450876.1 BRCA1-associated protein [Citrus clementina]
MFVLRVHSVDDNHPITIEEAGFCTVSSTATRSSANPNPKESERRGLVHLERGTSQSYQQNPNSRSTCIFVVAVPNYLSSD
EFVRECGSHIDHVEELIFIRNDAMEDRYSVLIKLVDQLTADEFYSNLNGKRFSPAEAEVCHMLEMLSVEYTELAEIASTP
PAGFTELPTCPICLERLDPDTSGILSTICDHSFQCSCTAKWTVLSCQVCRECHQQDERPTCSVCGIVENLWVCLICGFVG
CGRYKEGHAVRHWKDTQHWYSLDLRTQQIWDYVGDNYVHRLNQSKADGKLVEMNSPCMSHEAHCGTCECSEDSGISGALE
NSKVEAIVDEYNRLLATQLETQRQYYESLLAEAKSKRESLIPETVEKAVASKMQDIQNELDICEEAKKAVADVNSKLIKN
QEIMRKKEKEIEEREITSLRLRDATILDLEEQIRDLTVYIEAQKTLINMTDSDGIKGGTVLPVSYQQSSPTNTRRHKKSS
RRKN
>GAY45486.1 hypothetical protein CUMW_089840 [Citrus unshiu]
MFVLRVHSVDDNHPITIEEAGFCTVSSTATRSRANPNPKFSERRGLVHLERGTSQSYQQNPNSRSTCIFVVAVPNYLSSD
EFVRFCGSHIDHVEELIFIRNDAMEDRYSVLIKLVDQLTADEFYSNLNGKRFSPAEAEVCHMLEMLSVEYTELAEIASTP
PAGFTELPTCPICLERLDPDTSGILSTICDHSFQCSCTAKWTVLSCQVCRECHQQDERPTCSVCGTVENLWVCLICGFVG
CGRYKEGHAVRHWKDTQHWYSLDLRTQQIWDYVGDNYVHRLNQSKADGKLVEMNSPCMSHEAHCGTCECSEDSGISGALF
NSKVEAIVDEYNRLLATQLETQRQYYESLLAEAKSKRESLIPETVEKAVASKMQDIQNELDICEEAKKAVADVNSKLIKN
QEIMRKKFKEIEEREITSLRLRDATILDLEEQIRDLTVYIEAQKTLTNMTDSDGIKGGTVLPVSYQQSSPENTRRHKKSN
S
>XP_006475890.1 BRCA1-associated protein [Citrus sinensis]
MFVLRVHSVDDNHPITIEEAGFCTVSSTATRSRANPNPKFSERRGLVHLFRGTSQSYQQNPNSRSTCIFVVAVPNYLSSD
EFVRECGSHIDHVEELIFIRNDAMEDRYSVLIKLVDQLTADEFYSNLNGKRFSPAEAEVCHMLFMLSVEYTELAEIASTP
PAGFTELPTCPICLERLDPDTSGILSTICDHSFQCSCTAKWTVLSCQVCRFCHQQDERPTCSVCGTVENLWVCLICGFVG
CGRYKEGHAVRHWKDTQHWYSLDLRTQQIWDYVGDNYVHRLNQSKADGKLVEMNSPCMSHEARCGTCECSEDSGISGALE
NSKVEAIVDEYNRLLATQLETQRQYYESLLAEAKSKRESLIPETVEKAVASKMQDIQNELDICEEAKKAVADVNSKLIKN
QEIMRKKFKEIEEREITSLRLRDATILDLEEQIRDLTVYIEAQKTLTNMTDSDGIKGGTVLPVSYQQSSPTNTRRHKKSS
RRKN
>GAY45487.1 hypothetical protein CUMW_089840 [Citrus unshiu]
MFVLRVHSVDDNHPITIEEAGFCTVSSTATRSRANPNPKESERRGLVHLERGTSQSYQQNPNSRSTCIFVVAVPNYLSSD
EFVRECGSHIDHVEELIFISVLIKLVDQLTADEFYSNINGKRFSPARAEVCHMLEMLSVEYTELAEIASTPPAGFTELPT
CPICLERLDPDTSGILSTICDHSFQCSCTAKWTVLSCQVCRFCHQQDERPTCSVCGTVENLWVCLICGFVGCGRYKEGHA
VRHWKDTQHWYSLDLRTQQIWDYVGDNYVHRLNQSKADGKLVEMNSPCMSHEAHCGTCECSEDSGISGALENSKVEAIVD
EYNRLLATQLETQRQYYESLLAEAKSKRESLIPETVEKAVASKMQDIQNELDICEEAKKAVADVNSKLIKNQEIMRKKEK
EIEEREITSERLRDATILDLEEQIRDETVYIEAQKTLINMTDSDGIKGGTVLPVSYQQSSPTNTRRAKKSNS
>KDO80180.1 hypothetical protein CISIN_1g011525mg [Citrus sinensis]
MFVLRVHSVDDNHPITIEEAGFCTVSSTATRSRANPNPKFSERRGLVHLERGTSQSYQQNPNSRSTCIFVVAVPNYLSSD
EFVRFCGSHIDHVEELIFIRNDAMEDRYSVLIKLVDQLTADEFYSNLNGKRFSPAEAEVCHMLFMLSVEYTELAEIASTP
PAGFTELPTCPICLERLDPDTSGILSTICDHSFQCSCTAKWTVLSCQVCRECHQQDERPTCSVCGTVENIWVCLICGFVG
CGRYKEGHAVRHWKDTQHWYSLDLRTQQIWDYVGDNYVHRLNQSKADGKLVEMNSPCMSHEAHCGTCECSEDSGISGALE
NSKVEAIVDEYNRLLATQLETQRQVSTSFPDVKTPT
>KDO80177.1 hypothetical protein CISIN 1g011525mg [Citrus sinensis]
MSSNTVRNDAMEDRYSVLIKLVDQLTADEFYSNINGKRFSPARAEVCHMLEMLSVEYTELAEIASTPPAGFTELPTCPIC
LERLDPDTSGILSTICDHSFQCSCTAKWTVLSCQVCRFCHQQDERPTCSVCGTVENLWVCLICGFVGCGRYKEGHAVRHW
KDTQHWYSLDLRTQQIWDYVGDNYVHRLNQSKADGKLVEMNSPCMSHEAHCGTCECSEDSGISGALENSKVEAIVDEYNR
LLATQLETQRQYYESLLAEAKSKRESLIPETVEKAVASKMQDIQNELDICEEAKKAVADVNSKLIKNQEIMRKKEKEIEE
REITSLRLRDATILDLEEQIRDETVYIEAQKTLTNMTDSDGIKGGTVLPVSYQQSSPTNTRRHKKSSRRKN
>StBRAP2_PGSC0003DMP400053855 sequence match in blast db Potato PGSC DM v3.4
protein sequences
MFTLQIHTVDSPQPIPTTIATTSSAAHGPKPNSDLTSSSGSLHLSELRGIAHLFRHLPSSTSTTISNPISRTTTVFIVAA
PNYLSPDDELLFCGTHLADETHVMFLKNDGIEHSYSVLINIVNQLAADGFYCSENGKRFKPTEVEVCHIYFIQSVVYEES
AYITSTPPVGYTELPTCPVCLERLDQDTSGIQSTLCDHSFQCSCVSKWTYLACQVCRLCQQQDEKPACSECGTMKNLCVC
LICGFVGCGRYEKKHAIKHWTDAAHHYSLELETQQIWDYVGDKYVHRLNQSKGDSKLVTVNSRCTATEGECTTCGDDEDS
SFSGALFSSKVDSIVDEYNNLLASQLETQRQHYESLLAEAKSGKESSISRAVEKAVESKINDLQAKIEMYTEETKSIVER
NQVLLKNQELLQTKYRETAERERLLLKSKDENKLDLKEQIRDLKVYVEAQRKLSNMGISDGKGGTVLSVEPNKQSSSNSR
RRGKLGRRRN
CYP93 protein sequences
>PtCYP93_Ptrif.0003s2312.1.v1.3.1_Poncirus_trifoliata
MSIRIGSVLGVVTSSPDVTKELLKTNDVTFAARKSSAAIECLTYNSSFAF
APNGPYWQFMKKLTAVELLGSRTLCQFLPIRTNELRELIRFLFEKSKSGQ
SVNITDELLKFANNIISQMMLSIRCSSKGGQAEECRTLAREVTEIFGEVN
ISDIIWIEKSFDIQGFHRREKDIHRRYDSLLENIITNREKLRKEKKESEE
KVKDLLDILLDVLENPNSEIKLTRDHIKALCLDELTAGTDTSSTTLEWSL
AELINHPMVLQKAQQEIDQVVGRNRLVQESDVPHLPYIQAIIKESFRIHP
PIPLISRKAVETVKLATT-
>XP_006441830.1 licodione synthase [Citrus clementina]
MTLQPLIFYASLFILSALVLKAIKHSRRLPPSPWALPIVGHLHLLGPSLHHSFHKLSTRYGPLMSIRIGSVLGVVTSSPD
VTKELLKINDVTFAARNSSAAIECLTYNSSFAFAPNGPYWQFMKKLTTVELLGSRTLLQFLPIRTNELHELIRFLFEKSK
SGGSVNITDELLKFTNNIISQMMLSIRCSGKGGQAEECRTLAREVTEIFGEFNISDVIWVEKSEDIQGFRRRFKDIHRRF
DSLLENIITNREKLRKEKKESEEKVKDLLDILLDVLENQNSEIKLTRDHIKALFLDELTAGTDTSSMTVEWALAELINHP
MVLQKAQQEIDQVVGRNRLVQESDVPHLPYIQVIIKESFRIHPPIPLLNRRALEDCKIGNYIIPKGTLLFVNLWSMGRDP
ETWKNPLEFQPERFLSESNSEIDVRGLHYRLLPFGTGRRGCPGLSLAMQELPTTLAAMIQCENFKVTSPDGVVDMSERPG
LSSPRAQDLVCVPVARCAPSIVN
>KDO41683.1 hypothetical protein CISIN_1g010779mg [Citrus sinensis]
MTLQPLIFYASLFILSALVLKAIKHSRRLPPSPWALPIVGHLHLLGPSLHHSFHKLSTRYGPLMSIRIGSVLGVVTSSPD
VTKELLKINDVTFAARNSSAAIECLTYNSSFAFAPNGPYWQFMKKLTTVELLGSRTLLQFLPIRTNELHELIRFLFEKSK
SGGSVNITDELLKFTNNIISQMMLSIRCSGKGGQAEECRTLAREVTEIFGEFNISDIIWIFKSFDIQGFRRRFKDIHRRF
DSLLENIITNREKLRKEKKESEEKVKDLLDILLDVLENQNSEIKLTRDHIKALFLDFLTAGTDTSSMTVEWALAELINHP
MVLQKAQQEIDQVEGRNRLVQLKNHLPYIQAIIKESFRIHPPIPLISRKAVEDCKIGNYVIPKDTVLFVNLWSMGRDPKI
WKNPLEFQPERFLSQSNSEIDVKGLHYQFLPFGTGRRGCPGLSLAMQELPTTLAAMIQCFNFKVTSPDGVVDMSERPGLS
SPRAQDLVCVPVARCAPSILN
>XP_024953859.1 licodione synthase-like [Citrus sinensis]
MLSAHLNGSAQEPYRSFLAMTLQPLIFYASLFILSALVLKAIKHSRRLPPSPWALPIVGHLALLGPSLHASFHKLSTRYG
PIMSIRIGSVLGVVTSSPDVTKELLKINDVTFAARNSSAAIECLTYNSSFAFAPNGPYWQFMKKLTTVELLGSRTLLQFL
PIRINELHELIRFLFEKSKSGGSVSITDELLKFTNNIISQMMLSIRCSGKGGQAEECRTLAREVTEIFGEFNISDIIWIE
KSFDIQGFRRRFKDIHRREDSLLENIITNREKLRKEKKESEEKVKDLLDILLDVLENQNSEIKLTRDHIKALFLDELTAG
TDTSSMTVEWALAELINHPMVLQKAQQEIDQVVGRNRLVQESDFPRLPYIQAIIKESFRIHPPIPLISRKAVEDCKIGNY
VIPKDTVLFVNLWSMGRDPKIWKNPLEFQPERFLSQSNSEIDVRGLHYQLLPFGTGRRGCPGLSLAMQELPATLAAMIQC
FNFKVTSPDGVVDMSERPQLSSPRARDLMCVPVARCPLTSLLLSVQDELTAGTDTSSMTVEWALAELINHPMVLQKAQQE
IDQVVGRNRLVQESDFPRLPYIQAIIKESFRIHPPIPLLNRRALEDCKIGNYIIPKGTLLFVNLWSMGRDPKIWKNPLEF
QPERFFSQSNSEIDVRGLHYQLLPFGTGRRGCPGLSLAMQELPTALASMIQCFDFKVTSPDGVVDMSERPGLSSPRAQDL
VCVPVARCAPSIVNSDVR
>XP_006478300.1 licodione synthase-like [Citrus sinensis]
MTLQPLIFYASLFVLSALVLKAIKHSRRLPPSPWALPIVGHLHELGPSLHHSFHKLSTRYGPLMSVRIGSVLGVVTSCPD
VTKELLKINDVTFTGRKSSAAIECLTYNSSFAFAPYGPYWQFMKKLSAVELLGSRTLHQFLPVRTNELRELIRFLFEKSK
SGQSVNITDELLRFANNIISQMMLSIRCSGKGGQAEECRTLAREVTEIEGEFNISDIIWIFKSEDIQGENRRFKDIHRRY
DSQLENIITNREKLRKEKKESEEKVKDLLDILLDVLENQNSEIKLTRDHVKALCVDFLTAGTDTSSTSLEWSLAELINHP
MVLQEAQQELDQVVGRNRLVQESDVPHLPYIQAIIKESFRIHPPIPLISRKAVEDCKIGNYVIPKDTVLEVNLWSMGRDP
KIWKNPLEFQPERFLSQSNSEIDVKGLHYQFLPFGTGRRGCPGLSLAMQELPATLAAMIQCENFKVTSPDGVVDMSERPG
LSSPRAQDLVCVPVARCAPSILN
>GAY62273.1 hypothetical protein CUMW_216480 [Citrus unshiu]
MTLQPLIFYASLFILSALVEKAIKHSRRLPPSPWALPIVGHLHLLGPSLHHSFHKLSTRYGPLMSVRIGSVLGVVTSCPD
VTKELLKINDVTFTGRKSSAAIECLTYNSSFAFAPYGPYWREMKKLSAVELLGSRTLHQFLPVRTNELRELIRFLFEKSK
SGQSVNITDELLRFANNIISQMMLSIRCSGKGGQAEECRTLAREVTEIFGEFNISDIIWIEKSFDIQGFNRRFKDIHRRY
DSQLENIITNREKLRKEKKESEEKVKDLLDILLDVLENQNSEIKLTRDHVKALCVDELTAGTDTSSTSLEWSLAELINHP
MVLQEAQQELDQVVGRNRLVQESDVPHLPYIQAIIKESFRIHPPIPLISRKAVEDCKIGNYVIPKDTVLFVNLWSMGRDP
KIWKNPLEFQPERFLSQSNSEIDVKGLHYQFLPFGTGRRGCPGLSLAMQELPATLAAMIQCENFKVTSPDGVVDMSERPG
LASPRAQDEVCVPVARCAPSILN
>XP_006441833.1 licodione synthase [Citrus clementina]
MTLQPLIFYASLFILSALVLKAIKHSRRLPPSPWALPIVGHLHLLGPSLHHSFHKLSTRYGPLMSVRIGSVLGVVTSCPD
VTKELLKINDVTFTGRKSSAAIECLTYNSSFAFAPYGPYWRFMKKLSAVELLGSRTLHQFLPVRTNELRELIRFLFEKSK
SGQSVNITDELLRFANNIISQMMLSIRCSGKGGQAEECRTLAREVTEIFGEFNISDIIWIFKSEDIQGENRRFKDIHRRY
DSQLENIITNREKLRKEKKESEEKVKDLLDILLDVLENQNSEIKLTRDHVKALCVDELTAGTDTSSTSLEWSLAELINHP
MVLQEAQQELDQVVGRNRLVQESDVPHLPYIQAIIKESFRIHPPIPLISRKAVEDCKIGNYVIPKDTVLFVNLWSMGRDP
KIWKNPLEFQPERFLSQSNSEIDVKGLHYQFLPFGTGRRGCPGLSLAMQELPATLAAMIQCENFKVTSPDGVVDMTERPG
LASPRAQDLVCVPVARCAPSILN
>ESR55072.1 hypothetical protein CICLE_v10023526mg, partial [Citrus clementina]
SLFLLSALVLKAIKNSGRLPPSPWALLIVGHLHLLGPSLHHSFHKLSTCYGPLMSICIGSVLGVVTSSPDVTKERLKIND
VTFAARNSSAAIECLTYNSSFAFAPNGPYWLFMKKLTTVELLGSRTLRQFLPIRINKLHELIRFLFEKSKSGESVNIRDE
LLKFTNNIISPMMLSIRCSGKGGQAEECRTLAREVAEIFGEFKSEDIQGFHRRFKDIHRREDSLLENVITNREKLRKEKK
ESEEKVKGLLDILLDCSGESEFGDQVSVHLFRNIPFLYFGFPHSWHRYFIHDVQWALAELINHPMVLQKAQQEIDQVVGR
NGPVQESYVPHLPYIQAIIKESFRIHPPIPLLNRRALEDCKIGNYISPKGTLLFVNLWSMGRVLDHKLDPR
NDR1-like protein sequences
>PtNDR1-like Ptrif.0006s1395.1.v1.3.1_Poncirus_trifoliata
MSEKICDKHGCRRRKIFRTIIAGILIFVVIVLITILIVWAILRPTKPRFI
LQDATVYVFNVSNPNVLTSSFQVTISSRNPNDRIGIYYDRLDLYATYHSQ
QITYKTSLPTTYQGHKEINVWSPYVYGNAVPVAPYNAVSLTQDQSGGIIP
LMFKIDGRVRWKVGTFITGKYHLYVRCSAYINFGNKQAGNAVQNNAVCNN
AVKYQLVQSCSVSV-
>XP_006430080.1 NDR1/HIN1-like protein 1 [Citrus clementina]
MSEKVCDKHGCKRRKIERRIIAGILIFILIVLITILIVWAILRPTKPRFILQDATVYVENVSNPNVLTSSFQVTISSRNP
NDRIGIYYDKLDLYATYHNQQITYKTSLPTTYQGHKEINVWSPYVYGNAVPVAPYNAVSLTQDQSSGIIPLMFKIDGRVR
WKVGTFITGKYHLYVRCSAYINFGDRQAGTAVGNNAVKYQLVQSCSVSV
>KDO70596.1 hypothetical protein CISIN_1g028399mg [Citrus sinensis]
MSEKVCDKHGCKRRKIERRIIAGILIFILIVLITILIVWAILRPTKPRFILQDATVYVENVSNPNVLTSSFQVTISSRNP
NDRIGIYYDKLDLYATYHSQQITYKTSLPTTYQGHKEINVWSPYVYGNAVPVAPYNAVSLTQDQSSGIIPLMFKIDGRVR
WKVGTFITGKYHLYVRCPAYINFGDRQAGTAVGNNAVKYQLVQSCSVSV
>XP_006481585.1 NDR1/HIN1-like protein 1 [Citrus sinensis]
MSEKVCDKHGCKRRKIFRRIIAGILIFILIVLITILIVWAILRPTKPRFILQDATVYVENVSNPNVLTSSFQVTISSRNP
NDRIGIYYDKLDLYATYHSQQITYKTSLPTTYQGHKEVNVWSPYVYGNAVPVAPYNAVSLTQDQSSGIIPLTFKIDGRVR
WKVGTFITGKYHLYVRCPAYINFGDRQAGTAVGNNAVKYQLVQSCSVSV
>GAY31912.1 hypothetical protein CUMW_280830 [Citrus unshiu]
MSEKDCGHSHDDRKKLVRLILNAVGGLIIVVLLIIFLEWAITRPSKPSFILQDATLYAENLSTGPSPPNALTINLQVTIT
TRNPNDKIGIYYQKADVYASYRNQQISLATLLPATYQGHKDVIVWSPFLYGNSVPVSPFVAESLGQDLNAGMVMVNIKVD
GRIKWKVGTWISGRYHLHVNCPAYITFGDKSKGIASGASLKFQLVQSCSVDV
>XP_006424128.1 NDR1/HIN1-like protein 1 [Citrus clementina]
MSEKDCGHSHDDKKKLVRLILYAVGGLIIVVLLIIFLFWAITRPSKPSFILQDATLYAFNLSTGPSPPNALTTNLQVTIT
TRNPNDKIGIYYQKADVYASYRNQQISLATLLPATYQGHKDVIVWSPFLYGNSVPVSPFVAEALGQDLNAGMVMVNIKVD
GRIKWKVGTWISGRYHLHVNCPAYITFGDKSKGIASGASVKFQLVQSCSVDV
>XP_006481531.1 NDR1/HIN1-like protein 1 [Citrus sinensis]
MSEKDCGHSHDDRKKLVRLILYAVGGLIIVVLLIIFLFWAITRPSKPSFILQDATLYAFNLSTGPSPPNALTTNLQVTIT
TRNPNDQIGIYYQKADVYASYRNQQISLATLLPATYQGHKDVIVWSPFLCGNSVPVSPFVAEALGQDLNAGMVMVNIKVD
GRIKWKVGTWISGRYHLHVNCPAYITFGDKSKGIASGASLKFQLVQSCSVDV
>GAY32947.1 hypothetical protein CUMW_004890 [Citrus unshiu]
MTESLDLYATYHSQQITYKTSLPTTYQGHKEVNVWSPYVYGNAVPVAPYNAVSLTQDQSSGIIPLTFKIDGRVRWKVGTE
ITGKYHLYVRCPAYINFGDRQAGTAVGNNAVKYQLVQSCSVSV
>StNDR1-like PGSC0003DMP400048906 sequence match in blast db Potato PGSC DM v3.4
protein sequences
MSVKECTHHKDKKRKLVRRLFAGILIFLFVVLLTVLLVWAILQPKKPRFILQDATIFNENVSAPNIFSTSIQITIYSRNP
NDKIGVYYDKMKTYANYHKQQITYYTQIPSVYQGHKDVNIWSPFVFSNNVPISPINGPDLKEDQQNGGVWLDFKIDGRVK
WRVGTITTGHYHLHVTCTAYVPFGNHPGDGGLFVGNNAVKYQLARSCDVSV
PSL4 protein sequence
>PtPSL4.1_Ptrif.0002s0022.1.v1.3.1_Poncirus_trifoliata
MRVVLVDERFTYAIVLSLLWVSSSVIGRSNAASSLLNDPFYGISPQDENY
YKTSSNTIKCKDGSKKFAKTQLNDDYCDCPDGTDEPGTSACPNGKFYCQN
AGHSPLMIFSSKVNDGICDCCDGSDEYDGKVKCPNTCWEAGKVARDKLKK
KIATYQEGVLLRKKEIEQAKQNLVKDEAELSNLKNEEKILKGLVQQLKER
KEQIEKAEEKERLQREKEEKERKEAEENERKEKSESGEKDMQEKNKAEEN
AYSDDKPDDVRHDDKVGVLEEESFDQGKAGNVDEEPATEAKQIGTSQNLG
TPVNGVEQHATEEMEQSASSRSKDGSSTVPETSSDAENQMPPEAEKKEEK
NLENGVSENTEELSREELGRLVASRWTGEKTEKQSGEGGAIENDDQGEDV
PEYNHDDEEDRYATDTDDDSERYDTEKYDDNDVEDDIDETYREEDHDYTS
TSYKTDVDDDLDMSEMTTPSSPSWLEKVQQTVRNILQAVNLFQTPVDKSD
AARVRKEYDESSDKLSKIQSRISSLTQKLKHDFGPEKEFYSFHGHCFESK
QNKYVYKVCPYKKATQEEGHSTTRLGSWDKFEDSYHIMLESNGDKCWNGP
DRSMKVRLRCGLKNEVTDVDEPSRCEYVALLSTPAVCSEEKLQELEHKLD
ELNKKQPQHHDEL-
>PtPSL4.2_Ptrif.0002s0016.1.v1.3.1_Poncirus_trifoliata
MPEWKILLSECRTEPLMIFSSKVNDGICDCCDGSDEYDGKVKCPNTCWEA
GKVARDKLKKKIATYQEGVLLRKKEIEQAKQNLVKDEAELSNLKNEEKIL
KGLVQQLKERKEQIEKAEEKERLQREKEEKERKEAEENERKEKSESGEKD
MQEKNKAEENAYSDDKPDDVRHDDKVGVLEEESFDQGKAGNVDEEPATEA
KQIGTSQNLGTPVNGVEQHATEEMEQSASSRSKDGSSTVPETSSDAENQM
PPEAEKKEEKNLENGVSENTEELSREELGRLVASRWTGEKTEKQSGEGGA
IENDDQGEDVPEYNHDDEEDRYATDTDDDSERYDTEKYDDNDVEDDIDET
YREEDHDYTSTSYKTDVDDDLDMSEMTTPSSPSWLEKVQQTVRNILQAVN
LFQTPVDKSDAARVRKEYDESSDKLSKIQSRISSLTQKLKHDFGPEKEFY
SFHGHCFESKQNKYVYKVCPYKKATQEEGHSTTRLGSWDKFEDSYHIMLE
SNGDKCWNGPDRSMKVRLRCGLKNEVTDVDEPSRCEYVALLSTPAVCSRK
SERNWNIN-
>XP_006445558.1 glucosidase 2 subunit beta [Citrus clementina]
MRVVLVDFRFTYAIVLSLLWVSSSVIGRSNAASSLLNDPFYGISPQDENYYKTSSNTIKCKDGSKKFAKTQLNDDYCDCP
DGTDEPGTSACPNGKFYCQNAGHSPLMIFSSKVNDGICDCCDGSDEYDGKVKCPNTCWEAGKVARDKLKKKIATYQEGVL
LRKKEIEQAKQNLVKDEAELSNLKNEEKILKGLVQQLKERKEQIEKAEEKERLQREKEEKERKEAEENERKEKSESGEKA
MQEKNKAEENAYSDDKPDDVRHDDKVGVLEEESFDQGKAENVDEEPATEAKQIGTSQNLGTPVNGVEQHATEEMEQSASS
RSKDGSSTVPETSSDAESQMPPEAEKKEEKNLENGVSENTEELSREELGREVASRWTGEKTEKQSGEGGAIANDDQGEDV
PEYNHDDEEDRYATDTDDDSERYDTEKYDDNDVEDDIDEPYREEDHDYTSTSYKTDVDDDLDMSEMTTPSSPSWLEKIQQ
TVRNILQAVNLFQTPVDKSDAARVRKEYDESSDKLSKIQSRISSLTQKLKHEFGPEKEFYSFYGHCFESKQNKYVYKVCP
YKKATQEEGHSTTRLGSWDKFEDSYHIMLESNGDKCWNGPDRSMKVRLRCGLKNEVTDVDEPSRCEYVALLYTPAVCSEE
KLQELQHKLDELNKKQPQHHDEL
>KDO54514.1 hypothetical protein CISIN_1g006056mg [Citrus sinensis]
MRVVLVDERFTYAIVLSLLWVSSSVIGRSNAASSLUNDPFYGISPQDENYYKTSSNTIKCKDGSKKFAKTQENDDYCDCP
DGTDEPDCCDGSDEYDGKVKCPNTCWEAGKVARDKLKKKIATYQEGVLLRKKEIEQAKQNLVKDEAELSNLKNEEKILKG
LVQQLKERKEQIEKAEEKERLQREKEEKERKEAEENERKEKSESGEKAMQEKNKAEENAYSDDKPDDVRHDDKVGVLEEE
SFDQGKAENVDEEPATEAKQIGTSQNLGTPVNGVEQHATEEMEQSASSRSKDGSSTVPETSSDAESQMPPEAEKKEEKNL
ENGVSENTEELSREELGRLVASRWTGEKTEKQSGEGGAIANDDQGEDVPEYNHDDEEDRYATDTDDDSERYDTEKYDDND
VEDDIDEPYREEDHDYTSTSYKTDVDDDLDMSEMTTPSSPSWLEKIQQTVRNILQAVNLFQTPVDKSDAARVRKEYDESS
DKLSKIQSRISSLTQKLKHEFGPEKEFYSFYGHCFESKQNKYVYKVCPYKKATQEEGHSTTRLGSWDKFEDSYHIMLFSN
GDKCWNGPDRSMKVRLRCGLKNEVTDVDEPSRCEYVALLYTPAVCSEEKLQELQHKLDELNKKQPQHHDEL
>GAY57580.1 hypothetical protein CUMW_180530 [Citrus unshiu]
MRVVLVDERFTYAIVESLLWVSSSVIGRSNAASSLLNDPFYGISPQDENYYKTSSNTIKCKDGSKKFAKTQUNDDYCDCP
DGTDEPGTSACPNGKFYCQNAGHSPLMIFSSKVNDGICDCCDGSDEYDGKVKCPNTCWEAGKVARDKLKKKIATYQEGVL
LRKKEIEQAKQNLVKDEAELSNLKNEEKILKGLVQQLKERKEQIEKAEEKERLQREKEEKERKEAEENERKEKSESGEKA
MQEKNKAEENAYSDDKPDDVRHDDKVGVLEEESFDQGKAENVDEEPATEAKQIGTSQNLGTPVNGVEQENGVSENTEELS
REELGRLVASRWTGEKTEKQSGEGGAIANDDQGEDVPEYNHDDEEDRYATDTDDDSERYDTEKYDDNDVEDDIDEPYREE
DHDYTSTSYKTDVDDDLDMSEMTTPSSPSWLEKIQQTVRNILQAVNLFQTPVDKSDAARVRKEYDESSDKLSKIQSRISS
LTQKLKHEFGPEKEFYSFYGHCFESKQNKYVYKVCPYKKATQEEGHSTTRLGSWDKFEDSYHIMLESNGDKCWNGPDRSM
KVRLRCGLKNEVTDVDEPSRCEYVALLYTPAVCSEEKLQELQHKLDELNKKQPQHHDEL
>KDO54515.1 hypothetical protein CISIN_1g006056mg [Citrus sinensis]
MRVVLVDERFTYAIVLSLLWVSSSVIGRSNAASSLLNDPFYGISPQDENYYKTSSNTIKCKDGSKKFAKTQLNDDYCDCP
DGTDEPGTSACPNGKFYCQNAGHSPLMIFSSKVNDGICDCCDGSDEYDGKVKCPNTCWEAGKVARDKLKKKIATYQEGVL
LRKKEIEQAKQNLVKDEAELSNLKNEEKILKGLVQQLKERKEQIEKAEEKERLQREKEEKERKEAEENERKEKSESGEKA
MQEKNKAEENAYSDDKPDDVRHDDKVGVLEEESEDQGKAENVDEEPATEAKQIGTSQNLGTPVNGVEQHATEEMEQSASS
RSKDGSSTVPETSSDAESQMPPEAEKKEEKNLENGVSENTEELSREELGRLVASRWTGEKTEKQSGEGGAIANDDQGEDV
PEYNHDDEEDRYATDTDDDSERYDTEKYDDNDVEDDIDEPYREEDHDYTSTSYKTDVDDDLDMSEMTTPSSPSWLEKIQQ
TVRNILQAVNLFQTPVDKSDAARVRKEYDESSDKLSKIQSRISSLTQKLKHEFGPEKEFYSFYGHCFESKQNKYVYKVCP
YKKATQEEGHSTTRLGSWDKFEDSYHIMLESNGDKCWNGPDRSMKVTL
>GAY57579.1 hypothetical protein CUMW_180530 [Citrus unshiu]
MRVVLVDFRFTYAIVLSLLWVSSSVIGRSNAASSLLNDPFYGISPQDENYYKTSSNTIKCKDGSKKFAKTQLNDDYCDCP
DGTDEPGTSACPNGKFYCQNAGHSPLMIFSSKVNDGICDCCDGSDEYDGKVKCPNTCWEAGKVARDKLKKKIATYQEGVL
LRKKEIEQAKQNLVKDEAELSNLKNEEKILKGLVQQLKERKEQIEKAEEKERLQREKEEKERKEAEENERKEKSESGEKA
MQEKNKAEENAYSDDKPDDVRHDDKVGVLEEESFDQGKAENVDEEPATEAKQIGTSQNLGTPVNGVEQENGVSENTEELS
REELGRLVASRWTGEKTEKQSGEGGAIANDDQGEDVPEYNHDDEEDRYATDTDDDSERYDTEKYDDNDVEDDIDEPYREE
DHDYTSTSYKTDVDDDLDMSEMTTPSSPSWLEKIQQTVRNILQAVNLFQTPVDKSDAARVRKEYDESSDKLSKIQSRISS
LTQKLKHEFGPEKEFYSFYGHCFESKQNKYVYKVCPYKKATQEEGHSTTRLGSWDKFEDSYHIMLFSNGDKCWNGPDRSM
KENEFNYKCIVLQVRLRCGLKNEVTDVDEPSRCEYVALLYTPAVCSEEKLQELQHKLDELNKKQPQHHDEL
>XP_024958562.1 glucosidase 2 subunit beta isoform X2 [Citrus sinensis]
MRVVLVDERFTYAIVLSLLWVSSSVIGRSNAASSLLNDPFYGISPQDENYYKTSSNTIKCKDGSKKFAKTQLNDDYCDCP
DGTDEPGTSACPNGKEYCQNAGHSPLMIFSSKVNDGICDCCDGSDEYDGKVKCPNTCWEAGKVARDKLKKKIATYQEGVL
LRKKEIEQAKQNLVKDEAELSNLKNEEKILKGLVQQLKERKEQIEKAEEKERLQREKEEKERKEAEENERKEKSESGEKA
MQEKNKAEENAYSDDKPDDVRHDDKVGVLEEESFDQGKAENVDEEPATEAKQIGTSQNLGTPVNGVEQHATEEMEQSASS
RSKDGSSTVPETSSDAESQMPPEAEKKEEKNLENGVSENTEELSREELGREVASRWTGEKTEKQSGEGGAIANDDQGEDV
PEYNHDDEEDRYATDTDDDSERYDTEKYDDNDVEDDIDEPYREEDHDYTSTSYKTDVDDDLDMSEMTTPSSPSWLEKIQQ
TVRNILQAVNLFQTPVDKSDAARVRKEYDESSDKLSKIQSRISSLTQKLKHEFGPEKEFYSFYGHCFESKQNKYVYKVCP
YKKATQEEGHSTTRLGILFVLPP
>KDO54516.1 hypothetical protein CISIN_1g006056mg [Citrus sinensis]
MRVVLVDFRFTYAIVLSLLWVSSSVIGRSNAASSLLNDPFYGISPQDENYYKTSSNTIKCKDGSKKFAKTQLNDDYCDCP
DGTDEPGTSACPNGKFYCQNAGHSPLMIFSSKVNDGICDCCDGSDEYDGKVKCPNTCWEAGKVARDKLKKKIATYQEGVL
LRKKEIEQAKQNLVKDEAELSNLKNEEKILKGLVQQLKERKEQIEKAEEKERLQREKEEKERKEAEENERKEKSESGEKA
MQEKNKAEENAYSDDKPDDVRHDDKVGVLEEESFDQGKAENVDEEPATEAKQIGTSQNLGTPVNGVEQHATEEMEQSASS
RSKDGSSTVPETSSDAESQMPPEAEKKEEKNLENGVSENTEELSREELGRLVASRWTGEKTEKQSGEGGAIANDDQGEDV
PEYNHDDEEDRYATDTDDDSERYDTEKYDDNDVEDDIDEPYREEDHDYTSTSYKTDVDDDLDMSEMTTPSSPSWLEKIQQ
TVRNILQAVNLFQTPVDKSDAARVRKEYDESSDKLSKIQSRISSLTQKLKHEFGPEKEFYSFYGHCFESKQNKLLFLTLF
SSI
>GAY57581.1 hypothetical protein CUMW_180530 [Citrus unshiu]
MRVVLVDERFTYAIVLSLLWVSSSVIGRSNAASSLLNDPFYGISPQDENYYKTSSNTIKCKDGSKKFAKTQLNDDYCDCP
DGTDEPDCCDGSDEYDGKVKCPNTCWEAGKVARDKLKKKIATYQEGVLLRKKEIEQAKQNLVKDEAELSNLKNEEKILKG
LVQQLKERKEQIEKAEEKERLQREKEEKERKEAEENERKEKSESGEKAMQEKNKAEENAYSDDKPDDVRHDDKVGVLEEE
SFDQGKAENVDEEPATEAKQIGTSQNLGTPVNGVEQENGVSENTEELSREELGRLVASRWTGEKTEKQSGEGGAIANDDQ
GEDVPEYNHDDEEDRYATDTDDDSERYDTEKYDDNDVEDDIDEPYREEDHDYTSTSYKTDVDDDLDMSEMTTPSSPSWLE
KIQQTVRNILQAVNLFQTPVDKSDAARVRKEYDESSDKLSKIQSRISSLTQKLKHEFGPEKEFYSFYGHCFESKQNKYVY
KVCPYKKATQEEGHSTTRLGSWDKFEDSYHIMLESNGDKCWNGPDRSMKVRLRCGLKNEVTDVDEPSRCEYVALLYTPAV
CSEEKLQELQHKLDELNKKQPQHHDEL
>StPSL4_PGSC0003DMP400008210 sequence match in blast db Potato PGSC DM v3. 4
protein sequences
MELRFQEVFLLSCIFCICSIDRSVSLPSIVNLGIAPEDENYYKGLSSGAINCKDGSKKFTKAQLNDDECDCPDGSDEPGT
SACPSGKFYCKNAGHAPLFIYSSRVNDGICDCCDGSDEHDGKVKCPNTCWEVGRVARDKLKKKIATFQEGIIIRKKETEE
AKLAIAKEETEVSKLKNEQKILKGRVEQLQDKKEQIEKVEEEERLKREKEEKERKEADDAKLEASKVEEKTEVHEEAVKS
DIHDKIGLLEDSPPVKDVVEGHDKAADEEQHGDHSVKDEFPVDEVEQVPEDSSQHPEIKEASTNNNKADVSSRNEEKDAA
ENIESLSKEELGRVIGSRWLGKKSEQETESVEAGTDSNHDNHDEVPSDTHEEEYHGYDSDVDDRKYDDEHKYDDDENKYD
DDDNEDHVEDSVGEDHDSSSSYKSESDDDSDEADTTTTTSPSWTEKIQQTVKRIFRSVNLFQTPVNISDANCIRKEYDEA
SAKLTKIESRLSSLKQKLKHDFGPEKEFYSFHGQCFESKENKYTYKICPFKEATQVEGYSTTRLGNWDKFEDSYRTMQFT
NGDHCWNGPNRSVKVKLRCGLKNEVIDIDEPSRCEYLAFLSTPALCLEEKLKELQDKLEMMNREQPQDHDEL
LYM2 protein sequences
>PtLYM2_Ptrif.0008s0065.2.v1.3.1_Poncirus_trifoliata
MGNFQLKLVSLLFTVCAALSTLSTAQDFKCSAQTAARCQALVGYLPENKT
TISEIQSLFTVKNLRSILGANNFPPGTRRNFSVPAQKPIKVPIPCICSNG
TGVSNKLPVYTVKKDDGLDFIARTIFGQLLKYQKIVEANNISNPDLIQIG
QNLTIPLPCSCDDVDNAKVVHYAHVVEEGSSFAIIAQKFGTSRDTLMKLN
GIDDDSKLIAGEPLDVPLKACNSSIRADSFDSYLRVANGTYTETANSCVK
CQCDATNNWTLQCEPSQFQPSSTHSRWKTCPSMLCGGSESLSIGNATTSN
NCNRTTCEYAGYNNLSILTTINSLSTCPSPSNNASRIGSWNLLLISIFLVLLHFHLIQ-
>XP_006422460.1 lysM domain-containing GPI-anchored protein 2 isoform X2 [Citrus
clementina]
MRINKPKPRLFKLQTSNFKSLLSSSAQEEEQDSRGQPHYQCIYNKKLLTTSQRKVNKMGNFQLKLVLLLFTVCAALSTLS
TAQDFKCSTQTAARCQALVGYLPPNKTTISEIQSLFTVKNLRSILGANNFPPGTPRNFSVPAQKPIKVPIHCICSNGTGV
SDKVPVYTVKKDDGLDFIARTIFGQLLKYQKIVEANNISNPDLIQIGQNLTIPLPCSCDDVDNAKVVHYAHVVEEGSSFE
LIAQKFGTDRDTLMKINGIHDDSKLIAGEPLDVPLKACNSSIKADSFDNYLRVANGTYTFTANSCVKCQCDATNNWTLQC
KPSQFQPSSPNSRWKTCPSMLCGDSESLSIGNITTSNNCNRTTCEYAGYNNLSILTTENSLSTCPSPSNNASRIGSWNLL
LISIFLVLLHFHLIQ
>XP_024035093.1 lysM domain-containing GPI-anchored protein 2 isoform X1 [Citrus
clementina]
MRINKPKPRLFKLQTSNFKSLLSSSAQEEEQDSRGQPHYQCIYNKKLLTTSQRKVNKMGNFQLKLVLLLFTVCAALSTLS
TAQDFKCSTQTAARCQALVGYLPPNKTTISEIQSLFTVKNERSILGANNFPPGTPRNFSVPAQKPIKVPIHCICSNGTGV
SDKVPVYTVKKDDGLDFIARTIFGQLLKYQKIVEANNISNPDLIQIGQNLTIPLPCSCDDVDNAKVVHYAHVVEEGSSFE
LIAQKFGTDRDTLMKINGIHDDSKLIAGEPLDVPLKACNSSIKADSFDNYLRVANGTYTFTANSCVKCQCDATNNWTLQC
KPSQFQPSSPNSRWKTCPSMLCGDSESLSIGNTTTSNNCNRTTCEYAGYNNLSILTTINSLSTCPSKFKLSSHPLECINA
LQIYVLIVSLHWNTILSLQVHWHFI
>XP_006486627.1 lysM domain-containing GPI-anchored protein 2 isoform X2 [Citrus
sinensis]
MGNFQLKLVLLLFTVCAALSTLSTAQDFKCSAQTAARCQALVGYLPPNKTTISEIQSLFTVKNLRSILGANNEPPGTPRN
FSVPAQKPIKVPIHCICSNGTGVSDKVPVYTVKKDDGLDFIARTIFGQLLKYQKIVEANNISNPDLIQIGQNLTIPLPCS
CDDVDNAKVVHYAHVVEEGSSFALIAQKFGTDRDTLMKLNGIHDDSKLIAGEPLDVPLKACNSSIRADSFDNYLRVANGT
YTFTANSCVKCQCDATNNWTLQCEPSQFQPSSPNSRWKTCPSMLCGDSESLSIGNTTTSNNCNRTTCEYAGYNNLSILTT
LNSLSTCPSPSNNASRIGSWNLLLISIFLVLLAFHLIQ
>XP_006486626.1 lysM domain-containing GPI-anchored protein 2 isoform X1 [Citrus
sinensis]
MGNFQLKLVLLLFTVCAALSTLSTAQDEKCSAQTAARCQALVGYLPPNKTTISEIQSLFTVKNLRSILGANNEPPGTPRN
FSVPAQKPIKVPIHCICSNGTGVSDKVPVYTVKKDDGLDFIARTIFGQLLKYQKIVEANNISNPDLIQIGQNLTIPLPCS
CDDVDNAKVVHYAHVVEEGSSFALIAQKFGTDRDTLMKLNGIHDDSKLIAGEPLDVPLKACNSSIRADSEDNYLRVANGT
YTFTANSCVKCQCDATNNWTLQCEPSQFQPSSPNSRWKTCPSMLCGDSESLSIGNTTTSNNCNRTTCEYAGYNNLSILTT
LNSLSTCPSKFKLSSHPLFCLNALQIYVLIVSLHWNTILSLQVHWLFI
>KDO68285.1 hypothetical protein CISIN_1g0182902mg, partial [Citrus sinensis]
MGNFQLKLVLLLETVCAALSTLSTAQDFKCSAQTAARCQALVGYLPPNKTTISEIQSLFTVKNLRSILGANNFPPGTPRN
FSVPAQKPIKVPIHCICSNGTGVSDKVPVYTVKKDDGLDFIARTIFGQLLKYQKIVEANNISNPDLIQIGQNLTIPLPCS
CDDVDNAKVVHYAHVVEEGSSFALIAQKFGTDRDTLMKLNGIHDDSKLIAGEPLDVPLKACNSSIKADSFDNYLRVANGT
YTFTANSCVKCQCDATNNWTLQCKPSQFQPSSPNSRWKTCPSMLCGDSESLSIGNTTTSNNCNRTTCEYAGYNNLSILTT
LNSLSTCP
>GAY46120.1 hypothetical protein CUMW_094550, partial [Citrus unshiu]
TGVSNKVPVYTVKKDDGLDFIARTIFGQLLKYQKIVEANNISNPDLIQIGQNLTIPLPCSCDDVDNAKVVHYAHVVEEGS
SFVLIAQKFGTDRDTLMKLNGIHDDSKLIAGEPLDVPLKACNSSIRADSEDNYLRVANGTYTFTANSCVKCQCDATNNWT
LQCEPSQFQPSSPNSRWKTCPSMLCGDSESLSIGNTTTSNNCNRTTCEYAGYNNLSILTTLNSLSTCPSPSNNASRIGSW
NLLLISIFLVLLHFHLIQ
>GAY46119.1 hypothetical protein CUMW_094540 [Citrus unshiu]
MAPSLQFYLPNFIANRVSNKVPVYTVKKDDGLDFIARTIFGQLLKYQKIVEANNISNPDLIQIGQNLTIPLPCSCDDVDN
AKVVHYAHVVEEGSSFVLIAQKFGTDRDTLMKLNGIHDDSKLIAGEPLDVPLKACNSSIRADSEDNYLRVANGTYTFTAN
SCVKCQCDATNNWTLQCEPSQFQPSSPNSRWKTCPSMLCGDSESLSIGNTTTSNNCNRTTCEYAGYNNLSILTTINSLST
CPSPSNNASRIGSWNLLLISIFLVLLHFHLIQ
SOT12 protein sequences
>PtSOT12.1_Ptrif.0004s0884.1.v1.3.1_Poncirus_trifoliata
MACQEEGLLLDEYPKEKFWEILDLYQLDGYWYSGDVIPGMLAFKSEFEAL
SDDVILASSMKTGTTWLKALCICIMGNQRKNDGDEVDQLEVKNPHDHIKC
LEYLYYENLLSKLKDMQSPRVENTHLPYSALPELIKNSECKIVYIARNPK
DTFVSLWHFFNQILPPNTEPYPLEKAYNSFIKGIHLEGPFHDHVLEYWQE
SLKNPNKLLFLKYEDLKRDPKGEVRKLASFLGRPFGDDDNDEVDKVLWRS
SFERLKNLEVNKNGKLSDSGVPNSSFFRLGNVGDWQNCFTDEMKQGLDEI
TCKKFEGTGLDL-
>PtSOT12.2_Ptrif.0004s0882.1.v1.3.1_Poncirus_trifoliata
MATASSIPTQELLDQLPKHLHWEAYNIYQWEGFWYPAAVIRGMLAFRSNY
KARCDDVILASSLKTGTTWLKALCACIMDYHDDQLSSKNPHLVVKTLEYE
FAGETLNPDDLSGMSSPRLFHTHLPYSSLPESIKNSECKIVYITRNPSDT
MVSGWHYENRILRRNNQDPYPFEKEYNNFCAGVHSYGPFFYHVLQYWSGS
LKTPSKILFLKYEELKRDPKGQVKRLASFLGRPLAGEDEVDKVIWGSSFE
RLKNLEVNKNGELPFGNVPNSAFLRLGKVGDWENYFTAEMKQGLDEITRM
KLEGSGLDFES-
>XP_006485556.1 cytosolic sulfotransferase 12-like [Citrus sinensis]
MACQEEGLLLDEYPKEKFWEILDLYQLDGYWYSGDVIPGMLAFKSEFKALSDDVILASSMKTGTTWLKALCICIMGNQRK
NDRDEVDQLEVKNPHDHIKCLEYLYYENLLSKLKDMQSPRVENTHLPYSALPESIKNSECKIVYIARNPKDTFVSLWHFF
NQILPPNTEPYRLEKAYDSFIKGIHLFGPFHDHVLEYWQESLKNPNKLLFLKYEDLKRDPKGEVRKLASFLGRPFGDEDN
DEVDKVLWRSSFERLKNLEVNKNGKLSDSGVPNSSFFRLGNVGDWQNCETDEMKQGLDEITCKKFEGTGLDE
>KDO48723.1 hypothetical protein CISIN_1g037802mg [Citrus sinensis]
MACQEEGLLLDEYPKEKFWEILDLYQLDGYWYSGDVIPGMLAFKSEFEALSDDVILASSMKTGTTWLKALCICIMGNQRK
NDGDEVDQLEVKNPHDHIKCLEYFYYFNLLSKLKDMQSPRVENTHLPYSALPESIKNSECKIVYIARNPKDTFVSLWHFF
NQILPPNTEPYRLEKAYDSFIKGIHLFGPFHDHVLEYWQESLKNPNKLLFLKYEDLKRDPKGEVRKLASFLCRPFGDEDN
DEVDKVLWRSSFERLKNLEVNKNGKLSDSGVPNSSFERLGNVGDWQNCFTDEMKQGLDEITCKKFEGSGLDL
>XP_024047618.1 cytosolic sulfotransferase 12 [Citrus clementina]
MACQEEGLLLDEYPKEKFWEILDLYQLDGYWYSGDVIPGTTWLKALCICIMGNQRKNDRDEVDQLEVKNPHDHIKCLEYL
YYFNLLSKLKDMQSPRVENTHLPYSALPESIKNSECKIVYIARNPKDTFVSLWHFENQILPPNTEPYRLEKAYDSFIKGI
HLEGPFHDHVLEYWQESLKNPNKLLFLKYEDLKRDPKGEVRKLASFLGRPFGDEDNDEVDKVLWRSSFERLKNLEVNKNG
KLSDSGVPNSSFERLGNVGDWQNCFTDEMKQGLDEITCKKFEGTGLDL
SCE1 protein sequence
>PESCE1.1_Ptrif.0005s2463.1.v1.3.1_Poncirus_trifoliata
MSGGIARGRLTEERKAWRKNHPHGFVAKPETKDGSVNLMIWECIIPGKTG
TDWEGGYFPLTLYFSEDYPSKPPKCKFPQGFFHPNVYPSGTVCLSILNED
NGWRPAITVKQILVGIQDLLDQPNPADPAQTDGYQLFIQDPAEYKRRVRL
QAKQYPPVL-
>PtSCE1.2_Ptrif.0007s0463.1.v1.3.1_Poncirus_trifoliata
MSGGIARGRLAEERKSWRKNHPHGFVAKPETLPDGSVNLMVWHCTIPGKA
GIDWEGGFFPLTLHFSEDYPSKPPKCKFPQGFFHPNVYPSGTVCLSILNE
DNGWRPAITVKQILVGIQDLLDQPNPADPAQTEGYHLFIQDGAEYKRRVR
QQAKQYPALL-
>ESR47961.1 hypothetical protein CICLE_v10002741mg [Citrus clementina]
MSGGGIARGRLTEERKAWRKNHPHTDWEGGYFPLTLYESEDYPSKPPKCKFPQGFFHPNVYPSGTVCLSILNEDNGWRPA
ITVKQILVGIQDLLDQPNPADPAQTDGYQLFIQDPAEYKRRVRQQAKQYPPVL
>KDO84096.1 hypothetical protein CISIN_1g031420mg [Citrus sinensis]
MSGGGIARGRLTEERKAWRKNHPHTDWEGGYFPLTLYFSEDYPSKPPKCKFPQGFFHPNVYPSGTVCLSILNEDNGWRPA
ITVKQILVGIQDLLDQPNPADPAQTDGYQLFIQDPAEYKRRVRQQAKQYPPVI
>XP_006434720.1 SUMO-conjugating enzyme SCEL isoform X1 [Citrus clementina]
MSGGGIARGRLTEERKAWRKNHPHGFVAKPETKDGSVNLMIWECIIPGKTGTDWEGGYFPLTLYFSEDYPSKPPKCKFPQ
GFFHPNVYPSGTVCLSILNEDNGWRPAITVKQILVGIQDLLDQPNPADPAQTDGYQLFIQDPAEYKRRVRQQAKQYPPVL
>KDO84093.1 hypothetical protein CISIN_1g031420mg [Citrus sinensis]
MSGGGIARGRLTEERKAWRKNHPHGFVAKPETKDGSVNLMIWECIIPGKTGTDWEGGYFPLTLYFSEDYPSKPPKCKEPQ
GFFHPNVYPSGTVCLSILNEDNGWRPAITVKQILVGIQDLEDQPNPADPAQTDGYQLFIQDPAEYKRRVRQQAKQYPPVI
>XP_006473285.1 SUMO-conjugating enzyme SCE1-like isoform X1 [Citrus sinensis]
MSGGIARGRLTEERKAWRKNHPHGFVAKPETKDGSVNIMIWECIIPGKTGTDWEGGYFPLTLYFSEDYPSKPPKCKEPQG
FFHPNVYPSGTVCLSILNEDNGWRPAITVKQILVGIQDLLDQPNPADPAQTDGYQLFIQDPAEYKRRVRQQAKQYPPVI
>XP_006425960.1 SUMO-conjugating enzyme SCE1 [Citrus clementina]
MSGGIARGRLAEERKSWRKNHPHGFVAKPETLPDGSVNLMVWHCTIPGKAGTDWEGGFFPLTLHFSEDYPSKPPKCKFPQ
GFFHPNVYPSGTVCLSILNEDNGWRPAITVKQILVGIQDLLDQPNPADPAQTEGYHLFIQDAAEYKRRVRQQAKQYPALL
>ESR47962.1 hypothetical protein CICLE_v10002741mg [Citrus clementina]
MIWECIIPGKTGTDWEGGYEPLTLYFSEDYPSKPPKCKFPQGFFHPNVYPSGTVCLSILNEDNGWRPAITVKQILVGIQD
LLDQPNPADPAQTDGYQLFIQDPAEYKRRVRQQAKQYPPVL
>KDO84100.1 hypothetical protein CISIN_1g031420mg [Citrus sinensis]
MIWECIIPGKTGTDWEGGYFPLTLYFSEDYPSKPPKCKFPQGFFHPNVYPSGTVCLSILNEDNGWRPAITVKQILVGIQD
LLDQPNPADPAQTDGYQLFIQDPAEYKRRVRQQAKQYPPVI
>XP_024039998.1 SUMO-conjugating enzyme SCEL isoform X2 [Citrus clementina]
MSGGGIARGRLTEERKAWRKNHPHGFVAKPETKDGSVNLMIWECIIPGKTGTDWEGGYFPLTLYFSEDYPSKPPKCKFPQ
GFFHPNVYPSGTVCLSILNEDNGWRPAITVKQILVGIQDLLDQPNPADPAQTDGYQLFIQ
>KDO84097.1 hypothetical protein CISIN_1g031420mg [Citrus sinensis]
MSGGGIARGRLTEERKAWRKNHPHGFVAKPETKDGSVNLMIWECIIPGKTGTDWEGGYFPLTLYFSEDYPSKPPKCKEPQ
GFFHPNVYPSGTVCLSILNEDNGWRPAITVKQILVGIQDLLDQPNPADPAQTDGYQLFIQVLYWFI
>XP_024952076.1 SUMO-conjugating enzyme SCE1-like isoform X2 [Citrus sinensis]
MSGGIARGRLTEERKAWRKNHPHGFVAKPETKDGSVNLMIWECIIPGKTGTDWEGGYFPLTLYFSEDYPSKPPKCKFPQG
FFHPNVYPSGTVCLSILNEDNGWRPAITVKQILVGIQDLLDQPNPADPAQTDGYQLFIQ
>ESR39199.1 hypothetical protein CICLE_v10026671mg [Citrus clementina]
MVWHCTIPGKAGTDWEGGFFPLTLHFSEDYPSKPPKCKFPQGFFHPNVYPSGTVCLSILNEDNGWRPAITVKQILVGIQD
LLDQPNPADPAQTEGYHLFIQDAAEYKRRVRQQAKQYPALL
>KDO84099.1 hypothetical protein CISIN_1g031420mg [Citrus sinensis]
MSGGGIARGRLTEERKAWRKNHPHGFVAKPETKDGSVNLMIWECIIPGKTGTDWEGGYFPLTLYFSEDYPSKPPKCKFPQ
GFFHPNVYPSGTVCLSILNEDNVSSSCRFVIYEQIMYGSGLAKISSLDDMMAFF
>GAY50297.1 hypothetical protein CUMW_125510 [Citrus unshiu]
MSGGIARGRLAEERKSWRKNHPHDSIYASFSLGVLHFCHFEVNAVRSSSNQFAGMLGWRPAITVKQILVGIQDLLDQPNP
ADPAQTEGYHLFIQDAAEYKRRVRQQAKQYPALL
>GAY39565, 1 hypothetical protein CUMW_045300 [Citrus unshiu]
MSGGIARGRLTEERKAWRKNHPHGWRPAITVKQILVGIQDLLDQPNPADPAQTDGYQLFIQDPAEYKRRVRQQAKQYPPV
GLY1 protein sequence
>PtGLY1_Ptrif.0004s2683.1.v1.3.1_Poncirus_trifoliata
MAASNSIEPRLFLNPIFTTSTTINSPTSLHIQNFKLKLPRFPTKNPTLVF
TENSSSGSATTNNNNNDNTIINPYPDDPDPVRVSAVSSENRARDGRDRRK
IVKVAWEKLVRWSRTWRSKAKTDILERTNKVVVLGGGSFGTAMAAHLANR
KAQLKVYMLMRDPVVCQSINDKHCNCRYFPEQELPENVIATTDAKTALLG
ADYCLHAVPVQFSSSFLEGISDYVDPGLPFISLSKGLELNTLRMMSQIIP
QALRNPRQPFIALSGPSFALELMNKLPTAMVVASKDRKLANAVQQLLASK
HLRISTSSDVTGVEIAGALKNVLAIAAGIVVGMNLGNNSMAALVAQGCSE
IRWLATKMGAKPTTITGLSGTGDIMLTCFVNLSRNRTVGVRLGSGEKLDD
ILSSMNQVAEGVSTAGAVIALAQKYNVKMPVLTAVARIMDNELTPKKAVL
ELMSLPQLFAQPLNSQTISKKKKRNDKMSKKKETVKEILASCCESSLGLD
STLCDQLRERLGFEAPTKVQAQAIPVILSGRDVLVNAATGTGKTVAYLAP
IINHLQSYSPRIDRSSGTFALVLVPTSELCLLVYEILQKLLHRERWIVPG
YVMGGGNRSKEKARLRKGISILVATPGHLLDALKATSSFLATHVRWIIFD
EADRILELGFGKEIEEILDILGSRNIGSIGEGNEVSNVKRQNLLLSATIN
EKVIHLAKISLETPVLIGLDEKKLPEDKSNVHFGSLESDVKEEVEHPNTT
PSSSTEDFKLPAQLVHRYVKDIDRSNEDFDAFFNRLRSGSSVTTGSTSLK
GALPLCALGTKIKSQDSSPSGERGTLGTRKRKMGSLFSLPEDFIEGELDP
VANKKVSHLAEYVHYTSENITPEVAGEMDKLGLDERYNRALKASQDVTEL
LSRSLQDLAAIANVQLENEKLKNELQSYRSYEEKLSRENKTLKGRLNEVS
KEKARMVKDLKELQGKHEDLVSQQKEMIDSAFERIMTEVWSIDPGLVVPR
VEKWVDKSTILAAIETERESCLLQSGNLQRSSDVPRLLKLMLQLHPAL-
>ESR49174.1 hypothetical protein CICLE_v10031473mg [Citrus clementina]
MAASNSIEPRLFLNPIFTTSTTTNSSTSLHIQNFKLKLPHFPTKNPTLVETLNSSSGSATTNNNNDNTIITPYPDDPDPE
PVSAVSSETRTRDGRDRRKIVKVAWDKLVRWSRTWRSKAKTDILERTNKVVVLGGGSFGTAMAAHVANRKAQLKVYMLMR
DPVVCQSINEKHCNCRYFPEQKLPENVIATTDAKTALLGADYCLHAVPVQFSSSFLEGISDYVDPGLPFISLSKGLELNT
LRMMSQIIPQALRNPRQPFIALSGPSFALELMNKLPTAMVVASKDRKLANAVQQLLASKALRISTSSDVTGVEIAGALKN
VLAIAAGIVVGMNLGNNSMAALVAQGCSEIRWLATKMGAKPATITGLSGTGDIMLTCFVNLSRNRTVGVRLGSGEKLDDI
LSSMNQVAEGVSTAGAVIALAQKYNVKMPVLTAVARIIDNELTPKKAVLEIMSLPQVI
>ESR49175.1 hypothetical protein CICLE_v10031473mg [Citrus clementina]
MAASNSIEPRLFLNPIFTTSTTINSSTSLHIQNFKLKLPHFPTKNPTLVFTENSSSGSATTNNNNDNTIITPYPDDPDPE
PVSAVSSETRTRDGRDRRKIVKVAWDKLVRWSRTWRSKAKTDILERTNKVVVLGGGSFGTAMAAHVANRKAQLKVYMLMR
DPVVCQSINEKHCNCRYFPEQKLPENVIATTDAKTALLGADYCLHAVPVQFSSSFLEGISDYVDPGLPFISLSKGLELNT
LRMMSQIIPQALRNPRQPFIALSGPSFALELMNKLPTAMVVASKDRKLANAVQQLLASKHLRISTSSDVTGVEIAGALKN
VLAIAAGIVVGMNLGNNSMAALVAQGCSEIRWLATKMGAKPATITGLSGTGDIMLTCFVNLSRNRTVGVRLGSGEKLDDI
LSSMNQVAEGVSTAGAVIALAQKYNVKMPVLTAVARIIDNELTPKKAVLELMSLPQVEEV
>XP_006435935.2 glycerol-3-phosphate dehydrogenase [NAD(+)] 2, chloroplastic
[Citrus clementina]
MKKKIPILTLKSRSFICEQMAASNSIEPRLFLNPIFTTSTTINSSTSLHIQNFKLKLPHFPTKNPTLVETLNSSSGSATT
NNNNDNTIITPYPDDPDPEPVSAVSSETRTRDGRDRRKIVKVAWDKLVRWSRTWRSKAKTDILERTNKVVVLGGGSFGTA
MAAHVANRKAQLKVYMLMRDPVVCQSINEKHCNCRYFPEQKLPENVIATTDAKTALLGADYCLHAVPVQESSSFLEGISD
YVDPGLPFISLSKGLELNTLRMMSQIIPQALRNPRQPFIALSGPSFALELMNKLPTAMVVASKDRKLANAVQQLLASKHL
RISTSSDVTGVEIAGALKNVLAIAAGIVVGMNLGNNSMAALVAQGCSEIRWLATKMGAKPATITGLSGTGDIMLTCFVNL
SRNRTVGVRLGSGEKLDDILSSMNQVAEGVSTAGAVIALAQKYNVKMPVLTAVARIIDNELTPKKAVLEIMSLPQVEEV
>KDO67533.1 hypothetical protein CISIN_19012596mg [Citrus sinensis]
MAASNSIEPRLFLNPIFTTSTTTNSSTSLHIQNFKLKLPHFPTKNPTLVETLNSSSGSATTNNNNDNTIITPYPDDPDPE
PVSAVSSEIRTRDGRDRRKIVKVAWEKLVRWSRTWRSKAKTDILERTNKVVVLGGGSFGTAMAAHVANKKSQLKVYMLMR
DPAVCQSINEKHCNCRYFREQKLPENVIATTDAKTALLGADYCLHAMPVQFSSSFLEGISDYVDPGLPFISLSKGLEINT
LRMMSQIIPQALRNPRQPFIALSGPSFALELMNKLPTAMVVASKDRKLANAVQQLLASKALRISTSSDVTGVEIAGALKN
VLAIAAGIVVGMNLGNNSMAALVAQGCSEIRWLATKMGAKPATITGLSGTGDIMLTCFVNLSRNRTVGVRLGSGEKLDDI
LSSMNQVAEGVSTAGAVIALAQKYNVKMPVLTAVARIIDNELTPKKAVLEIMSLPQVI
>KDO67532.1 hypothetical protein CISIN_1g012596mg [Citrus sinensis]
MAASNSIEPRLFINPIFTTSTTTNSSTSLHIQNFKLKLPHFPTKNPTLVFTENSSSGSATTNNNNDNTIITPYPDDPDPE
PVSAVSSEIRTRDGRDRRKIVKVAWEKLVRWSRTWRSKAKTDILERTNKVVVLGGGSFGTAMAAHVANKKSQLKVYMLMR
DPAVCQSINEKHCNCRYFREQKLPENVIATTDAKTALLGADYCLHAMPVQFSSSFLEGISDYVDPGLPFISLSKGLELNT
LRMMSQIIPQALRNPRQPFIALSGPSFALELMNKLPTAMVVASKDRKLANAVQQLLASKHLRISTSSDVTGVEIAGALKN
VLAIAAGIVVGMNLGNNSMAALVAQGCSEIRWLATKMGAKPATITGLSGTGDIMLTCFVNLSRNRTVGVRLGSGEKLDDI
LSSMNQVAEGVSTAGAVIALAQKYNVKMPVLTAVARIIDNELTPKKAVLEIMSLPQVEEV
>KDO67534.1 hypothetical protein CISIN_1g012596mg [Citrus sinensis]
MAASNSIEPRLFLNPIFTTSTTTNSSTSLHIQNFKLKLPHFPTKNPTLVETLNSSSGSATTNNNNDNTIITPYPDDPDPE
PVSAVSSEIRTRDGRDRRKIVKVAWEKLVRWSRTWRSKAKTDILERTNKVVVLGGGSFGTAMAAHVANKKSQLKVYMLMR
DPAVCQSINEKHCNCRYFPEQKLPENVIATTDAKTALLGADYCLHAMPVQFSSSFLEGISDYVDPGLPFISLSKGLELNT
LRMMSQIIPQALRNPRQPFIALSGPSFALELMNKLPTAMVVASKDRKLANAVQQLLASKHLRISTSSDVTGVEIAGALKN
VLAIAAGIVVGMNLGNNSMAALVAQGCSEIRWLATKMGAKPATITGLSGTGDIMLTCFVNLSRNRTVGVRLGSGEKLDDI
LSSMNQVLVNPSMQPLLGKL
PAL1 protein sequences
>PtPAL1.1_Ptrif.0006s0395.1.v1.3.1_Poncirus_trifoliata
MERGATTENGHQNGSLGGLCKNNNYNYSSGDALNWGVMAETLKGSHLEEV
KRMVAEYRKPVVNLGGETLTVAQVAAIATSSTNVELSESAREGVKASSDW
VMESMNKGTDSYGVTTGFGATSHRRTKNGGALQKELIRFLNAGIEGNGTE
SSHTLPHSATRAAMLVRVNTLLQGYSGIRFEILEAITKLINHNITPCLPL
RGTITASGDLVPLSYIAGLLTGRPNSKATGPNGEIIDAQEASKQAGFGFF
ELQPKEGLALVNGTAVGSGLASMVLFEANNLALLSEILSAIFAEVMQGKP
EFTDHLTHKLKHHPGQIEAAAIMEHILDGSSYVNAAKKLHEIDPLQKPKQ
DRYALRTSPQWLGPQIEVIRFATKSIEREINSVNDNPLIDVSRNKALHGG
NFQGTPIGVSMDNTRLAIAAIGKLMFAQFSELVNDFYNNGLPSNLSGGRN
PSLDYGFKGAEIAMASYCSELQFLANPVTNHVQSAEQHNQDVNSLGLISS
RKTAEAVDILKLMSSTELVAICQAIDERHLEENLKHTVKNTVSQVAKKVL
TVGASGELHPSRECEKDLLKAADHEQVFAYIDDPCSATYPLMQKLRQVLV
EHALNNGENEKTANSSIFQKIAAFEEELKTVLPKEVENARQTVENGSPTI
PNRIKECRSYPLYRFVREELGSNFLTGEKVTSPGEEFDKVFTAMCQGKII
DPMLECLREWNGAPLPIC-
>PtPAL1.2_Ptrif.0006s0394.1.v1.3.1_Poncirus_trifoliata
MDRGAVIENGHQNGCLEGLCKNNNYSSGDALNWGVMAETLKGSHLEEVKR
MVAEYRKPVVNLGGETLTVAQVAAIATAGDVNAQVKVELSESAREGVKAS
SDWVMESMNKGTDSYGVTTGFGATSHRRTKNGGALQKELIRFLNAGIEGN
GTESSHMLPHSATRAAMLVRVNTLLQGYSGIRFEILEAITKLLNHSITPC
LPLRGTITASGDLVPLSYIAGLUTGRPNSKATGPNGEIIDAQEASKQAGF
GFFELQPKEGLALVNGTAVGSGLASMVLFDANNLALLSEILSAIFAEVMQ
GKPEFTDHLTHKLKHHPGQIEAAAIMEHILDGSSYVKAAKKLHEIDPLQK
PKQDRYALRTSPQWLGPQIEVIRFATKSIEREINSVNDNPLIDVSRNKAL
HGGNFQGTPIGVSMDNTRLAIAAIGKLMFAQFSELVNDFYNNGLPSNLSG
GRNPSLDYGFKGAEIAMASYCSELQFLANPVTNHVQSAEQHNQDVNSLGL
ISSRKTAEAVDILKLMSSTFLVALCQAIDERHLEENLKHTVKNTVSQVAK
KVLTVGASGELHPSRFCEKDLLKAADREHVFAYIDDPCSATYPLMQKLRQ
VLVDHALNNGENEKNANSSIFQKIAAFEEELKTVLPKEVENARQTVENGS
PTIPNRIKECRSYPLYRLVREELGTNFLTGEKVTSPGEEFDKVFTAMCQG
KIIDPMLECLREWNGAPLPIC-
>PtPAL1.3_Ptrif.0004s0590.2.v1.3.1_Poncirus_trifoliata
MELSHETCNGINNDRNGGTPSLGLCTGTDPLNWTVAADSLKGSHLDEVKR
MVDEHRRPVVKLGGESLTIGQVTAIAAHDSGVKVELAEAARAGVKASSDW
VMDSMMKGTDSYGVTTGFGATSHRRTKQGGALQKELIRFLNSGIFGNGTE
SSHTLPHSATRAAMLVRVNTLLQGYSGIRFEILETITKFLNANITPCLPL
RGTITASGDLVPLSYIAGLLTGRPNSKAVGPNGQVLNPTEAFNIAGVTSG
FFELQPKEGLALVNGTAVGSGLAATVLFEANILAIMSEVLSAIFAEVMNG
KPEFTDHLTHKLKHHPGQIEAAAIMEHILDGSSYVKAAQKLHEIDPLQKP
KQDRYALRTSPQWLGPQIEVIRAATKMIEREINSVNDNPLIDVSRNKALH
GGNFQGTPIGVSMDNTRLAIASIGKLMFAQFSELVNDFYNNGLPSNLTGG
RNPSLDYGFKGAEIAMASYCSELQFLANPVTNHVQSAEQHNQDVNSLGLI
SSRKTAEAVDILKLMSSTFLVALCQAIDLRHLEENLKNTVKNTVSQVAKR
VLTMGVNGELHPSRECEKDLIKVVDREYVFAYIDDPCSASYPLMQKLRQV
LVDHALDNGDREKNSTTSIFQKIGAFEDELKTLLPKEVEIARTELESGNA
AIPNRIKECRSYPLYKIVREEIGTSLLTGEKVRSPGEEFDKVFVAMCEGK
LIDPMLECLKEWNGAPLPIS-
>PtPAL1.4_Ptrif.0008s1965.1.v1.3.1_Poncirus_trifoliata
MEASLENQSGGNIPSGKLCTNIDPLNWVSASESLKGSHLDEVKRMVSEYR
KPVIRLGGETLTIAQVAAVASRDVGVTVELNEEARAGVKASSDWVMESIN
KGTDSYGITTGFGATSHRRTKQGATLQKELIRFLNAGIFGKGTESCQMLP
HTATRAAMLVRINSLLQGYSGIRFEILEAITKFLNRNITPCLPERASITA
SGDLIHFSYIAGLLTGRPNSVAVGPNGESLNAAEAFSQAGIDGGFFELQP
KEGLALVNGTGVGAGLASIVLFEANILTVLSEVLSAIFAEAMQGKPEFTD
HLTHKLKHHPGQIEAAAIMEHILAGSSCVKAAQILHEIDPLQKPKQDRYA
LRTSPQWLGPQAEVIRASTKSIEREINSVNDNPLIDVSRNKALHGGNFQG
TPIGVSMDNSRLAIASIGKLMFAQFSELVNDFYSNGLPSNLSGGRNPSLD
YGFKGAEIAMAAYCSELQFLANPVTNHVQSAEQHNQDVNSLGLISARKTA
EAVDILKLMSSTYLIALCQAIDLRHLEENLKSTVKNTISQVAKKVLTMGV
NGELHPSRECEKDLLKVVDREYVFSYADDPCSATYPLMQKLRQVLVDHAL
TNNEDLKNANASIFLKIGAFEEELKTLLPKEVESARSAFESGNLEMPNRI
KECRSYPLYRFVREQLGARYLTGEKAISPGEECDKVETAICQGKIIDPLL
ECLKEWDGSPLPIC-
>XP_006428759.1 phenylalanine ammonia-lyase [Citrus clementina]
MEIGATTENGHQNGGLEGLCKNNNYNYSSGDALNWGVMAETLKGSHLEEVKRMVAEYRKPVVNLGGETLTVAQVAAIATS
STNVELSESAREGVKASSDWVMESMNKGTDSYGVTTGFGATSHRRTKNGGALQKELIRFLNAGIEGNGTESSHTLPHSAT
RAAMLVRVNTLLQGYSGIRFEILEAITKLINHNITPCLPLRGTITASGDLVPLSYIAGLLTGRPNSKATGPNGEIIDAQE
ASKQAGFGFFELQPKEGLALVNGTAVGSGLASMVLFEANNLALLSEILSAIFAEVMQGKPEFTDHLTHKLKHHPGQIEAA
AIMEHILDGSSYVNVAKKLHEIDPLQKPKQDRYALRTSPQWLGPQIEVIRFATKSIEREINSVNDNPLIDVSRNKALHGG
NFQGTPIGVSMDNTRLAIAAIGKLMFAQFSELVNDFYNNGLPSNLSGGRNPSLDYGFKGAEIAMASYCSELQFLANPVTN
HVQSAEQHNQDVNSLGLISSRKTAEAVDILKLMSSTFLVALCQAIDLRHLEENLKHTVKNTVSQVAKKVLTVGASGELHP
SRFCEKDLLKAADREHVFAYIDDPCSATYPLMQKLRQVLVEHALNNGENEKTANSSIFQKIAAFEEELKTVLPKEVENAR
QTVENGSPTIPNRIKECRSYPLYRFVREGLGSNFLTGEKVTSPGEEFDKVFTAMCQGKIIDPMLECLREWNGAPLPIC
>KDO50673.1 hypothetical protein CISIN_1g005031mg [Citrus sinensis]
MERGATTENGHQNGGLEGLCKNNNYNYSSGDALNWGVMAETLKGSHLEEVKRMVAEYRKPVVNLGGETLTVAQVAAIATS
STNVELSESAREGVKASSDWVMESMNKGTDSYGVTTGFGATSHRRTKNGGALQKELIRFLNAGIFGNGTESSHTLPHSAT
RAAMLVRVNTLLQGYSGIRFEILEAITKLINHNITPCLPLRGTITASGDLVPLSYIAGLLTGRPNSKATGPNGEIIDAQE
ASKQAGEGFFELQPKEGLALVNGTAVGSGLASMVLFEANNLALLSEILSAIFAEVMQGKPEFTDHLTHKLKHAPGQIEAA
AIMEHILDGSSYVNAAKKLHEIDPLQKPKQDRYALRTSPQWLGPQIEVIRFATKSIEREINSVNDNPLIDVSRNKALHGG
NFQGTPIGVSMDNTRLAIAAIGKLMFAQFSELVNDFYNNGLPSNLSGGRNPSLDYGFKGAEIAMASYCSELQFLANPVTN
HVQSAEQHNQDVNSLGLISSRKTAEAVDILKLMSSTFLVALCQAIDLRHLEENLKHTVKNTVSQVAKKVLTVGASGELHP
SRFCEKDLLKAADREHVFAYIDDPCSATYPLMQKLRQVLVEHALNNGENEKTANSSIFQKIAAFEEELKTVLPKEVENAR
QTVENGSPTIPNRIKECRSYPLYRLVREELGSNFLTGEKVTSPGEEFDKVFTAMCQGKIIDPMLECLREWNGAPLPIC
>CAB42794.1 phenylalanine-ammonia lyase [Citrus clementina x Citrus reticulata]
MEIGATTENGHQNGGLEGLCKNNNYNYSSGDALNWGVMAETLKGSHLEEVKRMVAEYRKPVVNLGGETLTVAQVAAIATS
STNVELSESAREGVKASSDWVMESMNKGTDSYGVTTGFGATSHRTTKNGGALQKELIKELNAGIFGNGTKSSHTLPHSAT
RAAMLVRVNTLLQGYSGIRFEILKAITKLENHNITPCLPLRGTITASGDLVPLSYIAGLLTGRPNSKATGPNGQIIDPQE
ASKPAGFGFFELQPKEGLALVNGTAVGSGLASMVLFEANNLALLSEILSAIFAEVMQGKPEFTDHLTHKLKHHPGQTEAA
AIMEHILDGSSYVNVAKKLHEIDPLQKPKQDRYALRTSPQWLGPQIEMIRFATKSIEREINSVNDNPLIDVSRNKALHGG
NFQGTPIGVSMDNTRLAIAAIGKLMFAQFSELVNDFYNNGLPSNLSGGRNPSLDYGEKGAEIAMASYCSELQFLANPVTN
HVHSAEQHNQDVNSLGLISSRKTAEAVDILKLMSSTELVALCQAIDLRHLEENLKHTVKNTVSQVAKKVLIVGASGELHP
SRECEKDLLKAADREHVFAYIDDPCSATYPLMQKLRQVLVEHALNNGENEKTANSSIFQKIAAFEEELKTVLPKEVENAR
QTVENGSPTIPNRIKECRSYPLYRLVREGLGSNFLTGEKVTSPGEEFDKVETAMCQGKIIDPMLECLREWNGAPLPIC
>AIQ80958.1 phenylalanine ammonia-lyase [Citrus trifoliata]
MDRGAVIENGHQNGCLEGLCKNNNYSSGDALNWGVMAETLKGSHLEEVKRMVAEYRKPVVNLGGETLTVAQVAAIATAGD
VNAQVKVELSESAREGVKASSDWVMESMNKGTDSYGVTTGFGATSHRRTKNGGALQKELIRFLNAGIFGNGTESSHMLPH
SATRAAMLVRVNTLLQGYSGIRFEILEAITKLLNHSITPCLPLRGTITASGDLVPLSYIAGLLTGRPNSKATGPNGEIID
AQEASKQAGFGFFELQPKEGLALVNGTAVGSGLASMVLFDANNLALLSEILSAIFAEVMQGKPEFTDHLTHKLKHHPGQI
EAAAIMEHILDGSSYVKAAKKLHEIDPLQKPKQDRYALRTSPQWLGPQIEVIRFATKSIEREINSVNDNPLIDVSRNKAL
HGGNFQGTPIGVSMDNTRLAIAAIGKLMFAQFSELVNDFYNNGLPSNLSGGRNPSLDYGFKGAEIAMASYCSELQFLANP
VTNHVQSAEQHNQDVNSLGLISSRKTAEAVDILKLMSSTFLVALCQAIDLRHLEENLKHTVKNTVSQVAKKVLTVGASGE
LHPSRFCEKDLLKAADREHVFAYIDDPCSATYPLMQKLRQVLVDHALNNGENEKNANSSIFQKIAAFEEELKTVLPKEVE
NARQTVENGSPTIPNRIKECRSYPLYRLVREELGTNELTGEKVTSPGEEFDKVFTAMCQGKIIDPMLECLREWNGAPLPI
C
>XP_006481493.1 phenylalanine ammonia-lyase [Citrus sinensis]
MDRGAVIENGHQNGCLEGLCKDNNYSSGDALNWGVMAETLKGSHLEEVKRMVAEYRKPVVNLGGETLTVAQVAAIATAGD
VNAQVKVELSESAREGVKASSDWVMESMNKGTDSYGVTTGFGATSHRRTKNGGALQKELIRFLNAGIFGNGTESSHTLPH
SATRAAMLVRVNTLLQGYSGIRFEILDAITKLLNHSITPCLPLRGTITASGDLVPLSYIAGLLTGRANSKATGPNGEIID
AQEASKQAGFGFFELQPKEGLALVNGTAVGSGLASMVLFDANNLALLSEILSAIFAEVMQGKPEFTDHLTHKLKHHPGQI
EAAAIMEHILDGSSYVKAAKKLHEIDPLQKPKQDRYALRTSPQWLGPQIEVIRFATKSIEREINSVNDNPLIDVSRNKAL
HGGNFQGTPIGVSMDNTRLAIAAIGKLMFAQFSELVNDFYNNGLPSNLSGGRNPSLDYGFKGAEIAMASYCSELQFLANP
VTNHVQSAEQHNQDVNSLGLISSRKTAEAVDILKLMSSTFLVALCQAIDLRHLEENLKHTVKDTVSQVARKVLTVGANGE
LAPSRECEKDLLKAADREHVEAYIDDPCSATYPLMQKLRQVEVEHALNNGENEKNANSSIFQKIAAFEEELKAVLPKEVE
NARQTVENGNPTIPNRIKECRSYPLYRLVREELGTNFLTGEKVTSPGEKFDKVETAMCQGKIIDPMLECLREWNGAPLPI
C
>ESR41998.1 hypothetical protein CICLE_v10011134mg [Citrus clementina]
MATLGEPLVDLLSFTPTHYSSSWGIFCDCSNIYAKDMDRGAVIENGHQNGCLEGLCKDNNYSSGDALNWGVMAETLKGSH
LEEVKRMVAEYRKPVVNLGGETLTVAQVAAIATAGDVNAQVKVELSESAREGVKASSDWVMESMNKGTDSYGVTTGFGAT
SHRRTKNGGALQKELIRFLNAGIFGNGTESSHTLPHSATRAAMLVRVNTLLQGYSGIRFEILDAITKLENHSITPCLPLR
GTITASGDLVPLSYIAGLLTGRANSKATGPNGEIIDAQEASKQAGFGFFELQPKEGLALVNGTAVGSGLASMVLFDANNL
ALLSEILSAIFAEVMQGKPEFTDHLTHKLKHHPGQIEAAAIMEHILDGSSYVKAAKKLHEIDPLQKPKQDRYALRTSPQW
LGPQIEVIRFATKSIEREINSVNDNPLIDVSRNKALHGGNFQGTPIGVSMDNTRLAIAAIGKIMFAQFSELVNDFYNNGL
PSNLSGGRNPSLDYGFKGAEIAMASYCSELQFLANPVTNHVQSAEQHNQDVNSLGLISSRKTAEAVDILKLMSSTFLVAL
CQAIDERHLEENLKHTVKDTVSQVARKVLTVGANGELHPSRFCEKDLLKAADREHVFAYIDDPCSATYPLMQKLRQVLVE
HALNNGENEKNANSSIFQKIAAFEEELKAVLPKEVENARQTVENGNPTIPNRIKECRSYPLYRLVREELGTNFLTGEKVT
SPGEKFDKVETAMCQGKIIDPMLECLREWNGAPLPIC
>CAB42793.1 phenylalanine-ammonia lyase [Citrus clementina x Citrus reticulata]
MDRGAVIENGHQNGCLEGLCKDNNYSSGDALNWGVMAETLKGSHLEEVKRMVAEYRKPVVNLGGETLTVAQVAAIATAGD
VNAQVKVELSESAREGVKASSDWVMDSMNKGTDSYGVTTGFGATSHRRTQNGGALQKELIKELNAGIFGNGTKSSHTLPH
SATRAAMLVRVNTLLQGYSGIRFEILDAITKLLNHSITPCLPLRGTITASGDLVPLSYIAGLLTGRPNSKATGPNGETID
AQEASKQAGFGFFELQPKEGLALVNGTAVGSGLASMVLFDANNLALLSEILSAIFAEVMQGKPEFTDHLTHKLKHHPGQI
EAAAIMEHILDGSSYVKAAKKLHEIDPLQKPKQDRYALRTSPQWLGPQIEVIRFATKSIEREINSVNDNPLIDVSRNKAL
HGGNFQGTPIGVSMDNTRLAIAAIGKLMFAQFSELVNDFYNNGLPSNLSGGRNPSLDYGFKGAEIAMASYCSELQFLANP
VINHVQSAEQHNQDVNSLGLISSRKTAEAVDILKLMSSTELVALCQAIDLRHLEENLKHTVKDTVSQVARKVLTVGANGE
LHPSRFCEKDLLKAADREHVEAYIDDPCSATYPLMQKLRQVLVEHALNNGENEKNANSSIFQKIAAFEEELKAVLPKEVE
NARQTVENGNPTIPNRIKECRSYPLYRLVREELGTNFLTGEKVTSPGEKFDKVFTAMCQGKIIDPMLECEREWNGAPLPI
C
>AKA60049.1 phenylalanine ammonia-lyase [Citrus reticulata]
MDRGAVIENGHQNGCLEGLCKDNNYSSGDALNWGVMAETLKGSHLEEVKRMVAEYRKPVVNLGGETLTVAQVAAIATAGD
VNAQVKVELSESAREGVKASSDWVMESMNKGTDSYGVTTGFGATSHRRTKNGGALQKELIRYVFFYIFGNGTESSHTLPH
SATRAAMLVRVNTLLQGYSGIRFEILDAITKLLNHSITPCLPLRGTITASGDLVPLSYIAGLLTGRANSKATGPNGEIID
AQEASKQAGFGFFELQPKEGLALVNGTAVGSGLASMVLFDANNLALLSEILSAIFAEVMQGKPEFTDHLTHKLKHHPGQI
EAAAIMEHILDGSSYVKAAKKLHEIDPLQKPKQDRYALRTSPQWLGPQIEVIRFATKSIEREINSVNDNPLIDVSRNKAL
HGGNFQGTPIGVSMDNTRLAIAAIGKLMFAQFSELVNDFYNNGLPSNLSGGRNPSLDYGFKGAEIAMASYCSELQFLANP
VTNHVQSAEQHNQDVNSLGLISSRKTAEAVDILKLMSSTFLVALCQAIDLRHLEENLKHTVKDTVSQVARKVLTVGANGE
LHPSRECEKDLLKAADREHVFAYIDDPCSATYPLMQKLRQVLVEHALNNGENEKNANSSIFQKIAAFEEELKAVLPKEVE
NAGQTVENGNPTIPNRIKECRSYPLYRLVREELGTNFLTGEKVTSPGEKEDKVFTAMCQGKIIDPMLECLREWNGAPLPI
C
>XP_006436446.1 phenylalanine ammonia-lyase [Citrus clementina]
MELSHETCNGINNDRNGGTSSLGLCTGTDPLNWTVAADSLKGSHLDEVKRMVDEYRRPVVKLGGESLTIGQVTAIAAHDS
GVKVELAEAARAGVKASSDWVMDSMMKGTDSYGVTTGFGATSHRRTKQGGALQKELIRFLNSGIFGNGTESSHTLPHSAT
RAAMLVRVNTLLQGYSGIRFEILETITKELNHNITPCLPLRGTITASGDLVPLSYIAGLLTGRENSKAVGPNGQVLNPTE
AFNLAGVTSGFFELQPKEGLALVNGTAVGSGLAATVLFEANILAIMSEVLSAIFAEVMNGKPEFTDHLTHKLKHHPGQIE
AAAIMEHILDGSSYVKAAQKLHEIDPLQKPKQDRYALRTSPQWLGPQIEVIRAATKMIEREINSVNDNPLIDVSRNKALH
GGNFQGTPIGVSMDNTRLAIASIGKLLFAQFSELVNDFYNNGLPSNLTGGRNPSLDYGFKGAEIAMASYCSELQFLANPV
TNHVQSAEQHNQDVNSLGLISSRKTAEAVDILKLMSSTELVALCQAIDLRHLEENLKNTVKNTVSQVAKRVLTMGVNGEL
HPSRFCEKDLIKVVDREYVEAYIDDPCSASYPLMQKERQVLVDHALDNGDREKNSTTSIFQKIGAFEDELKTLLPKEVEI
ARTELESGNAAIANRIKECRSYPLYKIVREEIGTSLLTGEKVRSPGEEFDKVFAAMCEGKLIDPMLECLKEWNGAPLPIC
QN
>KDO46246.1 hypothetical protein CISIN_19004955mg [Citrus sinensis]
MELSHETCNGIKNDRNGGTSSLGLCTGTDPLNWTVAADSLKGSHLDEVKRMIDEYRRPVVKLGGESLTIGQVTAIAAHDS
GVKVELAEAARAGVKASSDWVMDSMMKGTDSYGVTTGFGATSHRRTKQGGALQKELIRFLNSGIFGNGTESSHTLPHSAT
RAAMLVRVNTLLQGYSGIRFEILETITKFLNHNITPCLPLRGTITASGDLVPLSYIAGLLTGRPNSKAVGPNGQVINPTE
AFNLAGVTSGFFELQPKEGLALVNGTAVGSGLAATVLFEANILAIMSEVLSAIFAEVMNGKPEFTDHLTHKLKHHPGQIE
AAAIMEHILDGSSYVKAAQKLHEIDPLQKPKQDRYALRTSPQWLGPQIEVIRAATKMIEREINSVNDNPLIDVSRNKALH
GGNFQGTPIGVSMDNTRLAIASIGKLLFAQFSELVNDFYNNGLPSNLTGGRNPSLDYGFKGAEIAMASYCSELQFLANPV
TNHVQSAEQHNQDVNSLGLISSRKTAEAVDILKLMSSTFLVALCQAIDLRHLEENLKNTVKNTVSQVAKRVLTMGVNGEL
HPSRECEKDLIKVVDREYVFAYIDDPCSASYPLMQKLRQVLVDHALDNGDREKNSTTSIFQKIGAFEDELKTLLPKEVEI
ARTELESGNAAIANRIKECRSYPLYKIVREEIGTSLETGEKVRSPGEEFDKVFAAMCEGKLIDPMLECLKEWNGAPLPIC
QN
>Q42667.1 RecName: Full = Phenylalanine ammonia-lyase [Citrus limon]
MELSHETCNGIKNDRNGGTSSLGLCTGTDPLNWTVAADSLKGSHLDEVKRMIDEYRRPVVKLGGESLTIGQVTAIAAHDS
GVKVELAEAARAGVKASSDWVMDSMMKGTDSYGVTTGFGATSHRRTKQGGALQKELIRFLNSGIFGNGTESSHTLPHSAT
RAAMLVRVNTLLQGYSGIRFEILETITKELNHNITPCLPLRGTITASGDLVPLSYIAGLLTGRENSKAVGSNGQVLNPTE
AFNLAGVTSGFFELQPKEGLALVNGTAVGSGLAATVLFEANILAIMSEVLSAIFAEVMNGKPEFTDHLTHKLKHHPGQIE
AAAIMEHILDGSSYVKAAQKLHETDPLQKPKQDRYALRTSPQWLGPQIEVIRAATKMIEREINSVNDNPLIDVSRNKALH
GGNFQGTPIGVSMDNTRLAIASIGKLMFAQESELVNDFYNNGLPSNLTGGRNPSLDYGEKGAEIAMASYCSELQFLANPV
TNHVQSAEQHNQDVNSLGLNSSRKTAEAVDILKLMSSTFLVALCQAIDERHLEENLKNTVKNTVSQVAKRVETMGVNGEL
HPSRFCEKDLIKVVDREYVFAYIDDPCSASSPLMQKLRQVLVDHALDNGDREKNSTTSIFQKIGAFEDELKTLLPKEVEI
ARTELESGNAAIPNRIKECRSYPLYKIVREDIGTSLLTGEKVRSPGEEFDKVFTAMCEGKLIDPMLECLKEWNGAPLPIC
QN
>XP_006424540.1 phenylalanine ammonia-lyase [Citrus clementina]
MEASHENQSGGNIPSGKLCTNIDPLNWVSASESLKGSHLDEVKRMVSEYRKPVVRLGGETLTIAQVAAVASRDDGVTVEL
NEEARAGVKASSDWVMESMNNGTDSYGVTTGFGATSHRRTKQGAALQKELIRFLNAGIFGKGTESCHMLPHTATRAAMLV
RINTLLQGYSGIRFEILEAITKFLNRNITPCLPLRASITASGDLVPFSYIAGLLTGRPNSVAVGPNGESLNAAEAFSQAG
IDGGFFELQPKEGLALVNGTGVGAGLASIVLFEANILTVLSEVISAIFAEAMHGKPEFTDHLTHKLKHHPGQIEAAAIME
HILDGSSYVKAAQKLHEIDPLQKPKQDRYALRTSPQWLGPQAEVIRASTKSIEREINSVNDNPLIDVSRNKALHGGNFQG
TPIGVSMDNSRLAIASIGKLMFAQFSELVNDFYSNGLPSNLSGGRNPCLDYGFKGAEIAMAAYCSELQFLANPVTNHVQS
AEQHNQDVNSLGLISARKTAEAVDILKLMSSTYLIALCQAIDLRHLEENLKSTVKSTISQVAKKVLTMGVNGELHPSRFC
EKDLLKVVDREYVESYADDPCSATYPLMQKLRQVLVDHALINNEDLKNANASIFLKIGAFEEELKTLLPKEVESARSAFE
SGNLEIPNRIKECRSYPLYRFVREELGARYLTGEKAISPGEECDKVFTAICQGKIIDPLLECLKEWDGSPLPIC
>XP_006424538.1 phenylalanine ammonia-lyase 1 [Citrus clementina]
MEASHENQSGGNIPSGKLCTNIDPLNWVSASESLKGSHLDEVKRMVSEYRKPVVRLGGETLTIAQVAAVASRDDGVTVEL
NEEARAGVKASSDWVMESMNNGTDSYGVTTGFGATSHRRTKQGAALQKELIRFLNAGIFGKGTESCHMLPHTATRAAMLV
RINTLLQGYSGIRFEILEAITKFLNRKITPCLPLRASITASGDLVPFSYIAGLLTGRPNSVAVGPNGESLNAAEAFSQAG
IDGGFFELQPKEGLALVNGTGVGAGLASIVLFEANILTVLSEVISAIFAEAMQGKPEFTDHLTHKLKHHPGQIEAAAIME
HILDGSSYVEAAQKFHEIDPLQKPKQDRYALRTSPQWLGPQAEVIRASTKSIEREINSVNDNPLIDVSRNKALHGGNFQG
TPIGVSMDNSRLAIASIGKLMFAQFSELVNDFYSNGLPSNLSGGRNPCLDYGFKGAEIAMAAYCSELQFLANPVTNHVQS
AEQHNQDVNSLGLISARKTAEAVDILKLMSSTYLIALCQAIDLRHLEENLKSTVKNTISQVAKKVLTMGVNGELHPSRFC
EKDLLKVVDREYVFSYADDPCSATYPLMRKLRQVLVDHALTNNEDLKNANASIFLKIGAFEEELKTLLPKEVESARSAFE
SGNLEIPNRIKECRSYPLYREVREELGARYLTGEKAISPGEECDKVFTAICQGKIIDPLLECLKEWDGSPLPIC
>XP_006488063.1 phenylalanine ammonia-lyase-like [Citrus sinensis]
MEASHENQSGGNIPSGKLCTNIDPLNWVSASESLKGSHLDEVKRMVSEYRKQVVRLGGETLTIAQVAAVASRDGGVTVEL
NEEARAGVKASSDWVMESMNKGTDSYGITTGFGATSHRRTKQGAALQKELIRFLNAGIEGKGTESCQMLPHTATRAAMLV
RINTLLQGYSGIRFEILEAITKFLNRNITPCLPLRASITASGDLIPFSYIAGLLTGRINSVAVGPNGESLNAAEAFSQAG
IDGGFFELQPKEGLALVNGTGVGAGLASIVLFEANILTVLSEVLSAIFAEAMLGKPEFTDHLTHKLKHHPGQIEAAAIME
HILDGSSYVKAAQKLHEIDPLQKPKQDRYALRTSPQWLGPQAEVIRASTKSIEREINSVNDNPLIDVSRNKALHGGNEQG
TPIGVSMDNSRLAIASIGKLMFAQFSELVNDFYSNGLPSNLSGGRNPSLDYGFKGAEIAMAAYCSELQFLANPVTNHVQS
AEQHNQDVNSLGLISARKTAEAVDILKLMSSTYLIALCQAIDLRHLEENLKSTVKNTISQVVKKVLTMGVNGELHPSREC
EKDLLKVVDREYVFSYADDPCSATYPLMQKLRQVLVDHALTNNEDLKNANASIFLKIGAFEEELKTLLPKEVESARSAFE
SGNLEIPNRIKECRSYPLYRFVREELGARYLTGEKAISPGEECDKVFTAICQGKIIDELLECLKEWDGSPLPIC
>KDO50672.1 hypothetical protein CISIN_1g037382mg [Citrus sinensis]
MLVRVNTLLQGYSGIRFEILEAITKLLNHSITPCLPLRGTITASGDLVPLSYIAGLLTGRPNSKATGPNGETIDAQEASK
QAGFGFFELQPKEGLALVNGTAVGSGLASMVLEDANNLALLSEILSAIFAEVMQGKPEFTDHLTHKLKHHPGQIEAAAIM
EHILDGSSYVKAAKKLHEIDPLQKPKQDRYALRTSPQWLGPQIEVIRFATKSIEREINSVNDNPLIDVSRNKALHGGNFQ
GTPIGVSMDNTRLAIAAIGKLMFAQFSELVNDFYNNGLPSNLSGGRNPSLDYGFKGAEIAMASYCSELQFLANPVTNHVQ
SAEQHNQDVNSLGLISSRKTAEAVDILKLMSSTELVALCQAIDERHLEENLKHTVKDTVSQVARKVLTVGANGELHPSRE
CEKDLLKAADREHVFAYIDDPCSATYPLMQKLRQVLVEHALNNGENEKNANSSIFQKIAAFEEELKAVLPKEVENARQTV
ENGNPTIPNRIKECRSYPLYRLVREELGTNFLTGEKVTSPGEEFDKVFTAMCQGKIIDPMLECLWEWNGAPLPIC
>GAY59766.1 hypothetical protein CUMW_197020, partial [Citrus unshiu]
MEPKEGLALVNGTGVGAGLASIVLFEANILTVLSEVISAIFAEAMHGKPEFTDHLTHKLKHHPGQIEAAAIMEHILDGSS
YVEAAQKFHEIDPLQKPKQDRGGNIPSGKLCTNIDPLNWVSASESLKGSHLDEVKRMRVPEAGGEARRRDPDDSSGGAVA
RVSRDGGVTVELNEEARAGVKASSDWVMESMNKGTDSYGITTGFGATSHRRTKQGAALQKELIRFLNAGIFGKGTESCQM
LPHTATRAAMLVRINTLLQGYSGIRFEILEAITKELNRNITPCLPLRASITASGDLIPFSYIAGLLTGRENSVAVGPNGE
SLNAAEAFSQAGIDGGFFELQPKEGLALVNGTGVGAGLASIVLFEANILTVLSEVLSAIFAEAMLGKPEFTDHLTHKLKH
HPGQIEAAAIMEHILDGSSYVKAAQKLHEIDPLQKPKQDRYALRTSPQWLGPQAEVIRASTKSIEREINSVNDNPLIDVS
RNKALHGGNFQGTPIGVSMDNSRLAIASIGKLMFAQFSELVNDFYSNGLPSNLSGGRNPSLDYGFKGAEIAMAAYCSELQ
FLANPVINHVQSAEQHNQDVNSLGLISARKTAEAVDILKLMSSTYLIALCQAIDERHLEENLKSTVKNTISQVVKKVLTM
GVNGELHPSRECEKDLLKVVDREYVESYADDPCSATYPLMQKLRQVLVDHALINNEDLKNANASIFLKWIKECRSYHCIG
WGSVSDRSEGDYHQARNVIRCLQRFVGKILIQ
WRKY70 protein sequences
>PtWRKY70_Ptrif.0006s1042.1.v1.3.1_Poncirus_trifoliata
MEAGQATSSSSWLENSSVSSDRRRAIEELIKGQEMALQLRNLIHKSTKSG
EGSKGMIINQDLVANILSSFTNSLSILKNGDSDEASQVQEHTQLSSPCWE
AYLKTEDSGESSKSSTVKDRRGCYKRRKCAESWTEHSSTLTDDGEAWRKY
GQKVILNAKFPRNYFRCTHKEDQGCLASKQVQKIQEEPPVHRTTYYGRHT
CKSLIKSSQLMLDSTTSDQCPMISFGSAHITENDENPESSSFPSIKQESN
KDDQAPLSDMTRNQSSSSDEYIVSPDFPAFESNEHMKVLSADHGDVISGV
NSSCTASAHSLDLAVDMSVNFDDVLEFNFDA-
>ESR42836.1 hypothetical protein CICLE_v10012055mg [Citrus clementina]
MLLTYLKASICLESFATPTWKKRGKKIIMKMEAGQATSSSSWLENSSDRRRAIEELIKGQEMALQLRNLIHKSTKSGEGS
KAMIINQDLVANILSSFTNSLSILKNGDSDEASQVQEHTQLSSPCWEAYLKTEDSGESSKSSTVKDRRGCYKRRKCAESW
TEHSSTLTDDGHAWRKYGQKVILNARFPRNYFRCTHKFDQGCQASKQVQRIQEEPPLHRTTYYGRHTCKSLIKSSQLMLD
STTSDQCPMISFGSAHITEKDENPFLSSEPSIKQESNKDDQGPLSDMTHNQSSSSDEYLVSHDFPAFESNEHMKVLSSDH
GDVISGVNSSCTASAHSLDLAVDMSVNFDDVLEENE
>GAY32270.1 hypothetical protein CUMW_001510 [Citrus unshiu]
MLLTYLKASICLESFATPTWKKRGKKIIMKMEAGQATSSSSWLENSSDRRRAIEELIKGQEMALQLRNLIHKSTKSGEGS
KAMIINQDLVANILSSETNSLSILKNGDSDEASQVQEHTQLSSPCWEAYLKTEDSGESSKSSTVKDRRGCYKRRKCAESW
TEHSSTLTDDGHAWRKYGQKVILNARFPRNYFRCTHKEDQGCQASKQVQRIQEEPPLYRTTYYGRHTCKSLIKSSQLMLD
STTSDQCPMISFGSAHITEKDENPFLSSEPSIKQESNKDDQAPLSDMTHNQSSSSDEYLVSHDFPAFESNEHMKVLSSDH
GDVISGVNSSCTASAHSLDLAVDMSVNEDDVLEFNE
>XP_006429596.2 probable WRKY transcription factor 70 [Citrus clementina]
MKMEAGQATSSSSWLENSSDRRRAIEELIKGQEMALQLRNLIHKSTKSGEGSKAMIINQDLVANILSSETNSLSILKNGD
SDEASQVQEHTQLSSPCWEAYLKTEDSGESSKSSTVKDRRGCYKRRKCAESWTEHSSTLTDDGHAWRKYGQKVILNARFP
RNYFRCTHKEDQGCQASKQVQRIQEEPPLHRTTYYGRHTCKSLIKSSQLMLDSTTSDQCPMISFGSAHITEKDENPELSS
FPSIKQESNKDDQGPLSDMTHNQSSSSDEYLVSHDFPAFESNEHMKVLSSDHGDVISGVNSSCTASAHSLDLAVDMSVNF
DDVLEENE
>KDO64116. 1 hypothetical protein CISIN_1g020291mg [Citrus sinensis]
MKMEAGQATSSSSWLENSSDRRRAIEELIKGQEMALQLRNLIHTSTKKGEGSKAMIINQDLVANILSSFTNSLSILKNGD
SDEASQVQEHTQLSSPCWEAYLKTEDSGESSKSSTVKDRRGCYKRRKCAESWTEHSSTLTDDGHAWRKYGQKVILNAREP
RNYFRCTHKEDQGCQASKQVQRIQEEPPLHRTTYYGRHTCKSLIKSSQLMLDSTTSDQCPMISFGSAHITEKDENPFLSS
FPSIKQESNKDDQGPLSDMTHNQSSSSDEYLVSHDFPAFESNEHMKVLSSDHGDVISGVNSSCTASAHSLDLAVDMSVNF
DDVLEENE
>ANA95961.1 WRKY transcription factor [Citrus maxima]
MKMEAGQATSSSSWLENSSDRRRAIEELIKGQEMALQLRNLIHTSTKKGEGSKAMIINQDLVANILSSFINSLSILKNGD
SDEASQVQEHTQLSSPCWEAYLKTEDSGESSKSSTVKDRRGCYKRRKCAESWTEHSSTLTDDGHAWRKYGQKVILNARFP
RNYFRCTHKFDQGCQASKQVQRIQEEPPVYRTTYYGRHTCKSLIKSSQLMEDSTTSDQCPMISFGSAHITEKEFNPFLSS
FPSKKQESNKDDQAPLSDMTHNQSSSSDEYLVSPDFPAFESNEHMKVLSSDHGDVISGVNSSCTASAHSLDLAVDMSVNF
DDVLEENEDA
>AKA59519.1 WRKY70 [Citrus japonica]
MKMEAGQATSSSSWLENSSDRRRAIEELIKGQEMALQLRNLIHTSTKKGEGSKAMIINQDLVANILSLFTNSLSILKNGD
SDEASQVQEHTQLSSPCWEAYLKTEDSGESSKSSTVKDRRGCYKRRKCAESWTEHSSTLTDDGFAWRKYGQKVILNSKEP
RNYFRCTHKEDQGCQASKQVQRIQGEPPLYRTTYYGRATCKSLIKSSQLMPDSTTSDQCPMISFGSAHITEKDENPELSS
FPSIKQESNKDDQAPLSDMTHNQSSSSDEYLVSHDFPAFESNEHMKVLSSDHGDVISGVNSSCTASAHSLDLAVDMSVNE
DDVLEFNE
EFR-like protein sequences
>PtEFR_Ptrif.0007s1284.1.v1.3.1_Poncirus_trifoliata
MCSNLYIINKALTACSLSILHEWLFLRVNTWEIPLEIGNLQNLEELSLGL
NKLIGTVPVAIFNVSTLKILELGGNSLSGSLSSIADVRLPNLEVLLLWGN
NESGTIPRFIFNASKLSILDLQYNSESSFIPNTEGNLRNLEGLYLQDNYL
TSSTPELSFLSSLSNCKSLTAIGLSNNPLDGILPKTSIGNHSYSLEDLEM
HNCNVSGGIPEEIGNETNLITIDLGGNKLNGSILITLSKLQKLQGLVLDD
NKLEGSIPDDICRLVELYKLELGGNKLSGSIPACESNLIALRILSLGSNE
LTSIPLTEWNLKDILYLNFSSNCFTGPLPLEIGNLKVLVGIDESMNNFSG
IIPKEIGGLNYLEYLELGYNRLQGSIPDSEGNLISLKELNLSNNNLSGAI
PASLEKLSYLEDINLSENKLEGEIPWGGSEGNESVESFEGNELLCGSPNL
QVPPCKTSIHHTSWKTSLLLGIVLPLSTTLMIVVIWLIWLAHMYRATDGE
SENNLIGRGGFGSVYKARLGDGMEVAVKVENLQCRRAFKSFDVECEIMKS
IRHRNLIKVISSCSNEDFKGLVLEYMPQGSLEKHLYSSNCILDIFQRLNI
MIDVASALEYLHFGCSTPVIHCDLKPSNVLLDDNMVAYLSDFGIAKLLIG
EDQSMTQTQTLATIGYMAPEYGREGRVSTNGDVYSFGIMLMETFTGKKPT
DEIFNGEMTLKHWVNDWLPISTMEVVDANLLSQEDVHFVAKEQCVSEVEN
LAMACTVESHEQRINAKEIVTKLLKIRDSLLRNVGGIRIRQPNLN-
>ESR49607.1 hypothetical protein CICLE_v10033608mg [Citrus clementina]
MLREHAVAGEQAEDMLQVLGVPTAGLEDTCQDLGGGERGVGPNLPDGIRYMETDGSGEIPLEIGNLQNLEVLDLGQNKLI
GTVPAAIFNVSTLKFIELQDNSLSGSLSSITDVRLPNLEKLHLWELSFLSSLSNCKSETLIDLSNNPLDGILPKTSIGNL
SHSLKDFKMHNCNVSGGIPEEISRLTNLTTIDLGGNKLNGSIPITLSKLQKLQGLGLEDNKLEGSIPDSICRLTELYDLE
LGGNKLFGSIPACFSNLASERILSLGSNELTSIPLTFWNINDILYLNFSSKFFTAPLPLEIGNLKVLVGMDFSMNNESGV
IPTKIGGLNNLEYLFLGYNKLQGSILDSFGDLISLKSENLSNNNLSGAIPASLEKLSYLEDENLSENKLEGEILMGGSEG
NFSAESFEGNELLCGSPNLQVPPCKTSIHRTSWKNSLLLGIVLPLSTIEMIVVILLGDEMEVAVKVENLQCGRAFKSFDV
ECEIMKSIRHRNLIKVISSCSNEELNIMIDVASALEYLHFGHSAPIIHCDLKPSNVLLDDNMVAHLSDESITKLLTREDQ
SMTQTQTFATIVYMAPEYGREGRVSANGDVYSFGDYVNGNFYWGRTHRWRGGSGAMTLKQWVVDVNLLSQEDVHFVAKEQ
CVSFVENLAMACTVESPEQRINAKEIVTMLLKIRGSLLRNCDLNY
>GAY66412.1 hypothetical protein CUMW_248560, partial [Citrus unshiu]
MIFSKLDRATARSSPRAGPPLLRMMSRFLLLHCLILISLFIAAATANTSSTITDRDALLALKAHITHDPTNFLAKNWNTS
TPVCNWTGVVCDVHSHRVTVENISSLNLTGTIPSQLGNLSSLQSLNLSCNRLSGSIPSAIFTIYTLKYVSFRENQVSGQI
PANICSNLPFLDYLSLAKNMEHGGIPSALSNCTYLQILHLSYNDESGAVPKDIGNLSKLKELYLGRNRLQGEIPREEGNL
TELEQMSLSENELQEHAVAGEQAEDMLQVLGVPTAGLEDTCQDLGGGERGVGPNLPDGIRYMETDGSGEIPLEIGNLQNI
EVLDLGQNKLIGTVPAAIFNVSTLKFIELQDNSLSGSLSSITDVRLPNLEKLHLWELSFLSSLSNCKSLTLIDLSNNPLD
GILPKTSIGNLSHSLKDFKMHNCNVSGGIPEEISRLTNLITIDLGGNKINGSIPITLSKLQKLQGLGLEDNKLEGSIPDS
ICRLTELYDLELGGNKLFGSIPACFSNLASLRILSLGSNELTSIPLTFWNINDILYLNESSNFFTAPLPLEIGNLKVLVG
MDFSMNNESGVIPTKIGGLKNLEYLFLGYNKLQGSILDSEGDLISLKSINLSNNNLSGAIPASLEKLSYLEDINLSENKL
EGEILMGGSEGNESAESFEGNELLCGSPNLQVPPCKTSIHRTSWKNSLLLGIVLPLSTIFMIVARLGDEMEVAVKVENLQ
CGRAFKSEDVECEIMKSIRHRNLIKVISSCSNEELNIMIDVASALEYLHFGHSAPIIHCDLKPSNVLLDDNMVAHLSDES
IAKLLTGEDQSMTQTQTFATIGYMAPEYGREGRVSANGDVYSEGIMLMETFTGKKPTDEIFNEEMTLKQWEDVHFVAKEQ
CVSFVFNLAMACTVESPEQRINAKEIVTKLLKIRGSLLRNVGGR
>GAY66414.1 hypothetical protein CUMW_248560 [Citrus unshiu]
MIFSKLDRATARSSPRAGPPLLRMMSRFLLLHCLILISLFIAAATANTSSTITDRDALLALKAHITHDPINFLAKNWNTS
TPVCNWTGVVCDVHSHRVTVLNISSLNLTGTIPSQLGNLSSLQSLNLSCNRLSGSIPSAIFTIYTLKYVSFRENQVSGQI
PANICSNLPELDYLSLAKNMFHGGIPSALSNCTYLQILHLSYNDESGAVPKDIGNLSKLKELYLGRNRLQGEIPREFGNL
TELEQMSLSENELQGEIPLEIGNLQNLEVLDLGQNKLIGTVPAAIFNVSTIKFIELQDNSLSGSLSSITDVRLPNLEKLH
LWEFSKNSESGFIPNTEGNERNLQKLRLYDNYLTSLTPELSFLSSLSNCKSLTLIDLSNNPLDGILPKTSIGNLSHSLKD
FKMHNCNVSGGIPEEISRLTNLTTIDEGGNKLNGSIPITLSKLQKLQGLGLEDNKLEGSIPDSICRLTELYDLELGGNKL
FGSIPACESNLASLRILSLGSNELTSIPLTFWNLNDILYLNFSSNFFTAPLPLEIGNLKVLVGMDFSMNNFSGVIPTKIG
GLKNLEYLFLGYNKLQGSILDSEGDLISLKSLNLSNNNLSGAIPASLEKLSYLEDLNLSENKLEGEILMGGSFGNFSAES
FEGNELLCGSPNLQVPPCKTSIHRTSWKNSLLLGIVEPLSTIFMIVVILLILRY
>GAY66413.1 hypothetical protein CUMW_248550 [Citrus unshiu]
MIFSKLDRATARSSPRAGPPLLRMMSRFLLLHCLILISLFIAAATANTSSTITDRDALLALKAHITHDPTNFLAKNWNTS
TPVCNWTGVVCDVHSHRVTVLNISSLNLIGTIPSQLGNLSSLQSLNLSCNRLSGSIPSAIFTIYTLKYVSFRENQVSGQI
PANICSNLPFLDYLSLAKNMEHGGIPSALSNCTYLQILHLSYNDESGAVPKDIGNLSKLKELYLGRNRLQGEIPREEGNL
TELEQMSLSENELQEHAVAGEQAEDMLQVLGVPTAGLEDTCQDLGGGERGVGPNLPDGIRYMETDGSGEIPLEIGNLQNL
EVLDLGQNKLIGTVPAAIFNVSTLKFIELQDNSLSGSLSSITDVRLPNLEKLHLWGNNFSGTIPRFIFNASKLSILEFSK
NSFSGFIPNTFGNLRNLQKLRLYDNYLTSLTPELSFLSSLSNCKSLTLIDESNNPLDGILPKTSIGNLSHSLKDEKMANC
NVSGGIPEEISRLTNETTIDIGGNKLNGSIPITLSKLQKLQGLGLEDNKLEGSIPDSICRLTELYDLELGGNKLFGSIPA
CFSNLASLRILSEGSNELTSIPLTFWNLNDILYLNFSSNFFTAPLPLEIGNLKVLVGMDFSMNNESGVIPTKIGGLKNLE
YLFLGYNKLQGSILDSFGDLISLKSLNLSNNNLSGAIPASLEKLSYLEDLNLSENKLEGEILMGGSFGNFSAESFEGNEL
LCGSPNLQVPPCKTSIHRTSWKNSLLLGIVEPLSTIEMIVVILLILRY
>GAY68230.1 hypothetical protein CUMW_262510 [Citrus unshiu]
MCSNLYITNKALTVCSLPILHEWLFFCVREIPPEIGNLRNLEELDLGHNKLVGTVPAAIFNLSTLKEFSIPNNSLSGCLS
SIADVRLPNLEVINEWELSFLSSLSNCKSLTYIDLSYNPLDSILPRTSVGNLSHSLEDFEMNNCNVSGGIPEEISNLTNL
TTIDLGGNKLNGLIPITLSKLQKLQGLVLYDNKLEGSIPDDICRLAELYELELDGNKLSGSIPACLSNLISLRILSLGSN
ELTSIPLTEWNLEDILYLNESSNFLTGPLPLEIGKLKVLVGIDESMNNESGVIPTKIGGLKNLEYLFLGYNRLQGSIPDS
FGDLTSLKSLNLSNNNLSGTIPASLEKLSYLENLNLSENKLEGEIPRGGSFGKFSAESFKGNELLCGSPNLQVPPCKTSI
HHTSWKNSLLLGIVLPLTRLGDGMEVAVKVENLECGRAFKSFDVECDMMKSIRHRNLIKVISSCSNEELNIMIDVASALE
YLHFGYSAPVIHCDLKPSNVLLDDNMVAHLSDFSIAKLLTGEDQSMTQTQTLATIGYMAPEYGREGRVSANGDVYSFGIM
LMETFTGKKPTDEIFNEEMTLKHWEDIHFVAKEQCVSEVENLAMACTVESPEQRINAKEIVTKLLKIRDSLLRNVGGRES
LVLSRFIEVSSFLEGKGKCYLLLTDYEYENLTCETVISAI
>GAY68422.1 hypothetical protein CUMW_263980 [Citrus unshiu]
MERAHSLMMMSRFLLLHCLILISLFIAAATANTSSTITDQDALLALKAHITHDPINFLAKNWNTSTPVCNWTGVACEVHS
QRVTVLNISSLNETGTIPSQLGNLSSLQSLNLSENRLEGSIPSAIFTTYTLKYVCLRGNQLSGTFPSFISNKSSLQHLDL
SSNALSGEIRANICSNLPFLEYLAFFKNMLHGGIPSTLSNCTYLRTLDFSYNDFSEAIPKDIGNITNLKELYLGRNRLQG
EIPREFGNLPELELMSLAANNLQGKIPLKIGNLRNLEKLDIGDNKLVGIAPIAIFNVSTLKILGLQDNSLSGCLSSIGYA
RLPNLEILSLWELSFLSSLSNCKELKYFDLSYNPLYRILPRTIVGNLSHSLEEFKMSNCNISGGIPEEISNLTNLRTIYL
GGNKINGSILITLSKLQKLQDLGLKDNKLEGSIPYDICNLAELYRLDLDGNKLSGSIPACESNLTSLRIVSLGSNELTSI
PLTFWNLKDILNENESSNELTGSLPLEIGSLKVLVGIDLSRNNESGVIPTEIGGLKNLEYLFLGYNRLQGSIPNSEGDLI
SLKFLNLSNNNLSGVIPASLEKLSYLEDINLSFNQLEGKIPRGGSFGNESAQSFEGNELLCGSPNLQIPPCKTSIHHKSW
KKSILLGIVLPLSTTFMIVASLGDGMEVAVKVFTSQCGRAFKSFDVECEIMKSIRHRNLIKVISSCSNEELNIMIDVASA
LEYLHFGYSAPVIHCDLKPSNVLLDDNMVAHLSDFSIAKMLTGEDQSMIQTQTLATIGYMAPEYGREGRVSANGDVYSFG
IMLMETFTGKKPTDEIFNGEMTLKHWEDIHFVAKEQCVSFVENLAMECTMEFPKQRINAKEIVTKLLKIRDSLLRNVGGR
CVMKE
>GAY66431.1 hypothetical protein CUMW_248700, partial [Citrus unshiu]
TTANTITITIDQDALLALKAHITHDPTNFLAKNWNTSTPVCNWTGVTCDVHSHRVTVLNISRLNLTGTIPSQLGNLSSLQ
SLNLSENREFGSIPSAIFTIYTLKYVSFRENQLSGTFPSLIENKSSLQHLDFTHNTLSGEIPANICSNLPFLEYESLFQN
MFHGGIPSTLSNCTYLRILSLSSNDESGPIPKEIGNLTKLKELYLGRNRLHGEIPQEFGNLVKLELMSLPENKLQGEIPS
EIGNEHNLEYLDLSLNKLVGVVPSAIENVSTLKYLGLQNNSLSGSLSSILDFRIENLEELHLWELSFLSSLSNCKSIRLI
DLSNNSLDGILPRTSVGNLSHSLEYFDMSYCNVSGGIPEEINNLTNLITIYLAGNKLNGSIPITLSKLQKLQGLGLQDNK
LKGLIPEDICRLAKLYELNLGGNMLSGSIPACESNLASLRTLSLGENELTSIPSTFWNLKDILYLNFSSNFFAGPLPLKI
GNLKVLIEIDESMNNFSGVIPTTIGGLKNLQYLSIGNNRLQGSIPNSVGDLISLKSLNLSNNNLSGAIPVSLEKLTYLKD
LDLSENKLEGEIPNGGSFGNESAESFEGNQLLCGLENLHVPPCKTSIHHTSWKNALLLGTFLPVSTIFMIVVIIEDGMDV
AVKVENLEYGRAFKSFDVECEIMKSIRHRNLIKVISSCSNEELNIMIDVALALEYLHFGCSASVIHCDLKPSNVLLDDNM
VAHLSDFGIAKLLTGEDQSMIQTQTLATIGYMAPEYGREGRVSANGDVYSFGIMLMETFTGKKPTDKIENGEMTLTHWED
VHFAAKEQCMSFVENLAMECTAESPEQRINAKEIVTRLLKIKDSLLRNVGGLITLCNNSWGV
>GAY63066.1 hypothetical protein CUMW_222610, partial [Citrus unshiu]
MERVHSLSMISRELLLHCLVLIFLFIAAATANTSTITTDQDALLALKAHISHDPTNFLAKNWNKSTPICNWTGVTCDVHS
HRVTVLNISSLNLTGTVPAQLGNLSSLQSLDLSENRLSGFIPSTIFTMYTLKRVSFRENQLSGTFPSFIENKSSLQHLDF
SHNTLSGEIPANICSNLPFLEYISLSQNMFHGRIPPTLSNCTYLRILGLSLNNESGAIPKEISYLTKLKELYLGVNRLQG
EIPREVGNLAELELMSLPENKLQGEIPQELGNLVGLEFLFLSDNFLIGTIPKEISNETNLQDLGLDSNRLQGEIPPEIGN
LRSLEWLLLGYNKLVGTIPAAIFNVSTLKQLDLØNNSLSGSLSSIADVRLPNLEMIYMWELSFLSSLSNCKSLTHIRLSD
NPLNGILPRTTVGNLSHSLELFDMSYCNISGSIPKEISNLTNLTTIYLVGNKLNGLIPITLGKLQKLQSLVLEDNKLKGS
IPDDICRLAELYELNLGGNKLSGSIPACESNLASLRTLSLSSNELTSIPLTLWNLKDILYLNESSNFLSGPLPLEIENLK
VLVGIDFSMNNFSSVIPTTIGSLKDLQYLLLAYNKLQGSIPDSVGDLISLKSLNLSNNNLSGAIPVSLEKVSYLENLDLS
ENKLEGEIPKGGSFGNFSAESFEGNELLCGSPNLQVPPCKISIHHASRKNALLLGTALPLTRIQDGIEVAVKVENLQCGR
AFKSFDVECQVMKSIRHRNLIKVISSCSNEELNIMIDVASALEYLHFGYSTPVIHCDLKPNNVLLDNNMVAHLSDFGIAK
LLTGEDQFVTQTQTLATIGYMAPEYGREGRVSINGDVYSFGIMLMETFTGKKPTDKIENGEMTLKRWVVDANLLSREDIH
FVAKEQCLSFVENLAMDCTVE
>GAY67254.1 hypothetical protein CUMW_255090, partial [Citrus unshiu]
ATANTSTITADQDALLSLKAHITHDPTNFLAKNWNTSISFCNWTGVTCDVHSHRVTILNISGLNLTGTIPSQLGNLSSLQ
SINLSENQLSGSIPSAIFTTYTLTYVSLCQNQLSGKIPANICSNLPFLEFLSLSKNMLYGGIPSTLSNCTYLRILRAIPK
EIGNLTKLKELSLGGNRLQGEIPREFGNLADLEQMSLWENNERGEIPLEIGNLQNLEELDLRQNKLVGIVPASIENVSTL
KLLQLQNNSLLGCLSSIADVRLPNLEELSLWELVGNSFSGFIPNTFGNLRNLERLNLQDNYLTSSTPELSFLSSLSNCKS
LTLIALSNNPLDGNLRKTSVGNLSHSLEIFLMYNCNISGGISEEISNETNLTTINLGGNKLNGSIPIALGKLQKLQYLGL
EDNKLEGSIPDDICRLDELYELELGGNKLSGSIPACFGNLIALRILSLGSNELTSIPLTFWNIKDILQLNESSNYFTGPL
PLEIGNLKVLIGIDESMNNFSGVIPTEIGGLKNLEYLELGYNRLRGSIPDSEGDLISLKFLNLSNNNLSGAIPASLEKLS
YLEDINLSENKLEGEIPRGGSFGNFSAESFEGNELLCGSSNLQVPPCKTRIAHTSWKKSLLLGIVLPLSTTLMIVVIWLI
LRYRQRGKQPSNDANMPLVATWRTFSYLELCQATDEFSENNLIGRGGFGSFISP
>ESR40316.1 hypothetical protein CICLE_v100269281mg, partial [Citrus clementina]
GNKLSGSLPACESNLTSLRIVSLGSNKLTSVPLTFWNIKDILNINFSSNFLTSPLPLEIGNLKVLIGIDESMNNFSGVIP
TEIGGLKNLEYLELGYNRLQGSIPDSFGDLISLKELNLSNNNLSGAIPASLEKLSYLENLNLSENKLEGEIPRGGSEGNF
SAESFEGNELLCGSPNLQVPPCKTSIHHTSWKNSLLLRIVLPLSTTFMIVVILLILRYQQRGKLKVFNLQCGRAFESFDV
ECEMMKNIRHRNLIKVISSCSNEELNIMIDVASALEYLHFEYQSMTQTQTLATIGYMAPEYGREGRVSANGDVYSFGIML
METFTGKKPTDEIFNGEMTLKHWVNDWLLISTMEVVDANLLSQEDIHFVAKEQCVSFVENLAMACTVESPEQRINAKEIV
KKLLKIRDSLLRNVGGRFCE
>GAY68231.1 hypothetical protein CUMW_262510 [Citrus unshiu]
MCSNLYITNKALTVCSLPILHEWLFFCVREIPPEIGNLRNLEELDLGHNKLVGTVPAAIFNLSTLKEFSIPNNSLSGCLS
SIADVRLPNLEVINEWGNNESGTIPRFIFNASKLSALDLDGNSFSGFIPNTFGNLRNLKWLILSDNYLTSSTPELSELSS
LSNCKSLTYIDLSYNPLDSILPRTSVGNLSHSLEDFEMNNCNVSGGIPEEISNLTNLTTIDLGGNKLNGLIPITLSKLQK
LQGLVLYDNKLEGSIPDDICRLAELYELELDGNKLSGSIPACLSNLISLRILSLGSNELTSIPLTFWNLEDILYLNESSN
FLTGPLPLEIGKLKVLVGIDESMNNFSGVIPTKIGGLKNLEYLFLGYNRLQGSIPDSFGDLTSLKSLNLSNNNLSGTIPA
SLEKLSYLENLNLSENKLEGEIPRGGSFGKFSAESFKGNELLCGSPNLQVPPCKTSIHHTSWKNSLLLGIVLPLSTTEMI
VVILLILRYRQRGKQPSNDANMPLVENLECGRAFKSFDVECDMMKSIRHRNLIKVISSCSNEELNIMIDVASALEYLHFG
YSAPVIHCDLKPSNVLLDDNMVAHLSDFSIAKLLTGEDQSMTQTQTLATIGYMAPEYGREGRVSANGDVYSEGIMLMETE
TGKKPTDEIFNEEMTLKHWVNDWLPISTMEVVDVNLLSQEDIHFVAKEQCVSFVENLAMACTVESPEQRINAKEIVTKLL
KIRDSLLRNVGGRFSLVLSRFIEVSSFLEGKGKCYLLLTDYEYENLTCETVLSAI
>ESR63269.1 hypothetical protein CICLE_v10013944mg [Citrus clementina]
MYTLKYVNERGNQLSGAFPSFIENKSSLQDLDFSYNALSGEIPANICSNLPFLESISLSQNMFHGGIPSTLSNCKYLEIL
SLSINNLLGAIPKEIGNLTKLKELYLGYSGLQGEIPREFGNLAELELMALQVSNLQGEIPQELANLTGLEVLKLDKIFLT
GEIPPEIHNLHNLKLLDLSHNKLVGAVPATIFNMSTETGLGLQSNSLSGSLSSIADVQLPNLEELRLWSNNESGTIPRVI
FNASKLSVLELGINSFSGFIPNTFGNLRNLSELSSESNCKSLTYIGLSNNPLDGILPRMSMGNLSHSLEYFDLSYCNVSG
GFPEEIGNETNLIGIYLRGNKUNGSIPITLGKLQKLQGLHLEDNKLEGPIPDDICRLTKLYELGLSGNKLSGSIPACFSN
LASLGTLSLGSNKLTSIPLTIWNLKSMLYLNFSSNFFTGPLPLDIGNLKVLIGIDFSINNESDVIPTVIGGLINLQYLEL
GYNRLQGSISESEGDLISLKSLNLSNNNLSRSIPISLEKLSYLEDLDLSFNKLKGEIPKGGSFGNFSAKSFEGNELLCGS
PNLQVPPCKTSIHHKSRKNVLLLGIVLPLSTIFIIVVILLIVRYRKRVAVKVFDLQCGRAFKSFDVECEIMKSIRHRNLI
KVISSCSTEELNIMVDVATALEYLHEGYSAPVIHCDLKPNNVLLDDNMVAHLSDFGIAKLLIGEDQSMTQTQTLATIGYM
APDVYSEGIILMETEKGKKPTDEIFNEEMTLKHWVNDWLPISIMKVIDANLLSREDMHFVAKEQCVSFVENLAMECTVES
PQQRINAKKIVTRLLKIRDSLLRNVGATSLLYYRPNCFY
>ESR40314.1 hypothetical protein CICLE_v10025188mg [Citrus clementina]
MNSFSGFIPSTEGNLRNLEWLTLYDNNLTSSTLDLSFLSSLSNCKSLTHISLSNNPLDGILPRTYVGNLSHSLKNFYMYN
CNVSGGIPEEITNLTDLTTIVIGGNKINGSIPITLGKLQKLQDVDLEYNQLEGSIPDSICLSVELYELELGGNKLSGSIP
ACESNMTFLKVLSLGSNELTSIPLNFWSLKDILDLNLSSNCFSGPLPLEIRNLKALIEIDESMNNESGIIPMEIGSLKNL
ENLFLEYNRLEGSIPDSFGDLISLKSLNLSYNNLSGTIPVSLEKLSYLKDLNLSENKLKGEIPRGGSFGNFSAESFKGNE
LLCGSPNLQVPPCKASIHRTSRKNALILGIVLPESTIFMTAIILFIIKYQKREKGPPNDPNMPPVENLQCGRAFKSEDVE
CAMMKSIRHRNLVKVISSCSNEELNIMIDVASALEYLHEGYSAPVIHCDLKPSNVLLDDNMVAHLSDFGIAKLLTGEDQS
MTQTQTLATIGYMAPEYGREGQVSTNGDVYSFGIMLMETFTRKKPTDELENGEMTLKHWVNDCLPISTMEVVDANLLSQE
DIHFVAKEQCVSFVENLALECTVESPEQRINAKEIVAKLLKIRDSLLRNVY
>GAY63064.1 hypothetical protein CUMW_222610 [Citrus unshiu]
MERVHSLSMISRFLLLHCLVLIFLFIAAATANTSTITTDQDALLALKAHISHDPTNFLAKNWNKSTPICNWTGVTCDVHS
HRVTVLNISSINLTGTVPAQLGNLSSLQSLDLSENRLSGFIPSTIFTMYTLKRVSFRENQLSGTFPSFIENKSSLQHLDE
SHNTLSGEIPANICSNLPFLEYISLSQNMFHGRIPPTLSNCTYLRILGLSENNESGAIPKEISYLTKLKELYLGVNRLQG
EIPREVGNLAELELMSLPENKLQGEIPQELGNLVGLEFLELSDNFLTGTIPKEISNETNLQDLGLDSNRLQGEIPPEIGN
LRSLEWLLLGYNKLVGTIPAAIFNVSTLKQLDLQNNSLSGSLSSIADVRLPNLEMIYMWGNNESGTIPRFIFNASKLSIL
SLEKNSFSGFIPNTEGNLRNLEQLDLSDNYLTSSTPELSFLSSLSNCKSLTHIRLSDNPLNGILPRTTVGNLSHSLELED
MSYCNISGSIPKEISNLTNLTTIYLVGNKLNGLIPITLGKLQKLQSLVLEDNKLKGSIPDDICRLAELYELNLGGNKLSG
SIPACESNLASLRTLSLSSNELTSIPLTLWNLKDILYLNESSNELSGPLPLEIENLKVLVGIDFSMNNFSSVIPTTIGSL
KDLQYLLLAYNKLQGSIPDSVGDLISLKSINLSNNNLSGAIPVSLEKVSYLENLDLSENKLEGEIPKGGSEGNFSAESFE
GNELLCGSPNLQVPPCKISIHHASRKNALLLGTALPLSTIFMIVVILLILKCRKRRKRPSDDANIPPVENLQCGRAFKSF
DVECQVMKSIRHRNLIKVISSCSNEELNIMIDVASALEYLHFGYSTPVIHCDLKPNNVLLDNNMVAHLSDFGIAKLLTGE
DQFVTQTQTLATIGYMAPEYGREGRVSTNGDVYSFGIMLMETFTGKKPTDKIENGEMTLKRWICDWIPISIMEVVDANLL
SREDIHFVAKEQCLSFVENLAMDCTVECPEQRINAKEIVTRLLKIRDSLLRNVEGRCIRQSNLN
>XP_024047981.1 probable LRR receptor-like serine/threonine-protein kinase
At3g47570 [Citrus clementina]
MSTLTGLGLQSNSLSGSLSSIADVQLPNLEELREWSNNESGTIPRVIFNASKLSVLELGINSFSGFIPNTFGNLRNLRLL
TLHYNYLTSSNLELSFLSSFSNCKSLTYIGLSNNPLDGILPRMSMGNLSHSLEYFDLSYCNVSGGFPEEIGNLTNLIGIY
LRGNKLNGSIPITLGKLQKLQGLHLEDNKLEGPIPDDICRETKLYELGLSGNKLSGSIPACESNLASLGTLSLGSNKLTS
IPLTIWNLKSMLYLNFSSNFETGPLPLDIGNLKVLIGIDESTNNFSDVIPTVIGGETNLQYLELGYNRLQGSISESEGDL
ISLKSLNLSNNNLSRSIPISLEKLSYLEDLDLSFNKLKGEIPKGGSFGNFSAKSFEGNELLCGSPNLQVPPCKTSIHHKS
RKNVLLLGIVLPENRESENNLIGRGGFGSVYKARIGEGMEVAVKVEDLQCGRAFKSFDVECEIMKSIRHRNLIKVISSCS
TEEFKALVLEYMPHGSLEKNLYSSNCILDIFQRLNIMVDVATALEYLHFGYSAPVIHCDLKPNNVLLDDNMVAHLSDFGI
AKLLIGEDQSMTQTQTLATIGYMAPEYGRE
>ESR40317.1 hypothetical protein CICLE_v10025171mg [Citrus clementina]
MSNCNVSGGIPEEISNLTHETTIILGGNKLNGSIPITLGKLQKLQGLGLGDNKLEGSIPDDICRLAELYRLELGGNKLYG
SIPTCFGNLASLRILSLGSNKLTSIPLTEWNLKDILQLNESSNFLTGPLPLEIGNLKVLIVIDESMNNFSGVISTEIGGL
KNLEYLFLGYNRLRGSIPDSFGDLISLKSINLSNNNLSGAIPTSLEKLSYLEDLNLSENKLEGEIPRGGSFANFSAESFE
GNELLCGSPNLQVPPCKTSIHHTSWKNSLLLGIVLPLSTTLLIVVIWLILRYRQRGKQPSNDANMSLVATWRKFSYLELC
RATDGFSENNLIGRGGFGSVYKARLGNGMEVAVKVENLQCGRAFKSFDVECEMMKSIRHRNLIKVISSCSNEEFKALVLE
YMPHGSLEKYLYSSNCILDIFQRLNIMIDVASALEYLHFGCSALVIHCDLKPSNVLLGDNMVAHLSDEGIAKLLIGEDQS
MTQTQTLGTIGYMAPEYGREGRVSANGDVYSFGIMLMETFTGKKPTDEIENGEMTLKHWVNELLPISTMEVVDANLLRQE
DIHFAAKEQCVSFIENLAMACTVESPEQRINAKEIVKKLLKIRDSLLRNVGGICIRQSNLN
>XP_006465577.1 probable LRR receptor-like serine/threonine-protein kinase
At3g47570 isoform X5 [Citrus sinensis]
MERLHSLRMMSRELLLHCLILISLFIAAATANTSSTITDRDALLALKAHITHDPINFLAKNWNTSTPVCNWTGVACDVHS
HRVTVLNISSLNLTGTIPSQLGNLSSLQSLNLSCNRLEGSIPSAIFTIYTLKYVSLRENQVSGQIPANICSNLPELDYLS
LGKNMFHGGIPSALSNCTYLQILHLSYNDFSGAVPKDIGNLSKLKELYLGRNRLQGEIPREFVNLTELERMSLSENELQG
GIPRELGNLTKLEGLQLFRNNLTGGIPRELGNLTKLERLQLFWNNLIGAIPKEIGNLTKLKELSLDGNRLQGEIPLEISN
LQNLEELDLRHNKLVGTVPAAIFNMSMLKLLHLQNNSLLGCLSSIADVRLPNLEALLLWDNPLDGILSKTSIGNLSHSLK
DFYMSNCNVSGGIPEEITNLINSITIDLGGNKLNGSIPITLSKLQKLQGLGLDDNKLEGSIPDSICRLTELYELELGGNK
LEGSIPACESNLASLRILSLSSNELTSIPLTFWNLKDILQLNESSNFLTGPLPLEIGNLKVLIGIDFSMNNESSVIPTEI
GGLKNLEYLFLGYNRLEGSIPDSFGDLISLKFLNLSNNNLSGAIPTSLEKLSYLEDLNLSENKLEGEIPRGGSFGNFAAE
SFEGNELLCGSPTLQVLPCKTSIHHTSWKNSLLLGIVLPLSTTLLIVVIWLILRYRKRGKQPSNDANMPLVATWRTFSYL
ELCRATNGFSENNLIGRGGFGSVYKARLGDGMEVAVKVENLQCGRAFKSFAVECEMMKSIRHRNLIKVISSCSNEEFKAL
VLEYKPHGSLEKYLYSSNCILDIFQRLNIMIDVASALEYLHEGCSAPVIHCDLKPDNVLLDDNLVAYLSDEGIAKLLIGE
DQSMTQTQTLATIGYMAPEYGREGRVSTNGDVYSFGIMLMETFTGKKPTDEIFNGEMTLKHWVNDWLPISTMEVVDANLL
SQEDVHEVAKEQCVSFVFNLAMACTVESHEQRINAKEIVTKLLKIRDSLLRNVGGRRISQPNLN
>KDO36487.1 hypothetical protein CISIN_1g047705mg, partial [Citrus sinensis]
EIPLEIGNLQNLEELDLRQNKLIGTVPVAIFNVSTLKLLGLQDNSLSGSLSSITDVRLPNLEELVLWGNNFSELNELSSL
SNCKSLTVIGLSNNPLDGILPKTSIGNLSHSLEDFQMHNCNVTGDIPEEIGNLTNLITIDLGGNKLNGSILITLSKLQKL
QGLVLDDNKLEGSIPDDICREVELYKLELGGNKLSRSIPACENNLIALRILSLGSNDPLPLEIGNLKVLVGIDESMNNES
GIIPKEIGGLKNLEYLFLGYNRLQGLIPDSFGNLISLKFLNLSNNNLSGAIPASLEKLSYLEDLNLSFNKLEGEIPRGGS
FGNFSAESFEGNELLCGSPNLQVPPCKTSIHHPSWNISLLLGIVLPLSTTLMIVVIWLILRYRQRGKQPSNDANMPLVAM
WRTFSYLELCRATDGESENNLIGRGGFGSVYKARLGDGMEVAVKVENLQCRRAFKSFDVECEIMKSIRHRNLIKVISSCS
NEEFKGLVLEYMPQGSLEKHLYSTNCILDIFQRLNIMIDVASALEYLHFGCSTPVIHCDLKPSNVLLDDNMIAYLSDFGI
AKLLIGEDQSMTQTQTLATIGYMAP
>XP_024036868.1 probable LRR receptor-like serine/threonine-protein kinase
At3g47570 [Citrus clementina]
MSRFLLLHCLILISLFIAAAAANTSSTITDRDGLLALKAHITHDPINFLAKNWNTSTPVCNWTGVACDVHSHRVTVLNIS
SLNLTGTIPSQLGNLSSLQSLNLSENRLSGSIPSAIFTMYTLKNVSFHENQLSGQIPANICSSLPFLEFFSLSKNMFHGG
IPSTLSNCTYLRILSLAYNDESGAVPREIGEIPREFCNLTELEQMSLAGGIPRELGNLTKLERLQLFENNLTGALPKEIG
NLTKLKHLSLDHNRLQGEIPREFGNLAELELLSLYENKLQGEIPLEIGNLQNLEELGLGQNKLIGTVPVAIFNVSTLKEL
ELQDNSLSGSLSSIVDVRLPNLEKLLLWGNNFSGTIPHFIFNASKLSILELSQNSFADFIPNTEGNLRNLQRLKLYDNYL
TSSTPELSELFSLSNCKSLTHLSLSNNPLDGILPRTSVGNLSHSLKEFYMSNCNVSGGIPEEITNLTNLTTIFFGGNKLN
GSIPITLGKLQKLQGLGLEDNKLEGSIPDNICRLTELYELELGGNKLSGSIPACENNLASLRILSLGSNELNSIPLTEWN
LKDILQINCSSNFFTGPLPSEIGNLKVLVGIDESMNNFSGVIPTEIGGLKNLEYLELGYNRLQGSIPNSEGDLISLKSEN
LSNNNLSGVIPASLEKLSYLEDLNLSENKLEGEIPTGGSFGNFSAESFEGNELLCGSPNLQVPPCKTSIHRTSWKNSLLL
GIVLPLSTTFMMVVILLILRYRQRGKRPSNDASMPLVAMWRTFSYLELCQATDEFSENNLIGKGGFGSVYKARLGDGMEV
AVKVENLQCGRALKSENVECEMMKSIRHRNLIKVISSCSNEEFKALVLEYMPHGSLEKYLYSSNCILDIFQRLNIMIDVA
SALEYLHFGYSAPIIHCDLKPSNVLLDDNMVAHLSDESIAKLLVGEDQSMTQTQTFATIGYMAPEYGREGRVSANGDVYS
FGIMLMETFTGKKPTDEIENGEMTLKHWVNDWLPISTLEVVDANLLSQEDIHFVAKEQCVSEVENLAMACAVESPEQRIN
AKEIVKKLLKIRDSLLRNVGGRCIRQSNLN
>GAY68466.1 hypothetical protein CUMW_264350 [Citrus unshiu]
MSRFLLVHYLILISLLIASATANISTITPDRDALLAMKAHITHDPTNFFAKNWNTSISFCNWTGVTCDVASHRVTVLNIS
RINLIGTIPSQLGNLSSLQSINLSENRLSGSIPSAIFTTYTLKYVSFRENQLSGAFPSFIYNKSSLQHLDFSENTLSGEI
PASICSNLPFLEYISLSKNMFHGGIPSALSKCTYLRILSLSYNDLSGAVPKDIGNLSKLKELYLGRNRLQGEIPRGEGNE
TELELMSLSENELQGGIPQELGNLTKLEMLQLEWNNLTGEIPLEIGNLQNLEELELGQNKLIGTVPVAIFNVSTLKFLEI
QNNSLSGSLSSIADVRLPNLEELLLWGNNFSGTIPRFIFNASKLSILDLQDNSFSSFIPNTEGNLRNLEWLNLQDNYLTS
STPELSFLSSLSNCKSLRLIGLSNNPLDGILPKTSVGNLSLSLEDFKMANCNISGGIPEEISNLINLITIDLGGNKINGS
ILITLSKLQKLQGLDLDDNKLEGSISDDICRLAELYELELDGNKLSGSIPACFSNLIALRILSLGSNELTSIPSTEWNLK
DILYLNFSSNFLTGPLPLEIGNLKVLVGIDFSMNNFSGVIPTEIGGLKNLEYLFLGYNRLQGSIPDSFGNLISLKFLNLS
NNNLSGAIPASLEKLSYLEDLNLSENKLEGEIPRGGSFGNFSAESFEGNELLCGSPNLQVPPCKTSIHHTSWKISLLLGI
VLPLSATLMIVVIWLILRYRQRGKQPSNDANMPLVATWRTFSYLELCRATNEFSENNLLGRGGFGSVYKARLGDGMEVAV
KVENLQCRRAFKSENVECEIMKSIRHRNLIKVISSCSNEEFKGLVLEYMPHGSLEKHLYSSNCILDIFQRLNIMIDVASA
LEYLHEGCSTPVIHCDLKPSNVLLDDNMVAYLSDFGIAKLLIGEDQSMTQTQTLATIGYMAPEYGREGRVSTNGDVYSFG
IMLMETFTGKKPTDEIENGEMTLKHWVNDWLPISTMEVVDANLLSQEDVHFVAKEQCVSFVENLAMACIVESHEQRINAK
EIVTKLLKIRDSLLRNVGGRRIRQPNLN
>XP_024958339.1 probable LRR receptor-like serine/threonine-protein kinase
At3g47570 [Citrus sinensis]
MMSRFLLLHCLIIIFLFISAAAANTSSTITDREALLALKAHITHDPINFLAKNWNTSTPVCNWTGVTCDVHSHRVTVLNI
SSLNLTGTIPSQLGNLSSLQSLNLSENRLSGSIPSAIFTMYTLKNVTFRENQLSGQIPANICSSLPFLEFLSLSQNMFHG
GIPSTLSNCTYLRILSLSYNDFSGAVPREIGNSTKLKILYLGRNRLQGEIPREFCNLTELEHMSLAGNNLQGGIPQELGN
LAKLEMLQLFQNNLTGAIPKEIGNLTKLKELSLNANRLQGEIPREFGNLAELELMWLSENNLQGGIPRELGNLTKLEILH
LWKNNLTGAIPKEIGNLTKLKELPLYSNRLQGEIPREFGNLAELEMLSLYENKLQGEIPLEISNLQKLEDLGLGQNKLIG
IVPVAIFNVSTLKFLELQDNSLSGSLSSIVDVLPNLEKLLLWGNNFSGTIPHFIFNASKLSILELSQNSFAGFIPNTEGN
LRNLQRLKLYDNYLTSSTPELSFLFSLSNCKSLTHLSLSNNPLDGILQRTSVGNLSHSLKEFYMSNCNVSGGIPEEITNL
TNLTTIFFGGNKINGSIPITLGKLQKLQGLGLEDNKLEGSIPDNICRLTELYELELGGNKLSGSIPACENNLASLRILSL
GSNELTSIPLTEWNIKDILQLNCSSNFLTGQLPSEIGNLKVLVGIDESMNNFSGVIPTEIGGLKNLEYLFLGYNRLQGSI
PNSFGDLISLKSENLSNNNLSGAIPASLEKLSYLEDLNLSENKLEGEIPTGGSFGNFSAESFEGNELLCGSPNLQVSPCK
TSIHRTSWKKSLLLGIVLPLSTTEMIVVILLILRYRQRGKRPSNDASMPLVAMWRTFSYLELCRATDEFSENNLIGKGGF
GSVYKARLGDGMEVVVKVENLQCGRAFKSFDVECEMMKSIRHRNLIKVISSCSNEEFKALVLEYMPHGSLEKYLYSSNCI
LDIFQRENIMIDVASTLEYLYFGHSAPIIHCDLKPSNVLLDDNMVAHLSDFSIAKLLTGDDQSMTQTQTFATIGYMAPEY
GREGRVSANGDVYSFGIMLMETETGKKPTDEIFNEEMTLKQWVNDWLPISTMEVVDANLLSPEDVHFVAKEQCVSEVENL
AMACTVESPEQRINAKEIVTKLLKIRGSLLRNVGGRCIRQSNLN
>XP_015386042.1 probable LRR receptor-like serine/threonine-protein kinase
At3g47570 [Citrus sinensis]
MEKRHSLSIMSRELLLRCLILISLFIAAATANTSTITTDRDALLALKAHITHDPTNFFAKNWNTSTPVCNWTGVTCDVHS
HRVTVLNISRLNITGTIPSQLGNLSSLQSLNLSCNRLSGSIPSAIFTTYTLKYVSERKNQLSGAFPSFAENTSSLQYLDE
GENTLSGEIPANICSNLPFLEYLALSQNMFHGGIPSALSNCAYLQRLGLSSNDFSGVVPKEICNETKLKGLYLGGNRLQG
EIPRESGNLAELELMSLSENELQGAIPREWGNLTGLGILQLSDNFLTGEIPLEIGNLQNLEELELGQNKLIGTVPVAIEN
VSTLRELDFQDNSLSGSLSSIADVRLLNLQELLLWGNKFSGTIPRFIFNASKLSILDLQDNSESSFIPNTEGNLRNLQRL
RLYDNYLTSSTPELSFLSSLSNCKSLTHLSLSNNPLDGILQRTSVGNLSHSLKEFYMSNCNVSGGIPEEITNLTNLTTIY
LGGNKLNGSIPITLGKLQKLQGLGLEDNKLQGSIPDNIFRETELYELELGGNKLSGSIPACENNLASLRILSLGSNELTS
IPLTFWNLKDILQLNCSSNFLTGPLPSEIGNLKVLVIIDESMNNFSGVIPTEIGGLKNLEYLFLGYNRLQGSIPNSFGDL
ISLKSLNLSNNNLSGAIPASLEKLSYLEDLNLSENKLEGEIPTGGSFGNFSAESFEGNELLYGTPNLQVPPCKTSIHRTS
WKNSLLLRIVLPLSTTFMIVVILLILRYRQRGKRPSNDASMPLVAMWRTFSYLELCRATDEFSENNLIGTGGFGSVYKAR
LGDGMEVAVKVENLQCGRAFKSEDVECEMMKSIRHRNLIKVISSCSNEEFKALVLEYMPHGSLEKYLYSSNCILDIFQRL
NIMIDVASALEYLHFGYSAPIIHCDLKPNNVLLDDNMVAHLSDESIAKLLAGEDQSMTQTQTFATIGYMAPEYGREGRVS
ANGDVYSFGIMLMETFTRKKPTDEIENGEMTLKHWVNDWLPISTMEVVDANLLSQEDIHFVAKEQCVSFVENLAMACTGE
SPEQRINAKEIVKKLLKIRDSLLRNVGGRCIRQSNLN
>XP_006465575.1 probable LRR receptor-like serine/threonine-protein kinase
At3g47570 isoform X3 [Citrus sinensis]
MERLHSLRMMSRFLLLHCLILISLFIAAATANTSSTITDRDALLALKAHITHDPINFLAKNWNTSTPVCNWTGVACDVHS
HRVTVENISSLNETGTIPSQLGNLSSLQSLNLSCNRLEGSIPSAIFTIYTLKYVSLRENQVSGQIPANICSNLPELDYLS
LGKNMFHGGIPSALSNCTYLQILHLSYNDESGAVPKDIGNLSKLKELYLGRNRLQGEIPREFVNLTELERMSLSENELQG
GIPRELGNLTKLEGLQLFRNNLIGGIPRELGNLTKLERLQLFWNNLTGAIPKEIGNLTKLKELSLDGNRLQGEIPLEISN
LQNLEELDERHNKLVDVRLPNLEALLLWGNNFSGTIPRFIFNASKLSILELSQNSESGFIPNTEGNLRNLEWLNLRDNYL
TSSTPELSFLSSLSNCKSLTFIHLSDNPLDGILSKTSIGNLSHSLKDFYMSNCNVSGGIPEEITNLINSITIDLGGNKLN
GSIPITLSKLQKLQGLGLDDNKLEGSIPDSICRLTELYELELGGNKLEGSIPACFSNLASLRILSLSSNELTSIPLTEWN
LKDILQLNESSNFLTGPLPLEIGNLKVLIGIDESMNNESSVIPTEIGGLKNLEYLFLGYNRLEGSIPDSFGDLISLKELN
LSNNNLSGAIPTSLEKLSYLEDINLSENKLEGEIPRGGSFGNFAAESFEGNELLCGSPTLQVLPCKTSIHHTSWKNSLLD
GIVLPLSTTLLIVVIWLILRYRKRGKQPSNDANMPLVATWRTESYLELCRATNGFSENNLIGRGGFGSVYKARLGDGMEV
AVKVENLQCGRAFKSFAVECEMMKSIRHRNLIKVISSCSNEEFKALVLEYKPHGSLEKYLYSSNCILDIFQRLNIMIDVA
SALEYLHFGCSAPVIHCDLKPDNVLLDDNLVAYLSDFGIAKLLIGEDQSMTQTQTLATIGYMAPEYGREGRVSTNGDVYS
FGIMLMETFTGKKPTDEIFNGEMTLKHWVNDWLPISTMEVVDANLLSQEDVHFVAKEQCVSEVENLAMACTVESHEQRIN
AKEIVTKLLKIRDSLLRNVGGRRISQPNLN
>GAY59873.1 hypothetical protein CUMW_197790 [Citrus unshiu]
MERLHSLSIMSRFLLLNRLLLISLFIAVATANTSTITTDRDALLAMKAHITHDPINFLAKNWNTSISFCNWTGVTCDVHG
HRVTALNISGLNLISTIPFQLGNLSSLQSLNLSCNRLSGSIPSAIFTIYTLKYVSFRENQLSGAFPSFIENKSSLQHLDF
SRNTLSGEIPANICSSLPFLEILSLSKNMFHGGIPSALSNCTYLQILSLSYNDESGAIPKDIGNLTKLKGLYLGRNSLQG
EIPREFGNLSEMELMSLSENKLRGGIPQELGNLTKLEMLQLFLNNLTGAIPKEIGNLTKLKELSLERNMLQGEIPREFGN
LSELELMSLSENELQGEIPREFGNLVELGLLSLYENKLQGAIPRELGNLTGLENLQLDENFLTGEIPLEIGNLQNLKELI
LADNKLVGTVPTAIFNVSTLKLLALYNNSLSGCLSSIGDDQLPNLEILYLWGNNENGTIPRFIFNASKLSYLSLGENSFS
GFIPNTEGNLRNLERLNFEDNYLTSSTPELSFLSSLSNCKSLTIIHLSNNPLDGILPKTSVSNLSLSFEEFYMYNCNISG
GIPEEISNLTNLTTIKLGGNKLNGSIPIALGKLQKLQYLGLEDNKLEGSIPNDICRLAKLYLLELGGNKLYGSIPACEGN
LASLRILSLGSNGLTSIPLTEWNLKDILYLNFSSNFFTGPLPLEIGNLKVLVGMDESMNNLSDVIPTEIGGLKNLEYLFL
GYNKLQGSIPDSFGDLISLKELNLSNNNLSGAIPASLEKLSYLEDLNLSENKLEGEIPRGGSEGNESAESFEGNELLCGS
PNLQVPPCKTRIHHASWKKSLLLGTILPLSTTEMIVVILLILRYRQRGKRPLNDANMPLVATWRMFSYLELCRATSGFSE
NNLIGRGGFGSVYKARLGDGIEVAVKVENLQCERAFKSFDVECEVMKSIRHRNLIKVISSCSNEEFKALVLEYMPHGSLE
KYLYSSNCILDIFQRLNIMIDVASALEYLHEGHSAPIIHCDLKPSNVLLDDNMVAHLSDFSIAKLLTGEDQSMTHTQTLA
TIGYMAPEYGREGRVSANGDVYSFGIMLMETFTGKKPTDEMENEEMTLKHWVNDWLPISTMEVVDANLLSQEDIHEVAKE
QCVSFVENLAMECTVESPEQRINAKEIVAKLLKIRDLLLRNVGGRCIRQSNLN
>XP_006465463.1 probable LRR receptor-like serine/threonine-protein kinase
At3g47570 isoform X1 [Citrus sinensis]
MERLHSLSIMSRERLLHCLILISLFIAAATANTSTITTDRDALLALKAHITHDPTNFFAKNWNTSISFCNWTGVTCDVHS
HRVTVLNISRLNLTGTIPSQLGNLSSLQSENLSENRLSGSIPSAIFTMYTLKYVSFRENQLSGAFPSFIENKSSLQHLDF
SQNTLSGEIPANICSNLPFLEYISLSKNMFHGGIPSALSKCTYLQILSLSENDESGAIPKDIGNLTKLMELYLGRNRLQG
EIPREFGSLAELELMSLRESNLQGGIPQELGNLAKLEMLQLFQNNLTGAIPKEIGNLTKLEELYLGINRLQGEIPREFSN
LAKLEMMSLSENNLQGEIPHELGNLSGLETLALYNNELTGEIPHEISNLQNLEELDLGHNKLVGTVPAAIFNVSTLKGES
VSNNSLSGCLSSIVDARLPNLEVLYLWGNNFSGTIPRFIFNVSKLSKLSLEKNSESGFIPNTEGNLRNLKWLILYDNYLT
SSTPGLSFLSSLSNCKSLTYIDLSHNPLDSILQRMSIGNLSHSLEEFQMYNCNVSGGIPEEIRNESNLTLIDLGGNKLNG
SIPITLSKLQKLQGLGLENNKLEGSIPDDICRLAELFRLELGGNKLSGSIPTCFSNLASLRILSLGSNELTSIPLTFWNL
KDILQLNFSSNELTGPLPLEIGNLKVLVGIDLSMNNESGVIPTEIGGLKNLEYLELGYNRLQGSIPNSEGDLINLKEINL
SNNNLSGAIPASLEKLSYLEDINLSFNKLEGEIPRGGSFGNFSAESFEGNELLCGSPNLQVPPCKTGIHHTSSKNSLLLG
IVLPLSTIFMIVVSLLILRYRQRGKRPSNDANMPLVATWRMVSYLELCRATDGESENNLIGKGGFGSVYKARLSDGMEVA
VKVFNLQCGRAFKSFDIECEMMKSIRHRNLIKVISSCSNEEFKALVLEYMPHGSLEKYLYSSNCILDIFQRLNIMIDVAS
ALEYLHFGHSAPIIHCDLKPSNVLLDDNMVAHLSDFSIAKLLIGEDQSMTHTQTLATIGYMAPEYGREGRVSTNGDVYSF
GIMLMETFTEKKPTDEIFNEEMTLKQWVNDWLPISTMEVVDGNLLSQEDIHFVAKEQCVSYVENLAMACTVESPKQRINA
KEIVTKLLKIRGSLLRNVGGRCIRQSNLN
>XP_006465464.1 probable LRR receptor-like serine/threonine-protein kinase
At3g47570 isoform X2 [Citrus sinensis]
MERLHSLSIMSRERLLACLILISLFIAAATANTSTITTDRDALLALKAHITHDPINFFAKNWNTSISFCNWTGVTCDVHS
HRVTVLNISRLNLTGTIPSQLGNLSSLQSLNLSFNRLSGSIPSAIFTMYTLKYVSFRENQLSGAFPSFIENKSSLQHLDE
SQNTLSGEIPANICSNLPFLEYISLSKNMFHGGIPSALSKCTYLQILSLSENDESGAIPKDIGEIPREFGSLAELELMSL
RESNLQGGIPQELGNLAKLEMLQLFQNNLTGAIPKEIGNLTKLEELYLGINRLQGEIPREFSNLAKLEMMSLSENNLQGE
IPHELGNLSGLETLALYNNFLTGEIPHEISNLQNLEELDLGHNKLVGTVPAAIFNVSTLKGFSVSNNSLSGCLSSIVDAR
LPNLEVLYLWGNNFSGTIPRFIENVSKLSKLSLEKNSESGFIPNTFGNERNLKWLILYDNYLTSSTPGLSFLSSLSNCKS
LTYIDISHNPLDSILQRMSIGNLSHSLEEFQMYNCNVSGGIPEEIRNLSNITLIDLGGNKLNGSIPITLSKLQKLQGLGL
ENNKLEGSIPDDICRLAELFRLELGGNKLSGSIPTCESNLASLRILSLGSNELTSIPLTFWNLKDILQLNESSNFLTGPL
PLEIGNLKVLVGIDLSMNNESGVIPTEIGGLKNLEYLFLGYNRLQGSIPNSEGDLINLKFLNLSNNNLSGAIPASLEKES
YLEDLNLSENKLEGEIPRGGSFGNFSAESFEGNELLCGSPNLQVPPCKTGIHHTSSKNSLLLGIVEPLSTIFMIVVSLLI
LRYRQRGKRPSNDANMPLVATWRMVSYLELCRATDGFSENNLIGKGGFGSVYKARLSDGMEVAVKVFNLQCGRAFKSFDI
ECEMMKSIRHRNLIKVISSCSNEEFKALVLEYMPHGSLEKYLYSSNCILDIFQRLNIMIDVASALEYLHFGHSAPIIHCD
LKPSNVLLDDNMVAHISDESIAKLLIGEDQSMTHTQTLATIGYMAPEYGREGRVSINGDVYSFGIMLMETFTEKKPTDEI
FNEEMTLKQWVNDWLPISTMEVVDGNLLSQEDIHEVAKEQCVSYVENLAMACTVESPKQRINAKEIVTKLLKIRGSLLRN
VGGRCIRQSNLN
>GAY68232.1 hypothetical protein CUMW_262510 [Citrus unshiu]
MCSNLYITNKALTVCSLPILHEWLFFCVREIPPEIGNERNLEELDLGANKLVGTVPAAIFNLSTLKEFSIPNNSLSGCLS
SIADVRLPNLEVINEWGNNESGTIPRFIFNASKLSALDLDGNSFSGFIPNTEGNLRNLKWLILSDNYLTSSTPELSELSS
LSNCKSLTYIDLSYNPLDSILPRTSVGNLSHSLEDFEMNNCNVSGGIPEEISNLTNLTTIDIGGNKLNGLIPITLSKLQK
LQGLVLYDNKLEGSIPDDICRLAELYELELDGNKLSGSIPACLSNLISLRILSLGSNELTSIPLTEWNLEDILYLNFSSN
FLTGPLPLEIGKLKVLVGIDFSMNNFSGVIPTKIGGLKNLEYLFLGYNRLQGSIPDSFGDLTSLKSLNLSNNNLSGTIPA
SLEKLSYLENLNLSENKLEGEIPRGGSFGKFSAESFKGNELLCGSPNLQVPPCKTSIHHTSWKNSLLLGIVEPLSTTEMI
VVILLILRYRQRGKQPSNDANMPLVAMWRMFPYLELCRATDGFSENNLIGKGGFGSVYKARLGDGMEVAVKVENLECGRA
FKSFDVECDMMKSIRHRNLIKVISSCSNEEFKALVLEYMPHGSLEKYLYSSNCILDIFQRLNIMIDVASALEYLHEGYSA
PVIHCDLKPSNVLLDDNMVAHLSDFSIAKLLTGEDQSMTQTQTLATIGYMAPEYGREGRVSANGDVYSFGIMLMETFTGK
KPTDEIFNEEMTLKHWVNDWLPISTMEVVDVNLLSQEDIHFVAKEQCVSEVENLAMACTVESPEQRINAKEIVTKLLKIR
DSYGCFLNLESE
>GAY69164.1 hypothetical protein CUMW_269900 [Citrus unshiu]
MMSRFLLLHCLILISLFIAAATANTSSTITDRDALLALKAHITHDPINFLAKNWNTSTPVCNWTGVACDVHSHRVTVLNI
SSLNLIGTIPSQLGNLSSLQSLNLSCNRLSGSIPYTIFTTYTLKAVSLGENQLSGQIPTNICSNLPFLEILFLSENMEHG
EIPSALSNCTYLRILSLAYNDESGAVPREIGNLTKLRELYLGRNRLQGGIPQELGNLAKLEGLQLLQNNLTGEIPLEISN
LKNLEELQLGQNKLIGTVPVAIFNVSTLKFLGLQNNSLSGSLSSIANVRLPNLEKLYLWGNNESGTIPRFIFNASKLSKL
SLGMNSFSGFIPSTEGNLRNLEQLGLDENYLTSSTPELSFLSSLSNCKSLTLIALSNNPLDGILPKTSISNLSRSLEEFY
MYNCNISGSIPEEISNLTNLVEIDLGGNKINGSIPITLGKLRKLQRLNLEDNILEGSIPDDICRLAELYRLELGSNKLYG
SIPACEGNLASLRILSLGSNKLTSIPLTFWNLKDILQLNESSNFLTGPLLLEIGNLKVLIGIDESMNNFSGVIPREIGGL
KNLEYLFLGYNRERGSIPDSEGDLISLKFLNLSNNNLSGAIPTSLEKLSYLEDLNLSENKLEGEIPRGGSEGNFSAESFE
GNELLCGSPNLQVPPCKTSIHHTSWKNSLLLGIVEPLSTTLLIVVIWLILRYRQRGKKPSNDANMPLVATWRTFSYLELC
RATDEFSENNLIGRGGFGSVYKARLGDGMEVAVKVENLQCGRAFKSFDVECEMMKSIRHRNLIKVISSCSNEEFKALVLE
YMPNGSLEKYLYSSNCILDIFQRLNIMIDVASALEYLHEGYSALVIHCDLKPSNVLLDDNMVAHLSDFSIAKLLTGEDQS
MTQTQTLGTIGYMAPEYGREGRVSINGDVYSFGIMLMETFAGKKPTDEIFNEEMTLKQWVNGWLPISTVEVVDPNLLSQE
DVHFVAKEQCVSEVENLAMACTVESPEKRINAKEIVTKLLKIRGSLLRNVGGRCIRQSNLN
>XP_024954373.1 probable LRR receptor-like serine/threonine-protein kinase
At3g47570 isoform X3 [Citrus sinensis]
MERLHSLSIMSRERLLACLILISLFIAAATANTSTITTDRDALLALKAHITHDPTNFFAKNWNTSISFCNWTGVTCDVHS
HRVTVLNISRLNETGTIPSQLGNLSSLQSLNLSENRLSGSIPSAIFTMYTLKYVSFRENQLSGAFPSFIENKSSLQHLDE
SQNTLSGEIPANICSNLPFLEYISLSKNMFHGGIPSALSKCTYLQILSLSENDESGAIPKDIGNITKLMELYLGRNRLQG
EIPREFGSLAELELMSLRESNLQGGIPQELGNLAKLEMLQLFQNNLTGAIPKEIGNLTKLEELYLGINRLQGEIPREFSN
LAKLEMMSLSENNLQGEIPHEISNLQNLEELDLGHNKLVGTVPAAIFNVSTLKGFSVSNNSLSGCLSSIVDARLPNLEVL
YLWGNNESGTIPRFIFNVSKLSKLSLEKNSESGFIPNTEGNERNLKWLILYDNYLTSSTPGLSFLSSLSNCKSLTYIDES
HNPLDSILQRMSIGNLSHSLEEFQMYNCNVSGGIPEEIRNISNLTLIDLGGNKLNGSIPITISKLQKLQGLGLENNKLEG
SIPDDICRLAELFRLELGGNKLSGSIPTCFSNLASLRILSLGSNELTSIPLTFWNLKDILQLNESSNFLTGPLPLEIGNL
KVLVGIDLSMNNFSGVIPTEIGGLKNLEYLFLGYNRLQGSIPNSFGDLINLKFLNLSNNNLSGAIPASLEKLSYLEDINL
SENKLEGEIPRGGSFGNFSAESFEGNELLCGSPNLQVPPCKTGIHHTSSKNSLLLGIVLPLSTIFMIVVSLLILRYRQRG
KRPSNDANMPLVATWRMVSYLELCRATDGESENNLIGKGGFGSVYKARLSDGMEVAVKVENLQCGRAFKSEDIECEMMKS
IRHRNLIKVISSCSNEEFKALVLEYMPHGSLEKYLYSSNCILDIFQRLNIMIDVASALEYLHFGHSAPIIHCDLKPSNVL
LDDNMVAHLSDFSIAKLLIGEDQSMTHTQTLATIGYMAPEYGREGRVSTNGDVYSFGIMLMETFTEKKPTDEIFNEEMTL
KQWVNDWLPISTMEVVDGNLLSQEDIHFVAKEQCVSYVFNLAMACTVESPKQRINAKEIVTKLLKIRGSLLRNVGGRCIR
QSNLN
>XP_024953035.1 probable LRR receptor-like serine/threonine-protein kinase
At3g47570 isoform X2 [Citrus sinensis]
MERLHSLRMMSRFLLLHCLILISLFIAAATANTSSTITDRDALLALKAHITHDPTNFLAKNWNTSTPVCNWTGVACDVHS
HRVTVLNISSLNETGTIPSQLGNLSSLQSLNLSCNRLFGSIPSAIFTIYTLKYVSLRENQVSGQIPANICSNLPFLDYLS
LGKNMEHGGIPSALSNCTYLQILHLSYNDESGAVPKDIGNLSKLKELYLGRNRLQGEIPREFVNETELERMSLSENELQG
GIPRELGNITKLERLQLFWNNLTGAIPKEIGNLTKLKELSLDGNRLQGEIPLEISNLQNLEELDERHNKLVGTVPAAIEN
MSMLKLLHLQNNSLLGCLSSIADVRLPNLEALLEWGNNESGTIPRFIFNASKLSILELSQNSFSGFIPNTEGNLRNLEWL
NERDNYLTSSTPELSFLSSLSNCKSLTFIHLSDNPLDGILSKTSIGNESHSLKDEYMSNCNVSGGIPEEITNLINSITID
LGGNKLNGSIPITLSKLQKLQGLGLDDNKLEGSIPDSICRLTELYELELGGNKLFGSIPACFSNLASLRILSLSSNELTS
IPLTEWNLKDILQLNESSNFLTGPLPLEIGNLKVLIGIDESMNNESSVIPTEIGGLKNLEYLFLGYNRLEGSIPDSFGDL
ISLKFINLSNNNLSGAIPTSLEKLSYLEDENLSENKLEGEIPRGGSFGNFAAESFEGNELLCGSPTLQVLPCKTSIHHTS
WKNSLLLGIVLPLSTTLLIVVIWLILRYRKRGKQPSNDANMPLVATWRTFSYLELCRATNGFSENNLIGRGGEGSVYKAR
LGDGMEVAVKVENLQCGRAFKSFAVECEMMKSIRHRNLIKVISSCSNEEFKALVLEYKPHGSLEKYLYSSNCILDIFQRL
NIMIDVASALEYLHFGCSAPVIHCDLKPDNVLLDDNLVAYLSDFGIAKLLIGEDQSMTQTQTLATIGYMAPEYGREGRVS
INGDVYSFGIMLMETFTGKKPTDEIFNGEMTLKHWVNDWLPISTMEVVDANLLSQEDVHFVAKEQCVSFVENLAMACTVE
SHEQRINAKEIVTKLLKIRDSLLRNVGGRRISQPNLN
>XP_006465573.1 LRR receptor-like serine/threonine-protein kinase EFR isoform X1
[Citrus sinensis]
MERLHSLRMMSRFLLLHCLILISLFIAAATANTSSTITDRDALLALKAHITHDPTNFLAKNWNTSTPVCNWTGVACDVHS
HRVTVLNISSLNLTGTIPSQLGNLSSLQSLNLSCNRLFGSIPSAIFTIYTLKYVSLRENQVSGQIPANICSNLPFLDYLS
LGKNMFHGGIPSALSNCTYLQILHLSYNDESGAVPKDIGNLSKLKELYLGRNRLQGEIPREFVNLTELERMSLSENELQG
GIPRELGNLTKLEGLQLFRNNLTGGIPRELGNLTKLERLQLEWNNLTGAIPKEIGNLTKLKELSLDGNRLQGEIPLEISN
LQNLEELDERHNKLVGTVPAAIFNMSMLKLLHLQNNSLLGCLSSIADVRLPNLEALLEWGNNESGTIPRFIFNASKLSIL
ELSQNSFSGFIPNTFGNLRNLEWLNLRDNYLTSSTPELSFLSSLSNCKSLTFIHLSDNPLDGILSKTSIGNLSHSLKDFY
MSNCNVSGGIPEEITNLINSITIDIGGNKLNGSIPITLSKLQKLQGLGLDDNKLEGSIPDSICRLTELYELELGGNKLFG
SIPACESNLASLRILSLSSNELTSIPLTFWNLKDILQLNESSNELTGPLPLEIGNLKVLIGIDESMNNESSVIPTEIGGL
KNLEYLELGYNRLEGSIPDSEGDLISLKFLNLSNNNLSGAIPTSLEKLSYLEDINLSENKLEGEIPRGGSFGNFAAESFE
GNELLCGSPTLQVLPCKTSIHHTSWKNSLLLGIVLPLSTTLLIVVIWLILRYRKRGKQPSNDANMPLVATWRTFSYLELC
RATNGFSENNLIGRGGFGSVYKARLGDGMEVAVKVENLQCGRAFKSFAVECEMMKSIRHRNLIKVISSCSNEEFKALVLE
YKPHGSLEKYLYSSNCILDIFQRLNIMIDVASALEYLAFGCSAPVIHCDLKPDNVLLDDNLVAYLSDEGIAKLLIGEDQS
MTQTQTLATIGYMAPEYGREGRVSTNGDVYSFGIMLMETFTGKKPTDEIENGEMTLKHWVNDWLPISTMEVVDANLLSQE
DVHFVAKEQCVSEVENLAMACTVESHEQRINAKEIVTKLLKIRDSLLRNVGGRRISQPNLN
>XP_024952125.1 probable LRR receptor-like serine/threonine-protein kinase
At3g47570 [Citrus sinensis]
MERVHSFRMMSRFLLLHCLILISLFIAAATANTSSTITDRDALLALKARITHDPINFLAQNWNTSTPVCNWTGVACDVHS
HRVTVLNISSLNETGTIPSQLGNLSSLQSLNLSENRLSSSIPSAIFTIYTLQNVSERKNQLTGTFPSFIENKSSLQHLDE
SENTLSGEIPANICSNEPFLEYLALSNNMFHGGILSALSNCTYLQKLDLVYNDFSGAVPREIGNLTKLKELHLGRNRFQG
EIPREFGNLAELEQMSLAENNLQGGIPQELGNLAKLKTLQLFQNNLTGEIPPEIGNLRNLEELDLGHNKLVGTVPAAIEN
LSTLKEESIPNNSLSGCLSSIADVRLPNLEVINEWGNNFSGTIPRFIFNASKLSALDLDGNSESGFIPNTEGNLRNLKWL
ILSDNYLTSSTPELSELSSLSNCKSLTYIDESYNPLDSILPRTSVGNLSHSLEDFEMNNCNVSGGIPEEISNLTNLTTID
LGGNKINGLIPITLSKLQKLQGLVLYDNKLEGSIPDDICRLAELYELELDGNKLSGSIPACLSNLISLRILSLGSNELTS
IPLIFWNLEDILYLNFSSNELTGPLPLEIGKLKVLVGIDFSMNNESGVIPTKIGGLKNLEYLFLGYNRLQGSIPDSEGDL
TSLKSLNLSNNNLSGTIPASLEKLSYLENINLSENKLEGEIPRGGSFGKFSAESFKGNELLCGSPNLQVPPCKTSIHHTS
WKNSLLLGIVEPLSTTFMIVVILLILRYRQRGKQPSNDANMPLVATWRMFPYLELCRATDGFSENNLIGKGGFGSVYKAR
LGDGMEVAVKVENLECGRAFKSFDVECDMMKSIRHRNLIKVISSCSNEEFKALVLEYMPHGSLEKYLYSSNCILDIFQRD
NIMIDVASALEYLHFGYSAPVIHCDLKPSNVLLDDNMVAHLSDFSIAKLLTGEDQSMTQTQTLATIGYMAPEYGREGRVS
ANGDVYSEGIMLMETFTGKKPTDEIFNEEMTLKHWVNDWLPISTMEVVDVNLLSQEDIHFVAKEQCVSEVENLAMACTVE
SPEQRINAKEIVTKLLKIRDSLLRNVGGRCIRQSNLN
>XP_006465462.1 receptor kinase-like protein Xa21 [Citrus sinensis]
MMSRFLLLHCLILISFFIAAATANTSSTITDRDALLALKARITHDPINFLAKNWNTSTHVCNWTGVACDVHSHRVTVLNI
SSLNLTGIIPSQLGNLSSLQSLNLSCNRLSGSIPSAIFTIYTLKYVSFRENQLSGAFSSFIENKSSLQHLDFSHNTLSGE
IPANICSSLPFLDFLSLQENMLHGGIPSTLSNCTYLQKLGLVYNNESGAIPKEIGNLTKLKILYLGGNRLQGEIPREFGN
LADLERMSLSENNLQGGIPRELGNLTKLEILQLERNNLTGAIPRELGNITGLGVLELSENFLTGEIPLEIGNLQNLEELE
LGHNKLIGTVPVAIFNVSTTLKLLGLQDNSLSGGLSSIANVRLPNLEELYLWGNNESGTIPRFIFNASKLSILDLDKNSF
SGFIPNTFGNLRNLEYLDLQYNYLTSETLELSFLSSLSNCKSLTLIGLSNNPLDGILPRTSVGNLSHSLKYFKMHNCNIS
GGIPEEISNLTNLMTIDLGGNKLNGSIPITLGKLQKLQWLSLDDNKLEGSIPDDICRLAELYLLELGGNKLYGLIPACFG
NLASLRILSLCSNELTSIPLTFWNLKDILHLYFSLNFFTGPLPLEIGNLKVLIGIDESMNNFSGVIPTEIGGLKNLESLE
LGYNRLRGSIPDSFGDLISLKFLNLSNNNLSGAIPTSLEKLSYLEDINLSENKLEGEIPRGGSEGNFLAESFEGNELLCG
SENDQVPPCKTSIHHTSWKKSLLLGTILPLSTTFMIVVIWLILRYRQRGKRPSNDANMPLVATWRMFSYLELCRATSGES
ENNLIGRGGFGSVYKARLGDGMEVAVKVENLQCERAFKSFDVECEVMKSIRHRNLIKVISSCSNEEFKALVLEYMPHGSL
EKYLYSSNCILDIFQRLNIMIDVASALEYLHFGCSTPVIHCDLKPNNVLLDDNMVAYLSDFGIAKLLIGEDQSMTQTQTL
ATIGYMAPEYGREGRVSTNGDVYSFGIMLMETFTGKKPTDEIENGEMTLKHWVNDWLPISTMEVVDANLLSQEDVHFVAK
EQCVSEVENLAMACTVESHEQRINAKEIVTKLLKIRDSLLRNVGGRRIRQPNLN
>XP_024957148.1 probable LRR receptor-like serine/threonine-protein kinase
At3g47570 [Citrus sinensis]
MERVHSLRMMSIFLLLHCLIIISLFIAATTANTSSNITDRDALLALKAHITHDPINFLAKNWNTSTPVCNWTGVACDIHS
HRVTVENISSLNERGTIPSQLGNLSSLQSLNLSCNRLSGSIPSAIFTIYTLKNVSLGKNQLSGQIPTNICSNLPFLEFLS
LSLNMFHGGIPSTLSNCTYLRILSLAYNDFSGAVPREIGNLTKLKVLYIGANRLQGEIPREFGNLTELEQFSLPINNLQG
GIPQELGNLAKLEILQLFQNNLIGPIPRELGNITGLGILALSDNELTGEIPTEISNLRNLEELDLARNKLVGIVPAAIEN
VSTLQHLGLQDNSLLGCLSSNGDVRLPNLEGLYLSGNNFSGTIPRFIFNASKLFKLSLQRNSLFGFIPNTEGNLRNLKWL
SLYDNYLTSSTPELSFLSSLSNSKSLTFIDLSNNPLDSVLPKTFVGNVSHSLEFFVMSYCNISGVIPEEITNLTKLTTII
LGGNKINGSIPITLSKLQKLQYLGLDDNKLEGSIPDSICRLAELYDLELGGNKLSGSIPACFSNLASLRTLSLDSNELTY
IPLTFWNLKDILYLNFSSNFLIGPLPLEVGNLKVLVGIDESMNNFSGVIPTEIGGLQNLEYLFLGYNRLQGSIPDSEGDL
ISLKSLNLSNNNLSGAIPASLEKLLYLEDINLSENKLEGEIPRGGSFGNFSAESFEGNELLCGSPNLQVPPCKTSIAPTS
SKNSLLLGIVEPLSTTFMIVVILLILRYRQRGKRPSNDANIPLVATWRMFSYLELCQATDEFSENNLIGKGGFGSVYKAR
LGDGMEVAVKVFNLQCGRAFKSFDIECEMMKSIRHRNLIKVISSCSNEEFKALVLEYMPHGSLEKYLYSSNCILDIFQRL
NIMIDVASTLEYLYFGHSAPIIHCDLKPSNVLLDDNMVAHLSDESIAKLLTGDDQSMTQTQTFATIGYMAPEYGREGRVS
ANGDVYSFGIMLMETFTGKKPTDEIFNEEMTLKQWVNDWLPISTMEVVDANLLSPEDVHFVAKEQCVSEVENLAMACTVE
SPEQRINAKEIVTKLLKIRGSLLRNVGGRCIRQSNLN
>KDO39003.1 hypothetical protein CISIN_1g046544mg, partial [Citrus sinensis]
EIPLEISNLQNLEELDLRHNKLVGTVPAAIFNMSMLKLLHLQNNSLLGCLSSIADVRLPNLEALLLWGNNESGTIPRFIF
NASKLSILELSQNSFSGFIPNTFGNLRNLEWINLRDNYLTSSTPELSFLSSLSNCKSLTFIHLSDNPLDGILSKTSIGNL
SHSLKDFYMSNCNVSGGIPEEITNLINSITIDLGGNKLNGSIPITLSKLQKLQGLGLDDNKLEGSIPDSICRLTELYELE
LGGNKLEGSIPACFSNLASERILSLSSNELTSIPLTFWNLKDILQLNESSNFLTGPLPLEIGNLKVLIGIDESMNNESSV
IPTEIGGLKNLEYLFLGYNRLEGSIPDSEGDLISLKFLNLSNNNLSGAIPTSLEKLSYLEDLNLSENKLEGEIPRGGSFG
NFAAESFEGNELLCGSPTLQVLPCKTSIHHTSWKNSLLLGIVLPLSTTLLIVVIWLILRYRKRGKQPSNDANMPLVATWR
TESYLELCRATNGFSENNLIGRGGFGSVYKARLGDGMEVAVKVENLQCGRAFKSFAVECEMMKSIRHRNLIKVISSCSNE
EFKALVLEYKPHGSLEKYLYSSNCILDIFQRLNIMIDVASALEYLHFGCLAPVIHCDLKPDNVLLDDNLVAYLSDEGIAK
LLIGED
>XP_024041864.1 probable LRR receptor-like serine/threonine-protein kinase
At3g47570 [Citrus clementina]
MCSNLYGINKALTVCSLSILHEWLFLCVSTGEIPTEISNLRNLEELDLARNKLVGIVPAAIFNVSTLQHLGLQDNSLFGC
LSSIGDVRLPNLEGLYLSGNNESGTIPRFIFNASKLFKLSLQRNSFFGFIPNTFGNLRNLKWLSLYDNYLTSSTPELSFL
SSLSNSKSLTFIDLSNNPLDSVLPKTFVGNVSHSLEFFVMSYCNISGSIPEEITNLTKLTTIILGGNKLNGSIPITLSKL
QKLQYLGLDDNKLEGSIPDSVCRLAELYDLELGGNKLFGSIPACESNLASLRTLSLDSNELTSIPLTEWNLKDILYLNES
SNFLIGPLPLEVGNLKVLVGIDESMNNESGVIPTEIGGLQNLEYLFLGYNRLQGSIPDSFGDLISLKSINLSNNNLSGAI
PASLEKLLYLEDENLSENKLEGEIPRGGSEGNFSAESFEGNELLCGSPNLQVPPCKTGIHHTSSKNSLLLGIVLPLSTIE
MIVVILLILRYRQRGKRPSNDANMPLVATWRMFSYLELCRATDGFSENNLIGKGGFGSVYKARLGDGMEIAVKVENLQCG
RAFKSFDIECEMMKSIRHRNLIKVISSCSNEEFKALVLEYMPHGSLEKYLYSSNCILDIFQRLNIMIDVASALEYLHFGH
SAPIIHCDLKPSNVLLDDNMVGHLSDFSIAKLLTGEDQSMTHTQTLATIGYMAPEYGREGRVSANGDVYSFGIMLMETFT
RKKPTDEMENGEMTLKHWVNDWLPISTMEVVDANLLSQEDIHEVAKEQCVSFVENLAMECTVESPKQRINAKEIVAKLLK
IRDSLERNVGGRCIRQSNLN
>ESR49610.1 hypothetical protein CICLE_v10033353mg, partial [Citrus clementina]
MCSNLYGTNKALTVCSLSILHEWLFLCVSTGNNCVIPTEISNLRNLEELDLARNKLVGIVPAAIFNVSTLQHLGLQDNSL
FGCLSSIGDVRLPNLEGLYLSGNNFSGTIPRFIFNASKLFKLSLQRNSFFGFIPNTFGNLRNLKWLSLYDNYLTSSTPEL
SELSSLSNSKSLTFIDLSNNPLDSVLPKTFVGNVSHSLEFFVMSYCNISGSIPEEITNLTKLTTIILGGNKLNGSIPITL
SKLQKLQYLGLDDNKLEGSIPDSVCRLAELYDLELGGNKLFGSIPACFSNLASLRTLSLDSNELTSIPLTEWNLKDILYL
NFSSNFLIGPLPLEVGNLKVLVGIDESMNNESGVIPTEIGGLQNLEYLELGYNRLQGSIPDSFGDLISLKSENLSNNNLS
GAIPASLEKLLYLEDINLSENKLEGEIPRGGSFGNFSAESFEGNELLCGSPNLQVPPCKTGIHHTSSKNSLLLGIVLPLS
TIFMIVVILLILRYRQRGKRPSNDANMPLVATWRMFSYLELCRATDGFSENNLIGKGGFGSVYKARLGDGMEIAVKVFNL
QCGRAFKSEDIECEMMKSIRHRNLIKVISSCSNEEFKALVLEYMPHGSLEKYLYSSNCILDIFQRLNIMIDVASALEYLH
FGHSAPIIHCDLKPSNVLLDDNMVGHLSDESIAKLLTGEDQSMTHTQTLATIGYMAPEYGREGRVSANGDVYSFGIMLME
TFTRKKPTDEMENGEMTLKHWVNDWLPISTMEVVDANLLSQEDIHFVAKEQCVSFVENLAMECTVESPKQRINAKEIVAK
LLKIRDSLLRN
>XP_006465518.1 probable LRR receptor-like serine/threonine-protein kinase
At3g47570 [Citrus sinensis]
MMSRFLLLHCLILISLFIAAATANTSSTITDRDALLALKAHITHDPINFLAKNWNTSTPVCNWTGVTCDVHSHRVTVLNI
SSLNLTGTIPSQLGNLSSLKSLNLSENRLSGSIPSTIFTTFTLTYVSLRQNQLSGQIAANICSNLPFLEVLSLSRNMFQG
GIPSTLSNCTYLQTLALSYNNESGTIPIEIGNLTKLKELYLGVNRLQGEIPREFGNLADLEQMSLAINNLQGGIPRELGN
LTKLEMLQLFENNLTGAIPKEIGNLTKLKELSLFGNRLQGAIPRELGNITRLGILALPNNFLTGEIPLEIGNLQNLEELD
LGLNKLIGTVPATIFNVSTLKLLLLEHNSLLGSLSSIANVRLPNLEELLLWGNNFSGTIPRFIFNASKLSILELSKNSES
GFIPNTEGNLRNINWLYLNDNNLASSTPELSFLSSLSNCKSLTHIALSHNPLDGILPRTSVGNLSHSLKEFYMSNCNVSG
GIPEEITNLTNLTTIYLGGNKLNGSIPITLSKLQKLQGLGLEDNKLEGSIPDSICHLTELYDLKLGGNKLFGSIPACENN
LDSLRILSLGSNELTSIPLTEWNLKDILYLNFSSNFFTDPLPLEIGNLKVLIGIDFSMNNFSGVIPTEIGGLKDLEYLFL
GYNRLQGSIPDSEGNLISLKELNLSNNNLSGAIPASLEKLSYLEDLNLSENKLEGEIPRGGSFGNFSAESFEGNELLCDS
PNLQVPPCKTSIHHTSWKISLLLGIVEPLSTTFMIVVILLILRYRQRGKQPSNDANMPLVATWRTFSYLELCRATDGFSE
NNLIGKGGFGSVYKARLGDGMEVAVKVENLQCRRAFKSFDVECEMMKSIRHRNLIKVISSCSNEEFKGLVLEYMPQGSLE
KHLYSSNCILDIFQRLNIMINIASALEYLHFGCSTPVIHCDLKPSNVLLDDNMVAHLSDFSIAKLLIGEDQSMTHRYKYF
LFLANFLIKYGREGRVSTNGDVYSFGIMLMETFTEKKPTDEIFNEEMTLKQWVNDWLPISTMEVVDANLLSQEDVHFVAK
EQCVSEVENLAMACTVESHEQRINAKEIVTKLLKIRGSLLRNVGGRCLRQSNLN
>XP_024949391.1 probable IRR receptor-like serine/threonine-protein kinase
At3g47570 [Citrus sinensis]
MYTAVEVHKEPDSVGEALQDTNWFTAMKNEYDALIENRTWSLVRRTENQKVVGNKWVYRIKYNTDGSVAKYKARLVAKGE
QQIEGVNYFDTFSPVIKPATVRVVLSLAVMNQWIVRQVDVNNAFLNGELSEEVFIQQPEGFVDKSNLTMFADYTKLYMVL
SKLLEPVTGLNSEELDSFIQQFNTVFALKDLGRLSYFLGIEVLYDQDCIYLSQKKYIRDLLAKVDMLECKRVTTPMCSGK
DSKLQKVVKGELGYYVEDATHYRSIVGGLQYLILTRPEIAYSVHKLSQYVSAPTMQHLMACKRVLKYLKETQDYGLKFVK
DGDLKITAFTDVDWGSDLDDRKSIGAYCVYLGNNLISWSSKKQTVVTKSSAESEYRAFASAASEIAWLKSLFLEMEVYCV
ERPTIWCDNISATELAKNPVFHSRTKHIEIDVHFIRDKVLSGDLKICYVPSEDQIADILTKPLSSPQFNYLRDKLNVESC
PLSLRGAVKIAHCAEVRKKSQRVKLPAVICATCQTAAFIQFYNFLHTGTIPSQLGNLSSLQSLNLSENRLSGSIPSTTFT
TYTLKFVGLQGNQLSGALPFFIENKSSLQDLDLSDNALSGEIPANICSSLPELEYISLSQNMFHGGIPSALSKCTYLQIL
GLSENDESGAIPKEIGNLTKLQELYLGRNRLRGEIPRELSNLAELEQMWLSENELQGGIPQDLGNLAKLKMLQLSQNNLT
VLSENDESGAIPKEIGNLTKLQELYLGRNRLRGEIPRELSNLAELELMSLFDNELQGEIPPEISNLSNLEQLELGSNKLV
GTVPTAIFNVSTLQALGLQNNSLSGSLSSIVDVRLPNLKMLQMWENNFSGTIPRFIFNASKLSILELSDNSFSGFIPNTF
GNLRNLQALRLSNNYLTSSTLEFSFLSSLSNCKSLTLISFSNNPLDGILPKTSVGNLSHSLEYFEMAYCNVSGGIPEEIG
NLTNLTGIYLGGNKLNGSIPSTLGKLQKLQGLGLENNKLEGSIPDSICHSDELYKLELGGNKLSGSIPECENNLASLRIL
LIGSNELTSIPLTFWNIKDILYLNFSSNFFTGPLPLEIGNLKVLVGIDESMNNFSGVIPMAIGGLKNLQNLFLGYNRLQG
SIPDSFGDLISLISLNLSNNNLSGAIPASLEKLSYLENLNLSENKLEGEIPRGGSFGNFSFESFEGNELLCGSPNLRVPP
CKTSIHHISRKNAFLLGIVLPLSTVEMIVVIFLIVKCRKRERGPPNDANMPPEAMQRMFSYLELCRATDGFSENNLIGRG
SFGSVFKARLGDGMEVAMKVENLQYGRVFKSFDVECEMMKSIRHRNIIKVISSCSNEEFKALVLEYMPHGSLEKYLHSSN
YILDIYQRLNIMIDVASALEYLHEGYSAQVIHCDLKPSNVLLDDNMVAHLSDFGIAKLLTREDQSTIQTQTLATIGYMAP
EYGKEGRVSANGDVYSFGIMLMETFTRKKPTDEIENGEMTLKHWVNDWLPISTKEIVDPNLLSREDINEVAKEQCVSFVE
NVAMECTVESPEQRINAKEIVTKLLKIRDSLLRNV
>XP_006465579.1 LRR receptor-like serine/threonine-protein kinase FLS2 isoform
X7 [Citrus sinensis]
MERLHSLRMMSRELLLHCLILISLFIAAATANTSSTITDRDALLALKAHITHDPINFLAKNWNTSTPVCNWTGVACDVHS
HRVTVLNISSLNLTGTIPSQLGNLSSLQSENLSCNRLFGSIPSAIFTIYTLKYVSLRENQVSGQIPANICSNLPELDYLS
LGKNMFHGGIPSALSNCTYLQILHLSYNDESGAVPKDIGNLSKLKELYLGRNRLQGEIPREFVNLTELERMSLSENELQG
GIPRELGNETKLEGLQLFRNNLTGGIPRELGNLIKLERLQLEWNNLTGAIPKEIGNLTKLKELSLDGNRLQGEIPLEISN
LQNLEELDLRANKLVGTVPAAIFNMSMLKLLHLQNNSLLGCLSSIADVRLPNLEALLLWDNPLDGILSKTSIGGNKINGS
IPITLSKLQKLQGLGLDDNKLEGSIPDSICRLTELYELELGGNKLFGSIPACFSNLASLRILSLSSNELTSIPLTFWNLK
DILQLNESSNFLTGPLPLEIGNLKVLIGIDFSMNNFSSVIPTEIGGLKNLEYLFLGYNRLEGSIPDSFGDLISLKELNES
NNNLSGAIPTSLEKLSYLEDLNLSENKLEGEIPRGGSFGNFAAESFEGNELLCGSPTLQVLPCKTSIHHTSWKNSLLLGI
VLPLSTTLLIVVIWLILRYRKRGKQPSNDANMPLVATWRTFSYLELCRATNGFSENNLIGRGGFGSVYKARLGDGMEVAV
KVENLQCGRAFKSFAVECEMMKSIRHRNLIKVISSCSNEEFKALVLEYKPHGSLEKYLYSSNCILDIFQRLNIMIDVASA
LEYLHEGCSAPVIHCDLKPDNVLLDDNLVAYLSDEGIAKLLIGEDQSMTQTQTLATIGYMAPEYGREGRVSTNGDVYSFG
IMLMETFTGKKPTDEIENGEMTLKHWVNDWLPISTMEVVDANLLSQEDVHFVAKEQCVSFVENLAMACTVESHEQRINAK
EIVTKLLKIRDSLLRNVGGRRISQPNLN
>GAY67779.1 hypothetical protein CUMW_259180 [Citrus unshiu]
MMSRELLLHCLILISLFIAASTANSSSTITDRDALLALKARITHDPINFLAKNWNTSTPVCNWTGVACDVHSHRVTVINI
SSLNLTGPIPSQLGNLSSLQSLNLSCNRLSGSIPSAIFTTYTLKYVSFRKNQLSGQIPANICSNLPVLEYLSLSQNMFQG
GIPSTLSNCTYLRILSLAYNDFSGAVPKDIGNLTKLKELYLGVNRLQGEIPREFGNLAEMELMSLSENKLRGGIPRELGN
LTKLEMLQLFQNNLTGKIPREFGNLADLEWMSLWENNLQGAIPRELGNLTGLGILELSHNFLTGKIPPEIGNLRNLEELV
LGANQLVGIVPAAIFNVSTLKLLKLQNNFLLGCLSPIEDVRLPNLEELSLWGNNFSGTIPRFIFNASKLSTLELGDNSES
GFIPNIFGNLRNLKWLNLPNNYLASSSPELSFLSSLSNCKSLTHLSLSNNPLDGILPRTSVGNLSHSLKKEDMSNCNVSG
GIPEEITSLTNLTTIYLGGNKLNGSIPITLSKLQKLQGLGLEDNKLEGSIPDDICRLVELYKVELGGNKLSGSIPACFGN
LIALRILSLGSNELTSIPLTFWNLKDILQLNESSNELTGPLPLEIGNLKVLIGIDESMNNFSGVIPTEIGGLKYLEYLFL
GYNRLQGLIPDSEGNLISLKELNLSNNNLSGAIPASLEKLLYLEDLNLSENKLEGEIPRGGSEGNFSAESFEGNELLCGS
PLQVPPCKTSIHHTSWKISLLLGIVLPLSTTLIIVVIWLILRYRLRGKQPSNDANMPLVATSRTESYLELCRATDGFNEN
NLIGRGGFGSVYKARLGDGMEVAVKVENLQCRRAFKSFDVECEIMKSIRHRNLIKVISSCSNEEFKGLVLEYMPQGSLEK
HLYSSNCILDIFQRLNIMIDVASALEYLHFGCSTPIIHCDLKPSNVMLDDNMVAYLSDFGIAKLLIGEDQSMTQTQTLAT
IGYMAPEYGREGRVSTNGDVYSFGIILMETFTGKKPTDEIFNGEMTLKHWVNDWLPISTMEVVDANLLSQEDIHFAAKEQ
CVSFVFNLAMVCTVESLEQRINAKEIVKKLLKIRDSLLRNVGGRCIRQSNLN
>GAY66422.1 hypothetical protein CUMW_248620, partial [Citrus unshiu]
TANTSSTITDRDALLALKAHITHDPINFLAKNWNTSTPVCNWTGVACDVHSHRVTVLNISSINLTGTIPSQLGNLSSLQS
LNLSCNRLSGSIPSAIFTIYTLKYVSFRENQLSGAFSSFIENKSSLQHLDFSHNTLSGEIPANICSSLPELDELSLQENM
FHGGIPSTLSNCTYLQKLGLVYNNESGAIPKEIGNLTKLKILYLGGGIPQELGNLAKLEMLQLFQNNLIGEIPLEIGNLQ
NLEELELAQNKLIGIVPVAIFNVSTLKILQLQINCLSGSLSSITDVQLPNLEKLDLWGNNESGTIPRFIFNASKLSILNL
GGNSESGFIPNTEGNLRNLKYLYLENNYLTSSTLELSELSSLSNCKSLTHIDLSNNPLDGILPKTSVGNLSHSLEDFHMY
NCNISGGIPEEISNLTNLITIDLGGNKLNGSILITLSKLQKLQGLVLDDNKLEGSIPDDICREVELYKLELGGNKLSRSI
PACENNLIALRILSLGSNELTSIPLTFWNIKDILYLNFSSNFLTGPLPLKIGNLKVLVGIDFSMNNFSGVIPTEIGGLKD
LEYLFLGYNRLQGSIPDSEGNLISLKFLNLSNNNLSGAIPAPLEKLSYLEDLNLSENKLEGEIPRGGSFGNESVESEKGN
ELLCGSPNLQVPPCKTSIHHTSWKISLLLGILLPLSTTLMIVVIWLILRYRQRGKQPSNDANMPLVAMWRTFSYLELCRA
TDGFSENNLIGRGGLGSVYKARLGDGMEVAVKVENLQCRRAFKSEDVECEIMKSIRHRNLIKVISSCSNEEFKGLVLEYK
PQGSLEKHLYSSNCILDIFQRLNIMIDVASALEYLHFGCSAPVIHCDLKPDNVLLDDNLVAYLSDEGIAKLLIGEDQSMT
QTQTLATIGYMAPEYGREGRISINGDVYSFGIMLMETFTGKKPTDEIFNEEMTLKQWVNDWLPISTMEVVDANLLSQEDV
HFVAKEQCVSFVENLAMACTVESPEQRINAKEIVTKLLKIRGSLLRNFGGRCIRQSNLN
>XP_006427077.2 probable LRR receptor-like serine/threonine-protein kinase
At3g47570 [Citrus clementina]
MKIRALPKEIGNLTKLKELSINHNRLQGEIPREFGNLAELELMWLSENNLQGGIPRELGNLTKLEILHLWKNNLTGALPK
EIGNLTKLKHLSLDHNRLQGEIPREFGNLAELELLSLYENKLQGEIPLEIGNLRNLKDLILSENKLVGIVPFAIFNVSTL
KLLQLQNNSLLGCLSSIANVRLPNLEELDLWANNESGTIPHFIENTSKLSRLDINSNSFSGFIPNTEDNERNLEWLSLRD
NYLTSSTPKLSFLSSLSNCNSLRFIDLSDNPLDGILPKTSIGNESHSLKEFYMSNCNVSGGIPEEISNLTHLTTIILGGN
KINGSIPITLGKLQKLQGLGLGDNKLEGSIPDDICRLAELYRLELGGNKLYGSIPTCFGNLASLRILSLGSNKLTSIPLT
FWNLKDILQLNESSNFLTGPLPLEIGNLKVLIVIDESMNNESGVISTEIGGLKNLEYLFLGYNRERGSIPDSFGDLISEK
SINLSNNNLSGAIPTSLEKLSYLEDLNLSENKLEGEIPRGGSFANFSAESFEGNELLCGSPNLQVPPCKTSIHHTSWKNS
LLLGIVLPLSTTLLIVVIWLILRYRQRGKQPSNDANMSLVATWRKFSYLELCRATDGFSENNLIGRGGFGSVYKARLGNG
MEVAVKVENLQCGRAFKSFDVECEMMKSIRHRNLIKVISSCSNEEFKALVLEYMPHGSLEKYLYSSNCILDIFQRLNIMI
DVASALEYLHFGCSALVIHCDLKPSNVLLGDNMVAHLSDFGIAKLLIGEDQSMTQTQTLGTIGYMAPEYGREGRVSANGD
VYSFGIMLMETFTGKKPTDEIENGEMTLKHWVNELLPISTMEVVDANLLRQEDIHFAAKEQCVSFIFNLAMACTVESPEQ
RINAKEIFVEGGKVDYVLP
>XP_006465578.1 probable LRR receptor-like serine/threonine-protein kinase
At3g47570 isoform X6 [Citrus sinensis]
MERLHSLRMMSRELLLHCLILISLFIAAATANTSSTITDRDALLALKAHITHDPINFLAKNWNTSTPVCNWTGVACDVHS
HRVTVLNISSLNETGTIPSQLGNLSSLQSLNLSCNRLFGSIPSAIFTIYTLKYVSLRENQVSGQIPANICSNLPFLDYLS
LGKNMFHGGIPSALSNCTYLQILHLSYNDESGAVPKDIGNISKLKELYLGRNRLQGEIPREFVNITELERMSLSENELQG
GIPRELGNITKLEGLQLFRNNITGGIPRELGNITKLERLQLFWNNITGAIPKEIGNLTKLKELSLDGNRLQGEIPLEISN
LQNLEELDLRHNKLVDVRLPNLEALLLWDNPLDGILSKTSIGNLSHSLKDFYMSNCNVSGGIPEEITNLINSITIDLGGN
KLNGSIPITLSKLQKLQGLGLDDNKLEGSIPDSICRETELYELELGGNKLFGSIPACFSNLASLRILSLSSNELTSIPLT
FWNLKDILQLNESSNFLTGPLPLEIGNLKVLIGIDESMNNESSVIPTEIGGLKNLEYLFLGYNRLEGSIPDSFGDLISLK
FLNLSNNNLSGAIPTSLEKLSYLEDLNLSENKLEGEIPRGGSFGNFAAESFEGNELLCGSPTLQVLPCKTSIHHTSWKNS
LLLGIVLPLSTTLLIVVIWLILRYRKRGKQPSNDANMPLVATWRTFSYLELCRATNGESENNLIGRGGFGSVYKARLGDG
MEVAVKVENLQCGRAFKSFAVECEMMKSIRHRNLIKVISSCSNEEFKALVLEYKPHGSLEKYLYSSNCILDIFQRLNIMI
DVASALEYLHFGCSAPVIHCDLKPDNVLLDDNLVAYLSDFGIAKLLIGEDQSMTQTQTLATIGYMAPEYGREGRVSTNGD
VYSFGIMLMETFTGKKPTDEIFNGEMTLKHWVNDWLPISTMEVVDANLLSQEDVHFVAKEQCVSFVENLAMACTVESHEQ
RINAKEIVTKLLKIRDSLLRNVGGRRISQPNLN
>GAY66430.1 hypothetical protein CUMW_248700, partial [Citrus unshiu]
MSRFLLLHCLTLISLFIAATTANTITITIDQDALLALKAHITHDPINFLAKNWNTSTPVCNWTGVTCDVHSHRVTVLNIS
RINLTGTIPSQLGNLSSLQSLNLSFNRFFGSIPSAIFTIYTLKYVSFRENQLSGTFPSLILNKSSLQHLDFTHNTLSGEI
PANICREIPQEFGNLVKLELMSLPENKLQGEIPSEIGNFHNLEYLDLSINKLVGVVPSAIFNVSTLKYLGLQNNSLSGSI
SSILDFRLPNLEELHLWGNNFSGTIPPFIFNASKLSILELGGNSESGFIPNAFGNLRNLNYLTLYNNYLTSSTPELSFLS
SLSNCKSLRLIDLSNNSLDGILPRTSVGNLSHSLEYFDMSYCNVSGGIPEEINNLTNLITIYLAGNKLNGSIPITLSKLQ
KLQGLGLQDNKLKGLIPEDICRLAKLYELNLGGNMLSGSIPACFSNLASLRTLSLGENELTSIPSTFWNLKDILYLNESS
NFFAGPLPLKIGNLKVLIEIDFSMNNFSGVIPTTIGGLKNLQYLSLGNNRLQGSIPNSVGDLISLKSLNLSNNNLSGAIP
VSLEKLTYLKDLDLSENKLEGEIPNGGSFGNFSAESFEGNQLLCGLPNLHVPPCKTSIHHTSWKNALLLGTFLPVSTIEM
IVVMLLIVRYRKRGKQALNDANMPPLAKWRMLSYLELCRATDGFSENNLLGRGGFGSVYKARIEDGMDVAVKVENLEYGR
AFKSFDVECEIMKSIRHRNLIKVISSCSNEEFKALVTEYMPHGSLEKYLYSSNYNLDIFQRLNIMIDVALALEYLHEGCS
ASVIHCDLKPSNVLLDDNMVAHLSDFGIAKLLTGEDQSMIQTQTLATIGYMAPEYGREGRVSANGDVYSEGIMLMETETG
KKPTDKIENGEMTLTHWVNNWLPISIMKVADANLISQEDVHFAAKEQCMSFVENLAMECTAESPEQRINAKEIVTRLLKI
KDSLLRNVGGLITLCNNSWGV
>GAY66432.1 hypothetical protein CUMW_248700, partial [Citrus unshiu]
TTANTITITIDQDALLALKAHITHDPINFLAKNWNTSTPVCNWTGVTCDVHSHRVTVENISRLNUTGTIPSQLGNLSSLQ
SINLSENRFFGSIPSAIFTIYTLKYVSFRENQLSGTFPSLILNKSSLQHLDFTHNTLSGEIPANICSNLPFLEYFSLFQN
MFHGGIPSTLSNCTYLRILSLSSNDFSGPIPKEIGNLTKLKELYLGRNRLHGEIPQEFGNLVKLELMSLPENKLQGEIPS
EIGNFHNLEYLDLSLNKLVGVVPSAIFNVSTLKYLGLQNNSLSGSLSSILDFRLPNLEELHLWGNNFSGTIPPFIFNASK
LSILELGGNSFSGFIPNAFGNLRNLNYLTLYNNYLTSSTPELSFLSSLSNCKSLRLIDLSNNSLDGILPRTSVGNLSHSL
EYFDMSYCNVSGGIPEEINNLTNLITIYLAGNKLNGSIPITESKLQKLQGLGLQDNKLKGLIPEDICRLAKLYELNLGGN
MLSGSIPACESNLASLRTLSLGENELTSIPSTFWNLKDILYLNFSSNFFAGPLPLKIGNLKVLIEIDFSMNNESGVIPTT
IGGLKNLQYLSLGNNRLQGSIPNSVGDLISLKSENLSNNNLSGAIPVSLEKLTYLKDLDLSENKLEGEIPNGGSFGNFSA
ESFEGNQLLCGLPNLHVPPCKTSIHHTSWKNALLLGTFLPVSTIFMIVVMLLIVRYRKRGKQALNDANMPPLAKWRMLSY
LELCRATDGESENNLLGRGGFGSVYKARIEDGMDVAVKVENLEYGRAFKSEDVECEIMKSIRHRNLIKVISSCSNEEFKA
LVTEYMPHGSLEKYLYSSNYNLDIFQRLNIMIDVALALEYLHFGCSASVIHCDLKPSNVLLDDNMVAHLSDFGIAKLLTG
EDQSMIQTQTLATIGYMAPEYGREGRVSANGDVYSFGIMLMETFTGKKPTDKIFNGEMTLTHWVNNWLPISIMKVADANL
ISQEDVHFAAKEQCMSFVENLAMECTAESPEQRINAKEIVTRLLKIKDSLLRNVGGLITLCNNSWGV
>GAY42605.1 hypothetical protein CUMW_068210 [Citrus unshiu]
MERVRSLSMTSRELLLHCLFLISLFIAAATANTSSITTDQDALLALKAHITHDPTNFLAKNWNTSTPVCNWTGVTCDVHS
HRVTVLDIFGLNLIGTVPSQLWNLSSLQSLDLGLNRERGSIPSAIFTTYTLKYVNERGNQLSGAFPSLIENKSSLQHLDF
SENTLSGEIPANICSNLPFLEYFSLSKNMFHGRIPSTLSNCTYLQILSLSYNNFSGAIPKEIGNLTELKELYLSTNRLQG
KIPREFSNLADLEQMTLSKNNLQGEIPPEIGNESNLGWLELGQNKLVGIVPAAIENVSTLKVLDLENNSLSGRLSSLADV
RLPNLVALYLWGNNFCGTIPRFIFNASKLSILELEDNSFSGFIPNTFGNLRNLKVLILYDNYLTSSTPELSFLSTLSNCK
SLQHIQLLNNPLDGILSRTSVSNLSHSLEYFDMSDCNVSGGIPEEIGNLTNLTTIFLGGNKLHGSIPFTLGKLQKLQYLG
LEDNKLEGSIPNDICHLVELFELELGGNKLSGSIPVCFSNLTSERILSLDSNELTSIPSTFWNLTDILYLNFSSNELTGP
LPLEIENLKVLVGIDFSMNTFSSVIPTAIGGLENLQSLFLAYNRLQGSIPNSFGDLISLISENLSNNNLSGSIPISLEKL
SYLKDLNLSENKLEGEIPRRGSFGNFSAESFEGNELLCGSPNLRVPPCKTSIHHKSRKNTLLLGIVLPLSTIFMIVVILL
IVKYGKREKGPPNDANMPPEATERRESYLELCQATDGESQNNLIGRGGFGSVYKARIRDGMEVAVKVENLQCGRAFKSED
VECEIMKSIRHRNLIKVISSCSNEEFKALVLEYMPHGSLEKYLYSSSCILDILQRENIMIDVASALEYLHEGYSAPIIHC
DLKPNNVLLDNNMVAHLSDFGIAKLLTREDQSMTQTQTLATIGYMAPEYGREGRISINGDVYSFGIMLIETFSGKKPTDE
IFNGEMTLKHWVNDWLPISIMEVVDANLLSREDIHFVAKEQCMSFVENLAMECTVESPEQRINAKEIVRRLLKIRDLLL
>XP_024952374.1 probable LRR receptor-like serine/threonine-protein kinase
At3g47570 [Citrus sinensis]
MESVHSLSIMSRFPLLHYLILISLFIAAETANTRTITTDQDALLALKAHITHDPTNFLAKNWNTSAPVCNWTGVTCDVHS
HKVTALNISGLNLTGTIPSQLGNLSSLQSLDLSENRLSGSIPSAIFTTYTLKDVSFRENQLTGVFPSFIENKSSLQHLDF
SRNTLSGEIPANICSSLPFLDYLYLSKNMLAGGIPSTLSNCTYLRILSLAYNDESGAVPKDIGNUTKLMGLYLGRNRLQG
EIPREFGNLAELEQMSLAENNLQGGIPQELGNLTKLEILELFESNLTGEIPSEIGNLRNLEELDLSHNKLVGTVPAAIEN
VSTLKRLGLQNNELSGCLSSISDARLINLEGLYLWGNNFSGTIPDFIFNASKLFQLSLAMNSFFGFIPNTFGNLRNLKWL
TLYDNYLTSSTPELSFLSSLSNCKSLTHLSLSNNPLDSVLSRTSVSNLSHSLKELYMSNCNVSGGILEEITNLTNLTAIN
LGDNKLNGSIPITLGKLQKLQYLGLENNKLEGSIPDGICCSVELYKLELGGNKLSGSIPACFSNLASLRILSLDSNKLTS
IPLNFWNLKDILYLNESSNELTGPLPLEIGNLKVEVGIDLSMNNFSGVIPTEIGGLKNLEYLELGYNRLQGPIPDSEGDL
ISLKFLNLSNNNLSGAIPASLEKLSYLENINLSFNKLEGEIPRGGPFRNESVESFEGNELLCGSPNLQVPPCKTSNHHTL
WKNSLLLRIVLPLSAIFMIVVILLILRYRQKGKRPSNDANMPSIATWRTFSHLELCRATDGESENNLIGRGGFGSVYKAR
LGDGMEVAVKVENLQCGRALKGEDVECEMMKSIRHRNLIKVISTCSNEEFKALVLEYMPHGSLEKYMYSSNYILDIFQRL
NIMIDVASALEYLHEGYSAPIIHCDLKPSNVLLDDNMVAHLSDFSIAKLLTGEDQSMTQTQTLATIGYMAPEYGREGRVS
TNGDVYSFGIMLMETFTGKKPTNEIENGEMTLKHWVNDWLPISTMEVVGANLLSQEDIHFVAKEQCVSCVENLAMECTVE
SPEQRINAREIDLAYIEQKQKEKLEKGKVVVGARTTKEKGNIWLWLELVVGAGSTKERGNIWLWLELVARARSTKEMKRK
IFGCGWSSWLKLDRRRKGKGKYLGGAPGTKKMGNIWQVKTATFLALTKRCV
>GAY42604.1 hypothetical protein CUMW_068210 [Citrus unshiu]
MERVRSLSMTSRELLLACLFLISLFIAAATANTSSITTDQDALLALKAHITHDPINFLAKNWNTSTPVCNWTGVTCDVHS
HRVTVLDIFGLNLIGTVPSQLWNLSSLQSLDLGLNRERGSIPSAIFTTYTLKYVNERGNQLSGAFPSLIENKSSLQHLDF
SENTLSGEIPANICSNLPFLEYFSLSKNMFHGRIPSTLSNCTYLQILSLSYNNESGAIPKEIGNLTELKELYLSTNRLQG
KIPREFSNLADLEQMTLSKNNLQGEIPQELGNLTGLETLLLYYNFLTGEIPPEIGNFSNLGWLELGQNKLVGIVPAAIEN
VSTLKVLDLENNSLSGRLSSLADVRLPNLVALYLWGNNFCGTIPRFIFNASKLSILELEDNSFSGFIPNTEGNLRNLKVL
ILYDNYLTSSTPELSFLSTLSNCKSLQHIQLLNNPLDGILSRTSVSNLSHSLEYEDMSDCNVSGGIPEEIGNLINLTTIF
LGGNKLHGSIPFTLGKLQKLQYLGLEDNKLEGSIPNDICHLVELFELELGGNKLSGSIPVCFSNITSLRILSLDSNELTS
IPSTFWNLTDILYLNFSSNFLTGPLPLEIENLKVLVGIDFSMNTFSSVIPTAIGGLENLQSLFLAYNRLQGSIPNSFGDL
ISLISINLSNNNLSGSIPISLEKLSYLKDLNLSENKLEGEIPRRGSFGNFSAESFEGNELLCGSPNLRVPPCKTSIHHKS
RKNTLLLGIVLPLSTIFMIVVILLIVKYGKREKGPPNDANMPPEATLRRFSYLELCQATDGFSQNNLIGRGGFGSVYKAR
IRDGMEVAVKVENLQCGRAFKSFDVECEIMKSIRHRNLIKVISSCSNEEFKALVLEYMPHGSLEKYLYSSSCILDILQRL
NIMIDVASALEYLHEGYSAPIIHCDLKPNNVLLDNNMVAHLSDFGIAKLLTREDQSMTQTQTLATIGYMAPEYGREGRIS
TNGDVYSFGIMLIETFSGKKPTDEIENGEMTLKHWVNDWLPISIMEVVDANLLSREDIHFVAKEQCMSFVENLAMECTVE
SPEQRINAKEIVRRLLKIRDELL
>GAY68421.1 hypothetical protein CUMW_263980 [Citrus unshiu]
MERAHSLMMMSRFLLLHCLILISLFIAAATANTSSTITDQDALLALKAHITHDPINFLAKNWNTSTPVCNWTGVACEVHS
QRVTVENISSLNETGTIPSQLGNLSSLQSLNLSENRLEGSIPSAIFTTYTLKYVCLRGNQLSGTFPSFISNKSSLQHLDL
SSNALSGEIRANICREIPREFGNLPELELMSLAANNLQGKIPLKIGNLRNLEKLDIGDNKLVGIAPIAIENVSTLKILGL
QDNSLSGCLSSIGYARLPNLEILSLWGNNESGTIPRFIFNASKLSILDLEGNSFSGFIPNTFGNLRNLSWLVLSDNYLTS
STQELSFLSSLSNCKELKYFDLSYNPLYRILPRTIVGNLSHSLEEFKMSNCNISGGIPEEISNLINLRTIYLGGNKINGS
ILITLSKLQKLQDLGLKDNKLEGSIPYDICNLAELYRLDLDGNKLSGSIPACFSNLTSLRIVSLGSNELTSIPLTFWNLK
DILNLNFSSNELTGSLPLEIGSLKVLVGIDLSRNNESGVIPTEIGGLKNLEYLFLGYNRLQGSIPNSFGDLISLKELNLS
NNNLSGVIPASLEKLSYLEDENLSENQLEGKIPRGGSFGNFSAQSFEGNELLCGSPNLQIPPCKTSIHHKSWKKSILLGI
VLPLSTTEMIVVILLILRYRQRGKRPSNDANGPLVASRRMFSYLELCRATDGFSENNLIGRGGFGFVYKASLGDGMEVAV
KVFTSQCGRAFKSFDVECEIMKSIRHRNLIKVISSCSNEEFKALVLEYMPHGSLEKYLYSSNCILDIFQRLNIMIDVASA
LEYLHEGYSAPVIHCDLKPSNVLLDDNMVAHLSDFSIAKMLTGEDQSMIQTQTLATIGYMAPEYGREGRVSANGDVYSEG
IMLMETFTGKKPTDEIENGEMTLKHWVNNWLPISTMEVVDANLLSQEDIHEVAKEQCVSFVENLAMECTMEFPKQRINAK
EIVTKLLKIRDSLLRNVGGRCVMKF
>GAY68423.1 hypothetical protein CUMW_263980 [Citrus unshiu]
MERAHSIMMMSRELLLACLILISLFIAAATANTSSTITDQDALLALKAHITHDPINFLAKNWNTSTPVCNWTGVACEVHS
QRVTVENISSLNLTGTIPSQLGNLSSLQSLNLSENRLEGSIPSAIFTTYTLKYVCLRGNQLSGTFPSFISNKSSLQHLDL
SSNALSGEIRANICSNLPFLEYLAFFKNMLHGGIPSTLSNCTYLRTLDESYNDFSEAIPKDIGNLTNLKELYLGRNRLQG
EIPREFGNLPELELMSLAANNLQGKIPLKIGNLRNLEKLDIGDNKLVGIAPIAIFNVSTLKILGLQDNSLSGCLSSIGYA
RLPNLEILSLWGNNESGTIPRFIFNASKLSILDLEGNSFSGFIPNTFGNLRNLSWLVLSDNYLTSSTQELSFLSSLSNCK
FLKYFDLSYNPLYRILPRTIVGNLSHSLEEFKMSNCNISGGIPEEISNLTNLRTIYLGGNKINGSILITLSKLQKLQDLG
LKDNKLEGSIPYDICNLAELYRLDLDGNKLSGSIPACESNLTSLRIVSLGSNELTSIPLTEWNLKDILNLNESSNFLTGS
LPLEIGSLKVLVGIDLSRNNESGVIPTEIGGLKNLEYLFLGYNRLQGSIPNSFGDLISLKFLNLSNNNLSGVIPASLEKL
SYLEDINLSFNQLEGKIPRGGSFGNFSAQSFEGNELLCGSPNLQIPPCKTSIHHKSWKKSILLGIVLPLSTTFMIVVILL
ILRYRQRGKRPSNDANGPLVASRRMFSYLELCRATDGFSENNLIGRGGFGFVYKASLGDGMEVAVKVFTSQCGRAFKSED
VECEIMKSIRHRNLIKVISSCSNEEFKALVLEYMPHGSLEKYLYSSNCILDIFQRLNIMIDVASALEYLHEGYSAPVIAC
DLKPSNVLLDDNMVAHLSDFSIAKMLTGEDQSMIQTQTLATIGYMAPEYGREGRVSANGDVYSFGIMLMETFTGKKPTDE
IFNGEMTLKHWVNNWLPISTMEVVDANLLSQEDIHFVAKEQCVSFVENLAMECTMEFPKQRINAKEIVTKLLKIRDSLLR
NVGGRCVMKE
>GAY63063.1 hypothetical protein CUMW_222620 [Citrus unshiu]
MERVHSLSMISRFLLLHCLVLIFLFIAAATANTSTITTDQDALLALKAHISHDPTNFLAKNWNKSTPICNWTGVTCDVHS
HRVTVLNISSLNLTGTVPAQLGNLSSLQSLDLSENRLSGFIPSTIFTMYTLKRVSFRENQLSGTFPSFIENKSSLQHLDE
SHNTLSGEIPANICSNLPFLEYISLSQNMFHGRIPPTLSNCTYLRILGLSENNFSGAIPKEISYLTKLKELYLGVNRLQG
EIPREVGNLAELELMSLPENKLQGEIPQELGNLVGLEFLFLSDNFLTGTIPKEISNFTNLQDLGLDSNRLQGEIPPEIGN
LRSLEWLLLGYNKLVGTIPAAIFNVSTLKQLDLQNNSLSGSLSSIADVRLPNLEMIYMWGNNESGTIPRFIFNASKLSIL
SLEKNSESGFIPNTFGNLRNLEQLDLSDNYLTSSTPELSFLSSLSNCKSLTHIRLSDNPLNGILPRTTVGNLSHSLELED
MSYCNISGSIPKEISNLTNETTIYLVGNKINGLIPITLGKLQKLQSLVLEDNKLKGSIPDDICRLAELYELNLGGNKLSG
SIPACESNLASLRTLSLSSNELTSIPLTLWNLKDILYLNFSSNFLSGPLPLEIENLKVLVGIDFSMNNESSVIPTTIGSL
KDLQYLLLAYNKLQGSIPDSVGDLISLKSLNLSNNNLSGAIPVSLEKVSYLENLDLSENKLEGEIPKGGSEGNFSAESFE
GNELLCGSPNLQVPPCKISIHHASRKNALLLGTALPLSTIFMIVVILLILKCRKRRKRPSDDANIPPVPTLRRFSYLELY
QATNGFGENNLIGRGGFGSVYKARIQDGIEVAVKVENLQCGRAFKSFDVECQVMKSIRHRNLIKVISSCSNEEFKALVLE
YMPHGSLEKYLYSSNCILDIFQRLNIMIDVASALEYLHFGYSTPVIHCDLKPNNVLLDNNMVAHLSDFGIAKLLTGEDQF
VTQTQTLATIGYMAPEYGREGRVSTNGDVYSFGIMLMETFTGKKPTDKIENGEMTLKRWICDWIPISIMEVVDANLLSRE
DIHFVAKEQCLSFVENLAMDCTVECPEQRINAKEIVTRLLKIRDSLLRNVEGRCIRQSNLN
>KDO39417.1 hypothetical protein CISIN_1g0020211mg, partial [Citrus sinensis]
KIPLKIGNLRNLEKLDIGDNKLVGIAPIAIFNVSTLKILGLQDNSLSGCLSSIGYARLPNLEILSLWGNNESGTIPRFIF
NASKLSILDLEGNSESGFIPNTFGNLRNLSWLVLSDNYLTSSTQELSFLSSLSNCKELKYFDLSYNPLYRILPRITVGNL
SHSLEEFKMSNCNISGGIPEEISNLTNLRTIYLGGNKLNGSILITLSKLQKLQDLGLKDNKLEGSIPYDICNLAELYRLD
LDGNKLSGSIPACFSNLTSLRIVSLGSNELTSIPLTFWNIKDILNLNESSNFLTGSLPLEIGSLKVLVGIDESRNNESGV
IPTEIGGLKNLEYLFLGYNRLQGSIPNSFGDLISLKFLNLSNNNLSGVIPASLEKLSYLEDINLSENQLEGKIPRGGSFG
NFSAQSFEGNELLCGSPNLQIPPCKTSIHHKSWKKSILLGIVLPLSTTFMIVVILLILRYRQRGKRPSNDANGPLVASRR
MFSYLELCRATDGFSENNLIGRGGFGSVYKASLGDGMEVAVKVFTSQCGRAFKSEDVECEIMKSIRHRNLIKVISSCSNE
EFKALVLEYMPHGSLEKYLYSSNCILDIFQRLNIMIDVASALEYLHFGYSAPVIHCDLKPSNVLLDDNMVAHLSDESIAK
MLTGEDQSMIQTQTLATIGYMAPEYGREGRVSANGDVYSFGIMLMETFTGKKPTDEIENGEMTLKHWVNDWLPISTMEVV
DANLLSQEDIHFVAKEQCVSFVENLAMECTMEFPKQRINAKEIVTKLLKIRDSLLRNVGGRCVRQSNLN
>GAY63065.1 hypothetical protein CUMW_222610 [Citrus unshiu]
MERVHSLSMISRELLLHCLVLIFLFIAAATANTSTITTDQDALLALKAHISHDPINFLAKNWNKSTPICNWTGVTCDVHS
HRVTVENISSLNETGTVPAQLGNLSSLQSLDLSFNRLSGFIPSTIFTMYTLKRVSFRENQLSGTFPSFIENKSSLQHLDE
SHNTLSGEIPANICSNLPFLEYISLSQNMFHGRIPPTLSNCTYLRILGLSENNESGAIPKEISYLTKLKELYLGVNRLQG
EIPREVGNLAELELMSLPENKLQGEIPQELGNLVGLEFLFLSDNELTGEIPPEIGNLRSLEWLLLGYNKLVGTIPAAIEN
VSTLKQLDLQNNSLSGSLSSIADVRLPNLEMIYMWGNNFSGTIPRFIFNASKLSILSLEKNSESGFIPNTEGNLRNLEQL
DLSDNYLTSSTPELSFLSSLSNCKSLTHIRLSDNPLNGILPRTTVGNLSHSLELFDMSYCNISGSIPKEISNLTNITTIY
LVGNKLNGLIPITLGKLQKLQSLVLEDNKLKGSIPDDICRLAELYELNLGGNKLSGSIPACFSNLASLRTLSLSSNELTS
IPLTLWNLKDILYLNFSSNFLSGPLPLEIENLKVLVGIDESMNNFSSVIPTTIGSLKDLQYLLLAYNKLQGSIPDSVGDL
ISLKSLNLSNNNLSGAIPVSLEKVSYLENLDLSENKLEGEIPKGGSFGNFSAESFEGNELLCGSPNLQVPPCKISIHHAS
RKNALLLGTALPLSTIFMIVVILLILKCRKRRKRPSDDANIPPVPTLRRESYLELYQATNGEGENNLIGRGGFGSVYKAR
IQDGIEVAVKVENLQCGRAFKSFDVECQVMKSIRHRNLIKVISSCSNEEFKALVLEYMPHGSLEKYLYSSNCILDIFQRL
NIMIDVASALEYLHFGYSTPVIHCDLKPNNVLLDNNMVAHLSDEGIAKLLTGEDQFVTQTQTLATIGYMAPEYGREGRVS
TNGDVYSEGIMLMETFTGKKPTDKIFNGEMTLKRWICDWIPISIMEVVDANLLSREDIHFVAKEQCLSFVENLAMDCTVE
CPEQRINAKEIVTRLLKIRDSLLRNVEGRCIRQSNLN
>XP_024036863.1 probable LRR receptor-like serine/threonine-protein kinase
At3g47570 [Citrus clementina]
MNSFSGFIPSTEGNLRNLEWLTLYDNNLTSSTLDLSELSSLSNCKSLTHISLSNNPLDGILPRTYVGNLSHSLKNFYMYN
CNVSGGIPEEITNLIDLTTIVIGGNKLNGSIPITLGKLQKLQDVDLEYNQLEGSIPDSICLSVELYELELGGNKLSGSIP
ACFSNMTFLKVLSLGSNELTSIPLNFWSLKDILDLNLSSNCFSGPLPLEIRNLKALIEIDFSMNNESGIIPMEIGSLKNL
ENLFLEYNRLEGSIPDSFGDLISLKSLNLSYNNLSGTIPVSLEKLSYLKDENLSENKLKGEIPRGGSFGNESAESEKGNE
LLCGSPNLQVPPCKASIHRTSRKNALILGIVLPFSTIFMTAIILFIIKYQKREKGPPNDPNMPPVATWRRFSYLELFQAT
DKFSENNLIGRGGFGSVYKARIRDGMEVAVKVENLQCGRAFKSFDVECAMMKSIRHRNLVKVISSCSNEEFKALVLEYMP
HGSLEKYLHSSNYSLDIFQRLNIMIDVASALEYLAFGYSAPVIHCDLKPSNVLLDDNMVAHLSDEGIAKLLTGEDQSMTQ
TQTLATIGYMAPEYGREGQVSTNGDVYSFGIMLMETFTRKKPTDELENGEMTLKHWVNDCLPISTMEVVDANLLSQEDIH
FVAKEQCVSFVENLALECTVESPEQRINAKEIVAKLLKIRDSLERNVGGRCIRQSNLN
>XP_006465576.1 probable LRR receptor-like serine/threonine-protein kinase
At3g47570 isoform X4 [Citrus sinensis]
MERLHSLRMMSRELLLHCLILISLFIAAATANTSSTITDRDALLALKAHITHDPINFLAKNWNTSTPVCNWTGVACDVHS
HRVTVLNISSLNLTGTIPSQLGNLSSLQSENLSCNRLFGSIPSAIFTIYTLKYVSLRENQVSGQIPANICSNLPELDYLS
LGKNMFHGGIPSALSNCTYLQILHLSYNDESGAVPKDIGNLSKLKELYLGRNRLQGEIPREFVNITELERMSLSENELQG
GIPRELGNETKLEGLQLFRNNLTGGIPRELGNLIKLERLQLEWNNLTGAIPKEIGNLTKLKELSLDGNRLQGEIPLEISN
LQNLEELDLRANKLVGTVPAAIFNMSMLKLLHLQNNSLLGCLSSIADVRLPNLEALLLWGNNFSGTIPRFIFNASKLSIL
ELSQNSESGFIPNTFGNLRNLEWINLRDNYLTSSTPELSFLSSLSNCKSLTFIHLSDNPLDGILSKTSIGGNKLNGSIPI
TLSKLQKLQGLGLDDNKLEGSIPDSICRLTELYELELGGNKLFGSIPACFSNLASLRILSLSSNELTSIPLTFWNLKDIL
QLNFSSNELTGPLPLEIGNLKVLIGIDFSMNNESSVIPTEIGGLKNLEYLFLGYNRLEGSIPDSFGDLISLKFLNLSNNN
LSGAIPTSLEKLSYLEDINISENKLEGEIPRGGSEGNFAAESFEGNELLCGSPTLQVLPCKTSIHATSWKNSLLLGIVEP
LSTTLLIVVIWLILRYRKRGKQPSNDANMPLVATWRTFSYLELCRATNGFSENNLIGRGGFGSVYKARLGDGMEVAVKVE
NLQCGRAFKSFAVECEMMKSIRHRNLIKVISSCSNEEFKALVLEYKPHGSLEKYLYSSNCILDIFQRLNIMIDVASALEY
LHFGCSAPVIHCDLKPDNVLLDDNLVAYLSDFGIAKLLIGEDQSMTQTQTLATIGYMAPEYGREGRVSTNGDVYSFGIML
METFTGKKPTDEIFNGEMTLKHWVNDWLPISTMEVVDANLLSQEDVHFVAKEQCVSEVENLAMACTVESHEQRINAKEIV
TKLLKIRDSLLRNVGGRRISQPNLN
>KDO48826.1 hypothetical protein CISIN_19040845mg [Citrus sinensis]
MPSIINNELTSTTPKEIDNISNLKVLYLYNNRLQGEIIHEIGHLHNLGELDLSQNKLLGTIPAAIFNVSTLKAFAVINNS
LSGCLSSITDVGLPNLEVLYLWGNNFSGTIPHFIFNASKLSKLALEMNSESGFIPSTEGNLRNLEWLTLYDNNLTSSTLD
LSFLSSLSNCKSLTHISLSNNPLDGILPRTYVGNLSHSLKNEYMYNCNVSGGIPEEITNLTDLTTIVIGGNKLNGSIPIT
LGKLQKLQDVDLEYNQLEGSIPDSICLSVELYELELGGNKLSGSIPACFSNMTFLKVLSLGSNELTSIPLNEWSLKDILD
LNLSSNCFSGPLPLEIRNLKALIEIDFSMNNFSGIIPMEIGSLKNLENLFLEYNRLEGSIPDSFGDLISLKSLNLSYNNL
SGTIPVSLEKLSYLKDLNLSFNKLKGEIPRGGSFGNFSAESFKGNELLCGSPNLQVPPCKASIHRTSRKNALILGIVLPF
STIFMTAIILFIIKYQKREKGPPNDPNMPPVATWRRFSYLELFQATDKFSENNLIGRGGFGSVYKARIRDGMEVAVKVEN
LQCGRAFKSEDVECAMMKSIRHRNLVKVISSCSNEEFKALVLEYMPHGSLEKYLHSSNYSLDIFQRLNIMIDVASALEYL
HEGYSAPVIHCDLKPSNVLLDDNMVAHLSDFGIAKLLTGEDQSMTQTQTLATIGYMAPEYGREGQVSTNGDVYSFGIMLM
ETFTRKKPTDELENGEMTLKHWVNDCLPISTMEVVDANLLSQEDIHFVAKEQCVSFVENLALECTVESPEQRINAKEIVA
KLLKIRDSLLRNVGGRCIRQSNEN
>GAY47648.1 hypothetical protein CUMW_106000 [Citrus unshiu]
MHKDSKVWTILAPQENCQEGGVGSANYSKISKGDTDILADDLQVAMKENIHELIVVGCQREEYSIYDLLYLTVRYHMKSL
SDNKLVGVVPATIFNLSTLRVFAVSNNSLLGSLQSSADVQLPNLEGIYLWGNNFSGTIPSFIENASKLSTLALEDNSFFG
FIPNTEGNLGNLRWENIENNYLTSSTPELNFLSSLSNSKYLKVLELSYNPLNGILPRTSMGNLSHSLEKEVMINCNVGGA
IPEEISNLTNLRMIGFSGNKLNGSIPITLCKLQKLQLLSFRDNKLEGSIPEDVCSLAELYQLHLGGNKESRSIPTCIGNL
TSLRTLSLGSNELISVIPSTLWNLEYIMNLNFSSNFLTGPLPLEIGNLKVLVGIDFSMNNESGAIPTTIGGLTDLQYLLL
GHNKLEGSIPNPIGDLISLEYLDLSNNNLSGPIHVSLEKLLYLKDLNLSENNLEGEIPKGGSFRNFSAKSFEGNKLLCGS
PNLQVPPCKTSIHHTSRKNALLLGIVLPLSIVSMIVVILLISRYRKRGKQLPNDANMPPVATWFKLFQATDGESENNLIG
RGSFGSVYKARIQDGMEVAVKVFHLQCGGVEKSEDVECEVMKSIRHRNLIKIISTCSNDDFKALVLEYMPHGSLEKCLYS
SNCILDIFQRLNIMIDIAVALEYLHFGYSAPVIHCDLKPSNVLLDDNMVAHLSDFGIAKLLTGEDQSKSQTQTLATIGYM
APEYGREGRVSTNGDVYSFGIMLMETFTKKKPTDKIFAGEMTLKYWVSNLLPISVMEIVDANLLSREDKHFAAKEQCVSF
VENFAMECTVESAEQRINAKEIVTRLLKIRDSLLICTRESKLN
>GAY65414.1 hypothetical protein CUMW_240950 [Citrus unshiu]
MSRSLLRHCLILISLFIAAATANTSTTTADQDGLLALKAHITHDPTNFLAKNWNTRTLVCNWTGVTCDVHSHRVTILNIS
RINLIGTIPSQLGNLSSLQSLDLSFNQLSGSIPSAIFSTYTLKYVNFRENQLSGAFPSLIENKSSLQLLDFAHNTLSDEI
PANICREIPQEFGNLAELEQMSLSENKLQGEIPHEIGNLRNLELLVLSHNRLVGVIPTKVENVSTLKVFEVSNNSLSGSL
SSIAGVRLPNLEVLRMRSNNFCGTIPHFIFNASKLSLLELGDNSESGFIPDTFGNLRNLNKVTLYNNYLTSSTSDLNFLS
SLSNCKTLTYIDLSDNPLDGILPGTSVGNLSHSLEYFYMPNCNVSGGIPEEISNETNLIIIYLGGNKLNGSIPITLSKLQ
KLQGLSLADNKLEGSIPNNICRLTELYELDLGSNKESRSIPACFSNLASLRTLSLGSNELTSIPLTFWNLKDILYLNESS
NFLTGPLPLEIENLKVLVGIDESVNNFSGVIPTTIGSLKGLQYLFVGYNRLQGSIPYSIGDLISLKSLNLSNNNLSGTIP
VSLEKLSYLEDINLSENKLAGEIPRGGSEGNFSAESFEGNELLCGSPNERVPPCKTSTHHTSWKNALLLGTVLPLSTIEM
IVVILLILRYRKRVKPPPNDANMPPVATWRRFSYLELCRATDRESENNLIGRGGFGSVYKARIQDGMEVAVKVFHLHCSG
AFKSEDVECNVMKNIRHRNLIKIISSCSNDDFKALVLEYMPHGSLEKCLYSSNCILDIFQRLSIMVDVASALEYLHENYS
APIIHCDLKPSNVLLDDNMVAHLSDFGIAKLLIGEDQSMTQTQTLATIGYMAPEYGREGRVSTNGDVYSFGIMLIEAFTR
KKPTDEMFSGEMTLKRWINDLLSVSVMEVVDANLLTREDRHFAAKQQCVSFVENLAMECTIESPERRINAKEIVTELSKI
RDSLERNVGADE
>GAY65413.1 hypothetical protein CUMW_240950 [Citrus unshiu]
MERVHSLSMMSRSLLRHCLILISLFIAAATANTSTTTADQDGLLALKAHITHDPINFLAKNWNTRTLVCNWTGVTCDVHS
HRVTILNISRLNETGTIPSQLGNLSSLQSLDLSFNQLSGSIPSAIFSTYTLKYVNFRENQLSGAFPSLIFNKSSLQLLDE
AHNTLSDEIPANICSNLPFLEILSLSQNMEHGGIPSTLSNCTYLQKLSLPYNDESGAIPKEIGNENKLKRLYLGRNRLQG
EIPQEFGNLAELEQMSLSENKLQGEIPHEIGNLRNLELLVLSHNRLVGVIPTKVENVSTLKVFEVSNNSLSGSLSSIAGV
RLPNLEVLRMRSNNFCGTIPHFIFNASKLSLLELGDNSFSGFIPDTFGNLRNLNKVTLYNNYLTSSTSDLNFLSSLSNCK
TLTYIDLSDNPLDGILPGTSVGNLSHSLEYFYMPNCNVSGGIPEEISNLTNLIIIYLGGNKLNGSIPITLSKLQKLQGLS
LADNKLEGSIPNNICRLTELYELDLGSNKFSRSIPACFSNLASLRTLSLGSNELTSIPLTFWNLKDILYLNESSNFLTGP
LPLEIENLKVLVGIDFSVNNESGVIPTTIGSLKGLQYLFVGYNRLQGSIPYSIGDLISLKSINLSNNNLSGTIPVSLEKL
SYLEDENLSENKLAGEIPRGGSFGNFSAESFEGNELLCGSPNLRVPPCKTSTHATSWKNALLLGTVLPLSTIFMIVVILL
ILRYRKRVKPPPNDANMPPVATWRRFSYLELCRATDRESENNLIGRGGFGSVYKARIQDGMEVAVKVFHLHCSGAFKSED
VECNVMKNIRHRNLIKIISSCSNDDFKALVLEYMPHGSLEKCLYSSNCILDIFQRLSIMVDVASALEYLHENYSAPIIHC
DLKPSNVLLDDNMVAHLSDFGIAKLLIGEDQSMTQTQTLATIGYMAPEYGREGRVSINGDVYSFGIMLIEAFTRKKPTDE
MESGEMTLKRWINDLLSVSVMEVVDANLLTREDRHFAAKQQCVSFVENLAMECTIESPERRINAKEIVTELSKIRDSLER
NEID
>XP_006468119.1 receptor kinase-like protein Xa21 isoform X1 [Citrus sinensis]
MERVHSLSMMSRFLFLHCLILISLLTAAATANTSSITTDQDALLALKAHITHDPTNFLAKNWNTSTPVCNWTGVTCDVHS
HRVKVLNISHLNLTGTIPSQLWNLSSLQSLNLGENRESGSIPSAIFTLYTLKYVNERGNQLSGAFPSFIENKSSLQHLDE
SYNALSGEIPANICSNLPFLESISLSQNMFHGRIPSALSNCKYLEILSLSINNLLGAIPKEIGNETKLKELYLGYSGLQG
EIPREFGNLAELELMALQVSNLQGEIPQELANLTGLEVLKLGKNELTGEIPPEIHNLHNLKLLDLSHNKLVGAVPATIEN
MSTLTRLGLQSNSLSGSLSSIADVQLPNLEELRLWSNNFSGTIPRFIFNASKLSVLELGRNSFSGFIPNTFGNLRNLRLM
TLHYNYLTSSNLELSFLSSFSNCKSLTYIGLSNNPLDGILPRMSMGNLSHSLEYFDMSYCNVSGGFPKEIGNLTNLIGIY
LGGNKINGSIPITLGKLQKLQGLHLEDNKLEGPIPDNICRLTKLYELELSGNKLSGSIPACESNLASLGTLSLGSNKITS
IPLTIWNLKGMLYLNFSSNFFTGPLPLDIGNLKVLVGIDFSMNNFSDVIPTAIGGLINLQYLFLGYNRLQGSIPESFGDL
ISLKSENLSNNNLSGSIPISLEKLSYLEYLDLSENKLKGEIPKGGSFGNFSAESFEGNELLCGSPNLQVPPCKTSIHHKS
RKNVLLLGIVLPLSTIFIIVVILLIVRYRKRVKQPPNDANMPPIATCRRESYLELCRATNRFSENNLIGRGGFGSVYKAR
IGEGMEVAVKVEDLQCGRAFKSFDVECEMMKSIRHRNLIKVISSCSTEEFKALILEYMPHGSLEKSLYSSNYILDIFQRL
NIMVDVATTLEYLHFGYSAPVIHCDLKPSNVLLDDNMVAHLSDFGIAKLLIGEDQSITQTQTLATIGYMAPEYGREGRIS
RNGDVYSFGIILMETFTGKKPTDEIFNEEMTLKHWVNDWLPISIMKVIDANMLSREDIHFVAKEQCVSFVENLAMECTVE
SPQQRINAKEIVTRLLKIRDSLLRNVGGRCIRQSNLN
>XP_006494782.2 probable LRR receptor-like serine/threonine-protein kinase
At3g47570 [Citrus sinensis]
MERAHSLMMMSRFLLLHCLILISLFIAAATANTSSTITDQDALLALKAHITHDPINFLAKNWNTSTPVCNWTGVACEVHS
QRVTVENISSLNETGTIPSQLGNLSSLQSLNLSENRLEGSIPSAIFTTYTLKYVCLRGNQLSGTFPSFISNKSSLQHLDL
SSNALSGEIRANICSNLPFLEYLAFFKNMLAGGIPSTLSNCTYLRTLDESYNDESEAIPKDIGNUTNLKELYLGRNRLQG
EIPREFGNLPELELMSLAANNLQGGIPHELGNLAKLEILELFENNLIGKIPLKIGNLRNLEKLDIGDNKLVGIAPIAIFN
VSTLKILGLQDNSLSGCLSSIGYARLPNLEILSLWGNNFSGTIPRFIFNASKLSILDLEGNSFSGFIPNTFGNLRNLSWL
VLSDNYLTSSTQELSFLSSLSNCKFLKYFDLSYNPLYRILPRTTVGNLSHSLEEFKMSNCNISGGIPEEISNLTNLRTIY
LGGNKUNGSILITLSKLQKLQDLGLKDNKLEGSIPYDICNLAELYRLDLDGNKLSGSIPACESNLTSLRIVSLGSNELTS
IPLTFWNLKDILNLNFSSNELTGSLPLEIGSLKVEVGIDLSRNNFSGVIPTEIGGLENLEYLELGYNRLQGSIPNSEGDL
ISLKFLNLSNNNLSGVIPASLEKLSYLEDENLSFNQLEGKIPRGGSFGNFSAQSFEGNELLCGSPNLQIPPCKTSIHHKS
WKKSILLGIVLPLSTTFMIVVILLILRYRQRGKRPSNDANGPLVASRRMFSYLELCRATDGFSENNLIGRGGFGSVYKAS
LGDGMEVAVKVFTSQCGRAFKSFDVECEIMKSIRHRNLIKVISSCSNEEFKALVLEYMPHGSLEKYLYSSNCILDIFQRL
NIMIDVASALEYLHFGYSAPVIHCDLKPSNVLLDDNMVAHLSDESIAKMLIGEDQSMIQTQTLATIGYMAPEYGREGRVS
ANGDVYSFGIMLMETFTGKKPTDEMENGEMTLKHWVNDWLPISTMEVVDANLLSQEDIHFVAKEQCVSEVENLAMECTME
FPKQRINAKEIFVERGKVDYALS
>GAY68700.1 hypothetical protein CUMW_266180 [Citrus unshiu]
MERVHSLSMMSRFILLHCLILISLLTAAATANTSSITTDQDALLALKAHITHDPINFLAKNWNTSTPVCNWTGVTCDAHR
HRVKVLNISHLNLTGTIPSQLWNLSSLQSLNLGENRLSGSIPSAIFTMYTLKYVNERGNQLSGAFPSFIENKSSLQRLDE
SYNALSGEIPANICSNLPFLEYFSLSQNMFHGGIPSTLSNCKYLEILSLSINNLLGAIPKEIGNETKLKELYLGYSGLQG
EIPREFGNLAELELMALQVSNLQGEIPQELANLTGLEVLQLDKNFLTGEIPPEIHNLHNLKLLDLSHNKLVGAVPATIEN
MSTLTGLGLQSNSLSGSLSSIADVQLPNLEELRLWSNNFSGTIPRFIFNASKLSVLELGINSESGFIPNTEGNLRNERLL
TLHYNYLTSSNLELSELSSESNCKSLTYIGLSNNPLDGILPRMSMGNLSHSLEYFDLSYCNVSGGFPEEIGNLTNLIGIY
LGGNKLNGSIPITLGKLQKLQGLHLEDNKLEGPIPDDICRETKLYELELSGNKLSGSIPACESNLASLGTLSLGSNKITS
IPLTIWNLKGMLYLNFSSNFFTGPLPLDIGNLKVLVGIDFSMNNFSDVIPTVIGGLINLQYLFLGYNRLQGSIPESEGDL
ISLKSLNLSNNNLSGSIPISLEKLSYLEDLDLSENKLKGEIPKGGSFGNFSAESFEGNELLCGSPNLQVPPCKTSIHHKS
RKNVLLLGIVLPLSTIFIIVVILLIVRYRKRVKQPPNDANMPPIATCRRFSYLELCRATDRESENNLIGRGGFGSVYKAR
IGEGMEVAVKVEDLQCGRAFKSFDVECEIMKSIRHRNLIKVISSCSTEEFKVLVLEYMPHGSLEKNLYSSNCILDIFQRD
NIMVDVATALEYLHFGYSAPVIHCDLKPSNVLLDDNMVAHLSDFGIAKLLIGEDQSMTQTQTLATIGYMAPEYGREGRIS
TNGDVYSEGIILMETFTGKKPTDEIFNEEMTLKHWVNDWLPISIMKVIDANLLSWEDIHFVAKEQCVSFVENLAMECTVE
SPQQRINAKEIVTRLLKIRDSLLRNVGGRCIRQSNLN
>KDO48988.1 hypothetical protein CISIN_1g036229mg [Citrus sinensis]
MERVHSLSMMSRFLFLHCLILISLLTAAATANTSSITTDQDALLALKAHITHDPINFLAKNWNTSTPVCNWTGVTCDVHS
HRVKVLNISHLNLTGTIPSQLWNLSSLQSENLGENRESGSIPSAIFTLYTLKYVNERGNQLSGAFPSFIENKSSLQHLDF
SYNALSGEIPANICSNLPFLESISLSQNMFHGRIPSALSNCKYLEILSLSINNLLGAIPKEIGNLTKLKELYLGYSGLQG
EIPREFGNLAELELMALQVSNLQGEIPQELANLTGLEVLKLGKNFLTGEIPPEIHNLANLKLLDLSHNKLVGAVPATIEN
MSTLTGLGLQSNSLSGSLSSIADVQLPNLEELRLWSNNFSGTIPRFIFNASKLSVLELGRNSFSGFIPNTEGNLRNLRLM
TLHYNYLTSSNLELSFLSSFSNCKSLTYIGLSNNPLDGILPRMSMGNLSHSLEYFDMSYCNVSGGFPKEIGNLTNLIGIY
LGGNKLNGSIPITLGKLQKLQGLHLEDNKLEGPIPDDICRLTKLYELGLSGNKLSGSIPACFSNLASLGTLSLGSNKLTS
IPLTIWNLKGMLYLNFSSNFFTGPLPLDIGNLKVLIGIDESTNNFSDVIPTVIGGLINLQYLFLGYNRLQGSISESFGDL
ISLKSINLSNNNLSRSIPISLEKLSYLEDLDLSENKLKGEIPKGGSFGNESAKSFEGNELLCGSPNLQVPPCKTSIHHKS
RKNVLLLGIVLPLSTIFIIVVILLIVRYRKRVKQPPNDANMPPIATCRRFSYLELCRATNRFSENNLIGRGGFGSVYKAR
IGEGMEVAVKVEDLQCGRAFKSFDVECEMMKSIRHRNLIKVISSCSTEEFKALILEYMPHGSLEKSLYSSNYILDIFQRL
NIMVDVATTLEYLHFGYSAPVIHCDLKPSNVLLDDNMVAHLSDFGIAKLLIGEDQSITQTQTLATIGYMAPGLFHVKYIL
FVVNFLTSYSFLMIFIGRGNYY
>XP_024953043.1 probable LRR receptor-like serine/threonine-protein kinase
At3g47570 isoform X8 [Citrus sinensis]
MERLHSLRMMSRELLLHCLILISLFIAAATANTSSTITDRDALLALKAHITHDPINFLAKNWNTSTPVCNWTGVACDVHS
HRVTVLNISSLNETGTIPSQLGNLSSLQSLNLSCNRLEGSIPSAIFTIYTLKYVSLRENQVSGQIPANICSNLPFLDYLS
LGKNMFHGGIPSALSNCTYLQILHLSYNDESGAVPKDIGNLSKLKELYLGRNRLQGEIPREFVNITELERMSLSENELQG
GIPRELGNLTKLEGLQLFRNNLTGGIPRELGNLTKLERLQLFWNNLTGAIPKEIGNLTKLKELSLDGNRLQGEIPLEISN
LQNLEELDLRHNKLVDVRLPNLEALLLWDNPLDGILSKTSIGGNKLNGSIPITLSKLQKLQGLGLDDNKLEGSIPDSICR
LTELYELELGGNKLEGSIPACFSNLASLRILSLSSNELTSIPLTEWNLKDILQLNESSNFLTGPLPLEIGNLKVLIGIDE
SMNNFSSVIPTEIGGLKNLEYLFLGYNRLEGSIPDSEGDLISLKFLNLSNNNLSGAIPTSLEKLSYLEDLNLSENKLEGE
IPRGGSEGNFAAESFEGNELLCGSPTLQVLPCKTSIHHTSWKNSLLLGIVEPLSTTLLIVVIWLILRYRKRGKQPSNDAN
MPLVATWRTFSYLELCRATNGFSENNLIGRGGFGSVYKARLGDGMEVAVKVENLQCGRAFKSFAVECEMMKSIRHRNLIK
VISSCSNEEFKALVLEYKPHGSLEKYLYSSNCILDIFQRLNIMIDVASALEYLHFGCSAPVIHCDLKPDNVLLDDNLVAY
LSDFGIAKLLIGEDQSMTQTQTLATIGYMAPEYGREGRVSINGDVYSFGIMLMETETGKKPTDEIENGEMTLKHWVNDWL
PISTMEVVDANLLSQEDVHFVAKEQCVSFVENLAMACTVESHEQRINAKEIVTKLLKIRDSLLRNVGGRRISQPNLN

1. A method of enhancing resistance of a plant to a disease caused by bacteria of a Liberibacter species, the method comprising genetically modifying the plant to decrease expression of an endogenous gene encoding a negative regulator of immune response polypeptide, wherein the negative regulator of immune response polypeptide is VAD1, PRT6, PUB26, PAO1, LIN2, CRWN, or GPX8

2. The method of claim 1, wherein the disease is HLB and the plant is a citrus plant.

3. The method of claim 2, wherein the plant is a Citrus maxima, Citrus medica, Citrus micrantha, Citrus reticulata, Citrus aurantiifolia, Citrus aurantium, Citrus latifolia, Citrus limon, Citrus limonia, Citrus paradisi, Citrus clementina, Citrus unshiu, Citrus sinensis, Citrus tangerina, Citrus ichangensis, Atalantia buxifolia, or Poncirus trifoliata plant.

4. The method of claim 1, wherein the disease is Potato Zebra Chip disease and the plant is a potato plant.

5. The method of claim 1, wherein decreasing expression of the negative regulator comprises contacting the plant with siRNA that targets an endogenous nucleic acid encoding the negative regulator.

6. The method of claim 1, wherein decreasing expression of the negative regulator comprises viral vector-mediated gene silencing.

7. The method of claim 1, wherein decreasing expression of the negative regulator comprises knocking out expression of the endogenous gene encoding the negative regulator.

8. The method of claim 1, wherein the method comprises gene editing the endogenous gene to decrease or knockout expression.

9. The method of claim 8, wherein the gene editing technique is CRISPR/CAS gene editing.

10. The method of claim 1, wherein the negative regulator of immune response polypeptide comprises an amino acid sequence having at least 70% identity to a VAD1, PRT6, PUB26, PAO1, LIN2, CRWN, or GPX8 polypeptide sequence as set forth in Table 3.

11. The method of claim 10, wherein the negative regulator of immune response polypeptide comprises an amino acid sequence having at least 90% or at least 95% identity to a VAD1, PRT6, PUB26, PAO1, LIN2, CRWN, or GPX8 polypeptide sequence as set forth in Table 3.

12. A method of enhancing resistance of a plant to a disease caused by bacteria of a Liberibacter species, the method comprising genetically modifying a plant to overexpress a gene encoding a positive defense regulator polypeptide set forth in Table 2, wherein the positive defense regulator peptide is BRAP2, NDR1-like, or PSL4.

13. The method of claim 12, wherein the disease is HLB and the plant is a member of the Citrus family.

14. The method of claim 13, wherein the plant is a Citrus maxima, Citrus medica, Citrus micrantha, Citrus reticulata, Citrus aurantiifolia, Citrus aurantium, Citrus latifolia, Citrus limon, Citrus limonia, Citrus paradisi, Citrus clementina, Citrus unshiu, Citrus sinensis, Citrus tangerina, Citrus ichangensis, Atalantia buxifolia, or Poncirus trifoliata plant.

15. The method of claim 12, wherein the disease is Potato Zebra Chip disease and the plant is a potato plant

16. The method of claim 12, wherein the method comprises genetically modifying a plant to overexpress a polypeptide comprising an amino acid sequence having at least 70% identity to a BRAP2, NDR1-like, or PSL4 polypeptide sequence set forth in Table 4.

17. The method of claim 16, wherein the method comprises genetically modifying a plant to overexpress a polypeptide comprising an amino acid sequence having at least 95% to a BRAP2, NDR1-like, or PSL4 polypeptide sequence set forth in Table 4

18. The method of claim 12, wherein the polypeptide is endogenous to the plant.

19. The method of claim 12, wherein the polypeptide is heterologous to the plant.

20. A plant having enhanced resistance to HLB generated by the method of claim 1.
